(19) United States
(12) Patent Application Publication
(10) Pub. No.: US 2005/0142539 A1
(43) Pub. Date: Jun. 30, 2005

(54) Targeted ligands

(76) Inventor: William Herman, Thornhill (CA)

Correspondence Address: BERESKIN AND PARR, 40 KING STREET WEST, BOX 401, TORONTO, ON, M5H 3Y2, CA

(21) Appl. No.: 10/943,918

(22) Filed: Sep. 20, 2004

Related U.S. Application Data
(63) Continuation-in-part of application No. PCT/CA03/00044, filed on Jan. 14, 2003.
(60) Provisional application No. 60/504,283, filed on Sep. 19, 2003.

(30) Foreign Application Priority Data
Jan. 14, 2002 (CA) CA 2,368,708
Aug. 13, 2002 (CA) CA 2,397,169
Sep. 19, 2002 (CA) CA 2,402,930
Jan. 14, 2002 (CA) CA 2,368,708

(51) Int. Cl. C12Q 1/70; G01N 33/574; C07K 16/30

(52) U.S. Cl. 435/5; 435/7.23; 530/388.3; 530/388.22

(57) ABSTRACT

The invention contemplates a composition containing a multispecific ligand containing at least a first ligand binding moiety and a second ligand binding moiety. The first ligand binding moiety specifically binds with a pre-selected first affinity to at least a first ligand. The first ligand has a first biodistribution. The second ligand binding moiety specifically binds with a pre-selected affinity to at least a second ligand. The second ligand has a second biodistribution. The affinities of first and second ligand binding moieties are selected to bias the biodistribution of the multispecific ligand in favour of a selected location of one or both of the ligands.

### CROSS-REFERENCE TO RELATED APPLICATIONS

This application claims priority from provisional application No. 60/504,283 filed Sep. 19, 2003, and is a continuation in part of PCT/CA03/00044 filed Jan. 14, 2003, which claims priority from PCT/CA02/00317 filed Mar. 11, 2002, which claims priority from U.S. provisional patent application Nos. 60/274,217 filed Mar. 9, 2001, 60/276,911 filed Mar. 20, 2001, 60/279,132 filed Mar. 28, 2001, 60/281,029 filed Apr. 7, 2001, and 60/306,148 filed Jul. 19, 2001. Application Nos. 60/504,283, PCT/CA03/00044, PCT/CA02/00317, 60/274,217, 60/276,911, 60/279,132, 60/281,029, and 60/306,148 are all incorporated herein by reference.

### FIELD OF THE INVENTION

The present invention relates to multispecific ligands, for example a heterofunctional ligand comprising at least first and second binding moieties which have cooperating functional affinities, for example, a bispecific antibody, having at least a first portion which binds, for example, to a cell-specific marker and a second portion which binds, for example, to a cell-surface receptor.

### BACKGROUND OF THE INVENTION

Immunotherapy has gained wide acceptance as a promising measure to address several disease states including autoimmune disease, transplant rejection, infectious disease and cancer. Despite rapid and exciting progress in approaches to treatment, the disease burden attributable to such illnesses has not significantly abated. The complex nature of the normal and pathologic immunologic processes associated with such diseases, coupled with logistical problems in evaluating and implementing methods for immunotherapy in human subjects, continue to be some of the obstacles to successful advances in treatment.

Successful approaches to immunotherapy are predicated on the ability of the immunotherapeutic molecule to be delivered in a therapeutic, sub-toxic dose at the desired therapeutic frequency. In the process of selection of a suitable therapeutic molecule, it is recognized that sub-toxic doses may be insufficient for the desired therapeutic effect, especially where the antibody binds incidentally to cell populations other than the target population. In the case of an injectable preparation and especially an intravenous mode of delivery, in contrast to readily self-administered modes of delivery, the optimal dosing frequency for therapeutic purposes could impose an undesirable burden on the patient and care-giver, assuming that such optimal frequency is to begin with deemed convenient for clinical trials.

Numerous research efforts are underway to identify and test ligands including antibodies, biologic effector ligands (e.g. cytokines, chemokines, growth factors colony stimulating factors, receptor agonists or antagonists etc.) which will bind to or otherwise interact with or trigger responses in or towards target entities, including pathogenic organisms, tissue specific cells, diseased cells, immune cells etc. A recent example is a renewed interest to find molecules and methods of triggering an interaction with CD45 (see for example Nature (2001) Vol. 409 p. 349-354). Evaluating the biological effect of interactions with such target ligands is often obfuscated and retarded by the biodistribution of such ligands on cells other than the target population which results in undesired and/or confusing pleiotropic effects. The present invention facilitates scientific assessment, development, role evaluation, therapeutic evaluation, and delivery, particularly targeted delivery of molecules that exert biologic functions, for example, immune-related functions. In particular, the targeting agents and methods which are the subject of the invention herein facilitate scientific evaluation of the biological effects of a more targeted biodistribution of such targeting agents, by limiting undesired or confusing side effects. In preferred aspects the invention contemplates compositions of matter and methods of delivery, in some cases using ligands that but for the targeting methods herein defined would be ineffective or have a broader effect than is desirable; or similarly, but for the severity of the disease or the absence of other therapeutic alternatives for which such ligands are useful, they would otherwise be inappropriate for therapeutic use. The present invention accommodates evaluation of the biological role and/or effects of such ligands for therapeutic or other scientific purposes using such targeting strategies. In particular, the present invention provides a vehicle to preferentially target, on a sub-population of cells for which there is a cell-associated marker, a receptor or receptor ligand which is present on a more heterogeneous population of cells.

### SUMMARY OF THE INVENTION

The invention contemplates a composition containing a multispecific ligand containing at least a first ligand binding moiety and a second ligand binding moiety. The first ligand binding moiety specifically binds with a first affinity to at least a first ligand. The first ligand has a first biodistribution in an organism. The second ligand binding moiety specifically binds with a second affinity to a second ligand. The second ligand has a second biodistribution in that organism. The affinity and/or on-rate of first and second ligand binding moieties are selected to bias the biodistribution of the multispecific ligand in that organism in favour of a selected location of one or both of the ligands.

In one non-limiting embodiment the first ligand binding moiety has a different affinity and a different biodistribution than the second ligand binding moiety (which has an affinity for entities, for example cells, which do not express bioavailable first ligands on their surface, the affinity of one or both ligand binding moieties being selected to bias the biodistribution in that organism in favour of entities, which express both the first and second ligands on their surface.

As defined below, the term selected, pre-selected and related terms, as used herein, with regard to the affinity of one or both of the first and second ligand binding moieties, practically means that a determination of at least the relative affinity, if not individual instrinsic affinity of a ligand binding moiety, individually or when linked to another moiety, is predicted or approximated (for example, based on a known or approximated or predicted divalent affinity, in the case of an antibody, the class of Ig, for example, IgG or IgM, the method of generating the monoclonal having regard to statistical or other indicators of the affinity of ligands made by that method, or by some estimate of the affinity of a variant ligand or is made directly via measurement (e.g. surface plasmon resonance), functional screening assay, or analysis (e.g. scatchard analysis, FACS analysis, fluorescence polarization), or indirectly, via competition assay or other in vitro assay, or biodistribution analysis in pre-clinical animal models, as determined by localization or biologic effect. Other parameters that contribute to the balance between the targeting and biologic function of the multispecific ligand, and particularly the second ligand binding moiety, will be described herein in terms of selection or pre-selection with similar meaning.

The term “predetermined” in reference to an affinity-informed choice of ligand, refers necessarily to a prospective choice of affinity, and means on the other hand, that the particular ligand moiety in question has been selected for evaluation of its biological effects on the basis of a non-mandatory (for purposes of the invention) but practical prior selection of its affinity, or relative affinity, optionally in accordance with the guidelines defined herein, which affinity may be predicted or approximated in some instances, but is preferably selected in a functional or indirect screening assay or measured, directly or indirectly. In one non-limiting embodiment, discussed below a library screening process may be systematically applied to a predetermined selection of ligand affinity. In one non-limiting embodiment the first ligand binding moiety has a higher intrinsic affinity and/or a higher on-rate. In another non-limiting embodiment, the second ligand binding moiety is substantially incapable of binding to the second ligand for an effectual duration (the first ligand binding enduringly, by contrast, but not acting at the target receptor to produce the biologic effect on the target cell), independently of the first ligand binding first or contemporaneously binding to the first ligand. The term “substantially incapable” means, with respect to the proportion of the multispecific ligand dose that binds to the non-target population of entities (e.g. cells, infectuous agents, molecules), that the multispecific ligand becomes only transiently bound for a duration and at a concentration which is biologically/therapeutically inconsequential when compared with the activity of normal levels of the natural receptor agonist or ligand, or when compared with the activity of the multispecific ligand at the target site; the foregoing commonly owing in part to the reduced overall dose required for achieving the benefits of enhanced targeting to the target cell population. This is not obvious in view of the direct role that this moiety plays to cause or mediate the biologic effect exerted on the target cell population in a dynamic in vivo environment, where quick turnover of molecules and shear forces are normally at play.

The invention is also directed to a pharmaceutical composition comprising a multispecific ligand of the invention, as defined above, including one or more excipients or ingredients suitable for in vivo administration in domesticated animals, non-human mammals or humans or both, and wherein said composition is of a pharmaceutical grade or standard (e.g. GMP) suitable for clinical use in a veterinary or human clinical trial, or both.

In another non-limiting embodiment, said pharmaceutical composition is packaged, for example, in vials, in a format suitable for immediate administration or admixture with a diluent.

In another non-limiting embodiment said package comprises a label approved of by a government agency or delegate, that regulates pharmaceutical standards.

The invention is also directed to an isolated DNA sequence which encodes said first or second ligand binding moiety, or both said first and second ligand binding moieties, and to an expression vector, host cell or expression system comprising one or two such DNA sequences (as the case may be), and adapted to produce said multispecific ligand in a quantity suitable for commercial sale, or on a scale suitable for a phase I or II clinical trials.

In another non-limiting embodiment said expression vector includes a tag for purification of the multispecific ligand or a component first or second ligand binding moiety. In a further non-limiting embodiment, the invention is directed to DNA or to a vector or other vehicle suitable for gene therapy comprising DNA, which DNA comprises a sequence that encodes a first or second ligand binding moiety of the invention, or both.

In a further non-limiting embodiment, said first or second ligand binding moiety or both are selected from or derived by selection from a library of variants of the respective ligand binding moiety, for example a library of antibodies in which one or more codons of the DNA encoding the VH or VL of said first or second ligand binding moiety is fully or partially randomized. The term “library” is used broadly to include, without limitation, a plurality of candidate binders, or fragments thereof, of suitable diversity with respect to the intended function of the library that can be screened for the ability of at least one candidate member to serve that function, the term “library” including, but not limited to, a repertoire of nucleic acids, each encoding at least a VL or VH of one such candidate binder.

In another non-limiting-embodiment, the second ligand binding moiety directly exerts or inhibits a biologic effect on or within the target cell to which it is bound, or on a second cell with which the target cell would otherwise interact through the second target ligand (e.g. the corresponding receptor ligand, for example, an adhesion molecule), for example, a change in the molecular constitution of the intracellular environment exerted or inhibited by the second target ligand, for example via a signal transduction event or the inhibition such event, transport to or from the cell via a transport protein (e.g. an ABC transporter), ion channel, receptor and in one non-limiting embodiment the second ligand binding moiety comprises both an extracellular and intracellular domain.

In another non-limiting embodiment the multispecific ligand comprises or is primarily constituted by a heterospecific minibody, divalent scFv or sdAb (two scFvs or sdAbs in tandem joined by a linker), a bivalent IgG or any other ligand, non-Ig or Ig based, as described or referenced herein or in the literature. Such scFvs are preferably temperature stable.

The invention is also directed to a method of making a multispecific ligand of the invention comprising at least the step of expressing a DNA sequence comprising one or more polypeptides constituting at least one or both of said first and second ligand binding moieties. The invention also contemplates that other recombinant technologies may be used for making the multispecific ligand.

It is understood that the choice of suitable expression system will depend on the construct sought to be expressed and whether the multispecific ligand will therefore be encoded in a single suitable expression vector as separate first and second ligand binding moieties and then recombined by a cell, interactive moieties (e.g. fos-jun or heterodynamic coiled coils) or chemical synthesis. Such choices and expression options are well known to the art and include bispecific disulfide stabilized Fv constructs.

Methods of making a multispecific ligand using tetroma technology are also contemplated by the invention, including methods comprising the step of fusing two suitable hybridomas.

According to another aspect, the invention is directed to a library for expression of bispecific ligands, said library comprising a repertoire of nucleic acid sequences each encoding a polypeptide comprising a first ligand binding moiety and a second ligand binding moiety and preferably a flexible peptide linker portion therebetween, said repertoire comprising a diversity of sequences which differ from one another in at least one subsequence coding for at least part of a binding region of one ligand binding moiety or the flexible linker portion, so as to provide nucleic acid encoding a repertoire of polypeptides differing at least in said binding region or flexible linker portion. In one non-limiting embodiment, the variant subsequence encodes a flexible linker and optionally a second variant subsequence is used to generate lower affinity (e.g. on-rate) second ligand binding moieties.

In addition to a library of nucleic acid molecules, the term expression library is understood to specifically include a phage, viral, bacterial, yeast or other cell surface display library, a ribosome display library or any other functional nucleic acid expression system which permits the expression products to be screened. The invention also extends to libraries of polypeptides comprising such variant multispecific ligands, which may optionally be displayed on phage etc., in a microarray or on any substrate.

In one non-limiting embodiment of the invention, the variant subsequence comprises at least a portion of the binding region of second ligand binding moiety, for example, in the case of an antibody, a CDR or FR, e.g. a CDR1, CD2 (on-rate) or CDR3). Screening such a library against both target and non-target cells in a cell based assay permits selection of members of the library which localize preferentially to the target cell population, while also individually producing a desired biologic effect on that population. Thus, in a first screen there is a subtraction of library members that bind to the non-target cell population, for example as segregated by FACS using colors allocated uniquely to the respect target and non-target populations, (optionally using a greater proportion of non-target cells to adsorb multispecific ligands comprising higher affinity second ligand moieties than desired and in a second screen, library members enriched and segregated for example from individual phage plaques are screened for quantitative biological activity on the target cell population (e.g. by competition assay or biological assay for agonist/antagonist activity, and in a third screen a plurality of such library members with the highest activities are rescreened for distribution among (percentage distribution) the respective target and non-target cell populations. Varying the linker length between the first and second ligands, permits screening for optimal contemporaneous binding geometrics, as well as stable bispecific ligands.

In one non-limiting embodiment, one or both of the first and second ligand binding moieties is an antibody and the variable subsequence encodes at least a portion of at least one or more CDR or FR. The invention contemplates than on-rate screening, as discussed below, can be conducted with such library.

In another non-limiting embodiment, the instrinsic on-rate of the first ligand moiety has, or is selected to have, a 2 to 100 times faster on-rate relative to the second ligand binding moiety, a 5 to 100 times faster on-rate, a 10 to 100 times faster on-rate, a 20 to 100 times faster on-rate or a 20 to 50 times faster on-rate; (or any increments therebetween or ranges using such an increments). Preferably, the off-rate of the first ligand binding moiety relative to the second, in the aforesaid differential on-rate instances or otherwise, has or is selected to have a 10 to 100,000 times slower off-rate, a 10 to 50,000 times slower off-rate, a 10 to 10,000 times slower off-rate, a 20 to 10,000 times slower off-rate, a 50 to 1000 times slower off-rate, a 100 to 1000 times slower off-rate, or a 100 to 500 times slower off-rate; (or any increments therebetween or ranges using such an increments). It will be appreciated that on or both of the first and second ligand binding moieties may be multivalent so that the differences in kinetics will be effective differences and not necessarily the intrinsic affinities of the individual components of the respective first and second moieties. It will also be appreciated that aforesaid guidelines and the degree to which affinity is engineered will be confirmed, on a case by case basis, in terms of the targeting and effectiveness profiles of the second ligand binding moiety in conjunction with the first ligand binding moiety.

The invention also contemplates a composition comprising a multispecific ligand in the form of an intrabody (namely multispecific ligand of the invention not limited to multispecific antibodies, which is adapted to function within the cell and which may include suitable trafficking signals (e.g. an ER retention signal). The design and engineering of intra-diabodies has also been developed (see Jendreyko N et al, Intra-diabodies: Bispecific, tetravalent antibodies for the simultaneous functional knockout of two cell surface receptors. J Biol Chem. Aug. 28, 2003 [Epub ahead of print] PMID:12947084). The multispecific ligand comprises at least a first ligand binding moiety and a second ligand binding moiety. The first ligand binding moiety specifically binds to a first ligand having a first biodistribution in a cell. The second ligand binding moiety specifically binds to a second ligand having a second different biodistribution in a cell. The affinity and/or on-rate of the first and second ligand binding moieties to their respective ligands are different and selected to bias the biodistribution in the cell to a location of said first ligand, for example to a particular cellular structure (e.g. the nuclear envelope) or organelle.

The invention is also directed to a pharmaceutical composition comprising a multispecific ligand of the invention, when suitable for clinical evaluation of the safety and/or effectiveness of the multispecific ligand.

In another aspect the invention is directed to a method of selecting a second ligand binding moiety, for evaluation of its biological or clinical effect in conjunction with a first ligand binding moiety, said method comprising at least the step of evaluating any one or combination of properties of the second ligand binding moiety, selected from the group of properties comprising:
a) its affinity;
b) its on-rate;
c) its off-rate;
d) its in-vivo biodistribution, including its distribution on a proposed target entity population (e.g. cell population) and d′) preferably also on non-target cells;
e) its hypothesized or demonstrated biologic or therapeutic effect on the proposed target cell population;
f) its hypothesized or demonstrated, ill-defined, biologic, or pathogenic effect on non-target cells;
g) its cell-surface density on a target cell population and g′) preferably also on the non-target cell population;
h) the availability of an entity-associated marker that is expressed exclusively or predominantly (i.e. to advantage for targeting purposes) on the target entity and h′) preferably also its cell surface density on the non-target cell population.

The evaluation of a), b), c), d), e), f), g), or h) or any combination of these steps or component steps (d′), g′) and h′) is preferably conducted empirically, by “in-vitro” assay or in an animal model, or based on an analysis of peer reviewed journals and/or patent publications. In one preferred embodiment at least steps a), d) and e) are evaluated and preferably also h), with or without their respective component sub steps.

The invention contemplates further, a composition containing a multispecific ligand. The multispecific ligand contains a first ligand binding moiety and a second ligand binding moiety. The first ligand binding moiety specifically binds with a predetermined first affinity to a first ligand. The first ligand has a first biodistribution. The second ligand binding moiety specifically binds with a predetermined second affinity to a second ligand. The second ligand has a second biodistribution. The invention is also directed to a method of treating a mammal comprising the step of administering a therapeutically effective amount of a multispecific ligand according the invention.

The invention is also directed to a method of making a multi-specific ligand or a first or second ligand binding moiety thereof, comprising the step of mutating at least a portion of a DNA sequence encoding said first or second ligand binding moiety. In one non-limiting embodiment said method further comprises the step of selecting a multispecific ligand or first or second ligand binding moiety having kinetic characteristics which differ from the parental characteristics for example, a higher or lower affinity or a higher or lower on-rate where a flexible linker is interposed between said first and second ligand binding moiety the method may further comprise the step of mutating at least a portion of a DNA sequence encoding said linker sequence from a coding sequence which translates into a first amino acid composition into a coding sequence which translates into a second amino acid composition, the mutation(s) resulting in an addition, deletion and/or amino acid substition, for example to lengthen, the linker, shorten it deimmunize it, reduce its susceptibility to proteolytic cleavage etc. The invention is also directed a multispecific ligand or component moiety made by the aforesaid method and to a multispecific ligand or component moiety thereof which is a variant of a parental or template such ligand or is a derivative of such ligand. The term derivative is used herein to mean that the physical or functional characteristics or both of a first multispecific ligand or binding moiety thereof were taken into account in designing a second multispecific ligand or binding moiety thereof that improves or or alters the physical characteristics but addresses same qualitative functionality. Of the first multispecific ligand in the context of the invention, and in one non-limiting embodient the derivative multispecific ligand or binding moiety thereof is a variant of the first and is optionally made using the first (typically a DNA sequence encoding the first) as a template, preferably using a library of variants of the first multispecific ligand or first or second binding moiety thereof. Such physical characteristics may include but are not limited to on-rate, off-rate, amino acid composition, stability, immunogenecity, solubility, expression yield, pI, etc.

The invention is disclosed in non-limiting embodients in our copending applications WO 02/072141 and WO 03/057732 which are hereby incorporated by reference.

The invention further contemplates a composition containing a multispecific ligand. The multispecific ligand specifically binds to a target ligand. The target ligand is specific to a selected sub-population of a heterogeneous cell population. This embodiment of the multispecific ligand contains a first ligand binding moiety and a second ligand binding moiety. The first ligand binding moiety specifically binds to a cell sub-population associated ligand. The second ligand binding moiety binds to the target ligand. In this embodiment, the first ligand binding moiety has an affinity and/or a higher on-rate for the sub-population associated ligand higher than the affinity and/or on-rate of the second ligand binding moiety for the target ligand.

The invention further contemplates a composition containing a bispecific ligand containing a first ligand and a second ligand. The first ligand binds to a first target ligand and the second ligand binds to a second target ligand. In this embodiment of the bispecific ligand, the affinity of the first ligand is selected to enable binding to the first target ligand independently of the ability of the second ligand to bind to the second target ligand. Further, the affinity and/or on-rate of the second ligand is selected to substantially reduce the probability of its binding to the second target ligand without the first ligand binding first or substantially contemporaneously to the first target ligand.

The invention further contemplates a composition containing a bispecific antibody containing a first antibody component and a second antibody component. The first antibody component binds to a first target ligand and the second antibody component binds to a second target ligand. In this embodiment, the affinity and/or on-rate and/or avidity of the first antibody component are selected to enable binding to the first target ligand independently of the ability of the second antibody component to bind to the second target ligand. The avidity or affinity and/or on-rate of the second ligand are selected to substantially reduce the probability of its binding to the second target ligand without the first ligand binding first or substantially contemporaneously to the first target ligand.

The invention further contemplates a multispecific ligand containing a first moiety and a second moiety. The first moiety binds to a first target ligand. The second moiety binds to a second target ligand. The affinity and/or avidity and/or on-rate of the first moiety are selected to enable the first moiety to bind to the first target ligand independently of the ability of the second moiety to bind to the second target ligand. The avidity and/or affinity and/or on-rate of the second moiety are selected to substantially reduce the probability of its binding to the second target ligand without the first moiety, first or substantially contemporaneously, binding to the first target ligand.

The invention further contemplates a multispecific ligand containing a first moiety and a second moiety. The first moiety binds to a first target ligand. The second moiety binds to a second target ligand. The affinity and/or avidity and/or on-rate of the first moiety are selected to enable the first moiety to bind to the first target ligand independently of the ability of the second moiety to bind to the second target ligand. The avidity and/or affinity and/or on-rate of the second moiety are selected to substantially reduce the probability of either moiety binding first or for a sufficient duration or series of durations to its respective target ligand to accomplish a therapeutic function without the other moiety, first or substantially contemporaneously, binding to its respective target ligand.

The invention further contemplates a composition containing a multispecific ligand containing a first moiety and a second moiety. The first moiety binds to a first target ligand. The second moiety binds to a second target ligand. The affinity or avidity or both the affinity and avidity of the first moiety are selected to enable the first moiety to bind to the first target ligand independently of the ability of the second moiety to bind to the second target ligand. The avidity or affinity or both the affinity and avidity of the second moiety are selected to enable the second moiety to bind to the second entity in preference to the first moiety binding to the first entity when both first and second moieties are substantially contemporaneously bound to the respective first and second entities.

The invention contemplates a composition containing a multispecific ligand containing a first moiety, a second moiety and a third ligand binding moiety. The first moiety binds to a first target ligand and the second moiety binds to a second target ligand. In this embodiment, the affinity or avidity or both the affinity and avidity of the first moiety are selected to enable the first moiety to bind to the first target ligand in preference to the second moiety binding to the second entity when both first and second moieties are substantially contemporaneously bound to the respective first and second entities, and the avidity or affinity or both the affinity and avidity of the second moiety are selected to enable the third target ligand to bind to the second entity in preference to the second moiety binding to the second entity when both the third target ligand and the second moiety are substantially contemporaneously bound to the second entity.

The invention further contemplates a composition comprising a first ligand e.g. an antibody, which specifically binds to an epitope on a second ligand. The second ligand recognized by the antibody exerts a biologic effect by binding to a target site on a target ligand, e.g. a cell receptor. The epitope bound by the antibody is proximal to the binding region of the second ligand for the target ligand, so that binding of the antibody to the second ligand reduces the affinity but does not prevent binding of the ligand to its target ligand, when so bound by the antibody. The invention contemplates methods of use of such first ligand, for example, an antibody which binds to TNF, to treat sub-acute conditions requiring TNF therapy. In this connection, the invention also contemplates that a high affinity neutralizing can be used to create a complex having a low affinity for a target ligand and thereby constituting a relatively low affinity second ligand moiety, which may be coupled to a relatively high affinity first ligand binding moiety. In this connection, see WO 03/048729 and WO 99/53049.

The invention is also directed to a method of generating such first ligands by panning against such cytokines while bound in situ to their receptors or by the method described in the aforesaid World Patent references.

The invention is also directed to method of combination therapy/evaluation comprising at least one multispecific ligand of the invention. In one non-limiting embodiment the therapy is directed to mitigate a disease phenotype which attributable to a multifactorially constituted responsible gentotype, for example, for bone metastasis, to which chemokine mediated targeting on cancer cells and rank receptor mediated bone resorption on osteoclasts are contributing factors, and where the genotype may influence the level of expression of these receptors. In one non-limitng embodient, at least one or both receptors are may be blocked using respective multispecific ligands which are targeted to respective cancer and osteoclast specific markers. The invention is also directed to a kit or to a pharmaceutical composition comprising at one such multispecific ligand, and preferably a plurality of such ligands for such combination therapy of phenotype-mitigating evaluations.

The invention further contemplates a composition containing a multispecific ligand containing a first ligand binding moiety and a second moiety. The first ligand binding moiety specifically binds to a lymphatic endothelial cell associated marker. The second moiety contains an independent therapeutic function.

The invention further contemplates a composition containing an immunocytokine containing an anti-idiotypic antibody component and a cytokine component. The anti-idiotypic antibody component recognizes the paratope of an antibody which binds to a lymphatic vessel associated ligand.

The invention further contemplates a composition containing a bispecific antibody containing an anti-idiotypic antibody component and an anti-CD3 antibody or an anti-CD28 antibody component. The anti-idiotypic antibody recognizes the paratope of an antibody which binds specifically to a lymphatic vessel associated ligand.

The invention additionally contemplates physiologically acceptable compositions of the compositions encompassed by the invention.

The invention likewise contemplates methods of use of the compositions encompassed by the invention.

A composition comprising a multispecific ligand comprising at least a first ligand binding moiety which specifically binds to a first ligand having a first biodistribution and a second ligand binding moiety which specifically binds to a second ligand having a second biodistribution different from that of the first ligand, and wherein the affinity of the first and second ligand binding moieties are different and selected to bias the biodistribution of the multispecific ligand.

### DETAILED DESCRIPTION OF PREFERRED EMBODIMENTS

As exemplified above, the dual “affinity” “on-rate” based targeting strategy of the invention, may be understood in one aspect, in terms of a strategic allocation of the respective affinity properties of the multispecific ligand to at least one “targeting” function and at least one “effector” function. Accordingly, with respect to some embodiments of the invention, the term “multifunctional” ligand is used interchangeably.

Thus according to one preferred embodiment, at least one of the ligand binding moieties is a “targeting” arm in the sense that it at least preferentially recognizes a marker that is associated with one or more specific target entities e.g. cell populations, and the other ligand binding moiety is an “effector” arm which binds with relatively less affinity or functional affinity and/or less quickly to a target ligand which optionally has a more diverse biodistribution. In this case, the biodistribution of the multispecific ligand is biased in favour of the location(s) of both ligands relative to the location(s) of the target ligand so as to limit the bio distribution to non-target entities.

Such binding or recognition is preferably understood throughout to be specific, in contrast to non-specific binding.

Accordingly, in some non-limiting embodiment the invention may be summarized in the following paragraphs:

1. A multispecific ligand which comprises, at least, a targeting moiety which specifically binds to a first ligand having a first cell surface biodistribution and an effector moiety which binds to a second ligand having a second cell surface biodistribution different from but overlapping that of the first ligand, and wherein the targeting moiety is intrinsically predisposed to bind to the first ligand in preference to the effector moiety binding to the second ligand so as to bias the distribution of the multispecific ligand to a target cell population in an organism on which both said first and seconds ligands are bioavailable for recognition by said multispecific ligand, said second ligand characterized in that it is bioavailable for recognition on at least one population of cells other than the target cell population. In one non-limiting embodiment, the effector moiety exerts or inhibits a cellular response. The term cellular response, in the context of the invention, means that the effector moiety through an agonist or antagonist effect causes or inhibits a biological change within the target cell. The multispecific ligand is adapted to limit the occurrence of an agonist or antagonist effect on the non-target cell. In one non-limiting embodiment, the effector moiety inhibits a soluble or cell-surface ligand from exerting its primary biologic function in relation to the target cell and the multispecific ligand is adapted to limit this inhibitory effect on the non-target cell. Primary biologic function means that the natural effect of the ligand in relation to the ligand to which the effector is bound.

2. A multispecific ligand according to paragraph 1, wherein the first ligand is pre-targeted to the surface of the target cell population.

3. A multispecific ligand according to paragraph 1, wherein the first ligand is expressed on the surface of the target cell population.

4. A multispecific ligand according to paragraph 1, 2 or 3 wherein the intrinsic affinity of the targeting moiety exceeds the intrinsic affinity of the effector moiety.

5. A multispecific ligand according to paragraph 1, 2, 3 or 4, wherein the on-rate of the targeting moiety exceeds (is faster) than the on-rate of the effector moiety.

6. A multispecific ligand according to paragraph 1, 2, 3, 4 or 5, wherein the intrinsic off-rate of the targeting moiety is less (slower) than the intrinsic off-rate of the effector moiety.

7. A multispecific ligand according to paragraph 1, 2, 3, 4, 5 or 6, wherein the intrinsic on-rate of the targeting moiety exceeds the intrinsic on-rate of the effector moiety.¹ For example, the effector moiety may be a “coybody” (as defined below), which may be generated, for example, by a process of mutagenesis in which one or more residues in at least one CDR or FR of the VH or the VL, are mutated or added, preferably residues which do not contribute to the specificity and off-rate of a reference antibody, for example an antibody of medium or high affinity and desired specificity. For example, one of the CDRs, more commonly the CDR2 of the reference antibody, may not be involved in binding to the target ligand in question, and by a process of random mutagenesis and/or by lengthening and/or by introducing large, hydrophobic, hydrophilic or charged amino acids (depending on the amino acid constitution of the epitope), one or more of a series of such modifications may be selected from a library of such CDR2 modifications, for example, under conditions in which the amount of antibody exceeds the amount required for saturation of binding sites on the available antigen (as may be determined through titration using the reference antibody or independently), so as to permit a rapid series of selections to take place under stringent conditions, for example, of shear force (more vigorous shaking than the degree of vigor required to ensure a uniform distribution of phage particles) with the later selections having a greater propensity to yield antibody with a slower on-rate. Amino acids and portions (CDRs) of the antibody variable region which don't contribute to specificity and off-rate may be determined via mutagenesis, for example, by alanine scanning mutagenesis, by assessing singly or in multiples, residues located in each CDR loop to ascertain those which when substituted do not significantly or measurably affect the off-rate of the antibody, as may be determined by surface plasmon resonance or microcalorimetry, or approximation through scatchard analysis, FACS, fluourescence polarization, fluorescence quenching. Optionally, the effect on a readable signal is determined at a concentration of antibody which is predetermined to have the greatest incremental impact on signal change (the sloped portion of a sigmoid dose response curve) so that the effect of mutations is more readily perceived through the readout. Residues which are most likely to border or otherwise affect the target ligand binding site may be predicted, for example, by well-known methods of three dimensional modeling or statistical plots of residues which are most variable or correspond to mutational hot-spots (these residues may also be residues in the neighboring FRs, and are optionally residues which are not conserved (and preferably therefore also do not positively contribute to the solubility, stability and expression characteristics of the reference antibody).
¹ optionally by at least 2, 3, 4, 5, 6, 7, 8, 9, 10, 11, 12, 13, 14, 15, 16, 17, 18, 19, 20, 21, 22, 23, 24, 25, 26, 27, 28, 29, 30-40, 40-50, 50-60, 60-70, 70-80, 80-90, or 90-100 (or more) fold. With respect to each of the foregoing at least 100 increments, the intrinsic off-rate of the targeting moiety is optionally at least 2, 3, 4, 5, 6, 7, 8, 9, 10, 11, 12, 13, 14, 15, 16, 17, 18, 19, 20, 21, 22, 23, 24, 25, 26, 27, 28, 29, 30-40, 40-50, 50-60, 60-70, 70-80, 80-90, or 90-100 (or more) fold less than the intrinsic off-rate of the effector moiety and may be in one non-limiting embodiment in a range from any of the preceding increments to 10,000 or any increments or ranges therebetween.

8. A multispecific ligand according to paragraph 7, wherein the intrinsic on-rate of the targeting moiety is selected so as to exceed the on-rate of the effector moiety.

9. A multispecific ligand according to paragraph 7, wherein the intrinsic on-rate of the effector moiety is selected so as to be less than the on-rate of the targeting moiety.

10. A multispecific ligand according to paragraph 9, wherein one or both of said effector and targeting moieties, comprises one or both of a VH or VL. There are many constructs and strategies that have been developed for the generating bispecific ligands, as detailed in the literature cited below, and summarized in numerous reviews (see for example Suresh, 2001, including methods of chemical synthesis, recombinant DNA synthesis and hybridoma cell fusion based syntheses (see for example, Suresh M R, et al Bispecific monoclonal antibodies from hybrid hybridomas. Methods Enzymol. 1986; 121:210-28) Constructs in which scFvs or single domain antibodies are linked via a flexible linker and Fabs linked via disulfide bonds (Fab′)2, leucine zippers and heterodimeric coiled-coil interactions have been well characterized. Bispecific IgGs have also been developed (see Carter P. Bispecific human IgG by Design. J Immunol Methods(2001);248(1-2):7-15). Diabodies have been shown to have geometry that may also be amenable to binding two epitopes on the same cell, as may be determined on a case-by-case basis. Recombinant fragments linked by flexible linkers of for example, 8-50 amino acids, optionally 10-25 amino acids offer the promise of greatest flexibility in cross-linking different antigens on the same or adjacent cells.

11. A multispecific ligand according to paragraph 9, wherein one or both of said effector and targeting moieties, consists essentially of one or both of a VH or VL.

12. A multispecific ligand according to any of the preceding paragraphs, wherein the first and/or second ligand is expressed predominantly on one or more cell populations of hemapoietic origin optionally primarily on a hemapoietic stem cell.

13. A multi specific ligand according to any of the preceding paragraphs, wherein the first and/or second ligand is expressed predominantly on endothelial cells.

14. A multispecific ligand according to any of the preceding paragraphs, wherein the first and second ligands are expressed primarily on diseased cells, disease causing or mediating cells, or disease responsive cells.

15. A multispecific ligand according to any of the preceding paragraphs, wherein the first and/or second ligand is an ABC transporter protein, a ligand that is over-expressed on tumor cells, an adhesion molecule, a ligand that is expressed exclusively or predominantly on one or more types of leucocytes, one or more sub-populations of T cells or B cells, a cytokine receptor, a growth factor receptor, a chemokine receptor, a vitamin receptor, a neuroreceptor, a pain-related receptor, a receptor or ligand having an intracellular and extracellular domain, that a molecule or complex comprising a transmembrane protein, or a CD molecule, other than CD59, a receptor is selected from the group consisting of tyrosine kinase type receptors, serine kinase type receptors, heterotrimeric G-protein coupled receptors, receptors bound to tyrosine kinase, TNF family receptors, notch family receptors, guanylate cyclase types, tyrosine phosphatase types, decoy receptors, inhibitory receptors and adhesion receptors (other than CD59) or any cell surface receptor-ligand cooperating with such a receptor.

16. A multispecific ligand according to any of the preceding paragraphs, wherein said second ligand binding moiety specifically recognizes said second ligand.

17. A multispecific ligand according to any of the preceding paragraphs, wherein the sum of bioavailable second ligands on said target cell population is less that the sum of bioavailable second ligands on cells other than said target cell population.

18. A multispecific ligand according to any of the preceding paragraphs, wherein the first ligand is present on a non-target cell population (the non-target cell population is understood to possibly comprise cells of more than one type, which respective types may cause different levels or risks of toxicity) and is preferentially bioavailable for targeting purposes on said target cell population.

19. A multispecific ligand according to paragraph any of the preceding paragraphs, wherein the first ligand is exclusively bioavailable for targeting purposes on said target cell population.

20. A multispecific ligand according to any of the preceding paragraphs, wherein the multispecific ligand is adapted to bind contemporaneously to said first and second ligands².
² This may be assessed for example by determining the extent to which the effector moiety out-competes a test ligand having the same specificity as the effector moiety but a somewhat higher on-rate and/or affinity in virtue of the superior (relative to the effector moiety and test ligand) affinity and/or on-rate of the targeting moiety. The effects of modifying the affinity of each of these three elements in either direction with suitable positive and negative controls may be tested to further refine this assessment. The choice of fist and second ligand can be also be influenced by the likelihood that both would be found within or unassociated with lipid rafts (in some cases this may be more important than others), as determined for example by the length of the transmembrane domain, if any, or in some cases whether the ligand is one which is anchored by GPI, whether the effector moiety is selected to agonize or antagonize a receptor and the functional sensitivity of the cell to a less than complete saturation of the second ligand (as influenced by dose and any related MTDs), especially when antagonist effects are being exerted on a receptor (as opposed to agonist effects which may require less binding events depending on the sensitivity of the cell to such events) etc. Although such predictions may have some influence on the a priori choice of ligands for testing, ultimately empirical in vitro assessments may be necessary in most cases.

21. A multispecific ligand according to paragraph 20, wherein the surface cell density of the first ligand on the target cell population exceeds the surface cell density of the second ligand on said target cell population.

22. A multispecific ligand according to paragraph 1, wherein the number of bioavailable second ligands in said mammal ligands is greater than the number of bioavailable first ligands and wherein the in-vivo site of action of said effector moiety is predominantly on the target cell population.

23. A multispecific ligand according to paragraph 1, 2, 3, 4 or 5, wherein the cell-surface density of the second ligand on the target cell population generally does not exceed the cell-surface density of the second ligand on a non-target cell population and where the in-vivo site of action of said effector moiety is predominantly on the target cell population.

24. A multispecific ligand according to any of the preceding paragraphs, wherein the first and second ligand binding moieties are small molecules³ that are conjugated to a fullerene, peptide or polypeptide or other linking substrate.

25. A multispecific ligand according to any of the preceding paragraphs, comprising a bispecific antibody in which the Fc portion of the molecule is absent or incapable of binding to an Fc receptor.

26. A multispecific ligand according to paragraph 10, 11 or 25, wherein at one or both of the targeting moiety and effector moieties comprises human sequences (non-chimeric) or includes at least primarily human FRs (commonly one or more non-human FR amino acids are retained or used for functional purposes (e.g. mainataining affinity).

27. A pharmaceutical composition comprising a multispecific ligand according to any of the preceding paragraphs and a pharmaceutically acceptable carrier.

The term “effector” is used to refer to the ability to effect a biological consequence through binding, for example effecting a signal transduction event by activating a receptor, or blocking the target ligand from associating with a complementary ligand, for example blocking a receptor from associating with a complementary ligand (e.g. its natural ligand whether soluble or on another cell) and thereby, for example, preventing a signal transduction, or for example in the case of a decoy receptor preventing the biological consequence (e.g. protective effect) associated with the function of such receptor, or blocking a ligand from associating with a complementary ligand e.g. receptor on another entity eg. a cancer cell, infectious agent or immune cell.

The term targeting moiety means capable of distinguishing at least in part between the target entities (e.g. cells according to one embodiment), which target entities are defined at least in part by a first ligand, typically found on the target cell population (and at least some entities e.g. cells other than the target population of entities) that bear the second ligand acted on by the effector moiety.

A biased biodistribution is preferably accomplished by the multispecific ligand contemporaneously recognizing both ligands on the same entity e.g. cell, and may be accomplished by such contemporaneous recognition occurring on adjacent entities or by increasing the propensity of the multi specific ligand to locate in proximity to a target entity in virtue of the relatively high affinity targeting arm. The targeting arm may itself be an effector.
³ The term small molecule typically connotes small synthetic or semi-synthetic organic molecules which are not macromolecules such as proteins, lipoproteins, glycoproteins, long chain fatty acids etc.

It will be appreciated that one or both ligand binding moieties may exert additional effector properties.

It will also be appreciated that any multispecific ligand of the invention or any component thereof may be fused or conjugated to a separate effector as exemplified below, including toxins, cytokines, adhesion molecules etc.

The ligand binding moiety is in three non-limiting embodiments an antibody, a peptide mimetic of the natural ligand or a sequence or sequences of amino acids etc., which are the natural ligand for the target ligand, for example where the ligand is a cytokine or lymphokine receptor, such as IL-2 receptor, the ligand binding moiety may comprise a sequence of amino acids which is IL-2 or a functional receptor binding portion thereof. Other suitable ligands include, weel-described and known Affibodies™, T cell receptors (TCRs) (see WO 01/48145A2 High Affinity TCR Proteins And Methods(2001)), anticalins, fibronectin type III domains, C-type lectin-like domains (see WO 02/48189A2., V-like domains (see CA 2,322,621), peptides and peptide fusions and conjugates and stabilized polypeptide loops (see also WO/070190). The ligand binding moiety may also be a mutated or a newly developed form of the natural ligand (e.g. developed through combinatorial libraries) or a natural or synthetic chemical ligand, for example as developed through combinatorial chemistry. In one aspect, the invention contemplates a composition containing a multispecific ligand containing at least a first ligand binding moiety and a second ligand binding moiety, the first ligand binding moiety specifically binding with a pre-selected first affinity to at least a first ligand, having a first biodistribution and the second ligand binding moiety specifically binding with a pre-selected affinity to at least a second ligand with a second biodistribution and wherein the affinity of first and second ligand binding moieties are selected to bias the in vivo site site of biologic activity of the multispecific ligand; and wherein the first ligand binding moiety preferably binds with high affinity (preferably nanamolar affinity or greater) to a specific cell associated marker (e.g. a CD marker, a marker associated with diseased cells, a marker associated cells in a particular physiological state (e.g. activated T cells, B cells) etc. (such markers may be associated with a particular class of cell or a subclass thereof (if applicable) or particular subpopulation within the subclass (if applicable), however classified, such as epithelial cells, endothelial cells, immune cells (lymphocytes, memory cells, effector cells) monocytes, T cells (CD4+, CD8+, CD45RO+), hepatocytes, stem cells, etc. and wherein said second ligand binding binding moiety binds with relatively low, or medium affinity (in one non-limiting embodiment 0.1 micromolar or less) to a receptor (e.g. chemokine, growth factor, cytokine) involved in cell signaling or a decoy receptor, a cell surface receptor ligand e.g. the ligand for such receptor which effects a signal or inhibits a signal (e.g. CTLA4), a ligand involved in cell adhesion, a receptor or channel (ion channel) for a molecule involved in cell regulation or homeostasis etc.

Methods of identifying target-associated markers, where none are reported, include routinely practiced methods of substractive screening with libraries, including ligand libraries in form peptide, scFv, Fab or sdAb (single-domain antibodies) displayed on phage, microarrays or other ‘substrates’ (see for example Belizaire A K, et al. Identification of a murine ICAM-1-specific peptide by subtractive phage library selection on cells. Biochem Biophys Res Commun. Sep. 26, 2003; 309(3):625-30. Tanaka T, et al. Single domain intracellular antibodies: a minimal fragment for direct in vivo selection of antigen-specific intrabodies. J Mol Biol. Aug. 29, 2003; 331(5):1109-20. Ruoslahti E. Drug targeting to specific vascular sites. Drug Discov Today. Nov. 15, 2002; 7(22):1138-43. J Biotechnol. June 2001; 74(4):257-75. Ruoslahti E, et al. An address system in the vasculature of normal tissues and tumors. Annu Rev Immunol. 2000;18:813-27.) Recent advances in making libraries include those disclosed in WO 03/064648.

The invention contemplates that the difference in affinity will in most cases be an essential element in biasing the location of action of the multispecific ligand to yield an acceptable or desired safety profile and that the high affinity of the first ligand binding moiety for the cell associated marker will be optimized for this purpose insofar as the safety profile of the multispecific ligand dictates maximizing its affinity characteristics. The invention also recognizes that choosing the relatively lower affinity of the second ligand binding moiety may assist in this regard up to a point where its effectiveness to bind to the second ligand is significantly compromised. In this regard, the invention also contemplates that factors other than the choice of affinity of the first and second ligand binding moieties (and of course the avidity effect resulting from having two ligands on the target cell and only one on the non-target cell) may be taken into consideration or optimized to balance the safety and effectiveness profiles of the multi specific ligand, especially if such careful balance is required. The invention further contemplates a composition containing an antibody which specifically binds to an epitope on a ligand. The ligand recognized by the antibody exerts a biologic effect by binding to a target site on a target ligand. The epitope bound by the antibody is proximal to the binding site of the ligand for the target ligand, so that binding of the antibody reduces but does not prevent the affinity of the ligand for its target ligand. The invention additionally contemplates physiologically acceptable compositions of the compositions encompassed by the invention.

The invention likewise contemplates methods of use of the compositions encompassed by the invention for example for testing biologic or therapeutic hypotheses.

A composition comprising a multispecific ligand comprising at least a first ligand binding moiety which specifically binds to a first ligand having a first biodistribution and a second ligand binding moiety which specifically binds to a second ligand having a second biodistribution different from that of the first ligand, and wherein the affinity of the first and second ligand binding moieties are different and selected to bias the biodistribution of the multispecific ligand.

### DETAILED DESCRIPTION OF PREFERRED EMBODIMENTS

As exemplified above, the dual “affinity”on-rate” based targeting strategy of the invention, may be understood in one aspect, in terms of a strategic allocation of the respective affinity properties of the multispecific ligand to at least one “targeting” function and at least one “effector” function. Accordingly, with respect to some embodiments of the invention, the term “multifunctional” ligand is used interchangeably.

Thus according to one preferred embodiment, at least one of the ligand binding moieties is a “targeting” arm in the sense that it at least preferentially recognizes a marker (a first target ligand) that is associated with one or more specific target entities e.g. cell populations (it is associated in the sense that it is uniquely or preferentially expressed on the target entities), and the other ligand binding moiety is an “effector” arm which binds with relatively less affinity or functional affinity and/or less quickly to a second target ligand which optionally, and in preferred non-limiting embodiments, has a more diverse biodistribution. In this case, the biodistribution of the multispecific ligand is biased in favor of the location of both target ligands, both being primarily on the target entities relative to the location only of the second target ligand of the target ligand so as to limit the bio distribution to non-target entities.

The term biodistribution means distribution of a target ligand or the multispecific ligand in a natural biological environment between bioavailable target entities, and bioavailable non-target entities, the object of the invention being to minimize or limit distribution to non-target entities, with a view to ascertaining an effect of such a biased distribution, and in a therapeutic context, improving the safety, improving the efficacy, or reducing the risk of side effects or confounding effects from the unwanted distribution to non-target entities.

Such binding or recognition is understood throughout to be preferably specific, in contrast to non-specific type binding.

Accordingly, in several non-limiting embodiments, the invention may be summarized in the following paragraphs:

1. A multispecific ligand which comprises, at least, a targeting moiety which specifically binds to a first ligand having a first cell surface biodistribution and an effector moiety which binds to a second ligand having a second cell surface biodistribution different from but overlapping that of the first ligand, and wherein the targeting moiety is intrinsically predisposed to bind to the first ligand in preference to the effector moiety binding to the second ligand so as to bias the distribution of the multispecific ligand to a target cell population in an organism on which both said first and seconds ligands are bioavailable for recognition by said multispecific ligand, said second ligand characterized in that it is bioavailable for recognition on at least one population of cells other than the target cell population. In one non-limiting embodiment, the effector moiety exerts or inhibits a cellular response. The term cellular response, in the context of the invention, means that the effector moiety through an agonist or antagonist effect causes or inhibits a biological change within the target cell. The multispecific ligand is adapted to limit the occurrence of an agonist or antagonist effect on the non-target cell. In one non-limiting embodiment, the effector moiety inhibits a soluble or cell-surface ligand from exerting its primary biologic function in relation to the target cell and the multispecific ligand is adapted to limit this inhibitory effect on the non-target cell. Primary biologic function means that the natural effect of the ligand in relation to the ligand to which the effector is bound.

2. A multispecific ligand according to paragraph 1, wherein the first ligand is pre-targeted to the surface of the target cell population. The invention contemplates that the first ligand may itself be administered and may provide a site for high affinity binding for a multispecific ligand which comprises a high affinity binding moiety (e.g. a second antibody fragment, biotin, avidin, or an L coil or K coil involved in heterodimeric coiled coil interactions; (De Crescenzo G, et al. Real-time monitoring of the interactions of two-stranded de novo designed coiled coils: effect of chain length on the kinetic and thermodynamic constants of binding. Biochemistry. Feb. 16, 2003; 42(6):1754-63; Chao H, et al. Kinetic study on the formation of a de novo designed heterodimeric coiled-coil: use of surface plasmon resonance to monitor the association and dissociation of polypeptide chains. Biochemistry. Sep. 17, 1996; 35(37):12175-85; Litowski J R, et al. Designing heterodimeric two-stranded alpha-helical coiled-coils. Effects of hydrophobicity and alpha-helical proprensity on protein folding, stability, and specificity. J Biol Chem. Oct. 4, 2002; 277(40):37272-9. Epub Jul. 22, 2002; Arndt K M, et al. Helix-stabilized Fv (hsFv) antibody fragments: substituting the constant domains of a Fab fragment for a heterodimeric coiled-coil domain. J Mol Biol. Sep. 7, 2001; 312(1):221-8.)

3. A multispecific ligand according to paragraph 1, wherein the first ligand is expressed on the surface of the target cell population.

4. A multispecific ligand according to paragraph 1, 2 or 3 wherein the intrinsic affinity of the targeting moiety exceeds the intrinsic affinity of the effector moiety.

5. A multispecific ligand according to paragraph 1, 2, 3 or 4, wherein the on-rate of the targeting moiety exceeds (is faster) than the on-rate of the effector moiety.

6. A multispecific ligand according to paragraph 1, 2, 3, 4 or 5, wherein the intrinsic off-rate of the targeting moiety is less (slower) than the intrinsic off-rate of the effector moiety.

7. A multispecific ligand according to paragraph 1, 2, 3, 4, 5 or 6, wherein the intrinsic on-rate of the targeting moiety exceeds the intrinsic on-rate of the effector moiety.⁴ For example, the effector moiety may be a “coybody” (as defined below), which may be generated, for example, by a process of mutagenesis in which one or more residues in at least one CDR or FR of the VH or the VL, are mutated or added, preferably residues which do not contribute to the specificity and off-rate of reference antibody, for example an antibody of medium or high affinity and desired specificity. For example, one of the CDRS, more commonly the CDR2 of the reference antibody, may not be involved in binding to the target ligand in question, and by a process of random mutagenesis, and/or by lengthening and/or by introducing large, hydrophobic, hydrophilic or charged amino acids (depending on the amino acid constitution of the epitope), one or more of a series of such modifications may be selected from a library of such CDR2 modifications, for example, under conditions in which the amount of antibody exceeds the amount required for saturation of binding sites on the available antigen (as may be determined through titration using the reference antibody or independently), so as to permit a rapid series of selections to take place under stringent conditions, for example, of shear force (more vigorous shaking than the degree of vigor required to ensure a uniform distribution of phage particles) with the later selections having a greater propensity to yield antibody with a slower on-rate. Amino acids and portions (CDRs) of the antibody variable region which don't contribute to specificity and off-rate may be determined via mutagenesis,i for example, by alanine scanning mutangenesis, by assessing singly or in multiples, residues located in each CDR loop to ascertain those which when substituted do not significantly or measurably affect the off-rate of the antibody, as may be determined by surface plasmon resonance or microcalorimetry, or approximation through scatchard analysis, FACS, fluourescence polarization, fluorescence quenching, Optionally, the effect on a readable signal is determined at a concentration of antibody which is predetermined to have the greatest incremental impact on signal change (the sloped portion of a sigmoid dose response curve) so that the effect of mutations is more readily perceived through the readout. Residues which are most likely to border or otherwise affect the target ligand binding site may be predicted, for example, by well-known methods of three dimensional modeling or statistical plots of residues which are most variable or correspond to mutational hot-spots (these residues may also be residues in the neighbouring FRs. A convenient group of residues from which to select are those residues which are not conserved (and preferably therefore also do not positively contribute to the solubility, stability and expression characteristics of the reference antibody).

⁴ optionally by at least 2, 3, 4, 5, 6, 7, 8, 9, 10, 11, 12, 13, 14, 15, 16, 17, 18, 19, 20, 21, 22, 23, 24, 25, 26, 27, 28, 29, 30-40, 40-50, 50-60, 60-70, 70-80, 80-90, or 90-100 (or more) fold. With respect to each of the foregoing at least 100 increments, the intrinsic off-rate of the targeting moiety is optionally at least 2, 3, 4, 5, 6, 7, 8, 9, 10, 11, 12, 13, 14, 15, 16, 17, 18, 19, 20, 21, 22, 23, 24, 25, 26, 27, 28, 29, 30-40, 40-50, 50-60, 60-70, 70-80, 80-90, or 90-100 (or more) fold less than the intrinsic off-rate of the effector moiety.

8. A multispecific ligand according to paragraph 7, wherein the intrinsic on-rate of the targeting moiety is selected so as to exceed the on-rate of the effector moiety.

9. A multispecific ligand according to paragraph 7, wherein the intrinsic on-rate of the effector moiety is selected so as to be less than the on-rate of the targeting moiety.

10. A multispecific ligand according to paragraph 9, wherein one or both of said effector and targeting moieties, comprises one or both of a VH or VL. There are many constructs and strategies that have been developed for the generating bispecific ligands, as detailed in the literature cited below, and summarized in numerous reviews (see for example) including methods of chemical synthesis, recombinant DNA synthesis and hybridoma cell fusion based syntheses (see for example, Suresh M R, et al Bispecific monoclonal antibodies from hybrid hybridomas. Methods Enzymol. 1986; 121:210-28) Constructs in which scFvs or single domain antibodies are linked via a flexible linker and Fabs linked via disulfide bonds (Fab′)2, leucine zippers and heterodimeric coiled-coil interactions have been well characterized. Bispecific IgGs have also been developed. Diabodies have been shown to have geometry that may also be amenable to binding two epitopes on the same cell. Recombinant fragments linked by flexible linkers of for example, 10-25 amino acids offer the promise of greatest flexibility in cross-linking different antigens on the same or adjacent cells.

11. A multispecific ligand according to paragraph 9, wherein one or both of said effector and targeting moieties, consists essentially of one or both of a VH or VL.

12. A multispecific ligand according to any of the preceding paragraphs, wherein the first and/or second ligand is expressed predominantly on one or more cell populations of hemapoietic origin.

13. A multispecific ligand according to any of the preceding paragraphs, wherein the first and/or second ligand is expressed predominantly on endothelial cells.

13b. A multispecific ligand according to any of the preceding paragraphs, wherein the first and/or second ligand is expressed primarily on a hemapoietic stem cell.

14. A multispecific ligand according to any of the preceding paragraphs, wherein the fill in

15. A multispecific ligand according to any of the preceding paragraphs, wherein the first and/or second ligand is an ABC transporter protein, a ligand that is over-expressed on tumor cells, an adhesion molecule, a ligand that is expressed exclusively or predominantly on one or more types of leucocytes, one or more sub-populations of T cells or B cells, a cytokine receptor, a growth factor receptor, a molecule or complex comprising a transmembrane protein, or a CD molecule, other than CD59, a receptor is selected from the group consisting of tyrosine kinase type receptors, serine kinase type receptors, heterotrimeric G-protein coupled receptors, receptors bound to tyrosine kinase, TNF family receptors, notch family receptors, guanylate cyclase types, tyrosine phosphatase types, decoy receptors, inhibitory receptors and adhesion receptors (other than CD59) or any cell surface receptor-ligand cooperating with such a receptor.

16. A multispecific ligand according to any of the preceding paragraphs, wherein said second ligand binding moiety specifically recognizes said second ligand.

17. A multispecific ligand according to any of the preceding paragraphs, wherein the sum of bioavailable second ligands on said target cell population is less that the sum of bioavailable second ligands on cells other than said target cell population.

18. A multispecific ligand according to any of the preceding paragraphs, wherein the first ligand is preferentially bioavailable for targeting purposes on said target cell population.

19. A multispecific ligand according to paragraph any of the preceding paragraphs, wherein the first ligand is exclusively bioavailable for targeting purposes on said target cell population.

20. A multispecific ligand according to any of the preceding paragraphs, wherein the multispecific ligand is adapted to bind contemporaneously to said first and second ligands⁵.
⁵ This may be assessed for example by determining the extent to which the effector moiety out-competes a test ligand having the same specificity as the effector moiety but a somewhat higher on-rate and/or affinity in virtue of the superior (relative to the effector moiety and test ligand) affinity and/or on-rate of the targeting moiety. The effects of modifying the affinity of each of these three elements in either direction with suitable positive and negative controls may be tested to further refine this assessment. The choice of fist and second ligand can be also be influenced by the likelihood that both would be found within or unassociated with lipid rafts (in some cases this may be more important than others), as determined for example by the length of the transmembrane domain, if any, or in some cases whether the ligand is one which is anchored by GPI, whether the effector moiety is selected to agonize or antagonize a receptor and the functional sensitivity of the cell to a less than complete saturation of the second ligand (as influenced by dose and any related MTDs), especially when antagonist effects are being exerted on a receptor (as opposed to agonist effects which may require less binding events depending on the sensitivity of the cell to such events) etc. Although such predictions may have some influence on the a priori choice of ligands for testing, ultimately empirical in vitro assessments may be necessary in most cases.

21. A multispecific ligand according to paragraph 20, wherein the surface cell density of the first ligand on the target cell population exceeds the surface cell density of the second ligand on said target cell population.

22. A multispecific ligand according to paragraph 1, wherein the number of bioavailable second ligands in said mammal ligands is greater than the number of bioavailable first ligands and wherein the in-vivo site of action of said effector moiety is predominantly on the target cell population.

23. A multispecific ligand according to paragraph 1, 2, 3, 4 or 5, wherein the cell-surface density of the second ligand on the target cell population generally does not exceed the cell-surface density of the second ligand on a non-target cell population and where the in-vivo site of action of said effector moiety is predominantly on the target cell population.

24. A multispecific ligand according to any of the preceding paragraphs, wherein the first and second ligand binding moieties are small molecules⁶ that are conjugated to a fullerene or a peptide.
⁶ The term small molecule typically connotes small synthetic or semi-synthetic organic molecules which are not macromolecules such as proteins, lipoproteins, glycoproteins, long chain fatty acids etc.

25. A multispecific ligand according to any of the preceding paragraphs, comprising a bispecific antibody in which the Fc portion of the molecule is absent or incapable of binding to an Fc receptor.

26. A multispecific ligand according to paragraph 10, 11 or 25, wherein at one or both of the targeting moiety and effector moieties comprises human sequences, and is non-chimeric or includes at least human FRs.

27. A pharmaceutical composition comprising a multispecific ligand according to any of the preceding paragraphs and a pharmaceutically acceptable carrier.

The term “effector” is used to refer to the ability to effect a biological consequence through binding, for example effecting a signal transduction event by activating a receptor, or blocking the target ligand from associating with a complementary ligand, for example blocking a receptor from associating with a complementary ligand (e.g. its natural ligand whether soluble or on another cell) and thereby, for example, preventing a signal transduction, or for example in the case of a decoy receptor preventing the biological consequence (e.g. protective effect) associated with the function of such receptor, or blocking a ligand from associating with a complementary ligand e.g. receptor on another entity e.g. a cancer cell, infectious agent or immune cell.

The term targeting moiety means capable of distinguishing at least in part between the target entities (e.g. cells according to one embodiment), which target entities are defined at least in part by a first ligand, typically found on the target cell population and at least some entities e.g. cells, other than the target population of entities, that bear only the second ligand acted on by the effector moiety.

A biased biodistribution is preferably accomplished by the multispecific ligand contemporaneously recognizing both ligands on the same entity, e.g. cell, and may be accomplished by such contemporaneous recognition occurring on adjacent entities or by increasing the propensity of the multi specific ligand to locate in proximity to a target entity in virtue of the relatively high affinity targeting arm. The targeting arm may itself be an effector.

In another embodiment, the biological consequence accomplished by the effector arm is, minimally, cooperative targeting, for example where the biodistributions of at least one of the first and second ligands extends to a diverse population of cells other than target cell population and where binding is only possible or consequential if both ligands are available for contemporaneous binding, in this case due to the affinities of the first and second ligand binding being individually insufficient for effective targeting (e.g. insufficient for other than ephemeral binding). In the context of this embodiment of the invention, the “cooperative targeting” is not simply ameliorated by the effector arm, it is predicated and reliant on this arm. One or both ligand binding moieties may exert additional effector properties.

It will also be appreciated that any multi specific ligand of the invention or any component thereof may be fused or conjugated to a separate effector as exemplified below, including toxins, cytokines, adhesion molecules etc.

The ligand binding moiety is in three non-limiting embodiments *** an antibody, a peptide mimetic of the natural ligand, or a sequence or sequences of amino acids etc. which are the natural ligand for the target ligand, for example where the ligand is a cytokine or lymphokine receptor, such as IL-2 receptor, the ligand binding moiety may comprise a sequence of amino acids which is IL-2 or a functional receptor binding portion thereof. Other suitable ligands include, Affibodies™, T cell receptors (TCRs), anticalins, fibronectin type III domains, C-type lectin-like domains, V-like domains (see CA 2,322,621), peptides and peptide fusions and conjugates and stabilized polypeptide loops. The ligand binding moiety may also be a mutated or a newly developed form of the natural ligand (e.g. developed through combinatorial libraries) or a natural or synthetic chemical ligand (developed through combinatorial chemistry).

In one aspect, the invention contemplates a composition containing a multispecific ligand containing at least a first ligand binding moiety and a second ligand binding moiety, the first ligand binding moiety specifically binding with a pre-selected first affinity to at least a first ligand, having a first biodistribution and the second ligand binding moiety specifically binding with a pre-selected affinity to at least a second ligand with a second biodistribution and wherein the affinity of first and second ligand binding moieties are selected to bias the biological site of biologic activity of the multispecific ligand; and wherein the first ligand binding moiety preferably binds with high affinity (preferably nanamolar affinity or greater) to a specific cell associated marker (e.g. a CD marker, a marker associated with diseased cells, a marker associated cells in a particular physiological state (e.g. activated T cells, B cells) etc. (such markers may be associated with a particular class of cell or a subclass thereof (if applicable) or particular subpopulation within the subclass (if applicable), however classified, such as epithelial cells, endothelial cells, immune cells (lymphocytes, memory cells, effector cells) monocytes, T cells (CD4+, CD8+, CD45RO+), hepatocytes, stem cells, etc. (expand) and wherein said second ligand binding binding moiety binds with relatively low, or medium affinity (preferably 0.1 micromolar or less) to a receptor (e.g. chemokine, growth factor, cytokine) involved in cell signaling or a decoy receptor, a cell surface receptor ligand e.g. the ligand for such receptor which effects a signal or inhibits a signal (e.g. CTLA4), a ligand involved in cell adhesion, a receptor or channel (ion channel) for a molecule involved in cell regulation or homeostasis etc.

The invention contemplates that the difference in affinity will in most cases be an essential element in biasing the location of action of the multispecific ligand to yield an acceptable or desired safety profile and that the high affinity of the first ligand binding moiety for the cell associated marker will be optimized for this purpose insofar as the safety profile of the multispecific ligand dictates maximizing its affinity characteristics. The invention also recognizes that choosing the relatively lower affinity of the second ligand binding moiety may assist in this regard up to a point where its effectiveness to bind to the second ligand is significantly comprimised. In this regard, the invention also contemplates that factors other than the choice of affinity of the first and second ligand binding moieties (and of course the avidity effect resulting from having two ligands on the target cell and only one on the non-target cell) may be taken into consideration or optimized to balance the safety and effectiveness profiles of the multispecific ligand, especially if such careful balance is required.

Examples of such factors, one or more of which can be employed in various combinations, are described hereafter, and will be appreciated to be parameters to be taken into account if not optimized, based on empirical evidence of their importance:

1) the selection of cell associated marker, in terms of its cell surface density relative to cell density of the second ligand. The number of first and second ligands on a cell surface is dynamic but can readily be approximated by radiolabelling studies or by flow cytometric methods relative to a standard. The selection of the cell surface marker in this respect will depend on the function of the relatively low affinity binding moiety, for example: 1) whether it causes a signal transduction or other agonist effect indirectly e.g. or directly through binding to a receptor, in which case, less emphasis on relative cell density may be warranted since fewer receptor binding events may be needed relative to an antagonistic function (agonist antibodies are well known in the art and include those described in U.S. Pat. No. 6,342,220, U.S. Pat. No. 6,331,302, U.S. Pat. No. 5,635,177, U.S. Pat. No. 6,099,841 see also Cancer Res Mar. 1, 2001; 61(5):1846-8 and can be made according to routine screening techniques, using assays for agonist function, as may be known or referenced herein or designed on a case by casebasis, optionally, in some cases, if required, by selecting for antibodies capable of cross-linking receptor components for example using second ligand binding moieties having divalent functionality (examples referenced herein) or using antibodies in which the VH and VL are capable of binding individually to different receptor components or otherwise (see also Zhou H X. J Mol Biol May 23, 2003; 329(1):1-8), 2) whether it binds to a decoy receptor; 3) whether it binds to an inhibitory receptor etc. 4) whether it selectively protects a tissue from the effects of another therapy, for example, immunotoxin therapy by blocking the target of the immunotoxin on vital non-target cells e.g. blocking Lewis Y, EPCAM, TAG-72 or another cancer antigen., for example, as expressed on intestinal epithelia, using another epithelial marker as the target for the high affinity binding arm, so as to inhibit binding of the cancer antigen targeted antibody from binding to vital areas other than the target lesions 5) prevents a signal transduction (directly or indirectly, e.g. binding to a receptor or binding to receptor ligand, for example a receptor ligand on a cell) and in the final analysis how many binding events per cell are required to cause or prevent the sought-after biological effect. This can be assayed in vitro through well known assay methods established in the art for measuring such biologic effects, for example assays that have been developed for measuring responses to external stimuli or the absence of such stimuli, for example release of/exposure to cytokines, chemokines, growth factors, colony stimulating factors or assays using various immunostaining/immunofluoresence techniques including flow cytometry (e.g. to measure apoptosis e.g. annexin V binding assay) signal transduction assays (e.g. using phosphospecific antibodies that detect phosphorylation of serine, tyrosine, threonine), differential gene expression, as measured for example by real time PCR, etc.) depending on the type of effect that is being measured (see for example Biosource Method Booklets at current URL:
http:www.biosource.com/content/techCornerContent/methodPDFs/index.asp; see also Amersham Bioscience catalogues, and those of other well known suppliers etc.) or via animal studies.

Antagonist effects may be quantified indirectly through competition with a natural ligand or another antibody, for example, by determining a saturating concentration of the test ligand or a known competitor and assessing the relative decrease in binding attributable to the other ligand.

Assays for Effectiveness of Effector Moiety

In vitro assays and animal models are well described in the literature. (see for example WO03/56301)

Assays for Screening of Multidrug Resistance

Garrigues A, et al. A high-throughput screening microplate test for the interaction of drugs with P-glycoprotein. Anal Biochem.(2002);305(1):106-14. PMID:12018951; Robey R, et al. Efflux of rhodamine from CD56+ cells as a surrogate marker for reversal of P-glycoprotein-mediated drug efflux by PSC 833. Blood. (1999);93(1):306-14. PMID:9864175; Quesada A R,et al. Chemosensitization and drug accumulation assays as complementary methods for the screening of multidrug resistance reversal agents. Cancer Lett. (1996);99(1):109-14. PMID:8564921; Lin J T, et al. A flow cell assay for evaluation of whole cell drug efflux kinetics: analysis of paclitaxel efflux in CCRF-CEM leukemia cells overexpressing P-glycoprotein. Drug Metab Dispos.(2001);29(2):103-10. PMID: 11159798; Tunggal J K, et al. Influence of cell concentration in limiting the therapeutic benefit of P-glycoprotein reversal agents. Int J Cancer.(1999);81(5):741-7. PMID:10328227.

Assays for Agonist are detailed in various literature, some of which are listed below:

WO 03/040303 Wong S, K-F, et al. Functional Assay For Agonist Activation Of Receptors(2003); WO 97/47970 Boime I, et al. Assay System For Glycoprotein Agonists And Antagonists(1997); WO 97/21731 Kopin A S. Assay For And Uses Of Peptide Hormone Receptor Ligands (1997); WO 00/02045A3 Bioluminiscent Assay For Agonists Or Antagonists Of A Calcium-Coupled Receptor(2000); Kunapuli P, et al. Development of an intact cell reporter gene beta-lactamase assay for G protein-coupled receptors for high-throughput screening. Anal Biochem.(2003);314(1):16-29.PMID:12633598; Brandish P E, et al. Scintillation proximity assay of inositol phosphates in cell extracts:High-throughput measurement of G-protein-coupled receptor activation.Anal Biochem.(2003);313(2):311-8.PMID:12605869; Stables J, et al.A bioluminescent assay for agonist activity at potentially any G-protein-coupled receptor.Anal Biochem.(1997);252(1):115-26. PMID:9324949; Szekeres P G. Functional assays for identifying ligands at orphan G protein-coupled receptors. Receptors Channels.(2002);8(5-6):297-308.Review.PMID:12690957; Conway B R, et al. The use of biosensors to study GPCR function-applications for high-content screening.Receptors Channels.(2002); 8(5-6):331-41. Review.PMID:12690960. See also WO 00/52474A1.

Assays for Antagonist are detailed in various literature, some of which are listed below:

WO 03/056301A2 Peritt, D, et al. A Novel Screening Method For Molecular Antagonist Using Flow-Cytometry(2003); WO 98/13513 Trueheart, J. Methods And Compositions For Identifying Receptor Effectors(1998); WO 98/47923 Paul, N I et al. IL-5R Antagonists For Treatment Of Inflammation, Asthma And Other Allergic Diseases (1998); WO 01/89566A1 Mass, R D, Gene Detection Assay For Improving The Likelihood Of An Effective Response To An ERBB Antagonist Cancer therapy(2001); WO 01/44818A3 Armbruster, F P, et al. Method For Determining Parathormone Activity In A Human Sample(2001); WO 03/056301A2 A Novel Screening Method For Molecular Antagonist Using Flow-Cytometry(2002; Pauwels P J.5-HT 1B/D receptor antagonists.Gen Pharmacol.(1997);29(3):293-303. Review.PMID: 9378233; Shen K, et al. Acquisition of a specific and potent PTP1B inhibitor from a novel combinatorial library and screening procedure.J Biol Chem. Dec. 14, 2001; 276(50):47311-9.Epub(2001)PMID:11584002; Gao J, et al. An enzyme-linked immunosorbent assay to identify inhibitors of activation of platelet integrin alpha IIb beta 3. J Immunol Methods.(1995);181(1):55-64. PMID:7537313

Methods for assessing affinity of the effector arm are well known to those skilled in the art and are referred to above. Recent effort to assess affinity by FACS previously described by Benedict et al. are described in WO 03/056296A2 Improved Methods for Determining Binding Affinities(2002).

Furthermore, it will be known or empirically ascertainable that some growth factors, lymphokines or molecules/ions required for homeostasis are in more delicate balance and can more easily disrupted, whereas others may require greater technological assertion to accomplish the desired effect. For example, IL-2 depletion will cause apoptosis of activated T cells, which can be measured. For example, it may be empirically determined to be necessary or desirable, on a case by case basis, for the cell specific marker to approximate (the number of cell specific markers on the target cell population which may be determined to be preferably no less in number than 50% more or no less than 90% in number relative to the second ligand—as stated above, which will depend on what degree of causation or prevention of the signaling/interaction will cause the desired biologic effect) or out-number (for example, by greater than 30%, 50%,100%, (greater than two fold), 200% (greater than 3 fold), or by greater than 300% (greater than 4 fold)) the target ligand for the relatively low affinity binding moiety, for example, if the goal is to block interaction of a receptor with a high affinity ligand that exerts a biological effect in low concentration (e.g. a cytokine).

2) Furthermore, in the latter case (which may exemplify a case in which the greatest number of technological factors need to be optimized) the affinity of the first binding moiety will preferably be selected to approximate (preferably as empirically determined, preferably no less than one order of magnitude, more preferably no less than 5 fold less, more preferably no less than three fold less, more preferably no less than two fold less, more preferably no less than one fold (100%) less), and may be determined to preferably equal or exceed the affinity of the natural ligand. It is understood in this case that avidity and dose may not necessitate any additional affinity maturation needed to exceed the affinity of the natural ligand.

3) Furthermore, or in the alternative, the concentration (in virtue of the choice of administered dose) of the multi specific ligand in the target cell microenvironment may also be selected to exceed that of the natural ligand (MTD permitting).

4) the choice of construct may be based on maximizing the steric blocking of the target (IgG or F(ab′)² vs diabody). Furthermore, in some mammalian systems (e.g. mice) the hinge region is naturally longer and this effect can be mimicked for human antibodies through a hinge extension on the N-terminal side of the hinge region using well known neutral linkers (gly4ser) or a repeat of all or a portion of the natural hinge sequence. This extension will also permit a greater span between first and second ligands to be bridged. As is appreciated in the art flexible linkers of varied lengths may be employed to link two antibody fragments or constructs;

5) the choice of construct may include an Fc portion or partial Fc portion (e.g. CH2 or minibody-CH3) or weighted Fc e.g. by pegylation (site specific pegylation is well known in the art) or IgG subtype naturally having additional Fc domains (e.g. an IgE) (which Fc if it includes the CH3 is preferably mutated to preclude its binding and/or increase its half life as is known in the art see U.S. Pat. No. 6,121, 022) so as to maximize the shear effects on the multispecific ligand which may be determined be most consequential in the case of univalent binding in order to minimize the duration of such binding (maximum shear force might also be determined to be preferred where there is an excess in the total number of bioavailable targets of the second ligand binding moiety relative to the total number of bioavailable targets of first ligand binding moiety (greater number of cells and/or greater number of targets per cell and/or increased bioavailablity of such targets e.g. on normal cells relative to cancer cells).

6) Optionally, the multispecific ligand may include a 3^{rd} binding moiety which for example, binds to and neutralizes the natural ligand for the receptor sought to be blocked. Such formats are well known in the art (see for example particularly Schoonjans R et al. A new model for intermediate molecular weight recombinant bispecific and trispecific antibodies by efficient heterodimerization of single chain variable domains through fusion to a Fab-chain. Biomol Eng. June 2001; 17(6):193-202. Schoonjans R et al. Fab chains as an efficient heterodimerization scaffold for the production of recombinant bispecific and trispecific antibody derivatives.J Immunol. Dec. 15, 2000; 165(12):7050-7. Schoonjans R, et al. Efficient heterodimerization of recombinant bi- and trispecific antibodies. Bioseparation. 2000;9(3):179-83. see also French R R. Production of bispecific and trispecific F(ab)2 and F(ab)3 antibody derivatives. Methods Mol Biol. 1998;80: 121-34; U.S. patent application No. 20020004587; Kortt A A, Dimeric and trimeric antibodies: high avidity scFvs for cancer targeting. Biomol Eng. Oct. 15, 2001; 18(3):95-108).

7) Optionally, two multispecific ligands each binding to different cell specific markers and each having a second ligand binding moiety which binds to the same second ligand e.g. a receptor, optionally to a different polypeptide/component of the receptor, may be employed to achieve the desired biologic effect. One or both may also be trispecific as discussed above. As discussed above, according to another non-limiting embodiment the multispecifc ligand may be used to protect a first target cell population in virtue of its high affinity (and/or faster on-rate) first ligand binding moiety from the effects of a therapeutic entity which desirably binds to a second target cell population via the second ligand but which therapeutic moiety also undesirably binds to the first target cell population. Therefore the second ligand binding moiety can be used to selectively block the binding of the therapeutic entity e.g. an interleukin, interferon, immunotoxin, etc. to the first target cell population in virtue of the relatively low affinity second ligand binding moiety. In this case, the multispecific ligand may also comprise a third ligand binding moiety which binds to the therapeutic entity, particularly where the multispecific ligand is first administered first, optionally an anti-idiotypic binding moiety component in a case where the therapeutic comprises an antibody component. Optionally, the multispecific ligand may also comprise an enzyme cleavage site, wherein the cleavage is effected at the second target cell population in virtue of the particular enzyme constitution in the vicinity of that target cell population (e.g. matrix metalloproteases within a tumor) and results in the inability of the multi specific ligand to block the therapeutic entity from binding to the second target cell population e.g. in the case of an antibody the cleavage site may be in a CDR3 or FR to as to interfere with binding capacity. Such enzyme cleavage sites are well known to those skilled in the art. With respect to protecting non-tumors cells from the effects of a therapeutic molecule that binds to cancer cells via the target of the second ligand binding moiety, the protective multispecific ligand will have a first ligand moiety which binds to the cells sought to be protected and the choice of multispecific ligand construct may be larger in size to relatively inhibit tumor penetration.

8) As discussed in general terms above, in one non-limiting embodiment, the first ligand binding moiety may initially be selected to have a 10 to 100 times faster on-rate than the second ligand binding moiety as well as off-rate which 10 to 1000 times slower off-rate, so that in combination with the expected avidity effect, the first ligand binding moiety (targeting arm,) will have an approximately 3 to 6 order of magnitude greater functional affinity than the that of the second ligand binding moiety (effector arm).

Some sample targets are listed immediately below, while others are listed later. Greater targeting using a high affinity (and/or faster on-rate) first ligand binding moiety which binds to a cell associated or specific marker may be imparted to a variety of existing antibodies with suitably diminished affinity including those marketed or in clinical trials or listed below which are the subject of the patent and scientific literature, including those listed in PharmaBusiness June 2002 No.51:

| | Functional | | | |
|---|---|---|---|---|
| | ligand for | | | |
| | low affinity | | | |
| | arm | | | |
| | (location of | Cell localizing ligand | | |
| Category | ligand) | for high affinity arm | Mode of action | Comments |
| Growth | IL-2. | CD4 T cells | Growth factor | IL-2 required |
| factor | (soluble) | | blockade for a | for naïve CD4 |
| blockade | | | specifically for CD4 | or CD8 and |
| | | | T cell subset; i.e. | memory CD4 |
| | | | selective | T cell |
| | | | immunosuppression | responses;. |
| | | | | Binding of |
| | | | | CD4 or CD8 |
| | | | | would also |
| | | | | block |
| | | | | interaction |
| | | | | with antigen |
| | | | | presenting |
| | | | | cells. |
| | IL-15 | CD8 T cells | Growth factor | IL-15 needed |
| | | | blockade for a | for memory |
| | | | specifically for CD8 | CD8⁺ T cell |
| | | | T cell subset; i.e. | responses |
| | | | selective | |
| | | | immunosuppression | |
| Chemokine | MCP-1 | CD11c | Monocyte/macrophage | At time of |
| blockade | (soluble) | (monocyte/macrophage) | chemokines; | percutaneous |
| | | | e.g. anti- | coronary |
| | | | inflammatory agent | intervention |
| | | | | (PCI) to limit |
| | | | | restenosis; |
| | | | | arthritis |
| Cell | Thrombin | CD31 (endothelial | Prevent thrombin | Limit |
| activation | (soluble) | cells) | binding to thrombin | thrombosis |
| blockade | | or | receptors on | and |
| | | P-selectin (thrombin | leukocytes or | endothelial |
| | | activated endothelial | endothelial cells at | activation, e.g |
| | | cells) | endothelial surface | at time of PCT |
| Cell | CD80/86 | CD83 (dendritic | Block interaction | At time of |
| activation | (dendritic | cells) | with CD28; | allografting to |
| blockade | antigen | | immunosuppression | induce |
| | presenting | | | tolerance |
| | cells) | | | |
| Inhibitory | Fc gamma | Fc epsilon RI | Enhance association | Rx of acute of |
| receptor | RII | (Mast cells) | of activating and | allergic |
| activation | (Mast cells) | | inhibitory receptors. | disease. |
| | | | | Advantages |
| | | | | over bispecific |
| | | | | Fc fusion |
| | | | | reagents |
| | | | | because of |
| | | | | more specific |
| | | | | cell targeting. |
| | | | | See Zhu D et |
| | | | | al. Nat Med |
| | | | | 2002 |
| | | | | May; 8(5): 518-21) |
| Inhibitory | CTLA-4 | CD8 | Block CD80/86 | Enhance |
| receptor | | | interaction with | specifically |
| blockade | | | CTLA-4 | CTL mediated |
| | | | | anti-tumor |
| | | | | responses |
| | | | | without global |
| | | | | T cell |
| | | | | activation. |
| | | | | Anti-CTLA4 |
| | | | | abs now in |
| | | | | therapeutic |
| | | | | trials to |
| | | | | enhance tumor |
| | | | | immunity |
| Adhesion | VCAM-1 | CD31 (endothelial | Block VLA-4- | Acute Rx of |
| molecule | (activated | cells) or E-selectin | dependent T cell | MS flare-up. |
| blockade | endothelial | (activated | and monocyte | Anti-VLA-4 is |
| | cell) | endothelium) | adhesion to VCAM- | in trial as Rx |
| | | | 1 on endothelial | for MS. |
| | | | cells. | |
| Adhesion | ICAM-1 | CD31 (endothelial | Block neutrophil | Acute Rx to |
| molecule | (activated | cells) or E-selectin | adhesion to | reduce |
| blockade | endothelial | (activated | activated | reperfusion |
| | cell) | endothelium | | injury |
| | | | | (myocardial |
| | | | | infarction, |
| | | | | bowel |
| | | | | ischemia/surgery) |
| Cell death | CD95L (Fas- | CD25 (activated T | Block activation | Both ligands |
| ligand | ligand) | cells) | induced cell death | on same cell; |
| blockade | (T cell) | | of T cells; enhance | CD95L |
| | | | anti-tumor | expressed on |
| | | | immunity | activated, not |
| | | | | resting T cells |
| Protect from | IFN-_{—} | CD31 | Block IFN-_{—} | Cell selective |
| another | (soluble) | | toxicity towards | block of IFN-_{—} |
| therapeutic: | | | endothelial cells | effects; |
| e.g. | | | during IL-2 therapy | |
| immunotoxins | | | for tumors | |
| Protect from | IFN-_R | CD31 | Block IFN-_{—} | Both ligands |
| another | (endothelial | (endothelial cell) | toxicity towards | on same cell. |
| therapeutic: | cell) | | endothelial cells | Cell selective |
| | | | during IL-2 therapy | block of IFN_{—} |
| | | | for tumors | effects; |
| Inhibitory | TGF-_{—} | CD4 or CD8 | Block | TGF-_mode |
| receptor | | | immunosuppressive | of |
| blockade | | | effects of tumors; | immunosuppression |
| | | | enhance anti-tumor | not |
| | | | immunity | clear |
| Cell type | CD3 | CD45RO | Trivalent ab so CD3 | Anti-tumor |
| specific | | (memory/effector T | can be cross-linked | Rx; selectively |
| activation | | cells) | | enhance |
| | | | | memory T |
| | | | | cells; reduce |
| | | | | nonspecific |
| | | | | activation of |
| | | | | irrelvent T |
| | | | | cells |
| TABLE Continued | | | | |
| | Functional | | | |
| | ligand for | | | |
| | low affinity | | | |
| | arm | Cell localizing | | |
| | (location of | ligand for high | | Possible therapeutic |
| Category | ligand) | affinity arm | Mode of action | uses |
| Growth | IL-15 | CD8 T cells | Growth factor | For Rx of allograft |
| factor | | | blockade for | rejection; T cell |
| blockade | | | specifically for | subset specific |
| | | | CD8 T cell subset; | suppression will limit |
| | | | i.e. selective | infectious |
| | | | immunosuppression | complications or |
| | | | | unintended inhibition |
| | | | | of CD4⁺ regulatory |
| | | | | T cells |
| Cell | CD80/86 | CD83 (dendritic | Block interaction | At time of |
| activation | (dendritic | cells) | with CD28; | allografting to induce |
| blockade | antigen | | immunosuppression | tolerance. Targeting |
| | presenting | | | to dendritic cells will |
| | cells) | | | enhance efficiency/ |
| Adhesion | VCAM-1 | CD31 | Block VLA-4- | Acute Rx of MS |
| molecule | (activated | (endothelial cells) | dependent T cell | flare-up. Anti-VLA- |
| blockade | endothelial | or E-selectin | and monocyte | 4 is in trial as Rx for |
| | cell) | (activated | adhesion to | MS. |
| | | endothelium) | VCAM-1 on | |
| | | | endothelial cells. | |
| Cell death | CD95L | CD25 (activated | Block activation | Enhance anti-tumor |
| ligand | (Fas-ligand) | T cells) | induced cell death | immunity |
| blockade | (T cell) | | of T cells; | |
| Protect | IFN-_R | CD31 | Cell selective | Block IFN-_toxicity |
| from | (endothelial | (endothelial cell) | block of IFN_{—} | towards endothelial |
| another | cell) | | | cells during IL-2 |
| therapeutic: | | | | therapy for tumors |
| | | | | without impairing |
| | | | | other useful IFN-_{—} |
| | | | | effects |
| Inhibitory | Fc gamma | Fc epsilon RI | Enhance | Rx of acute of |
| receptor | RII | (Mast cells) | association of | allergic disease. |
| activation | (Mast cells) | | activating and | Advantages over |
| | | | inhibitory | bispecific Fc fusion |
| | | | receptors. | reagents because of |
| | | | | more specific cell |
| | | | | targeting. See Zhu D |
| | | | | et al. Nat Med 2002 |
| | | | | May; 8(5): 518-21) |

Candidate targets which may be considered for application of the invention, for which antibodies are known in the art, include adhesion molecules (McMurray R W. Adhesion molecules in autoimmune disease. Semin Arthritis Rheum. February 1996; 25(4):215-33. PMID: 8834012; Dedrick R L, et al. Adhesion molecules as therapeutic targets for autoimmune diseases and transplant rejection. Expert Opin Biol Ther. February 2003; 3(1):85-95. PMID: 12718733; chemokine receptors (Saeki T, et al. chemokine receptor antagonist.Curr Pharm Des. 2003;9(15):1201-8. PMID:12769747. D'Ambrosio D, et al. Panina-Bordignon P, Sinigaglia F. Chemokine receptors in inflammation: an overview. J Immunol Methods. February 2003; 273(1-2):3-13. PMID:12535793; Izikson L, et al. Targeting monocyte recruitment in CNS autoimmune disease. Clin Immunol. May 2002; 103(2):125-31. PMID: 12027417; Westermann J, et al. Migration of T cells in vivo: molecular mechanisms and clinical implications. Ann Intern Med. Aug. 21, 2001; 135(4):279-95. PMID: 11511143; Gerard C, et al. Chemokines and disease.Nat Immunol. February 2001; 2(2):108-15. PMID: 11175802 ; Arimilli S, et al. Chemokines in autoimmune diseases. Immunol Rev. October 2000; 177:43-51. PMID:1 1138783; Horuk R, et al. Chemokine receptor antagonists. Med Res Rev. March 2000; 20(2): 155-68. PMID: 10723026); see also Nelson R P Jr, Immunomodulation and immunotherapy: drugs, cytokines, cytokine receptors, and antibodies. J Allergy Clin Immunol. February 2003; 111(2 Suppl):S720-43. PMID:12592317 [; Hermiston M L, et al CD45: a critical regulator of signaling thresholds in immune cells. Annu Rev Immunol. 2003;21:107-37. Epub Dec. 19, 2001. PMID:12414720; interleukin 1 receptor (see Arend W P et al. Interleukin 1-receptor antagonist: role in biology, Annu. Rev Immunol. 1998; 16:27-55); CCR1 (see Saeki T et al; CCR1 chemokine receptor antagonist; Curr Pharm Des. 2003;9(15):1201-8; and see Elices M J; BX-471 Berlex,; Curr-Opin Investig Drugs. June 2002; 3(6):865-9; and see Saeki T, Naya A., CCR1 chemokine receptor antagonist, Curr Pharm Des. 2003;9(15):1201-8; and see Krishnan B R.; Interleukin-1 receptor antagonist gene therapy for arthritis; Curr Opin Mol Ther. August 1999; 1(4):454-7; and see Chikanza iC; Juvenile rheumatoid arthritis: therapeutic perspectives; Paediatr Drugs>2002;4(5):335-48); interleukin 2 receptor (see Morris J C, Waldmann T A, Advances in interleukin 2 receptor targeted treatment; Ann Rheum Dis. November 2000; 59 Suppl 1:i109-14); CTLA-4 (see Chambers CA et al; CTLA-4-mediated inhibition in regulation of T cell responses: mechanisms and manipulation in tumor immunotherapy; Annu Rev Immunol. 2001;19:565-94); EGF receptor family; (see Mendelsohn J, Baselga J.; The EGF receptor family as targets for cancer therapy; Oncogene. Dec. 27, 2000; 19(56):550-65); Anti-nuclear envelope antibodies (see Nesher G. et al; Anti-nuclear envelope antibodies: Clinical associations, Semin Arthritis Rheum. April 2001; 30(5):313-20); IL-2/IL-15 (see Waldmann T A; the IL-2/IL-15 receptor systems: targets for immunotherapy; J Clin Immunol. March 2002; 22(2):51-6); IL-6 (see Naka T. et al; The paradigm of IL-6: from basic science to medicine; Arthritis Res. 2002;4 Suppl 3:s233-42.); chemokine antibodies and receptor antagonists (see Eng L F et al; Response of chemokine antagonists to inflammation in injured spinal cord; Neurochem Res. January 2003; 28(1);95-100); antibody-directed therapy (see Gelfand E W.; antibody-directed therapy: past, present and future; J Allergy Clin Immunol. October 2001; 108(4 Suppl):S111-6); beta-cell (see Tyrberg B. et al; Current and future treatment strategies for type 2 diabetes: the beta-cell as a therapeutic target; Curr Opin Investig Drugs, November 2001; 2(11):1568-74); natural killer T cells (see Sharif S. et al; Regulation of autoimmune disease by natural killer T cells; J Mol Med. May 2001; 80(5):290-300); vitamin D Receptor Ligands (see Pinette K V et al; Vitamin D receptor as a drug discovery target; Mini Rev Med Chem. May 2003; 3(3):193-203; and see Adorini L., Immunomodulatory effects of vitamin D receptor ligands in autoimmune diseases; Int Immunopharmacol. June 2002; 2(7):1017-28); agonistic anti-Fas monoclonal antibody (see Yonehara S.; Death Receptor Fas and autoimmune disease: from the original generation to therapeutic application of agonistic anti-Fas monoclonal antibody; Cytokine Growth Factor Rev. August-October 2002; 13(4-5):393-402); tool-like receptors (see Leadbetter E A. Et al; Toll-like receptors and activation of autoreactive B cells; Curr Dir Autoimmun. 2003; 6:105-22); anti-IL2 receptor (see Nussenblatt R B; Bench to bedside: new approaches to the immunotherapy of uveitic disease; Int Rev Immunol. March-June 2002; 21(2-3):273-89); soluble IL-6 receptor (see Kallen K J.; The role of transsignalling via the agonistic soluble IL-6 receptor in human diseases, Biochim Biophys Acta. Nov. 11, 2002; 1592(3):3223-43); IL-2 receptor (see Church A C; Clinical advances in therapies targeting the interleukin-2 receptor; QJM. February 2003; 96(2):91-102); CCR5 or CXCR4 (see Pohlmann S.; Evaluation of current approaches to inhibit HIV entry; curr Drug Targets Infect Disor. March 2002; 2(1):9-16); endothelial FGF receptor (see Blanckaert V D et al; Partial characterization of endothelial FGF receptor functional domain by monoclonal antibody VBS-1; Hybrid Hybridomincs. June 2002 121(3):153-9); see also Luger T.; Treatment of immune-mediated skin diseases: future perspectives; Eur J Dermatol. July-August 2001; 11(4):343-7.

The invention also contemplates that FAS can be selectively blocked on various different types of cells such as pancreas beta cells using markers such as GAD65, IA-2, IA2-B, ICA-12. Type 1 Diabetes is characterized by the destruction of insulin producing Beta Cells in the pancreas. One method in which Beta cells are destroyed is thought to be through apoptosis mediated by CD95 receptors on Beta cells. CD-95 seems to be upregulated in Beta cells of those with Type 1 diabetes (see Ann NY Acad Sci April 2002 958 297-304; J Clin Immunol January 2001; 21(1):15-8). Similarly, using Tg, TPO ligands as cell associated markers CD95, TRAILR1, TRAILR2 can be blocked on thyroid cells. Hashimoto's Thyroiditis (HT) is characterized by the destruction of thyroid hormone producing cells and therefore hypothyroidism. It has been observed that some of this cell destruction is due to apoptosis. The CD95 receptor which is responsible for apoptosis is up regulated in thyroids affected by HT. Blocking the CD95 receptor by the RLAA may reduce the amount of apoptosis. The HAA can target either Tg or TPO which are unique to thyroid tissue.¹ There are also 2 other receptors suspected to be involved with apoptosis in thyroid cells: TRAILR1 and TRAILR2 (see Nat Rev Immunol March 2002; 2(3):195-204).
¹ optionally by at least 2, 3, 4, 5, 6, 7, 8, 9, 10, 11, 12, 13, 14, 15, 16, 17, 18, 19, 20, 21, 22, 23, 24, 25, 26, 27, 28, 29, 30-40, 40-50, 50-60, 60-70, 70-80, 80-90, or 90-100 (or more) fold. With respect to each of the foregoing at least 100 increments, the intrinsic off-rate of the targeting moiety is optionally at least 2, 3, 4, 5, 6, 7, 8, 9, 10, 11, 12, 13, 14, 15, 16, 17, 18, 19, 20, 21, 22, 23, 24, 25, 26, 27, 28, 29, 30-40, 40-50, 50-60, 60-70, 70-80, 80-90, or 90-100 (or more) fold less than the intrinsic off-rate of the effector moiety and may be in one non-limiting embodiment in a range from any of the preceding increments to 10,000 or any increments or ranges therebetween.

Fas can also be selectively activated on distinct subsets of disease mediating immune cells associated with autoimmune and inflammatory disorders such as activated T cells, regulatory T cells, CD4+ cells, CD8+ cells etc.

Agonist and antagonist anti-CD95 antibodies are described in the literature. Among other embodiments hereinafter enumerated, the invention is also directed to multifunctional ligands which comprise antibodies which recognize cell type specific markers (hereinbefore or hereinafter exemplified), including those available commercially or published in the art, in any combination with those antibodies (hereinbefore or hereinafter exemplified, including those available commercially or published in the art) that recognize ligands on a broader population of cells including the cell population bearing the cell type specific marker, wherein the instrinsic affinity difference is maintained or adjusted to one, two, three, four, five, six or seven orders of magnitude. The invention contemplates that affinity differences (increases or decreases in affinity) can be readily generated by modifying amino acids that are expected a piori to result in changes in affinity (see for example Chowdhury P. et al. Improving antibody affinity by mimicking somatic hypermutation in vitro (1999) Nature Biotechnology June; 17 p.568-572), or by a variety of other well-known high throughput methods that can readily be applied to this task, for example by light chain shuffling, CDR grafting, parsimonious mutagenesis, shotgun scanning mutagenesis etc. Methods of affinity maturation are well known in the art. (See Huls G, et al. Tumor cell killing by in vitro affinity-matured recombinant human monoclonal antibodies. Cancer Immunol Immunother.(2001):163-71. PMID:11419184.

The invention also contemplates that two hybridoma derived antibodies can be digested e.g. with pepsin to create F(ab′)2 and can be chemically recombined to create bispecific antibodies. Hybridoma fusion technology can also be used to create to tetromas for this purpose. Furthermore, the invention contemplates that hybridoma derived IgG antibodies can be used for the relatively high affinity cell targeting moiety and that hybridoma derived IgMs can be used for the relatively low affinity binding arm. The invention also contemplates that the respective high and low affinity arms can be conjugated, fused etc according to well known methods to respective complementary ligands such as fas/jun strepavidin/biotin (making it possible to pre-target the high affinity arm independently, particularly where the affinity of the complementary ligands for each other is greater than that of the second ligand binding moiety for its target ligand.

According to one embodiment of the invention, the first ligand binding moiety can bind to a cell specific marker and said second ligand binding moiety binds to the extrracellular portion of a ligand involved involved in membrane transport across the cell membrane, for example an ion channel, vitamin receptor etc. to inhibit uptake or export. For example the first ligand binding moiety may bind to a cancer cell specific marker and the second ligand binding moiety can bind to p-glycoprotein. High affinity tumor specific antibodies are well known and anti-PGP antibodies are well known (see for example U.S. Pat. No. 6,479,639 Monoclonal antibody to a human MDR1 multidrug resistance gene product and uses U.S. Pat. No. 6,365,357 Methods and reagents for preparing and using immunological agents specific for P-glycoprotein U.S. Pat. No. 6,171,786 Methods for preventing multidrug resistance in cancer cells U.S. Pat. No. 6,030,796 Monoclonal antibody to a human MDR1 multidrug resistance gene product, and uses U.S. Pat. No. 5,994,088 Methods and reagents for preparing and using immunological agents specific for P-glycoprotein U.S. Pat. No. 5,972,598 Methods for preventing multidrug resistance in cancer cells U.S. Pat. No. 5,773,280 Monoclonal antibody to a human MDR1 multidrug resistance gene product, and uses U.S. Pat. No. 5,434,075 Monoclonal antibody to a human MDR1 multidrug resistance gene product, and uses; Chen Y et al. J Cell Biol March 6; 148(5):863-70; Mickisch G H et al. Cancer Res. 1992 52: 3768-3775 see also Cianfriglia M, Cenciarelli C, Barca S, Tombesi M, Flego M, Dupuis M L. Monoclonal antibodies as a tool for structure-function studies of the MDR1-P-glycoprotein.Curr Protein Pept Sci. October 2002; 3(5):513-30. Tang Y, Beuerlein G, Pecht G, Chilton T, Huse W D, Watkins J D. Use of a peptide mimotope to guide the humanization of MRK-16, an anti-P-glycoprotein monoclonal antibody.J Biol Chem. Sep. 24, 1999; 274(39):27371-8. Zhou Y, Gottesman M M, Pastan I. The extracellular loop between TM5 and TM6 of P-glycoprotein is required for reactivity with monoclonal antibody UIC2.Arch Biochem Biophys. Jul. 1, 1999; 367(1):74-80. Romagnoli G, Poloni F, Flego M, Moretti F, Di Modugno F, Chersi A, Falasca G, Signoretti C, Castagna M, Cianfriglia M. Epitope mapping of the monoclonal antibody MM12.10 to external MDR1P-glycoprotein domain by synthetic peptide scanning and phage display technologies.Biol Chem. May 1999; 380(5):553-9.Vasudevan S, Tsuruo T, Rose D R. Mode of binding of anti-P-glycoprotein antibody MRK-16 to its antigen. A crystallographic and molecular modeling study.J Biol Chem. Sep. 25, 1998; 273(39):25413-9. Naito M, Tsuruo T. Monoclonal antibodies to P-glycoprotein: preparation and applications to basic and clinical research. Methods Enzymol. 1998;292:258-65. Loo T W, Clarke D M. The minimum functional unit of human P-glycoprotein appears to be a monomer.J Biol Chem. Nov. 1, 1996; 271(44):27488-92.). Some anti-PGP antibodies function by interfering with ATP utilization.

It will also be appreciated that a cell specific marker need not differentiate between sub-populations of cells that do not express the second target ligand even though those cells are not the targets of action of the multispecific ligand. Examples of bispecific antibody technologies are described in Appendix C.

Examples of antibodies that bind to cell specific ligands, receptors, etc. are abundant and well known in the art (see for example Biosource International 2002 Research Products Catalog e.g. pages 178-193, Upstate Cell Signalling Solutions 2002 Catalog and other catalogs of well known to those skilled in the art) A list of CD antigens can be found at the NIH (see http://www.ncbi.nlm.nih.gov/prow/guide/45277084.htm) or from the literature (see Zola H et al. Human leucocyte differentiation antigen nomenclature: update on CD nomenclature. Report of IUIS/WHO Subcommittee. J Immunol Methods, Apr. 1, 2003; 275 (1-2):1-8.

Some examples are described in our co-pending Application Ser. No. 2,402,930 filed Sep. 19, 2002 and others are mentioned or referenced throughout the disclosure. See also Appendix D

According to one embodiment of a multispecific ligand of the invention, a first ligand binding moiety which recognizes a cell specific marker on a non-diseased cell is combined with a second ligand binding moiety which binds with a lower intrinsic affinity and/or slower on-rate to a ligand which is the target of a cytotoxic molecule e.g. a radiolabelled cytokine, cytokine-toxin fusion protein e.g. IL-2-diptheria toxin or PE fusion, an immunotoxin etc. The underlying principle of the invention is that such a strategy can be used to protect non-target cells while minimally protecting target cells which do not express the target of the first ligand binding moiety. Following this conceptual approach the invention is more generally directed to first ligands which protect non-diseased cells bearing a corresponding target ligand from the effects of a second cytotoxic ligand (e.g. an immunotoxin, cytokine-toxin fusion protein, radionuclide bearing ligand, etc.) having the same target specificity and which targets diseased cells through the instrumentality of the target ligand e.g. a cancer associated marker e.g. Lewis Y antigen, EPCAM, MK-1 antigen. In this case the first ligand which is intended to bind preferentially to non-diseased cells will be selected to have one or more and preferably all of the following characteristics:

1) as suggested above, a cell-specific binding moiety of higher affinity and/or on-rate which targets the non-diseased cells (e.g. through an epithelial cell marker which is expressed on normal epithelial cells);

2) increased molecular weight, for example an IgG, minibody or other format that does not as readily penetrate solid tumors;

3) through the introduction of enzyme cleavage sites which render the ligand unable to bind to the diseased cells in virtue of specific enzymes differentially expressed in the microenvironment of the diseased cells which can degrade the first ligand, for example in the case of a solid tumors, introducing a cleavage site for a matrix metalloprotease in a portion of the ligand that is essential for maintaining the binding properties of the ligand e.g. the portion of the polypeptide that binds to the cell surface ligand eg. a CDR3 or a more remote location responsible for the ligand's integrity (with respect to such cleavage sites see discussion for example in U.S. patent application 20020103133, WO02/060488, WO02/072620, WO 96/05863, U.S. Pat. No. 5,962,216, WO01/95943, WO02/00263, WO01/68145, WO01/36003, Dubois V, et al. CPI-0004Na, a new extracellularly tumor-activated prodrug of doxorubicin: in vivo toxicity, activity, and tissue distribution confirm tumor cell selectivity.Cancer Res. Apr. 15, 2002; 62(8):2327-31.Fernandez A M, et al. N-Succinyl-(beta-alanyl-L-leucyl-L-alanyl-L-leucyl) doxorubicin: an extracellularly tumor-activated prodrug devoid of intravenous acute toxicity.J Med Chem. Oct. 25, 2001; 44(22):3750-3. Denny W A. Prodrug strategies in cancer therapy.Eur J Med Chem. July-August 2001; 36(7-8):577-95. Trouet A, et al. Extracellularly tumor-activated prodrugs for the selective chemotherapy of cancer: application to doxorubicin and preliminary in vitro and in vivo studies. Cancer Res. Apr. 1, 2001; 61(7):2843-6.Chari R V. Targeted delivery of chemotherapeutics: tumor-activated prodrug therapy. Adv Drug Deliv Rev. Apr. 6, 1998; 31(1-2):89-104).

Definitions

The term “associated” in relation to markers that are dominantly distributed on one or more particular entities is used to mean exclusively expressed, primarily expressed, or over-expressed to advantage from a targeting standpoint.

The term “receptor ligand” means a target ligand which is a ligand for a receptor, for example, a receptor on a cell or infectious agent or a receptor which circulates independently of another entity.

The term affinity is contrasted to functional affinity which may result from avidity.

The term epitope though technically understood to be specific for a given antibody, is used in a preferred embodiments to refer to antigenic determinants that are situated proximally to one another so that two antibodies will be considered to bind to the same epitope if one competively inhibits the binding of the other through any probative competitive inhibition experiment known to those skilled in the art.

The invention contemplates that two antibodies with the same epitope specificity may have a similar amino acid composition ie. with possible exception of one or more additions, deletions or substitutions including conservative amino acid substitutions which do not substantially affect the specificity and amino acid composition of the paratope

The term approximately in the context of orders of magnitude variations in affinity refers a variability that is up to a half an order or magnitude.

Without limiting the scope of the claims it is generally understood that biodistribution of a multispecific ligand in contrast to that of a ligand will be predicated on the bioavailability of its target ligand.

The term “overlap” and related terms connote that notwithstanding the difference in distributions of the first and second ligands the first and second ligands are bioavailable for recognition on the same entity. This term and related terms, exemplified below, are intended to exclude a situation where both ligands are preferentially expressed on substantially the same entity, for example two different tumor associated antigens associated differentially with a differentiated population of cells within a tumor, most particularly in the case where they are individually suitable targets for delivery of a toxic payload. Thus the terms “different” in regard to biodistributions and “heterogeneous” and “diverse” in reference to populations of entities are similarly understood to exclude such a common distribution, in the appreciation that the invention primarily represents an improved strategy for targeting two different ligands, in which one ligand has a broader distribution than the other or both have distributions that may overlap but are different from that of the target population. It will also be appreciated that the invention has particular application to a situation in which at least one of the non-target populations is one on which one of said first and second ligands is substantially represented (in contrast to one on which it simply enjoys limited expression).

The term “receptor ligand” means a target ligand which is a ligand for a receptor, for example, a receptor on a cell or infectious agent or a receptor which circulates independently of another entity.

The term “antigen binding fragment” refers to a polypeptide or a plurality of associated polypeptides comprising one or more portions of an antibody including at least one VH or VL or a functional fragment thereof.

A moiety or molecule that exerts a biologic function is understood to be a “biologic effector” in the sense that its intended interaction in the organism has a biological consequence.

The tern neutralizing in regard to an an immune function is used broadly to refer to any interposition, interference or impediment which affects the function of the target entity

The terms modulating, mediating, neutralizing function etc. are not intended to be mutully exclusive and are each used broadly, for example, without limiting the generality of the scope accorder herein or by those skilled in the art the term modulating preferably refers to effecting a change, and the term mediating preferably connotes an indirect effect achieved through the instrumentality of another entity, for example a cell, cytokine, chemokine etc.

The term “preferentially binds” recognizes that a given ligand binding moiety might have some non-defeating cross-reactivities.

The term biologic effector ligands is used to refer to any ligand for which there is a complementary target ligand on a target entity, and wherein binding of the biologic effector ligand to the target ligand exerts a biologic effect. For example the target ligand is typically a receptor and the biologic effector ligand may be any complementary ligand such as a cytokine, chemokine, hormone, colony stimulating factor, growth factor, receptor inhibitor, agonistor antagonist, which binds to the receptor with resulting biologic effect. As defined above, the term “pre-selected” in reference to the affinity of ligand binding moiety refers to any selection or choice of differential or cooperative affinities relative to a second ligand binding moiety which is generated as a result of a mental or physical process or both, preferably through a process of prediction or post-facto validation of the effects of the choice of the first and second affinities and/or more preferably through an empirical evaluation of different choices for at least one of the first and second affinities, and preferably both.

The term multi means at least two and the term ligand is used broadly to refer to any entity or part thereof which can participate in an intermolecular interaction that can result in specific binding of suitable affinity for the interaction in question.

The term entity includes without limitation any molecule including without limitation, antibodies, complex or association of molecules, drugs, drug carriers (eg. vesicles eg. liposomes, nanoparticles, etc.) or any cell as well as any infectious agent or parasite (including, without limitation, spores, viruses, baceria, fungi) as well as any other immune or therapeutic target.

The term “low affinity” means an affinity of approximately (this term is defined herein) 10⁻⁴ molar to micromolar affinity, preferably (subject to safety considerations), approximately, 10⁻⁵ molar affinity, more preferably (subject to safety considerations), approximately micromolar affinity, the term “medium affinity” means approximately 10⁻⁷ to nanomolar affinity, preferably approximately 10⁻⁸ molar affinity (in most cases, dependant on the affinity differential, “preferably” only if the affinity in question is that of the first ligand binding moiety), more preferably approximately nanomolar affinity, and the term “high affinity” means approximately 10⁻¹⁰ affinity or greater. Thus is one embodiment the invention contemplates that the multispecific ligand comprises a “target-ligand” binding moiety (the second ligand binding moiety) which binds with low or medium affinity to a target ligand present on a diverse population of cells (preferably this moiety is an effector moiety ie. one which exerts a biological effect attributable to its binding eg. blocking or activating a receptor or blocking a cell membrane channel) and a “targeting”, first ligand binding moiety, which binds with medium or high affinity to a ligand associated with a sub-population of those cells, so as to bias the biodistribution of the multifunctional ligand in favor of said sub-population. In one non-limiting embodiment, the multi specific ligand is adapted to be bound contemporaneously to the same cell. In another unrelated embodiment the first and second ligands binding moieties each bind to ligands present on diverse overlapping populations of entities eg. cells (ie. neither ligand being preferentially associated with a target cell population) and are adapted to be bound contemporaneously and to both bind individually with low affinity, so as to bias the distribution of the multispecific ligand to the population of cells bearing both ligands.

As discussed elsewhere the term approximately, in reference to “order of magnitude” increments in affinity, refers to up to a half order of magnitude in affinity.

According to another embodiment, the invention is directed to an antibody termed a “coybody”. A “coybody” is an antibody in which the on-rate contribution to the affinity of the antibody is proportionally less than the off-rate contribution relative to a reference antibody of the same specificity and a greater affinity of up to several orders of magnitude, preferably a reference antibody of approximately one to three orders of magnitude greater instrinsic affinity, preferably a reference antibody of medium affinity or preferably high affinity, optionally a reference antibody with an optimally high on-rate, for example in which the intrinsic affinity or intrinsic on-rate has been optimized after an antibody of the desired specicity has been generated. With respect to making high affinity antibodies with optimal properties see for example WO 01/55217, WO 03/074679, Ewert S, et al. Structure-based improvement of the biophysical properties of immunoglobulin VH domains with a generalizable approach.Biochemistry. Feb. 18, 2003; 42(6):1517-28. Ewert S, et al. Biophysical properties of human antibody variable domains. J Mol Biol. Jan. 17, 2003; 325(3):531-53. Optionally, the reduction in on-rate is brought about by modifying amino acids that are known or engineered to so as contribute minimally to the specificity and slow off-rate of the antibody. Typically, the reference antibody may be derived from a hybridoma or from a phage, yeast bacerial or ribosome display library. As discussed above the reference antibody optionally comprises cooperating light and heavy variable regions in which 2 of 6, 3 of 6, 4 of 6 or 5 of 6 of the CDRs, optionally CDRs of both the VH and VL, including preferably at least one CDR3, optionally that of the heavy chain, are collectively made or selected to be primarily or exclusively responsible for the binding affinity of the coybody, such that alterations in the length (e.g. by additions of one or more amino acids) or amino acid composition, optionally by a parsimonious mutagenesis procedure (e.g. where the library is optionally partially biased in favour of the parental amino acid composition to introduce change more incrementally) of one or more other non-contributing or minimally contributing CDRs can be leveraged to diminish the on-rate, for example due to steric and/or electrostatic hindrance. For example, combinatorial libraries in which one more amino acids for example amino acids corresponding to locations of the greatest variability in residue identity for example by a Kabat-Wu plot (see Cellular and Molecular Immunology 4^{th} Ed. ISBN 0-7216-8233-2 page 49) or greatest probability of contact with the target ligand as determined by three dimensional modeling, alanine scanning or high throughput mutagenesis may be randomized or partially randomized in the one or both of the CDR1s and CDR2s or in one CDR1 and one CDR2 to permit selection of an antibody with a reduced intrinsic on-rate. In one embodiment, such contact or potentially affinity contributing amino acids may have been deleted or modified to residues with reduced capacity for intermolecular interaction in the initial selection of the relatively high affinity reference antibody or in a later step after generation of the parental template with the desired specificity but prior to a subsequent affinity maturation step to generate the reference antibody. This approach among others may be used to set up a scenario in which select portions of one or more CDRs are modified to organize that they contribute minimally to the specificity and affinity (slow off-rate) of the antibody. In one embodiment, in the case of a single domain antibody at least one and preferably two of the CDRs are primarily or exclusively responsible for the binding. In one embodiment the on-rate is reduced by a factor of 2 to 100×. In one embodiment the coybody binds to a ligand which is over-expressed on a target population of entities (eg. cells) relative to a non-target population of entities such that the biodistribution of the coybody to the non-target population (and target population) is diminished in a given increment of time following administration. This targeting strategy is understandably adapted to situations where the resulting delay in biodistribution is preferable for diminished toxicity attributable to reduced non-target entity binding in a given unit of time especially where the effectiveness threshold in that same amount of time is not significantly if at all compromised or is preferable due to a sustained release effect (for example using a larger antibody format that is not readily cleared). Examples of suitable targets for cobodies per se include hormones or other secreted growth regulatory factor, differentiation factor, or intercellular mediators (e.g., cytokine) such as TNF alpha, FAS L, IL-8, IL-6, IL-5, VEGFs, fibroblast growth factors. Numerous examples of antibodies which recognize such targets are described in patents classified in US Class 424/145.1 (see Appendix A). Methods of prolonging the half-life of antibodies, producing bispecifics, scfvs and dsFvs and altering Fc effector function are well known and noteworthy references include U.S. Pat. No. 6,277,375, U.S. Pat. No. 5,869,046, U.S. Pat. No. 5,624,821, U.S. Pat. No. 6,096,871, U.S. Pat. No. 4,479,895, U.S. Pat. No. 6,207,804, U.S. Pat. No. 5,681,566, U.S. Pat. No. 5,864,019, U.S. Pat. No. 5,869,620, U.S. Pat. No. 6,025,165; U.S. Pat. No. 6,027,725; U.S. Pat. No. 6,239,259; U.S. Pat. No. 6,121,424; WO00/09560; U.S. Pat. No. 6,420,140.

As discussed below, advantages accrue when this antibody is coupled to a higher affinity antibody (in the form of a multifunctional ligand) which binds to a different ligand associated with the target population. The invention contemplates that coybodies have multiple independent applications, including tempering the effects through antibody mediated neutralization of an over-production or sensivity to biologic effector ligands (eg. cytokines eg. TNFₐₗₚₕₐ, chemokines eg. IL-16 (crohns disease) etc. which are over-produced and/or mediate or aggravate eg. a chronic medical condition (which for example is not an acute phase) by binding to such ligands, over a prolonged periods, preferably using larger antibody formats which are not readily cleared, especially where such tempering has side effects which are better spread over time and/or where effectiveness is not a limiting factor and/or where a second therapeutic with different non-cumulative side-effects shares the therapeutic burden and/or where a the same antibody with a higher on-rate is used in combination.

Accordingly, in one embodiment the affinity of the first ligand binding moiety is comparable (equal to or no greater than one order of magnitude less that than, preferably no greater than five times less, preferably no greater than four times less, preferably no greater than three fold less, preferably no greater than two fold less etc.) or preferably greater than the affinity of the second ligand binding moiety and the on-rate of the first ligand binding moiety is greater than that of the second ligand binding moiety. Testing and selection of binders with high on-rates via BIACORE or other methods of on-rate measurement are well known in the art (with respect to making and evaluating high on-rate antibodies, see particularly WO 01/64751, WO 03/068801, WO 02/36615, US 2002/098189, US 2002/164326 ; see also WO 01/55217, WO 01/27160, and with respect to making high affinity and improved antibodies generally see WO01/55217 and WO 03/074679). It will be appreciated that best safety/effectivenss profile will be achieved by selecting the relative on-rates and/or off-rates of both binding moieties. For example: in one embodiment the on-rate of the first ligand binding moiety is five to 500 fold greater that the on-rate of the second ligand binding moiety (including all the increments therebetween), optionally one to two orders of magnitude greater (including all the increments therebetween). The on-rate of the first ligand binding moiety may also be 2, 3 or 4 times greater than the on-rate of the second ligand binding moiety. In any of the preceding examples the intrinsic affinity of the first ligand binding moiety is preferably anywhere between 2 to 100,000 times, optionally 5 to 1000 times, greater that of the second ligand binding moiety.

The term “antibody” is used broadly, unless the context dictates otherwise, to refer without limitation, to a whole antibody of any class or biologic origin, or chimeric combinations of antibody regions or domains (eg. FRs and CDRs) of different origins or species eg. humanized, any combination of one or more antibody fragments or recombinant reconstructions (scFvs) of antibodies including dimers, diabodies, triabodies, a myriad of known bispecific, trispecific, tetraspecific antibody formats or monovalent, divalent, trivalent, tetravalent or other multivalent antibody formats (see for example review in Kriangkum J, et al. Bispecific and bifunctional single chain recombinant antibodies. Biomol Eng September 2001; 18(2):31-40 and others herein directly or otherwise referenced) or any fragment, portion, or reconstruction of one or more portions of an antibody (scFv) or any truncated form a ligand binding entity, such antibody typically comprising at least a VH or VL portion or both or a functional portion of same (eg microbodies), including single domain antibodies, F(ab′)₂, Fab, Fab′, Facb, Fc, etc. The term antibody also includes fusions of such an antibody so defined and other functional moieties (eg. toxins, cytokines, chemokines, streptavidin, adhesion molecules).

According to one aspect, the invention is directed to a multispecific ligand with at least two different binding specificities for different target ligands on the same target entity eg. a cell and which is preferably adapted to bind contemporaneously to (ie. there are no geometric or other constraints which preclude both moieties from functionally interacting with their respective target ligands at the same time)the different target ligands, said multispecific ligand comprising a first target binding moiety which preferentially(some cross-reactivity(s) does not preclude the utility of the invention) recognizes a first target ligand and a second target binding moiety which preferentially recognizes a second target ligand, and wherein the ability of the second target binding moiety to bind to the second target ligand is diminished relative the ability of the first target binding moiety to bind to the first target ligand, the first target binding moiety having an ability to bind to the first target ligand which is at least sufficient for the first target moiety to bind to the first target ligand independently of the second target binding moiety binding to the second target ligand and an off-rate (with respect to the first target ligand) which at least sufficiently exceeds the on-rate of the second target binding moiety for the second target ligand to at least provide opportunity for the second target moiety to bind the second target ligand when the first target binding moiety is bound to first target ligand, the second target binding moiety having a relatively diminished ability to bind and/or stay bound to the second target ligand independently of the binding of the first target binding moiety to the first target ligand (such that a plurality of the multispecific ligand will bind to a population of cells bearing both target ligands in preference to a population of cells bearing only the second target ligand (ie. at least in part due to the first target binding moiety assisting (ie. providing opportunity) the second target binding moiety to bind to the second target ligand and preferably out of proportion to what could be statistically attributed to the presence of two targets ligands on the target cell eg. the binding of the first target binding moiety providing necessary assistance for the second target moiety to bind is relatively increased (ie. relative to the situation where both of are of comparable affinity).

It will be appreciated that relative number of bioavailable second target ligands relative to the number of the bioavailable first target ligands will influence the selection of affinities of the first and second target binding moieties. For example, from the standpoint of safety, the affinity of the first target binding moiety for the first target ligand may well be sufficient if initially approximating nanomolar affinity and the affinity of the second target binding moiety for the second target ligand will be selected to limit the number of effective binding events on the population of cells bearing only the second target moiety; an affinity which can be predicted to proportional to the number of bioavailable second target ligands on the population of cells bearing only the second target ligand ie. the non-target population (relative to the number of first target ligands on the target population of cells). For example, this may be assessed by determining the amount of labelled multispecific ligand on the target and non-target populations of cells in vivo (or in vitro where the number of bioavailable first and second target ligands can be roughly estimated). This selected affinity, from a effectiveness point of view, will then be assessed as to whether it is sufficient for the second ligand binding moiety to bind to the second target ligand on the target population of cells, with the benefit of the first ligand binding moiety bound or having been bound to first target ligand. For example, where the binding of the second target binding moiety may be assessed through an in vitro assay (eg. an assay in which the blocking or activating of a receptor is measurable eg. through inhibition of binding of the natural ligand for a target receptor or through some measurable parameter associated with effective binding for example the release of cytokines or other biologic effector ligand. The effect of binding may be also be assessed by comparing the effects over time relative to a higher affinity second binding moiety which is not associated with a first ligand binding moiety. It will be appreciated that a more ubiquitous second target ligand may require selecting a higher initial affinity of the first target binding moiety for the first target ligand eg. picomolar affinity, and selecting an affinity of the second target ligand which may for example be of micromolar affinity plus/minus approximately one order of magnitude. It will also be appreciated that the deleterious effects of non-target cell binding will vary as will the degree to which the first target ligand is uniquely found on the target population of cells. In the final analysis a suitable difference in affinity between the two binding affinities may well be at least, approximately, one, two, three, four, five, six, seven or eight orders of magnitude. In this connection the term approximately refers to ±up to a half order of magnitude (<5×). As discussed below, the invention contemplates that variants of a dual affinity multispecific ligand may be assessed in a high throughput screen or series of such screens with a view to selecting a variant that has one or more predefined properties, alluded to above such as a) the ability to mediate a biologic effect on a target population relative to a negative control; b) the ability to mediate an improved or diminished biologic effect on a target population relative to a positive control. This ability may also be assessed in a competition experiment of any probative type well-known to those skilled in the art; c) the inability or diminished ability to mediate a biologic effect on a non-target population relative to negative and positive controls. Such diminished ability may be also assessed in a competition experiment of any probative type well known to those skilled in the art; d) the ability to target a target population through binding relative to controls and in a competition; e) the inability or diminished ability to target a non-target population relative to controls and in such competition experiement. The invention also contemplates that the multispecific ligand may bind to a ligand which is cell specific in the sense that it binds to cells to which it has been delivered by prior administration (eg an antibody or fusion protein thereof which only binds to the target cells or at least to cells which do not have the ligand recognized by the second ligand binding moiety present in any significant amount), akin to the pre-targetting strategies well known in the art. For example, this strategy could be used to increase the number of first ligands relative to second ligands, where indicated.

In one embodiment, said first target binding moiety recognizes an entity-associated ligand eg. a target cell-associated target ligand, for example a ligand which is exclusively expressed, primarily expressed or over-expressed to advantage on the target cell population and said second target binding moiety recognizes a non-target cell-associated target ligand which is present on target cells and non-target cells, for example a receptor, including a decoy receptor eg. for TRAIL. The multispecific ligand is thereby adapted to block or activate the receptor primarily on the target population of cells. In this connection, the invention is also directed to methods of evaluating or implementing the effects of this enhanced selectivity for the receptor on the target cell population and can be employed to diminish the adverse consequences and evaluate the benefits associated with using a ligand binding moiety that would otherwise undesirably bind to receptors on non-target cells.

The invention contemplates that a variety of different strategies that can be used alone, or in any variety of compatible permutations to differentiate between target cells and/or between target and non-target cells. The choice of strategies, may depend at least in part on the circumstances, including the nature of the fluid environment in question, including the rapidity and pressure of flow and the direction(s) of this pressure, the method of delivery, the medical condition for which the molecule is being evaluated, whether the target is moving or stationary, or both, the location or various locations of the target, the targeting venue or venues that is/are most effective and the importance of the size of the molecule for reaching the target as well as bioavailablility, and the importance of creating immunoconjugates and immunofusions with other molecules (insofar as this affects the size and distribution of weight in the molecule). The invention contemplates that employing more than one type of construct may be desirable and the invention is therefore directed to the various combinations and permutation of constructs according to the invention, in combination with each other and other therapeutic molecules or modalities. One of constructs contemplated by the invention, is a multispecific antibody, for example a bispecific antibody having a configuration which allows for binding to two antigens on the same cell, for example a traditional four chain immunoglobulin configuration having a hinge region (including F(ab′)₂ minibodies etc.), a diabody configuration (depending on the relative positions of the target ligands) and others herein referenced and known to those skilled in the art. It will also be appreciated that the mode of action of the multifunctional ligand may be contributed to by fusing or conjugating the multifunctional ligand to another functional moiety, for example, as described in the literature referenced below. These supplementary strategies are set forth below:

Additional Strategies For Modifying Targeting Capabilities

According to one embodiment, the intrinsic affinity of the first target binding moiety for the first target is greater than the intrinsic affinity of the second target binding moiety for the second target. The term “intrinsic” affinity connotes a measure of the affinity of a given target binding moiety for its target ligand which is independent of the affinity of the at least one other target binding moiety for its target ligand and as used herein could theoretically be evaluated in the context of the multi specific ligand as a whole, if the other target binding moiety had an irrelevant specificity and therefore could not bind to its target ligand. The invention contemplates that at least approximately one, two, three, four, five, six, seven or eight orders of magnitude differences in “intrinsic affinity” may be required to accomplish the targeting objectives of the invention.

According to another embodiment, the relative on-rate of the first target binding moiety is greater than the relative on-rate of the second target binding moiety. The term relative on rate is used to connote an effective difference in on-rate that may be instrinsic to the individual target binding ligand or may attributable to its configuration or relationship vis-á-vis other parts of the molecule.

Where the intrinsic on-rate⁷ of the first target binding moiety is greater than the intrinsic on-rate of the second target binding moiety, the invention contemplates that the off-rate contribution to the affinity of the second target binding moiety may be proportionally greater than the off-rate contribution to the affinity of the first target binding moiety. The invention contemplates that the binding of the second target ligand binding moiety to its target ligand may be more effective if its lower affinity is attributable in part due its reduced on-rate. The invention contemplates methods for reducing the affinity a target binding moiety by reducing its on rate for example by mutating or adding amino acid residues in regions of the VH or VL that don't directly contribute to the off-rate (of a relatively high affinity binder for the target, for example, as determined by modeling and structural analysis, for example, by evaluating x-ray crystal structure and evaluating NMR data of the binding, or by mutagenesis, preferably by introducing a diversity of changes in a high-throughput manner (e.g. phage display, ribsome display, microarray or other expression library) including substitutions, additions and deletions within various regions of the VH or VL and determining their effect. For example, the invention contemplates that the second target binding moiety is generated using a library characterized by members in which one of the regions of VH or VL, including particularly the CDR1 and CDR2, for example the CDR1 of the VH or CDR2 of the VL, is shortened and/or mutated in a manner to reduce the probability of its having any direct contribution to the affinity of the selected molecule (through molecular interaction), for example mutated to introduce amino acids that are least important for intermolecular interactions, for example by minimizing the occurrence of amino acids that are important for electrostatic interactions and optionally also hydrogen binding, generating a binder whose affinity will be postulated to be independent of the contribution of the modified CDR, and then optionally evaluating the success of this latter step through further mutagenesis (this step is most revealing if the CDR is shortened but not mutated or mutated to introduce amino acids important for intermolecular interactions) and then using the library to incrementally lengthen the region and/or introduce amino acids important for intermolecular interaction at a distance (eg. electrostatic interactions and optionally also hydrogen binding) to introduce minimal steric hindrance or intermolecular repulsion. The invention also contemplates that introducing amino acids that have the greatest potential for hydrogen bonding may introduce an aqueous cushion into the interface region with the target ligand to diminish the on-rate contribution to affinity. The invention also contemplates modifying the amino acid composition of an existing binder by introducing or one or amino acids or mutations into a framework region at a location which is proximal to the binding region or a region which borders the interface of approach to the binding region or any interface between the target binding moiety and the target ligand. The invention contemplates that the on-rate and off-rate can be routinely measured using various technologies (eg. Biacore) known to those skilled in the art, including various techniques of measuring these rates in real-time, for example those that measure the deflection pattern of an incident form of radiation (eg. Biosite). In one embodiment of the method the antibodies each have unique preferably cleavable peptide tags that are generated for example through a random or partially random insertion of nucleotides into the DNA encoding the antibody and that serve to link them to their DNA eg a phage (as per techniques known to those skilled artisans or published in the art) and the antibodies are evaluated independently of a phage (eg. they may even be cleavable from the phage) or other expression system linkage which allows a more accurate measure of their true on rates and off-rates. The invention also contemplates that FR1 could be lengthened in a relatively high affinity second target binding moiety to reduce its on rate. The cleanable peptide could be a unique identifying CDR.
⁷ The actual on-rate if the on-rate was to be measured independently of the on-rate of the other binding moiety

In another aspect the invention contemplates that the multispecific ligand may comprise an Fc portion and a hinge portion and that one or both of a) the length, amino acid composition or* molecular weight (or various combinations of these interrelated factors) of the Fab or Fc portion; and b) the amino acid composition (including length) of the hinge portion (eg. any polypeptide segment that provides means for linking two typically heavy chains, eg. through one or more disulfide bonds, leucine zipper fos-jun, optionally a flexible hinge typical of an IgG1 or having one to several more disulfide bonds eg. IgG3) are selected to reduce the circumstantial (shear rate, presence of degrading enzymes) affinity of the second ligand binding moiety where the first ligand binding moiety is unbound relative to the circumstantial affinity of the second ligand binding moiety where the first ligand binding moiety is bound. The term circumstantial affinity broadly contemplates that the length and molecular weight of the Fc and the flexibility of the hinge region will individually and collectively contribute to the affinity of the molecule in proportion the shear rate of the fluid environment to a degree depending on whether the target is stationary or moving, once the multispecific ligand is bound. If bound via the second target binding moiety, any increase in the molecular weight especially a distribution of the molecular weight towards the Fc or first ligand binding moiety will serve as a lever in a moving fluid environment, to favor disengagement from binding especially since the off-rate of this binding arm is relatively low to begin with. This same lever effect will impinge on the binding of the first ligand binding moiety but to a lesser functional degree due to its higher affinity. To an extent depending on the context in which binding occurs, the invention also contemplates that the high affinity ligand binding moiety will draw the multispecific ligand from the circulation into a desired target tissue and that the low affinity binding arm will then have greater opportunity to bind even if it does not bind simultaneously with the high affinity binding arm. Where the hinge region is extra flexible or has several regions of flexibility (for example where the heavy chains are linked through several disulfide bonds with regions of flexibile linker therebetween) the disengaging effect on the individual and paired binding of both the first and second ligand binding moieties will be less Similarly, using a truncated Fc portion (CH3 deleted, F(ab′)₂ or minibody format) will assist the first ligand binding moiety to remain bound or foster binding of the second ligand moiety and will assist the second ligand binding moiety to remain bound. This construct may be preferred from an effectiveness standpoint (getting both ligand binding moieties bound), where the affinity of the second ligand binding moiety is low to begin with. On the other hand, decreasing the flexibility of the hinge region by alteration to its length and/or amino acid composition and increasing the molecular weight distribution towards the “free” end of the Fc will affect all binding scenarios to a greater extent. The latter strategy may be less desirable where the Fab of the first ligand binding moiety is lengthened (eg. has a longer hinge region at the N-terminus of the disulphide bond linking the heavy chains, than the low affinity binding arm) to increase its propensity for individual binding. For example, in a conventional four chain or heavy chain antibody (two heavy chains but no light chains) the hinge region could be lengthened or shortened on the amino terminus side of the disulfide bond linking the heavy chains to an extent that does interfere with the simultaneous binding to both the first and second target binding moieties. The invention also contemplates that the target cell environment, naturally or through intervention, is a fluid environment (low shear rate) or enzyme environment which will favor a greater impact on disengagement of the second ligand binding moiety, in the case of an enzyme, one which will cleave off an Fc into which a cleavage site has been introduced so that disengagement due to the lever effect will primarily impinge on binding of the second ligand moiety to the non-target cell population (eg. low shear rate or presence of MMP type enzymes in a targeted solid tumor environment).

The invention also contemplates that second ligand binding moiety may be selected in an environment in which there is a selective pressure (moderate fluid flow eg. using live cells or tissue, candidate ligand binding molecules or pairs of the target ligands on latex beads, where the substrate to which they are bound is on an incline or otherwise subject to fluid flow (optionally with rigid or high mol. weight Fc), for simultaneous binding so that the affinity of the second ligand binding moiety is selected on the basis of its ability to augment the binding affinity of a first ligand binding moiety of preselected affinity for the first target ligand (after or optionally before its affinity maturation, depending on the shear force and affinity in question) and thereby augment the affinity of the multispecific binding ligand as a whole, while the first ligand binding moiety is bound. In this way, the strength of the binding affinity of the second ligand may be predicated on the first ligand moiety being bound. The foregoing strategy may have accentuated or at least equal application where the first ligand binding moiety has a longer Fab or for example where both the first and second ligand binding moiety are devoid of a light chain i.e. where having the correct binding interface for the second target binding moiety might be more acute. The invention contemplates that the individual affinity of second ligand binding moiety selected in the above manner would be tested to ensure that its individual affinity was not sufficient for substantial independent targeting.

The invention also contemplates that engineering a suitable affinity antibody for solid tumor targeting in which the on-rate contribution to affinity is reduced (according to the strategy suggested above) may assist a dose of such antibody in achieving better tumor penetration. An antibody having a reduced on rate could be fused to a toxin such as a truncated version of PE or conjugated to a radionuclide, etc. the reduced on-rate contribution ensuring that the antibody will be less likely to bind at sites proximal to the point of entry to relieve congestion in that area and better ensure its diffusion throughout a tumor. The invention contemplates that the strategies decribed above will better permit the affinity to be more suitably apportioned between the on-rate and the off-rate. The invention contemplated that a higher on-rate lower off-rate Ab could be delievered in alternating days or other cycles of treatment. Thus the invention is directed to an antibody conjugated or fused to a functional moiety, wherein the on-rate contribution to the affinity of the antibody is anywhere between 3x and two order of magnitudes less than typical molecules having suitable properties for tumor penetration through diffusion, for example molecules having anywhere (any increments) between 10⁻⁷ and 10⁻¹⁰ molar affinities (eg. 5×10⁻⁷, 3×10⁻⁸) preferably increments between 10⁻⁸ to 10⁻¹⁰ (molecules where the on rate is normally approx. 10⁻⁵) molar affinities, more preferably increments between 5×10⁻⁸ and 5×10⁻⁹.

It will be appreciated that the foregoing strategies could be employed for designing a multispecific ligand which will primarily target cells which have both the first and second target ligand (eg. where the ligands together are present primarily on the target cell population) even where neither target ligand is individually found primarily on the target cell population, by employing a multi specific ligand in which neither target ligand is of sufficient affinity in the circumstances to effectively (with effect) bind or remain bound without the other target ligand being available for simultaneous binding. As suggested above, it will be appreciated that a relatively higher affinity ligand could initially be employed on one of the ligand binding arms to select a second ligand binding arm which improves the binding properties of the multispecific ligand under a suitable biologically relevant shear stress and which is selected or later modified so that it is individually insufficient for targeting its target on non-target cells in the circumstances in which it will be employed, and that the high affinity ligand binding arm can subsequently be reduced to moderate affinity with similar lack of individual effect. In one embodiment, this construct can be employed to evaluate the effect of blocking two receptors on the same cell, for example chemokine receptors eg. CCR7 and CXCR4 on a breast cancer cell. In one embodiment, the off-rate of one or optionally both ligand binding moities is sufficient in the circumstances to permit the moiety to remain bound for a sufficient duration for the other moiety to bind ie. it exceeds its effective or intrinsic on-rate. In one embodiment, both arms of such multispecific ligand, bind to their respective ligands with low affinity. In one embodiment, one such arm is a “coybody”.

In connection with the foregoing and ensuing strategies it will also be appreciated that the hinge region may be lengthened on the N-terminal side of the most N-terminus linker between the heavy chains so as to permit greater flexibility in the binding of different antigens at different possible proximities to one another.

The invention also contemplates that the two heavy chains of an IgG (with or without light chains and/or CH1/CL domains), minibody/F(ab′)₂ (with or without light chains and/or CH1/CL domains), may be linked (whether they have a full size or fully truncated Fc or elongated hinge regions) through a flexible peptide linker (such as used for making scFvs i.e. multiples of gly4ser) in order to ensure correct pairing of the heavy chains by expressing the linked heavy chains in E. Coli, for example, as inclusion bodies, which are refolded in refolding solution according to well established techniques in the art. In a construct employing light chains, the light chains may be linked through a disulphide bond linking according to well known methods of making disulphide stabilized Fvs (dsFvs) and the same light chain may be employed for both the high and low affinity arms.

With respect to each of the preceding aspects of the invention, the invention also directed to a multispecific ligand comprising a first ligand moiety which recognizes a first target ligand that is over-expressed on a disease associated entity for example a diseased or disease-causing or mediating cell or infectious agent and a second ligand binding moiety that recognizes a target ligand and wherein the first target ligand is characterized in that it does not lend itself to facilitating or permitting internalization of the second ligand binding moiety.

The invention also contemplates that a target ligand can be distributed in various concentrations for testing purposes on cell sized latex beads, columnar packing materials or flat substrates having a high density dispersion of both target ligands.

The invention is also directed to combination therapies with the foregoing multispecific ligands including, without limitation, immunotoxins, drugs, therapies with other multispecific ligands herein described and particularly for cancer therapies directed at interfering with the integrity of tumor cell vasculature.

Delivering Biologic Effector Ligands to a Target Entity

With respect to each of the preceding aspects of the invention, the invention also contemplates that the second ligand binding moiety may be constituted in whole or in part by a ligand which binds to a biologic effector ligand (such as a cytokine, colony stimulating factor, chemokine, growth factor etc. or related extracellularly expressed regulatory molecules that control their expression such as inhibitors, agonists, antagonists of same, which may have corresponding biological receptors), the ligand optionally having a higher affinity for the biologic effector ligand than the affinity of that biologic effector ligand for its receptor, and wherein the ligand, combined with the biologic effector ligand (ie. bound thereto), has a relatively diminished ability to bind and/or stay bound to the receptor (the second target ligand) independently of the binding of the first target binding moiety to the first target ligand eg. a lower affinity of approximately one, two, three, four, five, six, seven or eight orders of magnitude. The invention contemplates that the foregoing construct can be used to deliver the biologic effector ligand more selectively to the target cell population recognized by the first ligand binding moiety. The second ligand binding moiety may be an antibody portion of a multispecific ligand of the invention and the invention contemplates that a library of second ligand binding moieties, recognizing multiple different epitopes on the biologic effector ligand, can be screened for their ability to bind to the biologic effector ligand, while it is bound in situ to its receptor, for example, using a microarrary of such antibodies, and the affinities of the binders can be evaluated. The invention also contemplates that suitable antibodies could be generated by “panning” (with an expression library, e.g. phage display, ribosome display, or other similar display systems including yeast, bacterial, viral, cell based or cell-free display systems) or otherwise screening (eg. using antibody microarrays) against the biologic effector ligand while bound to its receptor and screening for their ability to bind to the biologic effector ligand independently of its receptor. Again, the affinities of the antibody coupled to the-biologic effector ligand for the target receptor could be evaluated. More generally, the invention contemplates that an array of antibodies which recognize all different epitopes on a given biologic effector ligand could be generated and tested for their ability to accommodate binding of a biologic effector ligand to a first but not a second in a related family of receptors. This could be accomplished by screening the array for one or more members that bind to the biologic effector ligand (BEL) while bound to its receptor, and testing the identified members for their ability to bind to the second receptor, preferably by loading the biologic effector ligand onto an array of those members pre-bound with BEL and detecting those BEL bound members for those which do and do not bind to the second receptor. Therefore the invention is also directed to an antibody which accommodates binding of the BEL to one receptor but hinders the binding to at least one second receptor, preferably by steric, charge or other inter-molecular hindrance, attributable to the proximity of the antibody epitope on the BEL to the BEL's receptor binding site and optionally also the amino acid composition of the antibody at that interface.

The invention contemplates that fluid flow can be simulated in a purification or immunoaffinity column packed with one or more known packing materials to simulate flow over a ligand coated substrate.

The invention also contemplates an apparatus and method for testing ligand binding in a circulating fluid environment in which the multispecific ligands of the invention can be tested and wherein a continous flow of ligands, including target ligands, ligands of the invention and/or ligand bearing entities (eg. cells or synthetic eg. latex spheres which can be adjusted to a cell size) to which one or types of ligands have been affixedly associated accordingly to known methods) can be generated. The fluid contact interface of the apparatus has a generally circular shape and is convex or otherwise capable of containing the fluid and thereby preferably permits fluid to flow around the surface continuously. For example, this surface may be enclosed with a bagel-shaped cylinder which is optionally open at a location opposite the fluid contact surface for introducing and/or removing its contents, or it may completely enclosed with the exception of an access port, from which any air may optionally be displaced or evacuated. The invention contemplates that the apparatus (at least the fluid contact vessel) can be rotated or oscillated (eg. in an elliptical, oval or similar shape well known to those skilled in the arts of fluid mechanics and related engineering arts) in a variety of different planes or with rocking-like motion in multiple planes or subject to peristaltic pressure (ie. where flexible tubing is used) to generate a continuous, optionally turbulence free fluid flow over the fluid contact surface at selected rates simulating the various shear rates of arterial, venous, intra-lymphatic flow (including different diameters of such vessels) or interstitial flow. The invention also contemplates that the fluid contact surface may be provided with a 1) substrate for linking ligands of the invention or target ligands or ligand bearing entities to permit fluid flow across the substrate in a plane substantially parallel or conforming to the axis of flow.

In another aspect the invention is directed to methods of making a multispecific antibody in which:
a) the light chains are the same for both the VL domains. For example, the light chains (assuming the construct has two light chains) are generated for a first target binding moiety eg. in one aspect of the invention, the relatively high affinity binder, optionally from a light chain germline sequence, and this light chain is then coupled with a diversity of heavy chains to select a pair of chains which bind to the second target ligand, thereby constituting the second ligand binding moiety, which may be a relatively low affinity binder. An alternate or concomitant strategy to generate a lower affinity second ligand binding moiety would simply be to substitute the light chain of the first ligand binding moiety for that of the second ligand binding moiety and to test the affinity. In the case of a multi specific which target BELs to particular target cells, where for example, two high affinity binders are preferred, the heavy chain and light chain binding to the BEL can be truncated correspondingly at the CH1/CL region so that the VH/VL interfaces and cysteines pairing these heavy and light chains are similarly spaced but spaced differently from the other VH/VL chains. By linking the heavy chains as explained above, all chains will pair correctly. It will be appreciated that the foregoing production strategies could be applied to the production of heavy chain antibodies (two chains structures without associated light chains), wherein the heavy chains are from human or other species and that production in this case could be adapted to E. Coli. It will also be appreciated that deletion of a substantial part of the CH1 and CL domains can be measured to provide a space for the BEL to sit in line with the other Fab which can be lengthened in the linker or CH1 domain, as shown in Figure C. The invention contemplates that evaluation of a diversity of the first target binding moiety can be accomplished with the BEL place to best accommodate selection in the context of the entire structure as a whole.
b) With respect to other methods to make bispecific and bispecific fusions see Antibody Fusion Proteins Wiley-Liss 1999 (infra) eg. particularly p 131 et seq., and Chapter 7 and the discussion, Methodologies improving the correct pairing of heavy chains are well-known in the art.

Such a construct could also be employed in conjunction with other functional moieties fused or conjugated thereto, for example toxins, cytokines, enzymes, prodrugs, radionuclides etc.

In one preferred embodiment, the invention is directed to a multispecific ligand* with at least two different binding specificities for different target ligands* on the same target cell* and adapted to bind contemporaneously to the different target ligands, said multispecific ligand comprising a first target binding moiety which preferentially* recognizes a first target ligand and a second target binding moiety which preferentially recognizes a second target ligand, and wherein said first target binding moiety recognizes a target cell-associated* target ligand and said second target binding moiety recognizes a non-cell-associated target ligand which is present on target cells and non-target cells; and wherein the ability of the second target binding moiety to bind to the second target is diminished relative the ability of the first target binding moiety to bind to the first target ligand, the first target binding moiety having an ability to bind to the first target ligand which is at least sufficient for the first target moiety to bind to the first target ligand independently of the second target binding moiety binding to the second target ligand and an off-rate which at least sufficiently exceeds the on-rate of the second target binding moiety for the second target ligand to provide opportunity for the second target moiety to bind the second target ligand when the first target binding moiety is bound to first target ligand, the second target binding moiety having a relatively diminished ability to bind or stay bound to the second target ligand independently of the binding of the first target binding moiety to the first target ligand, such that the multifunctional ligand will bind to the target population of cells in preference to the non-target population of cells. As suggested above, the strategy embodied in this preferred embodiment can also be employed in connection with any one or any combination of compatible strategies referred to above, to diminish in degree the requirement of using a low affinity second ligand binding moiety.

In another aspect the invention is directed to heterofunctional ligand comprising a first moiety which binds to a first target ligand and a second moiety which binds to a second target ligand, and wherein the affinity or avidity or both the affinity and avidity of said first moiety are selected to enable the first moiety to bind to the first target ligand independently of the ability of said second moiety to bind to the second target ligand and wherein the relative avidity or affinity or both the affinity and avidity of said second moiety are selected or adjusted to substantially reduce the probability of the second moiety binding to the second target ligand without the first moiety, first or substantially contemporaneously, binding to the first target ligand. For example, in one embodiment the first moiety is divalent and the second moiety is monovalent. In one embodiment the affinity of the first moiety for its target ligand is for example up to several orders of magnitude greater than the affinity of the second moiety for its target ligand, as discussed below. In a preferred embodiment both moieties are capable of binding to different target ligands on the same cell, for example as hereinafter specified, although in the case of tumor cell targeting, particularly with respect to cells that are growing adjacent to another the invention contemplates that the first moiety may bind to one cell and the second moiety may bind to a neighbouring cell. Accordingly, in the case of receptors requiring cross-linking for biological activity the invention contemplates that such same cell interactions and adjacent cell interactions are optionally accomplished when the second moiety is bivalent. In one embodiment, at least one of said first and second moities comprise one or more antibody components. In another embodiment, said first moiety binds to at least one cell-surface ligand which differentiates between cells of the same population or sub-population, for example, at least one ligand which diffentiates which between populations or sub-populations of immune cells (eg. see WO 01/21641, U.S. Pat. No. 6,156,878), for example, activated vs. non-activated, disease-associated or non-disease-associated (over-expressing or uniquely expressing certain receptors or other ligands [for example cytokine or growth factor receptors, particular immunoglobulin like molecules or MHC peptide complexes] or other differentiating markers hereinafter exemplified or apparent to those skilled in the art), and said second moiety, in virtue of its binding to the second target ligand, directly or indirectly exerts a biologic effect eg. a therapeutic effect, for example an immune modulating effect. In a further preferred embodiment said second moiety has a broader target cell population than said first moiety Eg. see Wiley H. et al. Expression of CC Chemokine Receptor-7 and Lymph Node Metastasis . . . , J. Natl. Cancer Inst. 93:1638-1643; Moore M A Bioessays August 2001; 23(8):674-6. (The invention contemplates that by targeting CCR7 receptor selectively on tumor cells, for example using a relatively high affinity binding moiety for a tumor associated antigen and a relatively low affinity moiety which binds to and blocks CCR7 receptor, eg. when combined in therapy with a chemotherapeutic agent or an immunotixun for the same tumor, metastasis can be inhibited). For example, in one embodiment said first moiety binds to a tumor associated antigen on a tumor cell and said second moiety binds to a receptor which is found on the tumor cell but also on a broader population of cells. In another embodiment said first moiety binds to an antigen associated with particular population of leukocytes and said second moiety binds to a receptor which is found on that population of cells but also on a broader population of cells (eg. apoptosis mediating receptors Journal of Immunology 1998 160:3-6, Nat Med August 2001; 7(8)954-960, WO 01/85782; ICAM-R WO 00/29020; see also WO 01/85768, WO 01/85908; WO 01/83755, WO 01/83560, WO 01/29020; Vitale et al. Prpc. Nat. Acad. Sci. May 8, 2001; 98(10):5754-5769; CCR2 see also U.S. Pat. No. 6,312,689; U.S. Pat. No. 6,294,655 Anti-interleukin-1 receptor antagonist antibodies and uses thereof; U.S. Pat. No. 6,262,239; U.S. Pat. No. 6,268,477). In another embodiment the second moiety does not necessarily bind with lower affinity to its target however it may bind to a first ligand which in turn binds to a second ligand on a target cell (eg. a receptor on the target cell eg. a cytokine, chemokine or growth factor receptor), for example the receptor being on the same cell to which he first moiety binds, and it binds in a manner in which it partially interferes with the binding of the first ligand to the second ligand and thereby directs or retargets that first ligand to the second ligand in a manner which accomplishes the intended interaction of the the first with the second ligand (eg a signal transduction or blocking interaction ie. the second moiety causes the eg. cytokine to bind to its receptor without engendering the biological effects attributable to receptor binding eg. signal transduction, which may be assessed by assaying for effects of eg. signal transduction according to well established techniques in the art) but less competitively relative to the first moiety so that the first moiety exerts a targeting function ie. where the first ligand bound by the second moiety binds to a broader than desired population of cells. The binding of the second moiety may also be compatible with the first ligand binding to one cell surface ligand but not another eg. see WO 00/64946 the contents of which are hereby incorporated by reference. The ability to identify ligand residues of importance to binding or residues other these, the alteration of which might interfere with binding is well established in the art. The invention contemplates varying, by high throughput techniques e.g. phage display, residues of an antibody that are not involved in first ligand binding to create variants which can be tested for partial interference with first ligand binding to the second ligand eg. receptor binding.

Examples of receptors for blocking or activation by the targeting methods described herein include tyrosine kinase type recptors, serine kinase type receptors, heterotrimeric G-protein coupled receptors, receptors bound to tyrosine kinase, TNF family receptors, notch family receptors, guanylate cyclase types, tyrosine phosphatase types, adhesion receptors etc. (for example receptors see those discussed in Cancer: Principles and Practice of Oncology 6^{th} Ed. De Vita et al. Eds Lippincott 2001, including particularly Chapter 3, 7 and 18, The Autoimmune Diseases, Academic Press Third Edition, Rose/Mackay ISBN: 0125969236, Immunology 6^{th} Edition, Mosby 2001 Roitt et al. Eds; Molecular Mimicry, Microbes & Autoimmunity by Madeleine W. Cunningham (Editor), Robert S. Fujinami (Editor) December 2000, among other references hereininbelow identified). Further mention may also be made of interleukin and interferon type receptors, HGF receptor (see for example U.S. Pat. No. 6,214,344), CD45, CXC family receptors including CXCR1 and CXCR2 receptors including IL-8 receptor, EGFRs, receptors for molecules with functions in apoptosis or homeostasis, receptors such as FGF which sensitize tumor cells to chemotherapeutic agents, etc. It is known for example to modify receptor ligands in a way which does not interfere with a signaling function (the residues important for signaling may be known or can be readly ascertained eg. see Retargeting interleukin 13 for radioimmunodetection and radioimmunotherapy of human high-grade gliomas. Debinski W, Thompson J P.Clin Cancer Res October 1999; 5(10 Suppl):3143s-3147s) but reduces the affinity of the ligand for this receptor (see also WO 01/19861). Alternatively, the second moiety may be an antibody which is agonistic or antagonistic and used to block, activate, neutralize etc the receptor. With respect to EGFR family, TNF family and other receptor targeting antibodies which are capable of causing apoptosis directly or indirectly, see U.S. Pat. No. 5,876,158, WO 00/20576, WO96/08515, WO 01/44808 (P75AIRM1), WO 00/29020 (ICAM-R), WO 99/12973, CA 2236913 etc. The invention also contemplates that the second moiety may also be targeted to a specific portion of a receptor which differentiates it from other receptors of its class and more generally contemplates that the second moiety may contribute to the targeting ability of the multifunctional ligand. Examples of antibodies that bind to receptors for application of the are also listed in Appendix B.

In another aspect, the invention also contemplates that the first moiety binds to a target cell and said second moiety binds to a ligand, for example a natural ligand, (eg. a cytokine or chemokine circulating at normal levels or at higher levels attributable to a disease or treatment of a disease with another therapeutic molecule) and retargets that ligand (for example, the ligand may be retargetted from circulation) to a targeted cell. For example the invention comtemplates that IL-2 may be retargeted to LAK cells or CTLs via a high affinity Leu-1 9 binding first moiety. For example, antibodies including fragments thereof which bind to cytokines or other natural ligands for retargeting purposes (eg. single domain antibodies) can be made by phage display against the cytokine or ligand while bound in situ to its receptor. The invention also contemplates that the affinity for the cytokine may be adjusted to regulate the degree of targeting and that serum samples may be evaluated to assess the degree of bound cytokine and the relative degree of bound and unbound cytokine. Among other methods, for example, the invention contemplates that a radiolabelled multifunctional ligand may be used assess the amount of label associated with the multifunctional ligand when bound to the cytokine, by capturing the ‘complex’ with an antibody that recognizes both antigenic determinants on both the cytokine and an adjacent portion of the ligand binding thereto ie. forming a composite epitope), such as may be generated by phage display and assessing the amount of label relative to the amount of captured complex. The invention also contemplates administration of supplemental amounts of natural ligand to compensate for the degree in which the ligand is retargeted insofar as such retargeting might impact negatively on immune or other physiological processes.

In another aspect the invention contemplates that patients treated with antibodies to a particular biologic effector ligand eg. a natural ligand eg. a cytokine, for example TNFα, may preferably be treated with a multifunctional ligand having a first moiety which binds to at least one cell type and a second moiety which binds to a natural ligand such as a cytokine for retargeting that cytokine to that cell type, as in a preventative method for treating a disease, eg. cancer. In this respect the invention contemplates that the antibody is capable of binding to the cytokine but once bound the cytokine, the cytokine is incapable and/or only weakly capable of binding to its receptor and/or that the multifunctional ligand also comprises a higher affinity receptor blocking moiety to minimize retargeting of the primary disease site. In one embodiment, the first moiety binds with relatively higher functional affinity (ie. avidity, affinity, and/or relatively advantageous binding capacity in virtue of multiple ligand binding arms, each binding to different ligands on the target cell) to ensure binding to the retarget cell. In another embodiment the bound cytokine is capable of binding to the cytokine receptor at the retarget site but incapable of binding to the receptor at the disease site owing to differences in the receptors at the two sites. The nvention also contemplates using antibodies which interfere but do not preclude binding of the biologic effector to provide a less toxic effect.

For example, patients with Crohn's disease that are treated with anti-TNFα (see for example, Expert Opin Pharmacother May 2000; 1(4):615-22 and references cited therein) may be treated according to the invention with a bispecific antibody having, in addition to an anti-TNFα binding moiety, which reduces the affinity of the bound TNF for the receptor, but also an antibody moiety which binds to tumor antigen which is expressed on many different tumor types or optionally a trispecific antibody which additionally binds to a second multi-carcinomic antigen, preferably one which broadens the range of targeting against prevalent cancers. With respect to tumor antigens mention may be made of EGFR, EPCAM, MUCINs, TAG-72, CEA, H11 among other known multicarcinomic antigens (see also Cancer: Principles and Practice of Oncology 6^{th} Ed. De Vita et al. Eds Lippincott 2001 Chapters 18 and 20.5). In another embodiment, the second moiety differentially retargets a cytokine to one receptor in preference to another, for example, to a TNF receptor over-expressed on tumor cells in preference to a TNF receptor associated with Crohns disease. In a related but also independent aspect, the invention contemplates a method of screening for an antibody which preferentially binds to a ligand when bound to a first receptor relative to another second receptor by screening for antibodies (e.g. by phage display, ribosome display, etc.) which bind to the ligand eg. a cytokine, when bound in situ to the first receptor, and selecting among them those that bind to the ligand eg. cytokine but do not bind (substractive screening) or bind with lesser affinity to the cytokine when bound to the second receptor, as well as to antibodies and multifunctional ligands created by this method (see also U.S. Pat. No. 6,046,048 and WO 99/12973 and references cited therein with respect to TNF family of receptors). Variations in the extracellular domains of such receptors are known and can be ascertained by methods known to those skilled in the art.

Further with respect to multifunctional ligands having a higher affinity targeting moiety relative to the second ie. effector moiety, the second moiety may be for example an antibody or other ligand which interferes with the binding of the regular ligand for this receptor. For example, the invention comtemplates a first ligand binding moiety which recognizes activated T-cells and a second ligand binding moiety which blocks the IL-16 receptor for testing the effect on Crohns disease (or alternatively activates an IL-16 receptor on those cells eg. by using a high affinity IL-16 bound second moiety which becomes relatively low affinity IL-16 receptor ligand when bound to the antibody, again to test the effect on Crohn's disease (see Gut December 2001 49(6) 795-803) For example, in one embodiment, the invention contemplates that the second moiety blocks a receptor that are found on cells other than the target cell, the blockage of which leads to the apoptosis of or destruction of the cell eg. CD95 (eg. see Jung G. et al.,Target cell-restricted triggering of the CD95 (APO-1/Fas) death receptor with bispecific antibody fragments Cancer Res Mar. 1, 2001; 61(5):1846-8). With respect to blocking insulin like growth factor receptor, insulin receptor etc. see The IGF system in thyroid cancer: new concepts. Vella V., Mol Pathol June 2001; 54(3):121-4; Insulin receptor isoform A, a newly recognized, high-affinity insulin-like growth factor II receptor in fetal and cancer cells. Mol Cell Biol May 1999; 19(5):3278-88; Expression of the insulin-like growth factors and their receptors in adenocarcinoma of the colon. Freier S Gut May 1999; 44(5):704-8; Pandini G., Insulin and insulin-like growth factor-I (IGF-I) receptor overexpression in breast cancers leads to insulin/IGF-I hybrid receptor overexpression: evidence for a second mechanism of IGF-I signalingClin Cancer Res July 1999; 5(7):1935-44. With respect to targeting beta-1 integrins see eg. Masumoto A, et al Role of beta1 integrins in adhesion and invasion of hepatocellular carcinoma cells. Hepatology. January 1999; 29(1):68-74. Arao S, et al. Beta1 integrins play an essential role in adhesion and invasion of pancreatic carcinoma cells. Pancreas. March 2000; 20(2):129-37. Xie Y, Xie H. Characterization of a novel monoclonal antibody raised against human hepatocellular carcinoma. Hybridoma. October 1998; 17(5):437-44. Peng H, et al Production and characterization of anti-human hepatocellular carcinoma monoclonal antibodies. Hua Xi Yi Ke Da Xue Xue Bao. September 1990; 21(3):259-62; Whittard J D, Akiyama S K. Activation of beta1 integrins induces cell-cell adhesion. Exp Cell Res. Feb. 1, 2001; 263(1):65-76 Nejjari M, et al. alpha6beta1 integrin expression in hepatocarcinoma cells: regulation and role in cell adhesion and migration. Int J Cancer. Nov. 12, 1999; 83(4):518-25; Yao M et al Expression of the integrin alpha5 subunit and its mediated cell adhesion in hepatocellular carcinoma. J Cancer Res Clin Oncol. 1997;123(8):435-40.

The invention also contemplates a method of optimizing the cooperative affinities of respective binding ligands of a multifunctional ligand described herein and the length of a linker therebetween for the above and applications described below by phage or ribosome display etc. in which the multifunctional ligand is a single polypeptide chain, for example, two single chain Fvs or single domain antibodies linked in sequence, or a diabody (see U.S. Pat. No. 5,837,242), by varying the DNA sequence corresponding to amino acids that represent linker and/or for example CDR regions that are postulated to impact on affinity according to methods and strategies that well known in the art for affinity maturation. These same strategies can be employed for engineering lower affinity molecules. Accordingly, more generally the invention is directed to a phage display or similar library (eg. a ribosome display library or a microarray) in which the population of variants is a multispecific ligand, including a multispecific ligand according to the invention herein defined. (See Irving R A, et al. Ribosome display and affinity maturation: from antibodies to single V-domains and steps towards cancer therapeutics. J Immunol Methods. (2001);248(1-2):31-45.Review. PMID:11223067; Adams G P, Schier R. Generating improved single-chain Fv molecules for tumor targeting. J Immunol Methods.(1999)231(1-2):249-60. Review. PMID:10648942. The term ligand binding moiety includes any ligand that can be used as a targeting arm or to bind to the second ligand for example cytokines, chemokines, etc and optionally those that can be modified, preferably by high throughput means to adjust its affinity and/or specificity, including scaffolds comprising a polypeptide, a peptide, a carbohydrate, a ribonucleic acid, a lipid and a small molecule, or a binding moiety consisting essentially of a combination of such types of molecules. Example include non-immunoglobulin protein scaffolds such affibodies (see U.S. Pat. No. 5,831,012, U.S. Pat. No. 5,958,736, EP 0486525) Trinectin domains (Phylos Inc.), TCRs, peptide mimetics, peptides located within a antibody variable region scaffold (eg. see WO 02/44197, especially with respect to selectively targeting interferon alpha to specific cell populations) peptide fusion proteins and stabilized polypeptide loops, disulfide stabilized loops (eg. see WO 99/23222); all known in the art for such uses. These may be linked by peptide linkers of for example 10 to 50 amino acids. A variety of linkers including flexible linkers such as multiples of gly4ser are well known in the art.

In another embodiment blockage of a receptor does not necessarily lead to cell death but may lead only to decreased or increased release of certain cytokines etc, for example as mediated via the IL-6 receptor. In another embodiment the second moiety may achieve the desired therapeutic effect by constituting the normal ligand for that receptor or a functional substitute. The multispecific ligand may also be fused or conjugated to a toxic moiety or other effector. In another or further preferred embodiment, said first moiety comprises two binding ligands (eg. one or both of which may be an antibody) which respectively bind to two different target ligands each of which contributes to its total binding capacity and neither of which are sufficient to efficiently target the the cell, for example a ligand which binds to a specific MHC peptide complex and a second reduced affinity ligand which binds to a ligand on an APC. This approach also obviates the need to create high affinity ligand for a particular MHC petide complex, although this can been accomplished. In another or further preferred embodiment the target cell is an immune cell and the second moiety binds to a molecule involved in cellular adhesion, a cytokine receptor, a ligand which stimulates the activity of said immune cell, a ligand which inhibits the activity of said immune cell, a ligand which causes one or more cytokines to be released, a ligand which prevent one or more cytokines from being released, a ligand which causes or facilitates apoptosis of said immune cell or a ligand which permits internalization of said multispecific ligand. In another preferred embodiment the heterofunctional ligand is fused or conjugated to a therapeutic agent or a moiety that binds to a therapeutuc agent (exemplified below) or a ligand which effects binding to another immune cell, for example a T cell. In another preferred embodiment, the multispecific ligand is a bispecific antibody, a trispecfic antibody or a tetraspecific antibody. In a further preferred embodiment the first moiety binds to but is incapable of modulating the activity of said immune cell and said second moiety modulates the activity of said immune cell independently of said first moiety. In other aspects the invention is directed to a pharmaceutical composition comprising such a multispecific ligand and a pharmaceutically acceptable carrier, a method of using the heterofunctional ligand in the preparation of a pharmaceutical composition for treating a disease, and to a method of treating a subject by administering same in a therapeutically effective amount.

The invention is also directed to a multispecific ligand which comprises a first ligand binding moiety which neutralizes a ligand eg. a natural ligand such as a cytokine, chemokine, colony stimulating factor or growth factor and a second ligand binding moiety which binds to a cell marker associated with a cell through which the natural ligand exerts a deleterious affect. Preferably the affinity for the first ligand binding moiety for the natural ligand will be greater than that of the second ligand binding moiety for the cell associated marker. Optionally the construct will be selected so that the binding interfaces are pointed in opposite directions and the lever effect is maximized, for example a bispecific construct where the heavy chains are joined directly or through an inflexible linker and are optionally linked to their respective (optionally common respective light chains) through a disulphide linker via framework residues as is well known in the art. Optionally, the molecular weight of the first ligand binding moiety is substantially less or more (by at least 10%, preferably at least 20%, more preferably at least 25%) and preferably less than that of the second ligand binding moiety as may be effected or maximized through mutating from higher to lower (on the first ligand binding moiety) and lower to higher mol. wt residues (on the second ligand binding moiety), the native residues which are not exposed (to avoid immunogenicity) and not essential for proper folding and function of the VH/VL (as may be determined from the degree of conservation of such residues among immunoglobulins of the species, through % frequency tables available through the Kabat database and well known published determinations in this regard (see for example WO 02/40545). Examples include neutralizing IL-2 via a marker on activated T cells, blocking IL-15 via CD8+ T cells, blocking TNF alpha on mast cells; binding thrombin via activated endothelial cells etc.

The term heterofunctional is used broadly to refer to a ligand: 1) comprising at least two functional moieties that have different functions or different capacities to perform the same function and 2) which is typically and preferably heterospecific (having two binding specificities).

Unless the context dictates otherwise the term avidity when used in a comparative, quantifiable or controllable sense is used to refer the valency of the binding entity or moiety. The term functional affinity is used a composite term referring to a quantitative and contollable (though not necessarily quantifiable, especially when it consists of both avidity and affinity components) propensity to specific binding attributable to one or both of avidity and affinity effects.

In another aspect, the invention contemplates that cells, particularly immune cells, that are expected to be present at or proximal to a disease site (eg. at the site where an immune cell crosses the vascular endothelial cell wall), in virtue of the disease or a therapeutic modality which is employed in relation to the disease or a concurrent disease, including cells that directly mediate the disease, may be targetted in virtue of a marker associated with such cells, eg. markers associated with activated immune cells or disease mediating immune cells eg. LEU-19, a marker associated with activated or killer T-cells, etc for example with an antibody, which is linked to a moiety that is capable of exerting a therapeutic effect in relation to the disease, for example, an immunoliposome or an antibody linked to another therapeutic delivery system (for example example streptavidin or biotin fused, coated or conjugated entities or other payload carrying entities (see for example U.S. Pat. Nos. 5,439,686, 6,007,845, 5,879,712, 5,456,917, 6,165,502, 5,079,005, 5,888,500, 5,861,159, 6,193,970, 6,190,692, 6,077,499, WO 00/69413, WO 01/07084, etc.). For example, an immunoliposome may carry one of or a combination of cytokines, chemokines, toxins or other therapeutic molecules suitable for treating the disease directly or indirectly, for example by attracting or preventing the attraction, activating, anergizing or otherwise modulating the activity of immune cells for therapeutic or related purposes. Thus according to another aspect, the invention is directed to a multifunctional ligand characterized in that it exerts an independent biologic function said multifunctional ligand comprising a ligand which binds to a non-diseased disease associated cell and: a) a therapeutic entity; b) a ligand which binds to a therapeutic entity; or c) a ligand which binds to a disease mediating entity eg. a biologic effector molecul which is released by the disease mediating entity or the diseased cell eg. a cytokine or other BEL which mediates or aggravates a disease process. Preferably said multifunctional ligand comprises at least two of a), b) or c) and preferably all three.

The term “cancer” refers to or describes the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, blastoma, gastrointestinal cancer, renal cancer, pancreatic cancer, glioblastoma, neuroblastoma, cervical cancer, ovarian cancer, liver cancer, stomach cancer, bladder cancer, hepatoma, breast cancer, colon cancer, colorectal cancer, endometrial carcinoma, salivary gland carcinoma, kidney cancer, liver cancer, prostate cancer, vulval cancer, thyroid cancer, hepatic carcinoma and various types of head and neck cancer.

Typically, an immunoglobulin has a heavy and light chain. Each heavy and light chain contains a constant region and a variable region, (the regions are also known as domains). Light and heavy chain variable regions contain a framework region interrupted by three hypervariable regions, also called complementarity-determining regions or “CDRs”. The extent of the framework region and CDRs have been defined and can be routinely demarcated for any given antibody of interest (see, Kabat, E., et al., SEQUENCES OF PROTEINS OF IMMUNOLOGICAL INTEREST, U.S. Department of Health and Human Services, (1991), which is hereby incorporated by reference. The Kabat database is now maintained online at http://immuno.bme.nwu.edu/. The sequences of the framework regions of different light or heavy chains are relatively conserved within a species. The framework region of an antibody, that is the combined framework regions of the constituent light and heavy chains, serves to position and align the CDRs in three dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. References to VH or a VH refer to the variable region of an immunoglobulin heavy chain, including that of an Fv, scFv, dsFv or Fab. References to VL or a VL refer to the variable region of an immunoglobulin light chain, including that of an Fv, scFv, dsFv or Fab. Variants and portions of an antibody known in the art such as, Fab, fragments, Fab′ fragments, F(ab′)2 fragments, single chain Fv proteins (scFv), single domain antibodies (sdAbs) and disulfide stabilized Fv proteins (dsFv). The term antibody covers intact monoclonal antibodies, polyclonal antibodies, chimeric antibodies, humanized antibodies, multispecific antibodies (e.g. bispecific, trispecific and tetraspecific antibodies in any of the multitude of construct designs known in the art eg. diabodies, formed from at least two intact antibodies, and antibody fragments so long as they exhibit the desired biological activity. The term also includes any antigen binding polypeptide which competively inhibits binding of the antibody in question to its target antigen.

Functionally, the term “antibody” may be used in the broadest sense to cover the function of specific recognition and binding to an epitope and may additionally include, without limitation, agonist, antagonist, blocking or neutralizing functions as well as polyepitopic specificity and polyfunctionality. “Antibody fragments” comprise one or more portions of an intact antibody, typically comprising at least one of the antigen binding or variable regions of the intact antibody. Examples of antibody fragments are provided above and include linear antibodies (see Example 2 of U.S. Pat. No. 6,066,719); and multispecific antibodies formed from antibody fragments (e.g. bispecific sdAbs or scFvs, which may disulfide stabilized, linked through a flexible linker etc.).

As referred to herein, the term expressing typically employs expression control sequences The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers, and it will be appreciated that proteins can be expressed in a cell-free system.

As the invention is directed to use or administration or use of effective amounts of a multispecific ligand of the invention, it will be appreciated that in a therapeutic or investigational context, an “effective amount” is an amount sufficient to effect a beneficial or desired clinical result. An effective amount can be administered in one or more doses. In terms of treatment, an effective amount is the amount that is sufficient to palliate, ameliorate, stabilize, reverse or slow the progression of the disease, or otherwise reduce the pathological consequences of the disease. In terms of an adjuvant, an effective amount is one sufficient to enhance the immune response to the immunogen. The effective amount is generally determined by the physician on a case-by-case basis and is within the skill of one in the art. Several factors are typically taken into account when determining an appropriate dosage. These factors include age, sex and weight of the patient, the condition being treated, the severity of the condition and the form of the antibody being administered. For instance, the concentration of scFv need not be as high as that of native antibodies in order to be therapeutically effective.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. An “isolated” DNA sequence or nucleic acid molecule is a nucleic acid molecule that is identified and separated from at least one contaminant nucleic acid molecule with which it is ordinarily associated in the natural source of the polypeptide nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

The term “mammal” as used herein refers to any mammal classified as a mammal, including humans, cows, horses, dogs and cats.

The term parental antibody is used interchangeably with template antibody and means any antibody of interest which is to be mutated or varied to obtain antibodies or fragments thereof which bind to the same epitope as the parental antibody, but which have different properties, for example enhanced affinity or stability or a lower pl. The term template or parent have a similar meaning in relation to the broader universe of polypeptide ligands and includes templates in the form of any invariable portion for the development of any ligand.

Pharmaceutically acceptable carrier or excipient refers to any of the standard pharmaceutical carriers, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in REMINGTON'S PHARMACEUTICAL SCIENCES, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intratumoral, peritumoral, intravenous or intraperitoneal injection; or topical, transdermal, or transmucosal administration).

As used herein, “recombinant” includes reference to a protein produced using cells that do not have, in their native state, an endogenous copy of the DNA able to express the protein. The cells produce the recombinant protein because they have been genetically altered by the introduction of the appropriate isolated nucleic acid sequence. The term also includes reference to a cell, or nucleic acid, or vector, that has been modified by the introduction of a heterologous nucleic acid or the alteration of a native nucleic acid to a form not native to that cell, or that the cell is derived from a cell so modified. Thus, for example, recombinant cells express genes that are not found within the native (non-recombinant) form of the cell, express mutants of genes that are found within the native form, or express native genes that are otherwise abnormally expressed, underexpressed or not expressed at all.

In one aspect, the invention contemplates populations of genetic packages having a genetically determined outer surface protein including genetic packages that collectively display a plurality of potential binding fragments in association with such outer surface protein, such as phage display libraries, yeast display libraries, bacterial display libraries etc. Libraries generated by ribosome display are also contemplated. Also contemplated are libraries of variants allocated to individual wells or spots in an array, including variants generated by the methods of mutagenesis disclosed by Diversa Corporation in U.S. Pat. Nos. 6,358,709; 6,238,884; 6,361,974; 6,352,842; 6,171,820.

For example, in phage display and similar display technologies, each variant in the library includes a nucleic acid construct coding for a fusion protein which includes at least a portion of the outer surface protein of the phage or phagemid and a variant of at least one parental binding-fragment. Preferably the variable region is only partly randomized in that it is biased in favour of encoding the amino acid constitution of the parental binding-fragment such that the plurality of different potential binding domains are adapted to express characteristics of the parent.

Furthermore, a method for biasing a library in favor of obtaining selected percentages of wild type amino acid residues is achieved by creating residue substitutions by using different spiking levels of the various dNTPs as described below. When creating a phage or similar library, the randomization of amino acids is often achieved by DNA synthesis. A primer is annealed next to DNA encoding for the variable region, and nucleotides are randomly added to synthesize randomized variable regions. Normally, at the step of synthesizing the DNA used to produce the variable region of the phage library, one uses a nucleotide ratio of 1:1:1:1, which generates a totally random variable region. By contrast, during synthesis of the variable region, the likelihood of preserving a particular tumor specificity or other desirable traits found in the wild type may be enhanced as follows. At each step of adding a nucleotide to the DNA variable region, one selects a dNTP ratio which is biased in favor of producing amino acids which reflect the DNA of the parental (wild type) species (see Deng S J, et al. Basis for selection of improved carbohydrate-binding single-chain antibodies from synthetic gene libraries.Proc Natl Acad Sci USA May 23, 1995; 92 (11):4992-6).

Variations in amino acid composition of a ligand may be introduced for a variety of reasons, including:

i) introducing cysteine substitutions for example to make a dsFv (see U.S. Pat. No. 6,147,203)

ii) introducing one or more substitutions with amino acids that have the highest frequencies of representation within antibodies, optionally within human antibodies to introduce variability in the FR while preserving human characteristics of the framework regions, for example, as determined by the percent frequency table shown in WO 02/40545, for example, amino acids that have at least a 5-10% representation in such surveyed antibodies, amino acids having a 10-20% representation, amino acids having a 20-30% representation, amino acids having a 30-50% representation, or amino acids having a 50-100% representation in such antibodies. The invention contemplates a ligand comprising any one or more substitutions in accordance with any one or more of the preceding categories of representation. According to one embodiment, one or more substitutions based on frequency of representation is based on a percent frequency table in which the antibodies are all of the same class (for classifcation of antibodies in terms of their various types of heavy and light chain variable regions and effecting changes based on such classifications see Ewert S, et al. Structure-based improvement of the biophysical properties of immunoglobulin v(h) domains with a generalizable approach. Biochemistry. Feb. 18, 2003; 42(6):1517-28. Ewert S, et al. Biophysical properties of human antibody variable domains.J Mol Biol. Jan. 17, 2003; 325(3):531-53. Jung S, et al. The importance of framework residues H6, H7 and H10 in antibody heavy chains: experimental evidence for a new structural subclassification of antibody V(H) domains. J Mol Biol. Jun. 8, 2001; 309(3):701-16. Marget M, et al. Bypassing hybridoma technology: HLA-C reactive human single-chain antibody fragments (scFv) derived from a synthetic phage display library (HuCAL) and their potential to discriminate HLA class I specificities. Tissue Antigens. July 2000; 56(1):1-9. Knappik A, et al. Fully synthetic human combinatorial antibody libraries (HuCAL) based on modular consensus frameworks and CDRs randomized with trinucleotides. J Mol Biol. Feb. 11, 2000; 296(1):57-86. Hanes J, et al. Picomolar affinity antibodies from a fully synthetic naive library selected and evolved by ribosome display. Nat Biotechnol. December 2000; 18(12):1287-92. Honegger A, et al. Yet another numbering scheme for immunoglobulin variable domains: an automatic modeling and analysis tool. J Mol Biol. Jun. 8, 2001; 309(3):657-70. Krebs B, et al. High-throughput generation and engineering of recombinant human antibodies. J Immunol Methods. Aug. 1, 2001; 254(1-2):67-84.); Matsuura T, Pluckthun A. Selection based on the folding properties of proteins with ribosome display.FEBS Lett. Mar. 27, 2003; 539(1-3):24-8; PMID: 12650920; Amstutz P, Forrer P, Zahnd C, Pluckthun A. In vitro display technologies: novel developments and applications Curr Opin Biotechnol. August 2001; 12(4):400-5. Review. PMID: 11551470; 54; Schaffitzel C, Hanes J, Jermutus L, Pluckthun A. Ribosome display: an in vitro method for selection and evolution of antibodies from libraries, J Immunol Methods. Dec. 10, 1999; 231(1-2):119-35. Review. PMID: 10648932: Hanes J, Jermutus L, Weber-Bornhauser S, Bosshard H R, Pluckthun A. Ribosome display efficiently selects and evolves high-affinity antibodies in vitro from immune libraries.Proc Natl Acad Sci USA. Nov. 24, 1998; 95(24):14130-5. PMID: 9826665.

iii) introducing one or more substitutions to reduce the immunogenicity of the antibody (see for example WO 02/34779; WO 00/34317; U.S. Pat. No. 6,407,213; U.S. patent application No.20020034765; see also Kashmiri S V, et al. Development of a minimally immunogenic variant of humanized anti-carcinoma monoclonal antibody CC49. Crit Rev Oncol Hematol. April 2001; 38(1):3-16. Tamura M, et al. Structural correlates of an anticarcinoma antibody: identification of specificity-determining residues (SDRs) and development of a minimally immunogenic antibody variant by retention of SDRs only. J Immunol. Feb. 1, 2000; 164(3):1432-41).

(v) substituting framework regions and/or select amino acids to improve thermal stability of scFvs (see U.S. application No. 2002/0146846; Ewert S, et al., Biophysical properties of human antibody variable domains. J Mol Biol. Jan. 17, 2003; 325(3):531-53.; Jermutus, L., et al. (2001). Tailoring in vitro evolution for protein affinity or stability. Proc. Natl. Acad. Sci. U.S.A. 98, 75-80; Wörn, A., et al. (2001) Stability engineering of antibody single-chain Fv fragments. J. Mol. Biol. 305, 989-1010; der Maur A A, et al., Direct in vivo screening of intrabody libraries constructed on a highly stable single-chain frame J Biol Chem Nov. 22, 2002; 277(47):45075-85; Wörn, A., et al. (1999). Different equilibrium stability behavior of scFv fragments: Identification, classification, and improvement by protein engineering. Biochemistry 38, 8739-8750.

Immunogenicity

For example, in the process of Deimmunisation™ (Biovation Ltd.) disclosed in WO 00/34317, VH and VL sequences from the starting antibody are analysed to identify human T cell epitopes. The result is a human T cell epitope “map” from each V region showing the location of epitopes in relation to complementarity-determining regions (CDRs) and other key residues within the sequence. Then, individual T cell epitopes from the T cell epitope map are analysed in order to identify alternative amino acid substitutions with a low risk of altering activity of the final antibody. A range of alternative VH and VL sequences are typically “designed” comprising combinations of amino acid substitutions and these sequences are subsequently incorporated into a range of modified antibodies that are tested for function. For a typical Antibody DeImmunisation™ between 12 and 24 variant antibodies are generated and tested.

Expression vectors generally are replicable polynucleotide constructs that contain a polynucleotide encoding a ligand polypeptide of interest. The polynucleotide encoding ligand polypeptide is operatively linked to suitable transcriptional controlling elements, such as promoters, enhancers and terminators. For expression (i.e., translation), one or more translational controlling elements are also usually required, such as ribosome binding sites, translation initiation sites, and stop codons. These controlling elements (transcriptional and translational) are typically derived from the VB1 heterologous genes (i.e., derived from other genes or other organisms). A polynucleotide sequence encoding a signal peptide can also be included to allow a ligand polypeptide to cross or lodge in cell membranes or be secreted from the cell. A number of expression vectors suitable for expression in eukaryotic cells including yeast, avian, and mammalian cells are known in the art. One example of an expression vector is pcDNA3 (Invitrogen, San Diego, Calif.), in which transcription is driven by the cytomegalovirus (CMV) early promoter/enhancer. This vector also contains recognition sites for multiple restriction enzymes for insertion of a ligand polynucleotide of interest. Another example of an expression vector (system) is the baculovirus/insect system. Yeast expression systems are also well established Shi X, Karkut T, Chamankhah M, Alting-Mees M, Hemmingsen S M, Hegedus D. Optimal conditions for the expression of a single-chain antibody (scFv) gene in Pichia pastoris. Protein Expr Purif. April 2003; 28(2):321-30. Orr B A, Carr L M, Wittrup K D, Roy E J, Kranz D M. Rapid Method for Measuring ScFv Thermal Stability by Yeast Surface Display.Biotechnol Prog. March-April 2003; 19(2):631-8. Robin S, Petrov K, Dintinger T, Kujumdzieva A, Tellier C, Dion M. Comparison of three microbial hosts for the expression of an active catalytic scFv. Mol Immunol. January 2003; 39(12):729-38.

The term disease associated cell means, unless the context implies otherwise, diseased cells or disease causing, mediating (ie. having a role whicn is known to be intermediary or indirectly facilitating eg. antigen presenting cells) or mitigating cells (cells, typically immune cells, which directly or indirectly counteract the diseased or disease causing or mediating cells).

In other aspects the invention is directed to a pharmaceutical composition comprising such a heterofunctional ligand and a pharmaceutically acceptable carrier, a method of using the heterofunctional ligand in the preparation of a pharmaceutical composition for treating a disease, and to a method of treating a subject by administering same in a therapeutically effective amount.

In other aspects the invention is directed to a pharmaceutical composition comprising such aforementioned heterofunctional ligand and a pharmaceutically acceptable carrier, a method of using the heterofunctional ligand in the preparation of a pharmaceutical composition for treating a disease, and to a method of treating a subject by administering same in a therapeutically effective amount. As suggested below, the foregoing strategy could be used in combination with other targeting strategies herein mentioned or known in the art.

The invention contemplates making antibodies to second entities, for example, while bound to their natural receptor, by phage or ribosome display, by methods as hereinafter disclosed.

In another aspect the invention is directed to a heterofunctional ligand comprising at least a first moiety which specifically binds to a first target ligand on a cell and a second moiety which specifically binds to at least a second target ligand on the same cell, and wherein the affinity or avidity or both the affinity and avidity of said first moiety and the affinity or avidity or both the affinity and avidity of the second moiety are selected to substantially reduce the probability of the either moiety singly binding to its respective ligand for a sufficient duration or series of durations to accomplish the function of said heterofunctional ligand unless both first and second moieties are substantially contemporaneously bound to the cell. In a preferred embodiment the first moiety binds to at least one target ligand which differentiates between populations or sub-populations of immune cells and the second moiety in virtue of its binding to the second target ligand, directly or indirectly exerts a therapeutic effect, for example by modulating the activity of said immune cell. In another or further preferred embodiment the first moiety is incapable of modulating the activity of said immune cell and said second moiety modulates the activity of said immune cell independently of said first moiety. In another or further preferred embodiment the second moiety binds to a BEL, for example a molecule involved in cellular adhesion, a cytokine receptor, a ligand which stimulates the activity of said immune cell, a ligand which inhibits the activity of said immune cell (eg. via anergy or tolerance mechanisms), a ligand which causes one or more cytokines to be released, a ligand which prevent one or more cytokines from being released, a ligand which causes or facilitates apoptosis of said immune celll, a ligand which permits internalization of said heterofunctional ligand. In another preferred embodiment the heterofunctional ligand is fused or conjugated to a therapeutic agent or a moiety (eg. biotin, avidin) that binds to a therapeutuc agent (exemplified below) or a ligand which effects binding to another immune cell, for example a T cell. In another preferred embodiment, the heterofunctional ligand is a bispecific antibody, a trispecfic antibody or a tetraspecific antibody.

In other aspects the invention is directed to a pharmaceutical composition comprising such a heterofunctional ligand and a pharmaceutically acceptable carrier, a method of using the heterofunctional ligand in the preparation of a pharmaceutical composition for treating a disease, and to a method of treating a subject by administering same in a therapeutically effective amount.

In other aspects the invention is directed to a method of in vivo modeling or testing using one or more foregoing targeting strategies by administering a heterofunctional/multifunctional ligand as hereinabove disclosed In related aspects the invention is directed to a test ligand in the form of such a heterofunctional/multifunctional ligand and compositions thereof.

It is to be understood that targeting strategies employing the cooperative action of ligands with different affinities for their targets exemplified above, may preferably have affinities which differ, depending on the application and their resulting avidity, by a factor of 20% up to a number of orders of magnitude which may one, two, three, four, five, six and even seven or eight order of magnitude, in order to achieve substantial advantage, as herefter detailed in connection with one such strategy.

1. The term disease is used broadly to refer to any undesirable condition. The term diseased cell includes but is not limited to a cancerous (in the broadest sense of that term) cell and a virally infected cell (these examples are given inasmuch as the invention in a preferred embodiment involves targeting such cells for destruction) and the term disease causing cell includes but is not limited to a virus or other infectious agent and as well as immune cell which is directly or indirectly involved in mediating or causing a undesired, deleterious or pathologic consequence, including but not limited to autoimmune disorders, transplant rejection, and other immune system linked diseases. The term disease causing entity is used to refer, without limitation, to any molecule, atom, peptide, ligand, complex, chemical, component, epitope etc. that is directly or indirectly involved or associated in mediating or causing a disease or disease causing event including an antibody. Such binding to the entity may be effected through the instrumentality of one or more (same or different) multifunctional ligands and through binding to any ligand or set of ligands, including receptors, multi-component epitopes etc, including for example, tumor “associated” (ie. differentially expressed to advantage for targeting purposes) epitopes which may or may not or may only be partially present on tumor associated antigens, or commonly, for example antigens/epitopes/ligands/receptors etc. which are over-expressed in association with cancer cells; or for example, antigens/epitopes/ligands/receptors etc. involved in immune signaling, stimulatory, co-stimulatory, inhibitory, adhesion or other interactions, including without limitation, cytokine receptors, ligands associated with immune cell adhesion (see for example U.S. Pat. No. 5,747,035), ligands to which binding results in stimulation, activation, apoptosis, anergy or costimulation, or ligands which differentiate between different populations or subpopulations or immune cells, including sub-populations of B cells and T cells, activated versus non-activated lympocytes, diseased or disease-causing cells versus non-diseased/disease causing lymphocytes and specific immune cell clones for example those having specific Ig type and MHC-peptide type ligands/and correlative ligands. Examples of such ligands include CCR5, CTLA-4, LFA-1, LFA-3. ICAMs eg. ICAM-1, ELAM-1, CD2, CD3, CD4 (eg see U.S. Pat. No. 6,136,310), CD5, CD6, CD18, CD22, CD40, CD44; CD80, CD86, CD134 and CD154, to name only a few (see also U.S. Pat. No. 6,087,475: PF4A receptor, U.S. Pat. No. 6,135,941, WO 01/13945. Such ligand may also selectively be targeted using any dual affinity strategy according to the invention.

2. The invention is also directed to a multifunctional ligand and a method which comprises using the multifunctional ligand to assess the toxicity of directly or indirectly targeting cells having well known markers that are associated with immune cells, for example, those exclusively associated with activated immune cells, in-so-far as such targeting has a role in prolonging or counteracting the activated state, destroying the cell (eg. where the multifunctional ligand is a immunotoxin) causing the cell to be destroyed (eg. through apoptosis (eg. see WO 01/19861, fas-fasL, U.S. Pat. No. 6,046,048) or assisting another molecule or cell for example a T-cell or other killing or immune modulating cell to do the modulation or killing (markers such as CD23, CD25, CD26, CD28, CD30, CD38, CD49a, CD69, CD70, are just some of the markers associated with activated immune cells) etc. (for a complete listing of marker associated with activated immune cells see for example Roitt I et al. Immunology, sixth edition, Mosby 2001 referenced below and Encyclopedia of Immunology (1998), Abbas et al. Cellular and Molecular Immunology 2000, Harcourt & Brace, the contents of which are incorporated by reference herein). Antibodies for many such ligands are known or could be readily made by e.g. phage display (see references herein including J Immunol Methods Dec. 10, 1999; 231(1-2):65-81), and natural ligands for such markers or functional analogues thereof are in some cases known or could be made by recombinant DNA technologies referenced herein (see also Cellular & Molecular Immunology 4^{th} Edition, Abbas Ak et al. WB Saunders and Company 2000, Antibody Fusion Proteins, Steven M Chamow, Avi Ashkenazi Eds. ISBN 047118358X May 1999 Wiley; Kontermann, R., et al.(Eds.) Antibody Engineering, Springer 2001. ISBN 3-540-41354-5; Antibody Engineering, Carl A. Borrebaeck Oxford University Press, 1995; Antibody Engineering:A Practical Approach David J. Chiswell, Hennie R. Hoogenboom, John McCafferty Oxford University Press,1996; Antibody Engineering Protocols, Sudhir Paul (1995) Humana Press; Antibody Expression & Engineering (1998) Henry Y. Wang, Tadayuki Imanaka, American Chemical Society). The term modulation is used broadly to refer to any change, directly or indirectly, in an immune function or effect, as broadly understood. Many such forms of modulation are well known in the art (some are exemplified herein), and therefore these need not be specifically recited (for a review of such effects see for example Roitt I et al. Immunology, sixth Edition, Mosby 2001; Encyclopedia of Immunology; (1998) Morgan Kaufmann Publishers, ISBN:0122267656).

3. The invention contemplates a variety of different size multifunctional ligands (MRU, single domain, scFv, Fab, minibodies, F(ab)₂, F(ab′)₂, substantially whole antibodies etc. and known or obvious multimers thereof referenced herein and in the referenced literature) that are most suitable (eg. small enough or, for example, having longest half life in circulation) for particlar modes of administration to the extent that this is a limitation.

With respect to technologies being developed for selection of successful binders by phage or ribosome display (see for example WO 01/00866; Adv Protein Chem 2000; 55:367-403).

It is also contemplated that a multispecific contruct as described in WO99/37791 could be used with respect to various aspects aspects of the invention.

With respect to tetravalent constructs see also WO 02/096948

In preferred embodiments the invention is directed to multifunctional ligands that comprise immune function exerting moieties having functionalities of molecules currently in clinical trials or proposed for clinical trials (see for example Glennie M J et al. August 2000, Immunology Today 408 Vol 21(8); see also Journal of Immunological Methods 237 (2000) 131-145; Mol Immunol June 2000; 37(9) 515-526; Annu Rev Med 2001; 52:125-145; Annu Rev Med 2001 52:63-78; Q J Nucl Med September 2000; 44(3) 268-83) including those that have an anti-CD2 functionality (see U.S. Pat. No. 5,795,572) anti-CD4 functionality (see for example U.S. Pat. No. 6,136,310 Herzyk D, J Infect Immun February 2000; 69(2): 1032-1043) anti-CD3 functionality (for example WO 00/41474; WO 98139363; U.S. Pat. No. 6,113,901; Transplantation Dec. 27, 2000 70 (12) 1707-12); Anti-CD44 functionality see for example Weiss L, et al., Proc Nat Acad Sci USA Jan. 4, 2000; 97(1) 285-290; Sugiyama K, Immunol Invest (1999) March-May 28(2-3) 185-200; Brocke S. et al. Proc Nat Acad Sci USA Jun. 8, 1999 96(12) 6896; Mickecz K et al. Nat Med 1 June 1995; 1(6); 558-63; Ahrens T et al., J Invest Dermatol. January 2001 116(1) 93-101); with respect to control of migration of T-cell lymphocytes see Nohara C, et al. J Immunol. Feb. 1, 2001; 166(3) 2108-2115), anti-CD20 functionality (see Crit Rev Oncol Hematol January 2001 37(1):13-25) etc. anti-CD22 functionality see for example Newton D L, et al. Blood Jan. 15, 2001; 97 (2): 528-535, U.S. Pat. No. 5,184,892; Anti-CD40/CTLA-4 see for example J Immunol Oct. 1, 2000; 165(7):3612-9; Microsurgery 2000; 2c (8); 448-452; U.S. Pat. No. 5,874,082; U.S. Pat. No. 6,056,959; U.S. Pat. No. 5,801,227; U.S. Pat. No 6,004,552; U.S. Pat. No. 5,677,165; U.S. Pat. No. 6,087,329; U.S. Pat. No. 5,961,974; U.S. Pat. No. 6,051,228; White C A, et al. Annu Rev Med. 2001; 52: 63-78 (see also reviews and specific applications referred to in Ditzel et al., Immunol Res. 2000; 21(2-3):185-93; U.S. Pat. No. 6,010,902, U.S. Pat. No. 5,876,950; U.S. Pat. No. 5,876,718; U.S. Pat. No. 5,601,819, U.S. Pat. No. 5,981,251, U.S. Pat. Nos. 5,885,579 and 5,885,796; Cancer Immunol Immunother June 2000; 49(3):173-80; Omar K, J Neuroimmunol Feb. 1, 2001 113(1)129-141; Bellido M, Eur J. Haematol February 2001 66(2) 100-106; Broeren et al. J Immunol (2000) December 15 165(12) 6908-14; Alexandroff A B et al Mol Immunol June 2000 37(9) 515-526; Werkerle T J Immunol. Feb. 15, 2001 166(4) 2311-2316; Howard L M J Immunol February 2001; 116(3) 1547-53 anti-CD154; J Pharmacokinet Biopharm August 1999; 27(4):397-420, J Clin Oncol April 2000; 18(8):1622-36, Leukemia March 2000; 14(3):474-5, Clin Cancer Res February 2000; 6(2):372-80, Leukemia January 2000; 14(1):129-35, J Nucl Med November 1999; 40(11):1935-46, Blood Nov. 15, 1999; 94(10):3340-8, Blood Aug. 15, 1999; 94(4):1237-47, Cancer Res May 1, 1999; 59(9):2096-101, Vaccine Apr. 9, 1999; 17(15-16):1837-45, Blood Dec. 1, 1998; 92(11):4066-71, J Rheumatol November 1998; 25(11):2065-76, Clin Pharmacol Ther September 1998; 64(3):339-46, Mult Scler July 1996; 1(6):339-42, Cancer Immunol Immunother July 1997; 44(5):265-72, Transplant Proc December 1996; 28(6):3210-1, Arthritis Rheum. July 1996; 39(7):1102-8, Immunology May 1996; 88(1):13-9 and U.S. Pat. No. 5,876,718).

Antibody Structure and Function

Antibody structure and function has bee extensively described in the literatue. For example see Antibody Engineering 2^{nd} ed. Carl A. K. Borrebaeck, Oxford University Press 1995 p 3-44.

Production of Bispecific Antibodies

A variety of different constructs have been developed for the production of bispecific antibodies including conventional four chain antibodies (including truncated version thereof such minibodies (see U.S. Pat. No. 5,837,821), F(ab′)₂ (see Antibody Fusion Proteins, Steven M Chamow, Avi Ashkenazi Eds. ISBN 047118358X May 1999 Wiley p.136-144; or using CH3-truncated heavy chains), diabodies (see U.S. Pat. No. 5,837,242 Multivalent and multispecific binding proteins, their manufacture and use) constructs in which of one or two diabody molecules are heterodimerized by creating a fusion protein with the CL and CH1 immunoglobulin constant domains (see WO 02/02781).

In recent years, a variety of chemical and recombinant methods have been developed for the production of bispecific and/or multivalent antibody fragments. For review, see: Kriangkum J, et al. Bispecific and bifunctional single chain recombinant antibodies. Biomol Eng September 2001; 18(2):31-40, Holliger P. and Winter, G., Curr. Opin. Biotechnol. 4, 446-499 (1993); Carter, P. et al., J. Hematotherapy 4, 463-470 (1995); Pluckthan, A. and Pack, P., Immunotechnology 3, 83-105 (1997). Bispecificity and/or bivalency has been accomplished by fusing two scFv molecules via flexible linkers, leucine zipper motifs, C_{H}C_{L}-heterodimerization, and by association of scFv molecules to form bivalent monospecific diabodies and related structures. Multivalency has been achieved by the addition of multimerization sequences at the carboxy or amino terminus of the scFv or Fab fragments, by using for example, p53, streptavidin and helix-turn-helix motifs. For example, by dimerization via the helix-turn-helix motif of an scFv fusion protein of the form (scFv1)-hinge-helix-turn-helix-(scFv2), a tetravelent bispecific is produced having two scFv binding sites for each of two target antigens.

Production of IgG type bispecific antibodies, which resemble IgG antibodies in that they posses a more or less complete IgG constant domain structure, has been achieved by chemical cross-linking of two different IgG molecules or by co-expression of two antibodies from the same cell. Both methods result in production of significant amounts of undesired and non-functional species due to mispairing among the component heavy and light chains. Methods have been employed to reduce or eliminate mispairing.

One strategy developed to overcome unwanted pairings between two different sets of IgG heavy abd light chains co-expressed in transfected cells in modification of the Cₕ3 domains of two heavy chains to reduce homodimerization between like antibody heavy chains. Merchant, A. M., et al., (1998) Nat. Biotechnology 16, 677-681. In that method, light chain mispairing was eliminated by requiring the use of identical light chains for each binding site of those bispecific antibodies.

To produce bispecific antibodies, Kostelny et al (J. Immunology 148:1547 (1992)) fused Fab fragments of antibodies to the leucine zipper portions of fos and jun proteins in the absence of a single chain construct for the antigen combining region. These methods are well described in the literature and summarized with references in Antibody Fusion Proteins, Steven M Chamow, Avi Ashkenazi Eds. ISBN 047118358X May 1999 Wiley; Kontermann, R., et al.(Eds.) particularly at pages 139-145. Pack and Pluckthun, fused a single chain antibody to amphipathic helices from a four helix bundle or from leucine zipper proteins.

Bispecific antibodies that are in a conventional IgG-like and Fab-like format have been developed by Zhu as tetravalent or bivalent molecules, respectively with each of the chains serving to anchor a binding moiety (see WO 01/90192 and FIG. 1 therein), preferably consisting of a scFv. In the bispecific IgG-like construct, each side of the molecule comprises a CH1 domain and a CL domain and each CH and CL domain is linked through its N-terminus to a scFv of different specificity. The invention herein contemplates that this construct can readily be adapted to have each each half of the molecule associated with a polypeptide eg. a scFv of the same specificity so that each half of the molecule is monospecific (or to have each half of the molecule associated with different pairings of scFvs) so that each half of the molecule is effectively monospecific. The invention herein contemplates that a bivalent relatively low affinity second ligand binding moiety is used to activate receptors that require cross-linking for activity. The invention also contemplates that numerous permutations in which the functional affinity of the first ligand binding moiety whether monospecific or bispecific can be accentuated relative the functional affinity of the second ligand binding moiety including employing a first ligand high affinity scFvs for a single antinstances in which the second ligand binding moiety is effectively monovalent (has one, or one useful binding moiety). The invention also contemplates that this construct can have a truncated Fc portion and various known methods in the art for improving the pairing efficiency of the heavy chains. The invention also contemplates that the CH1 and CL domains of the second ligand binding moiety can be truncated as in camelid antibodies for efficient delivery eg. of biologic effector ligands.

Methods of Generating Antibodies that Bind to Selected Target Ligands

A variety of technologies for generating antibodies with desired specificity have been extensively developed and become well known to and routinely practiced by those skilled in the art including phage display (see review in Basic Methods in Antibody Production & Characteriztion G. C. Howard et al. eds. CRC Press 2001 p. 105) and other display systems (ribosome display, display on the surface of various cells), immunizing mice, including particularly mice having human Ig genes, and antibody microarray technologies. These methods have also been extended to making antibodies with dual specificites such as diabodies (U.S. Pat. No. 5,837,242 Multivalent and multispecific binding proteins, their manufacture and use) and are the subject of extensive scientific and patent literature. For example, see U.S. patents of Winter et al. U.S. Pat. Nos. 6,291,650; 6,291,161; 6,291,158; 6,017,732; 6,225,447; 6,172,197; 6,140,471, 6,010,884, 5,969,108, 5,871,907, 5,858,657; 5,733,743, 5,723,287 and those of Dyax, Morphosys, and Cambridge Antibody Technology.

Affinity Maturation

Methods of codon based mutagenesis have been extensively developed for engineering the antibody binding site. For example, the use of such methods in a filamentous phage display system is described in Antibody Engineering 2^{nd} ed. Carl A. K. Borrebaeck, Oxford University Press 1995 p117-128 see also pp.53-84 with respect to techniques of phage display of antibodies (see also Kontermann, R.: Dübel, S., (Eds.): (2001)Antibody Engineering ISBN: 3-540-41354-5; Dong L, et al. Generation of affinity matured scFv antibodies against mouse neural cell adhesion molecule L1 by phage display. Biochem Biophys Res Commun.(2003);301(1):60-70. PMID:12535641 Dall' Acqua W F, et al. Increasing the affinity of a human IgG1 for the neonatal Fc receptor: biological consequences. J Immunol.(2002);169(9):5171-80.PMID:12391234; Fujiwara K, et al. A single-chain antibody/epitope system for functional analysis of protein-protein interactions. Biochemistry.(2002);41(42):12729-38.PMID:12379115; Iwasaki A, Doi T, Umetani M, Watanabe M, Suda M, Hattori Y, Nagoya T. Affinity improvement of the high-affinity immunoglobulin E receptor by phage display. Biochem Biophys Res Commun.(2002);293(1):542-8.PMID:12054635; Chen Y, et al. Selection and analysis of an optimized anti-VEGF antibody: crystal structure of an affinity-matured Fab in complex with antigen. J Mol Biol.(1999)293(4):865-81.PMID:10543973.]; Dwyer M A, Lu W, Dwyer J J, Kossiakoff A A. Biosynthetic phage display: a novel protein engineering tool combining chemical and genetic diversity. Chem Biol. April 2000; 7(4):263-74. PMID:10780926; Iwasaki A, Doi T, Umetani M, Watanabe M, Suda M, Hattori Y, Nagoya T. Affinity improvement of the high-affinity immunoglobulin E receptor by phage display. Biochem Biophys Res Commun. Apr. 26, 2002; 293(1):542-8. PMID: 12054635 [PubMed—indexed for MEDLINE]; Van den Beucken T, Pieters H, Steukers M, van der Vaart M, Ladner R C, Hoogenboom H R, Hufton S E.; Affinity maturation of Fab antibody fragments by fluorescent-activated cell sorting of yeast-displayed libraries. FEBS Lett. Jul. 10, 2003; 546(2-3):288-94. PMID:12832056; Valjakka J, Hemminki A, et al. Crystal structure of an in vitro affinity- and specificity-matured anti-testosterone Fab in complex with testosterone.

Improved affinity results from small structural changes within the variable domains. J Biol Chem. (2002);277(46):44021-7. Epub Aug. 23, 2002. PMID:12196551. Li Y, et al. X-ray snapshots of the maturation of an antibody response to a protein antigen. Nat Struct Biol.(2003);10(6):483-8.PMID:12740607; Wu H, et al. Tailoring kinetics of antibodies using focused combinatorial libraries. Methods Mol Biol,(2003);207:213-33.

Methods of Generating Single Domain Ligands

The ability of a single variable fragment of an antibody to bind with specificity and suitable selected affinities in the nanomolar+range has been extensively demonstrated using camelid and human VH fragments. Methods of generating VHs with the desired specificity have been extensively described (see U.S. Pat. No. 6,248,516 Single domain ligands, receptors comprising said ligands methods for their production, and use of said ligands and receptors). (see also literature referenced herein on this subject).

Methods of Making Antibodies in E. Coli

The expression of recombinant antibodies, including diabodies in E. Coli has become routine. General precepts, and methods are discussed in Antibody Engineering 2^{nd} ed. Carl A. K. Borrebaeck, Oxford University Press 1995 p229-266 see also Antibody Therapeutics W J Harris et al. eds. CRC Press 1997 p. 221; see also review in Biotechnology, Volume 5A, Recombinant Proteins, Monoclonal Antibodies, and Therapeutic Genes A. Mountain. U. Ney. Dietmar Schomburg ISBN: 3-527-28315-3, January 1999, Antibody Production: Essential Techniques Peter J. Delves ISBN: 0-471-97010-7 Wiley June 1997 and Antibody Therapeutics Production, Clinical Trials, and Strategic Issues, By Rathin C. Das, Ph.D., M.B.A. & K. John Morrow, Jr., Ph.D., D&MD Publications October 2001 Chapter 3.

Eukaryotic & Other Expression & Production Systems

Approaches for the eukaryotic expression of antibodies and antibody fusion proteins and the preparation of vectors for use in such methods are well known and extensively described in the literature. General precepts, and methods are discussed is Antibody Engineering 2^{nd} ed. Carl A. K. Borrebaeck, Oxford University Press 1995 p267-293 (see also Antibody Therapeutics W J Harris et al. eds. CRC Press 1997 p. 183-220; see also review in Biotechnology, Volume 5A, Recombinant Proteins, Monoclonal Antibodies, and Therapeutic Genes A. Mountain., U. Ney, Dietmar Schomburg ISBN: 3-527-2831 5-3. Wiley, January 1999 and Antibody Production: Essential Techniques Peter J. Delves ISBN: 0-471-97010-7 Wiley June 1997 and Antibody Therapeutics Production, Clinical Trials, and Strategic Issues, By Rathin C. Das, Ph.D., M.B.A. & K. John Morrow, Jr., Ph.D., D&MD Publications October 2001 Chapter 3.

With respect to a review of immunotoxins see also Antibody Therapeutics W J Harris et al. eds. CRC Press 1997 p 33

With respect to Methods for producing recombinant vectors see also U.S. Pat. No. 5,962,255 Methods for producing recombinant vectors

Formulation, purification and analytic methods involving antibodies are well known to those skilled in the art and have been extensively reviewed. With respect to formulation, purification and analytic methods see for example, reviews in Antibody Therapeutics Production, Clinical Trials, and Strategic Issues, By Rathin C. Das, Ph.D., M.B.A. & K. John Morrow, Jr., Ph.D., D&MD Publications October 2001, Chapter 4.

With respect to methods of generating antibodies against self-antibodies see U.S. Pat. No. 5,885,793 Production of anti-self antibodies from antibody segment repertoires and displayed on phage

Antibody Conjugates

Methods of chemical manipulation of antibodies for attachment of ligands (eg. biotin), radionuclides etc. are well known in the art and have been extensively reviewed (for example see review in Basic Methods in Antibody Production & Characteriztion G. C. Howard et al. eds. CRC Press 2001, p. 199; with respect to therapeutic principles see for example, Antibody Therapeutics W J Harris et al. eds. CRC Press 1997 p 53-88).

The applications of bispecific antibodies, including methods of making and using them have been extensively reviewed (ee for example van Spriel A B, van Ojik H H, van De Winkel J G. Immunotherapeutic perspective for bispecific antibodies. Immunol Today. August 2000; 21(8):391-7; Weiner L M. Bispecific antibodies in cancer therapy. Cancer J Sci Am. May 2000; 6 Suppl 3:S265-71. Barbet J, et al. Pretargeting with the affinity enhancement system for radioimmunotherapy. Cancer Biother Radiopharm. June 1999; 14(3):153-66. de Wolf F A, Brett G M. Ligand-binding proteins: their potential for application in systems for controlled delivery and uptake of ligands. Pharmacol Rev. June 2000; 52(2):207-36.: Wang H, Liu Y, Wei L, Guo Y. Bi-specific antibodies in cancer therapy Adv Exp Med Biol. 2000; 465:369-80; Staerz U D, Lee D S, Qi Y. Induction of specific immune tolerance with hybrid antibodies. Immunol Today. April 2000; 21(4):172-6: December 1999; 43(4):336-43. Elsasser D, Stadick H, van de Winkel J G, Valerius T. GM-CSF as adjuvant for immunotherapy with bispecific antibodies. Eur J Cancer. August 1999; 35 Suppl 3:S25-8. Molema G, Kroesen B J, Helfrich W, Meijer D K, de Leij L F. The use of bispecific antibodies in tumor cell and tumor vasculature directed immunotherapy. J Control Release. Feb. 14, 2000; 64(1-3):229-39. Bodey B, Bodey B, Siegel S E, Kaiser H E. Genetically engineered monoclonal antibodies for direct anti-neoplastic treatment and cancer cell specific delivery of chemotherapeutic agents. Curr Pharm Des. February 2000; 6(3):261-76. Kudo T, Suzuki M, Katayose Y, Shinoda M, Sakurai N, Kodama H, Ichiyama M, Takemura S, Yoshida H, Saeki H, Saijyo S, Takahashi J, Tominaga T, Matsuno S. Specific targeting immunotherapy of cancer with bispecific antibodies. Tohoku J Exp Med. August 1999; 188(4):275-88. Koelemij R, et al. Bispecific antibodies in cancer therapy, from the laboratory to the clinic. J Immunother. November 1999; 22(6):514-24. Segal D M, Weiner G J, Weiner L M Bispecific antibodies in cancer therapy Curr Opin Immunol. October 1999; 11(5):558-62. Hudson P J. Recombinant antibody constructs in cancer therapy. Curr Opin Immunol. October 1999; 11(5):548-57. Barth R F et al, Boron neutron capture therapy of brain tumors: an emerging therapeutic modality. Neurosurgery. March 1999; 44(3):433-50; Fleckenstein G, Osmers R, Puchta J. Monoclonal antibodies in solid tumours: approaches to therapy with emphasis on gynaecological cancer, Med Oncol. December 1998; 15(4):212-21. Guyre C A, Fanger M W. Macrophage-targeted killing and vaccines. Res Immunol. September-October 1998; 149(7-8):655-60 Cao Y, Suresh M R. Bispecific antibodies as novel bioconjugates. Bioconjug Chem. November-December 1998; 9(6):635-44. Farah R A, et al, The development of monoclonal antibodies for the therapy of cancer. Crit Rev Eukaryot Gene Expr. 1998; 8(3-4):321-56.: Volm M. Multidrug resistance and its reversal.Anticancer Res. July-August 1998; 18(4C):2905-17. Rouard H, et al, Fc receptors as targets for immunotherapy.Int Rev Immunol. 1997; 16(1-2):147-85. Fan Z et al. Therapeutic application of anti-growth factor receptor antibodies; Curr Opin Oncol. January 1998; 10(1):67-73. de Gast G C, et al,Clinical perspectives of bispecific antibodies in cancer. Cancer Immunol Immunother. November-December 1997; 45(3-4):121-3. Carter P, Merchant A M. Engineering antibodies for imaging and therapy.Curr Opin Biotechnol. August 1997; 8(4):449-54. Pluckthun A, et al, New protein engineering approaches to multivalent and bispecific antibody fragments. Immunotechnology. June 1997; 3(2):83-105. Rihova B. Targeting of drugs to cell surface receptors. Crit Rev Biotechnol. 1997; 17(2):149-69. Molema G et al,Tumor vascular endothelium: barrier or target in tumor directed drug delivery and immunotherapy. Pharm Res. January 1997; 14(1):2-10. Bodey B, et al, Human cancer detection and immunotherapy with conjugated and non-conjugated monoclonal antibodies. Anticancer Res. March-April 1996; 16(2):661-74 Hartmann F et al, Treatment of Hodgkin's disease with bispecific antibodies. Ann Oncol. 1996; 7 Suppl 4:143-6. Wels W, et al, Intervention in receptor tyrosine kinase-mediated pathways: recombinant antibody fusion proteins targeted to ErbB2. Curr Top Microbiol Immunol. 1996; 213 (Pt 3): 113-28.: Kairemo K J. Radioimmunotherapy of solid cancers: Acta Oncol. 1996; 35(3):343-55. Verhoeyen M E, et al, Antibody fragments for controlled delivery of therapeutic agents. Biochem Soc Trans. November 1995; 23(4):1067-73. Haagen I A. Performance of CD3×CD 19 bispecific monoclonal antibodies in B cell malignancy. Leuk Lymphoma. November 1995; 19(5-6):381-93. Hudson P J. et al. Engineered Antibodies. Nat Med. January 2003; 9(1):129-34 PMID:12514726; Cao Y. et al. A rapid non-selective method to generate quadromas by microelectrofusion. J Immunol Methods. Nov. 16, 1995 :187(1):1-7. PMID:7490445; Presta L G. Engineering Antibodies for therapy. Curr Pharm Biotechnol. September 2002; 3(3): 237-56.

With respect to generation of anti-tumor antibodies and other antibody fragments for application herein as well as important related technologies see also WO 00/50008; WO 01/01137; WO 97/37791; WO99/37791; WO 97/10003; Hoogenboom et al. Nat. Biotechnology 15(2) February 1997 p125-126; Fell H. et al. Journal Of Immunolgy Vol 146(7) April 1991 p2446-2452; Anderson D. et al Bioconjugate Chemistry 14(1) January 1993 p10-18; U.S. Pat. No. 6, 172,197; U.S. Pat. No. 6,171,782; Immunological Investigations 2000 29(2) entire issue). Optionally the tumor binding portion internalizes and/or delivers a toxic payload, for example a radionuclide, or other toxin, or a cytokine to the tumor cell (with respect to selection of tumor internalizing human antibodies see for example Pool M et al. J Mol Biol. Sep. 1, 2000; 301(5):1149-61, see also Kohl M D et al. J Mol. Biol. Biotechniques (2000) Vol 28(1) p162

With respect to internalizing antibodies see eg Biological Effects of Anti-ErbB2 Single Chain Antibodies Selected for Internalizing Function.; Biochem Biophys Res Commun. Jan. 12, 2001; 280(1):274-279 and references cited therein, Immunoconjugates of geldanamycin and anti-HER2 monoclonal antibodies: antiproliferative activity on human breast carcinoma cell lines J Natl Cancer Inst. Oct. 4, 2000; 92(19):1573-81; Foulon C F, et al., Radioiodination via D-amino acid peptide enhances cellular retention and tumor xenograft targeting of an internalizing anti-epidermal growth factor receptor variant III monoclonal antibody. Cancer Res. Aug. 15, 2000; 60(16):4453-60. Poul M A, Becerril B, Nielsen U B, Morisson P, Marks Selection of tumor-specific internalizing human antibodies from phage libraries J Mol Biol. Sep. 1, 2000; 301(5):1149-61.Vrouenraets M B, et al.,Targeting of a hydrophilic photosensitizer by use of internalizing monoclonal antibodies: A new possibility for use in photodynamic therapy. Int J Cancer. Oct. 1, 2000; 88(1):108-14. With respect to target receptors related to the inventions defined herein see also U.S. Pat. No. 6,277,962.

The invention contemplates research and treatments using multi-functional ligands of the invention with respect to non-human mammals, including preferably agricultural animals, canine species, primates and mice having similar receptors/antigens. Models of metastasis in animals are well known in the art (see for example Chirgwin J M, Guise T A. Molecular mechanisms of tumor-bone interactions in osteolytic metastases. Crit Rev Eukaryot Gene Expr. 2000;10(2):159-78. 3: Kobaek-Larsen M, et alReview of colorectal cancer and its metastases in rodent models: comparative aspects with those in humans. Comp Med. February 2000; 50(1):16-26. 5: Magnano M, et al.A physical-based model for the simulation of neoplastic growth and metastasis. J Surg Oncol. June 2000; 74(2): 122-9. 6: Hoffman R M. Orthotopic metastatic mouse models for anticancer drug discovery and evaluation:a bridge to the clinic. Invest New Drugs. 1999;17(4):343-59. Russo J, Russo I H. The pathway of neoplastic transformation of human breast epithelial cells.Radiat Res. January 2001; 155(1 Pt 2):151-154. Duffy M J, McCarthy K. Matrix metalloproteinases in cancer: prognostic markers and targets for therapy(review).Int J Oncol. June 1998; 12(6):1343-8. 22: Banerjee A, Quirke P. Experimental models of colorectal cancer. Dis Colon Rectum. April 1998; 41(4):490-505. Wu T T et al.Establishing human prostate cancer cell xenografts in bone: induction of osteoblastic reaction by prostate-specific antigen-producing tumors in athymic and SCID/bg mice using LNCaP and lineage-derived metastatic sublines. Int J Cancer. Sep. 11, 1998; 77(6):887-94.61: Molpus K L, et alCharacterization of a xenograft model of human ovarian carcinoma which produces intraperitoneal carcinomatosis and metastases in mice. Int J Cancer. Nov. 27, 1996; 68(5):588-95.65: Pages J C, Sordat B, Bautista D, Costa J, Benhattar J. Detection of rare circulating human colon tumor cells in a nude mouse xenograft model. Cancer Lett. Aug. 23, 1996; 106(1):139-44.66: Sakakibara T, et al.Doxorubicin encapsulated in sterically stabilized liposomes is superior to free drug or drug-containing conventional liposomes at suppressing growth and metastases of human lung tumor xenografts. Cancer Res. Aug. 15, 1996; 56(16):3743-6.

With respect to modifying an antibody to increase its affinity see also Crystal structure of Fab198, an efficient protector of the acetylcholine receptor against myasthenogenic antibodies. Eur J Biochem. July 2001; 268(13):3685-3693.

For example, in one embodiment the invention contemplates a bispecific antibody comprising an antigen binding component specific for a tumor cell associated antigen and a relatively low affinity anti-IL-6 receptor antibody component. With respect to the anti-tumor role of IL-6 see Wei L H et al. Interleukin-6 in cervical cancer: the relationship with vascular endothelial growth factor. Gynecol Oncol. July 2001; 82(1):49-56.

The invention contemplates that TCRs and modified TCRs (see for example, WO 01/48145) may be used as ligands, in place of antibody fragments for binding to target ligands such as peptide/MHC ligands.

Techniques for generating antibodies, and methods, for example of subtractive screening useful to identify other tissue specific antibodies, including those optionally having smaller scFv, Fab and dAb (single domain antibody or functional fragment thereof) component by phage or ribosome display are well known in the art (see for example Hoogenbom H R et al. Immunol. Today (August. 2000) Vol 8 p 371; Schaffitzel C. et al. J Immunol. Methods (Dec. 10, 1999) 231(1-2) p. 119; Roberts R W et al. Curr Opin Chem Biol. June 1999; 3(3):268-73; Winter G. et al. Annu Rev Immunol 1994 12:433-55; Kontermann R E et al. Nat Biotechnol. July 1997; 15(7):629-31; Phage Display of Peptides and Proteins, A Laboratory Manual Kay B K et al. Eds 1996 Academic Press; Immunology Methods Manual Lefkovits, I ed. 1997 Academic Press;Hoogenboom et al. Immunotechnology 4 (1998)1-20;

With respect to making single domain antibodies see for example U.S. Pat. No. 5,824,520, U.S. Pat. No. 5,622,836, U.S. Pat. No. 5,702,892, U.S. Pat. No. 5,959,087, Unique single-domain antigen binding fragments derived from naturally occurring camel heavy-chain antibodies.J Mol Recognit. March-April 1999; 12(2): 131-40. An antibody single-domain phage display library of a native heavy chain variable region: isolation of functional single-domain VH molecules with a unique interface. J Mol Biol. Jul. 16, 1999; 290(3):685-98 and references cited in these references.

Methods for making antibody fusion proteins and bi-specific antibodies including diabodies etc. and fusion proteins thereof are well established in the art (for reviews and particular applications see for example Adams G P et al. Journal of Immunological Methods 231 (1999) 249-260; U.S. Pat. Nos. 6,121,424, 6,027,725 and 6,025,165; EP 0654085; Hudson P. Exp. Opin. Invest. Drugs (2000) 9(6): 1231-1242; Antibody Fusion Proteins Steven M Chamow, Avi Ashkenazi Eds. ISBN 047118358X May 1999 Wiley; Antibody Engineering, Carl A. Borrebaeck Oxford University Press, 1995; Antibody Engineering:A Practical Approach David J. Chiswell, Hennie R. Hoogenboom, John McCafferty Oxford University Press, 1996; Antibody Engineering Protocols, Sudhir Paul (1995) Humana Press; Antibody Expression & Engineering (1998) Henry Y. Wang, Tadayuki Imanaka, American Chemical Society; Zhu Z. Biotechnology (NY) February 1996; 14(2): 192-6; Nielsen U B et al. Cancer Res. Nov. 15, 2000; 60(22):6434-40; Lawrence L J. Et al Febs Lett. Apr. 3, 1998; 425(3) 479-84; Hollinger et al., Cancer Immunol Immunother November-December 1997 45 (3-4) 128-30; Immunotargeting of tumors: state of the art and prospects in 2000 Bull Cancer. November 2000; 87(11):777-91; Hellfrich Wet al Int. J. cancer Apr. 13, 1998 76(2): 232-9; Wu A M, Q J Nuc Med. September 2000; 44(3):268-83 Krebs B. Et al. J Interferon cytokine Res September 1998 18(9): 783-91; Takemura Si, et al. Protein Eng. August 2000; 13(8): 583-8; Cochlovius B et al. J Immunol. Jul. 15, 2000; 165(2):888-95; Atwelll J L et al. Protein Eng. July 1999; 12(7): 597-604; kiprivanov S M et al. J. Mol Biol. Oct. 15, 1999 293 (1): 41-56; Alt M. et al FEBS LeH. July 2 454 ( ) 1-2) 90-4. Hudson P J et al. J Immunol Methods Dec. 10, 1999; 231(1-2):177-89 Ardnt M A et al. Blood Oct. 15, 1999 94(8): 2562-8; Lu D. et al. J Immunol. Methods Nov. 19, 1999; 230(1-2):159-171; Santos A D et al, Clin Cancer Res Oct. 5, 1999 (10 suppl): 31185-31235 Kontermann R E et al. Nat Biotechnol. July 1997; 15(7):629-31; Dolez et al. Protein eng. (2000) August 13 (8): 565-74; Adams G P et al. Nucl. Med. Biol (2000) May 27 (4); 339-46; Williams L E et al. Med phys May 2000 27(5) 988-94; Fitzgerald K. Protein Eng October 1997 10(10): 1221-5 and the various references cited therein) as are various methods for identifying internalizing antibodies and creating toxin, radionuclide and cytokine fusions/conjugates (see ao Y et al Bioconj. Chem November-December 1998; 9(6): 635-44) for fully exploiting various aspects of the invention herein defined (see for example Becerril B et al. Biochem Biophys Res Comm Feb. 16, 1999; 255(2):386-93 see also additional references below.

Triabodies and other known multivalent antibodies etc. (see for example Iliades P et al. FEBS Lett. Jun. 16, 1997; 409(3):437-41) etc. could advantageously be employed to provide additional functionalities, as well as variation in avidity etc. for the purposes of variously exploiting the invention herein.

Technologies for rendering the multifunctional ligands of the invention less immunogenic (eg such as employed by Biovation) are preferably applied to the multifunctional ligands of the invention.

With respect to targeting Fas-L see U.S. Pat. No. 6,068,841 :Antibodies to Fas-L for treatment of hepatitis.

With reference to modulating binding of leucocytes to endothelial adhesion molecules see for example U.S. Pat. No. 6,123,915 and the references therein cited.

It is well known to those in the art to make bispecific antibodies which are adapted to bind two different ligands on the same cell, for example so called antigen-forks as disclosed in U.S. Pat. No. 5,705,614 (see also Shi T et al. Murine bispecific antibody 1A10 directed to human transferrin receptor and a 42-kDa tumor-associated glycoprotein also Clin Immunol Immunopathol February 1996; 78(2):188-95; Amoroso A R et al., Binding characteristics and antitumor properties of 1A10 bispecific antibody recognizing gp40 and human transferrin receptor Cancer Res Jan. 1, 1996; 56(1):113-20; Ring D B et al., Antigen forks: bispecific reagents that inhibit cell growth by binding selected pairs of tumor antigens, Cancer Immunol Immunother July 1994; 39(1):41-8; Lu D et al., Complete inhibition of vascular endothelial growth factor (VEGF) activities with a bifunctional diabody directed against both VEGF kinase receptors, fins-like tyrosine kinase receptor and kinase insert domain-containing receptor. Cancer Res Oct. 1, 2001; 61(19):7002-8; Schmiedl A, Breitling F, Dubel S. Expression of a bispecific dsFv-dsFv′ antibody fragment in Escherichia coli. Protein Eng October 2000; 13(10):725-34 see also Park S S, et al., Generation and characterization of a novel tetravalent bispecific antibody that binds to hepatitis B virus surface antigens Mol Immunol December 2000; 37(18):1123-30; Kriangkum J et al., Bispecific and bifunctional single chain recombinant antibodies Biomol Eng September 2001; 18(2):31-40; U.S. Pat. Nos. 4,474,893, 5,989,830; WO 00/29431).

With respect to antibodies to autoantigens, ADEPT, use of anti-eotaxin antibodies, DeImmunization, antibody-cytokine fusions, ribosome display, xenomouse technology; cutting edge phage display techniques, construction of human antibody fragment based phage display libraries, selection of internalizing antibodies by phage-display, cancer targeting antibodies, antibody arrays, plantibodies, design of mutant IGSF domains of CD2, CD58 and TCR; oligopeptide eg. paratope mimetics, diabodies, minibodies, triabodies, tetrabodies and related size/kinetics issues, caspase activatable pro-drugs, delivery of Bismuth-213 via scFv and diabodies, anti-angionenesis marker strategies, immunoenzype therapy of cancer (eg. with Rnases) pancarcinomic antigens like CEA (TAG”)-72; and related technologies see the papers and references in Proceedings of IBC's 11^{th} Annual International Conference on Antibody Engineering, State of the Art, Science, Technology and Applications Dec. 3-6, 2000 La Jolla, Calif.

With respect to recent developments with respect to target ligands and/or immunotherapy having application herein see also WO 01/12224, WO 01/14550, WO 01/11059, WO 01/10205, WO 01/00679, WO029445 WO 01/14885, WO/14564, WO 01/14558, WO 01/14224, WO 01/13945, WO 01/12840, WO 01/12781, Wo 01/12674, WO 01/12670, WO 01/12224, WO 01/12646; WO 01/12223, WO 01/12218, WO 01/12217, WO 01/12216, WO 01/12154, WO 01/14557, WO 01/11059, WO 01/10912, WO 01/11040, WO 01/10888, WO 01/10460, WO 01/10205, WO 01/09611, WO 01/09328, WO 01/09186, WO 01/09192, WO 01/08635, WO 01/07481, WO 01/07082, WO 01/07084, WO 01/07081, WO 01/07484, WO 01/07466, Triggering Fc alpha-receptor I (CD89) recruits neutrophils as effector cells for CD20-directed antibody therapy. J Immunol. Nov. 15, 2000; 165(10):5954-61. CD47 engagement inhibits cytokine production and maturation of human dendritic cells. J Immunol. Feb. 15, 2000; 164(4):2193-9.

The invention also contemplates use of recently published antibodies in the context of the invention (see WO 01/19861, WO 01/19990, WO 01/19860, WO 01/19987, WO 01/19990, WO 99/58678, WO 00/59943, WO 01/18014, WO 01/18016, WO 01/18204, WO 01/18042, WO 01/18021, WO 01/18014, WO 01/18046, WO 01/16166, WO 01/15731, WO 01/15728, WO 01/16183, WO 01/16170, WO 01/15732.

With respect to the display of functional peptides on an antibody type scaffold see Nuttal S D; et al., Proteins (1999) 36: 217-227; see also Skerra A., J. Mol. Recognition July-August 2000 13(4): 167-187.

As described above, the invention also contemplates that the lower affinity ligand binding arm of the aforementioned multifunctional ligand (ie. having a high affinity targeting arm and a lower affinity effector arm) is constituted by a high affinity ligand, for example an high affinity antibody or functional fragment thereof, which binds to a target biological effector (eg. a cytokine, chemokine, growth factor, hormone or other biological response modifier or drug) with high affinity, in a manner which permits the effector to continue to bind to its desired target receptor while bound to the antibody (ie. the antibody binds to a portion of the effector which is not critically involved in the effector binding to its receptor) provided that when bound to the effector the antibody or fragment thereof has, when combined with the effector, a suitably lower affinity for the receptor than the ligand binding arm which functions as the high affinity binder has for its target cell marker. In one embodiment the binding moiety which binds to the biological effector binds to it with higher affinity than the affinity that the effector has for the effector receptor. The invention also contemplates that this binding arm can bind to biological effector in a manner which permits it to bind to one receptor but not a related receptor to which the effector would otherwise bind (see examples below). The invention also contemplates that antibody arrays are used to screen for antibodies which are capable of binding to such biological effectors, while bind in situ to their receptors. The invention also contemplates that such binders, when bound to the biological effector, can be used to test their ability to bind to related receptors, such as those within the same family eg. within the same family of TNF like receptors. With respect to antibody microarrays see for example Cahill D J.Protein and antibody arrays and their medical applications.J Immunol Methods. April 2001; 250(1-2):81-91. MacBeath G. Proteomics comes to the surface.Nat Biotechnol. September 2001; 19(9):828-9. Clewley J P.Recombinant protein arrays.Commun Dis Public Health. December 2000; 3(4):311-2; Holt L J, Enever C, de Wildt R M, Tomlinson I M. The use of recombinant antibodies in proteomics.Curr Opin Biotechnol. October 2000; 11(5):445-9. Walter G, et al.Protein arrays for gene expression and molecular interaction screening.Curr Opin Microbiol. June 2000; 3(3):298-302. de Wildt R M, Mundy C R, Gorick B D, Tomlinson I M.Antibody arrays for high-throughput screening of antibody-antigen interactions.Nat Biotechnol. September 2000; 18(9):989-94.Holt L J, et al. By-passing selection: direct screening for antibody-antigen interactions using protein arrays. Nucleic Acids Res. Aug. 1, 2000; 28(15):E72 and the references cited therein. The term receptor as used herein for greater certainty includes decoy receptors. Examples of decoy receptors include TRAIL decoy receptors (APO-2L), CD44 decoy like receptors (hyaluronan), interleukin receptor like protein (IL-17) (see J Biol Chem Nov. 12, 2001), CD95-Fc decoy receptor, TRAMP, IL-1 RII receptor, osteoprotegerin (OPG), IL13Ralpha2.

Affinity Maturation

Techniques for affinity maturation using high throughput screening techniques to evaluate mutants are well known in the art. Femtomolar affinities have been achieved and it is quite common to obtain nanomolar to picomolar affinities as a result of an affinity maturation process. For example it well known to use techniques of parsimonious mutagenesis to engineer amino acid change at selected “hotspots”. With respect to affinity maturation see for example Coia G, Hudson P J, Irving R A. Protein affinity maturation in vivo using E. coli mutator cells. J Immunol Methods. May 1, 2001; 251(1-2):187-93. Manivel V, Sahoo N C, Salunke D M, Rao K V. Maturation of an antibody response is governed by modulations in flexibility of the antigen-combining site. Immunity. November 2000; 13(5):611-20. Boder E T, Midelfort K S, Wittrup K D. Directed evolution of antibody fragments with monovalent femtomolar antigen-binding affinity.Proc Natl Acad Sci USA. Sep. 26, 2000; 97(20):10701-5. Holler P D, Holman P O, Shusta E V, O'Herrin S, Wittrup K D, Kranz D M. In vitro evolution of a T cell receptor with high affinity for peptide/MHC. Proc Natl Acad Sci USA. May 9, 2000; 97(10):5387-92. Daugherty P S, Chen G, Iverson B L, Georgiou G. Quantitative analysis of the effect of the mutation frequency on the affinity maturation of single chain Fv antibodies. Proc Natl Acad Sci USA. Feb. 29, 2000; 97(5):2029-34. VanAntwerp J J, Wittrup K D. Fine affinity discrimination by yeast surface display and flow cytometry.Biotechnol Prog. January-February 2000; 16(1):31-7. Adams G P, Schier R. Generating improved single-chain Fv molecules for tumor targeting. J Immunol Methods. Dec. 10, 1999; 231(1-2):249-60. Daugherty P S, Chen G, Olsen M J, Iverson B L, Georgiou G. Antibody affinity maturation using bacterial surface display. Protein Eng. September 1998; 11(9):825-32. Wong Y W, Kussie P H, Parhami-Seren B, Margolies M N. Modulation of antibody affinity by an engineered amino acid substitution. J Immunol. Apr. 1, 1995; 154(7):3351-8. Balint R F, Larrick J W. Antibody engineering by parsimonious mutagenesis. Gene. Dec. 27, 993; 137(1):109-18. Schillbach J F, Near R I, Bruccoleri R E, Haber E, Jeffrey P D, Novotny J, Sheriff S, Margolies M N. Modulation of antibody affinity by a non-contact residue. Protein Sci. February 1993; 2(2):206-14.Chames P, Baty D. Engineering of an anti-steroid antibody: amino acid substitutions change antibody fine specificity from cortisol to estradiol.Clin Chem Lab Med. June 1998; 36(6):355-9. Kussie P H, Parhami-Seren B, Wysocki L J, Margolies M N. A single engineered amino acid substitution changes antibody fine specificity. J Immunol. Jan. 1, 1994; 152(1):146-52, as well as references cited therein; (Griffin M D, Holman P O, Tang Q, Ashourian N, Korthauer U, Kranz D M, Bluestone J A. Development and applications of surface-linked single chain antibodies against T-cell antigens. J Immunol Methods. Feb. 1, 2001; 248(1-2):77-90. Review. PMID:11223070)

With respect to generation of high affinity antibodies and affinity maturation of antibodies see also Hanes J. Nat. Biotechnol. December 2000; 18(12): 1287-92; references in Hudson P J Exp. Opin. Invest. Drugs (2000) 9(6) 1231-1242; Toran J L et al Evr. J. Immunol. January 2001; 31(1) 128-137. Nielson V B et al. Cancer Res Nov. 15, 2000; 60 (22) 6434-40 Adams Gp, Journal of Immunological Methods (1999) 249-260; Chowdhury P S et al (June 1999) Nature Biotechnology Vol 17 p. 568 With respect to strategies and recent technologies which have application to the invention see references in Hudson P J Exp. Opin. Invest. Drugs (2000) 9(6) 1231-1242 and in particular references relating to strategies to achieve multivalency and multispecificity; recruitment of viruses, ADEPT, photoactivation of cytotoxic radionuclides; surface receptor cross-linking; (see also Eur. J. Immunol 2000 30(10) 3006), use of anti-B antibodies; immunocytokines (see also Lode H N Immul. Res. 2000, 21 (2-3) 279-88; Gillies S D Cancer Research 59 2159-2166 May 1999; Lode H N et al Drugs of Today 2000 36(5) 3221-336).

With respect to making high affinity antibodies by panning on whole cells (as previously described in the literature for finding new cancer antigens), see also WO 03/068916; WO 02/46238; WO 03/025019; WO 02/00005 and WO 02/46238. High throughput methods for making high affinity antibodies described above include those described in WO 01/27160; WO 02/36738; WO 03/068801 (high on-rate); WO 01/64751; WO 01/32712; WO 02/034886; WO 01/32712 and WO 02/036738. Specific technologies that are useful in this regard are also described in WO 02/034886; WO 01/32712; WO 98/47343; WO 98/45332 (high throughput humanization); WO 99/36569; WO 02/085945; WO 01/25492; WO 02/48674 (array based); WO 99/40434 (array based); WO 02/061087 (peptide immutype based); WO 03/048729 (epitope categorization); WO 02/084277; WO 99/53049 (epitope based); WO 00/12566; WO 00/31537; WO 00/29584; WO 99/45962; WO 02/092780; WO 02/073180 (microarray based); WO 02/50120; WO 02/46233 (hybridoma based); WO 02/055718 Yeast Display);. WO 99/06834 array based, WO 98/45332 (humanization directed), WO 03/055978, WO 03/023032, WO 02/090496, WO 98/49286, WO 00/53744 (saturation mutagenesis). See also WO 03/068201, U.S. Patents: 20030143682 Antibodies and methods for generating genetically altered antibodies with high affinity; 20030120044 Methods of optimizing antibody variable region binding affinity; 20030054497 Increasing antibody affinity by altering glycosylation of immunoglobulin variable region; 20030036092 Directed evolution of enzymes and antibodies; 20030022240 Generation and affinity maturation of antibody library in silico; 20030008355 High-affinity antibodies; 20020177170 Structure-based selection and affinity maturation of antibody library; 20020164326 Ultra high affinity neutralizing antibodies; 20030166303 Novel screening method for molecular antagonist using flow-cytometry; 20030165988 High throughput generation of human monoclonal antibody against peptide fragments derived from membrane proteins; 20030153013 Antibody-based protein array system; 20030120044 Methods of optimizing antibody variable region binding affinity; 20030092125 Epitope-driven human antibody production and gene expression profiling; 20030082814 Secretion of T cell receptor fragments from recombinant escherichia coli cells; 20030091995 HUMAN ANTIBODIES; 20030054407 Structure-based construction of human antibody library; 20030049683 Rationally designed antibodies; 20030044772 Methods for identifying ligand specific binding molecules; 20030040606 Reducing immunogenicities of immunoglobulins by framework-patching; 20030039649 Super humanized antibodies; 20030022240 Generation and affinity maturation of antibody library in silico; 20030008355 High-affinity antibodies; 20020177170 Structure-based selection and affinity maturation of antibody library; 20020155502 Affinity maturation by competitive selection; 20020164326 Ultra high affinity neutralizing antibodies 20020127223 Method for reducing side effects of a drug; 20020098189 High potency recombinant antibodies and method for producing them; 20020048578 ANTIBODY VARIANTS; U.S. Pat. No. 6,610,472 Assembly and screening of highly complex and fully human antibody repertoire in yeast; U.S. Pat. No. 6,602,661 Methods and arrays for detecting biomolecules; U.S. Pat. No. 6,531,580 Anti-.alpha.v.beta.3 recombinant human antibodies and nucleic acids encoding same; U.S. Pat. No. 6,590,079 Anti-.alpha.v.beta.3 recombinant human antibodies, nucleic acids encoding same; U.S. Pat. No. 5,945,311 Method for producing heterologous bi-specific antibodies; U.S. Pat. No. 5,932,448 Bispecific antibody heterodimers (leucine zippers)

With respect to deimmunization of such antibodies, see WO 03/002607; WO 02/34779; WO 00/34317 and WO 98/52976.

With respect to practicial size limitations and pharmacokinetics of various types of antibodies and fragments see Colcher D. et al. G. J. Nucl. Med (1999) 43:132-139; Wu A M et al G. J. Nucl. Med September 2000; 44(3): 268:83; Williams L E et al Med Phys May 2000 27(5): 988-941 Ikomi F lymphology 32 (1999) 90-102.

With respect to the construction of diabodies see also Takemura SI et al. Protein Eng. August 2000; 13(8) 583-8; Biomol. Eng. September 2001; 18(2):31-40.

With respect to anti-cancer antibodies see also U.S. Pat. No. 6,180,357.

Other References and Important Antibodies:

Agonist Antibodies see U.S. Pat. No. 6,342,220; U.S. Pat. No. 6,475,487; WO 00/75348; Affinity maturation U.S. Pat. No. 6,127,524; WO 01/14424; WO 01/009192; Pre-targetting WO 02/082041, US 2002/0114808; U.S. Pat. No. 6,458,933; EP 1194167; WO 0074718; WO 99/66951; U.S. Pat. No. 6,492,496; WO 02/058717; WO01/62905; WO02/079232; WO00/78815; EP 0654085; U.S. Pat. No. 6,492,497; EP1251695; WO 0202641; WO1/667754

With respect to technologies to produce multivalent and/or multispecific antibodies see also U.S. Pat. No. 6,172, 197; WO 92/01047; WO 93/11161; WO 94/07921; WO 94/13804; Helfrich W. et al. Journal of Immunological Methods 237 (2000) 131-145. Proceedings of 11^{th} IBC Conference on Antibody Engineering; WO 01/85795; Monoclonal antibodies may be routinely produced as taught by Harlow, E. and D. Lane, (1988) ANTIBODIES: A Laboratory Manual, Cold Spring Harbor Laboratory, Cold Spring Harbor N.Y. Humanized antibodies may be routinely produced as taught, for example, by U.S. Pat. No. 5,585,089 and U.S. Pat. No. 5,530,101. Techniques for engineering antibodies are well known and described in Winter and Millstein (1991) Nature 349:293, and Larrich and Fry (1991) Hum. Antibod. and Hybridomas 2:17. One having ordinary skill in the art may use well known techniques and starting materials and/or commercially available expression vectors and systems that are readily available and known in the art. See e.g., Sambrook et al., Molecular Cloning a Laboratory Manual, Second Ed. Cold Spring Harbor Press (1989).

Examples of radionuclides useful as toxins in radiation therapy are well known. Some examples are referred to below. Auger emitters may be preferred for internalizing antibodies. As suggested above, the term antibody is used interchangeably with antibody fragment and antigen binding fragment and includes a whole antibody; antibody fragment a portion of an antibody such as a scFV F(ab′)₂ F(ab)₂. Fab′, Fab, dAb, microbodies (WO00 29004) or the like or multivalent such fragments, including those itemized or referenced herein. Regardless of structure, an antibody fragment can be made to bind with the same antigen that is recognized by the intact antibody. More particularly, in addition to fragments formed by enzymaic digestion of an intact Ab the term antibody or “antibody fragment” unless otherwise stated also includes any synthetic or genetically engineered protein that acts like an antibody by binding to a specific antigen to form a complex including/as applicable, cysteine noose peptides and minimal recognition units consisting of the amino acid residues that mimic the hypervariable region. Although fully human antibodies, for example, antibodies generated via human-human hybridomas or through phage display using human antibody based libraries, are preferred, the invention does not preclude other strategies to avoid a HAMA type response.

A chimeric antibody is a recombinant protein that contains the variable domains and complementary determining regions derived from, for example, a rodent antibody, while the remainder of the antibody molecule is derived from a human antibody.

With respect to stability engineering of scFv fragments for enhanced mulfunctional ligands comprising scFvs see J Mol Biol Feb. 2, 2001; 305(5):989-1010.

Humanized antibodies are recombinant proteins in which murine LDR's of a monoclonal antibody have been transferred from heavy and light variable chains of the murine immunoglobulin into a human variable domain.

The term therapeutic agent as used herein, is a molecule or atom which is conjugated etc. to an antibody moiety to produce combination including a conjugate which is useful for therapy. Examples of therapeutic agents include drugs, toxins, immunomodulators, chelators, boron compounds, photoactive agents or dyes, and radioisotopes.

The term “a naked antibody” may be used to refer specifically to an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

The term immunoconjugate may be used to refer a conjugate of an antibody component with a therapeutic agent.

As used herein, the term antibody fusion protein refers to a recombinant molecule that comprises an antibody component and a second functional component for example a therapeutic agent. Examples of therapeutic agents suitable for such fusion proteins include immunomodulators (“antibody-immunomodulator fusion protein”) and toxins (“antibody-toxin fusion protein”).

Production of Antigen—Specific Monoclonal Antibodies, Rodent monoclonal antibodies to antigen can be obtained by methods known to those skilled in the art. See generally, for example, Kohler and Milstein, Nature 256:495 (1975), and Coligan et al. (eds.), Current Protocols in Immunology, Vol. 1, pages 2.5.1-2.6.7 (John Wiley & Sons 1991) [“Coligan”]. Briefly, monoclonal antibodies can be obtained by injecting mice with a composition comprising the antigen in a question (Ag), verifying the presence of antibody production by removing a serum sample, removing the spleen to obtain B-lymphocytes, fusing the B-lymphocytes with myeloma cells to produce hybridomas, cloning the hybridomas, selecting positive clones which produce anti-Ag antibodies, culturing the clones that produce antibodies to the antigen, and isolating the antibodies from the hybridoma cultures. Transgenic mice having for example engineered immune systems to create human antibodies such those used by Medarex and Abgenix are also contemplated for use herein to create suitably targeted antibodies.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in Methods in Molecular Biology, Vol. 10, pages 79-104 (The Humana Press, Inc. 1992).

With respect to relevant molecular biology techniques See also, for example, Ausubel et al., (eds.), CURRENT PROTOCOLS IN MOLECULAR BIOLOGY, pages 8.2.8 to 8.2.13 (1990) [“Ausubel”]. Also, see Wosnick et al., Gene 60:115 (1987); and Ausubel et al. (eds.), Short Protocols in Molecular Biology, 3rd Edition, pages 8-8 to 8-9 (John Wiley & Sons, Inc. 1995). Established techniques using the polymerase chain reaction provide the ability to synthesize genes as large as 1.8 kilobases in length. Adang et al., Plant Molec. Biol. 21:1131 (1993) Bambot et al., PCR Methods and Applications 2:266 (1993); Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, (Humana Press, Inc. 1993).

Techniques for constructing chimeric antibodies are well-known to those of skill in the art. As an example, Leung et al., Hybridoma 13:469 (1994).

In yet another embodiment, an antibody of the present invention is a “humanized” monoclonal antibody. That is, mouse complementarity determining regions are transferred from heavy and light variable chains of the mouse immunoglobulin into a human variable domain, followed by the replacement of some human residues in the framework regions of their murine counterparts. Humanized monoclonal antibodies in accordance with this invention are suitable for use in therapeutic methods. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies are described, for example, by Jones et al., Nature 321:522 (1986), Riechmann et al., Nature 332:323 (1988), Verhoeyen et al., Science 239:1534 (1988), Carter et al., Proc. Nat'l Acad. Sci. USA 89:4285 (1992), Sandhu, Crit. Rev. Biotech. 12:437 (1992), and Singer et al., J. Immun. 150:2844 (1993). The publication of Leung et al., Mol. Immunol. 32:1413 (1995), describes the construction of humanized LL2 antibody.

In a preferred embodiment of the invention the multifunctional ligand has a unique portion which differentiates it from other antibodies and preferably other co-administered different multifunctional ligands, which unique portion, allows the multifunctional ligand to be efficiently segregated on an immunoaffinity column. In the case of differentiating a single multifunctional ligand an anti-idiotype (assuming the first portion consists of an antibody) or other antibody uniquely recognizing the first portion could be employed. Modifying a portion of the first portion, for example in the case where it is antibody component and creating a antibody thereto, for example by phage display, is a matter of routine skill in the arts of antibody engineering and phage display.

In another embodiment, an antibody of the present invention is a human monoclonal antibody. Such antibodies are obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994).

Examples of Production of Antibody Fragments

Antibody fragments can be prepared, for example, by proteolytic hydrolysis of an antibody or by expression in E. coli of the DNA coding for the fragment.

Antibody fragments can be obtained by pepsin or papain digestion of whole antibodies by conventional methods. For example, antibody fragments can be produced by enzymatic cleavage of antibodies with pepsin to provide a 5 S fragment denoted F(ab′)₂. This fragment can be further cleaved using a thiol reducing agent, and optionally a blocking group for the sulfhydryl groups resulting from cleavage of disulfide linkages, to produce 3.5 S Fab′ monovalent fragments. Alternatively, an enzymatic cleavage using pepsin produces two monovalent Fab fragments and an Fc fragment directly. These methods are described, for example, by Goldenberg, U.S. Pat. Nos. 4,036,945 and 4,331,647 and references contained therein. Also, see Nisonoff et al., Arch Biochem. Biophys. 89:230 (1960); Porter, Biochem. J. 73:119 (1959), Edelman et al., in Methods in Enzymology Vol 1, page 422 (Academic Press 1967), and Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, for example, Sandhu, supra.

Preferably, the Fv fragments comprise V_{H} and V_{L} chains which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991). Also see Bird et al., Science 242:423 (1988), Ladner et al., U.S. Pat. No. 4,946,778, Pack et al., Bio/Technology 11:1271 (1993), and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

Preparation of Immunoconjugates

The present invention contemplates immunoconjugates to assess and effect treatment of various disease conditions. Such immunoconjugates can be prepared by indirectly conjugating a therapeutic agent to an antibody component. For example, general techniques are described in Shih et al., Int. J. Cancer 41:832-839 (1988); Shih et al., Int. J. Cancer 46:1101-1106 (1990); and Shih et al., U.S. Pat. No. 5,057,313. The general method involves reacting an antibody component having an oxidized carbohydrate portion with a carrier polymer that has at least one free amine function and that is loaded with a plurality of drug, toxin, chelator, boron addends, or other therapeutic agent. This reaction results in an initial Schiff base (imine) linkage, which can be stabilized by reduction to a secondary amine to form the final conjugate.

The carrier polymer is preferably an aminodextran or polypeptide of at least 50 amino acid residues, although other substantially equivalent polymer carriers can also be used. Preferably, the final immunoconjugate is soluble in an aqueous solution, such as mammalian serum, for ease of administration and effective targeting for use in therapy. Thus, solubilizing functions on the carrier polymer will enhance the serum solubility of the final immunoconjugate. In particular, an aminodextran will be preferred.

The process for preparing an immunoconjugate with an aminodextran carrier typically begins with a dextran polymer, advantageously a dextran of average molecular weight of about 10,000-100,000. The dextran is reacted with an oxidizing agent to effect a controlled oxidation of a portion of its carbohydrate rings to generate aldehyde groups. The oxidation is conveniently effected with glycolytic chemical reagents such as NaIO.sub.4, according to conventional procedures.

The oxidized dextran is then reacted with a polyamine, preferably a diamine, and more preferably, a mono- or polyhydroxy diamine. Suitable amines include ethylene diamine, propylene diamine, or other like polymethylene diamines, diethylene triamine or like polyamines, 1,3-diamino-2-hydroxypropane, or other like hydroxylated diamines or polyamines, and the like. An excess of the amine relative to the aldehyde groups of the dextran is used to insure substantially complete conversion of the aldehyde functions to Schiff base groups.

A reducing agent, such as NaBH₄, NaBH₃ CN or the like, is used to effect reductive stabilization of the resultant Schiff base intermediate. The resultant adduct can be purified by passage through a conventional sizing column to remove cross-linked dextrans.

Other conventional methods of derivatizing a dextran to introduce amine functions can also be used, e.g., reaction with cyanogen bromide, followed by reaction with a diamine.

The aminodextran is then reacted with a derivative of the particular drug, toxin, chelator, immunomodulator, boron addend, or other therapeutic agent to be loaded, in an activated form, preferably, a carboxyl-activated derivative, prepared by conventional means, e.g., using dicyclohexylcarbodiimide (DCC) or a water soluble variant thereof, to form an intermediate adduct.

Alternatively, polypeptide toxins such as pokeweed antiviral protein or ricin A-chain, and the like, can be coupled to aminodextran by glutaraldehyde condensation or by reaction of activated carboxyl groups on the protein with amines on the aminodextran.

Chelators for radiometals or magnetic resonance enhancers are well-known in the art. Typical are derivatives of ethylenediaminetetraacetic acid (EDTA) and diethylenetriaminepentaacetic acid (DTPA). These chelators typically have groups on the side chain by which the chelator can be attached to a carrier. Such groups include, e.g., benzylisothiocyanate, by which the DTPA or EDTA can be coupled to the amine group of a carrier. Alternatively, carboxyl groups or amine groups on a chelator can be coupled to a carrier by activation or prior derivatization and then coupling, all by well-known means.

Boron addends, such as carboranes, can be attached to antibody components by conventional methods. For example, carboranes can be prepared with carboxyl functions on pendant side chains, as is well known in the art. Attachment of such carboranes to a carrier, e.g., aminodextran, can be achieved by activation of the carboxyl groups of the carboranes and condensation with amines on the carrier to produce an intermediate conjugate. Such intermediate conjugates are then attached to antibody components to produce therapeutically useful immunoconjugates, as described below.

A polypeptide carrier can be used instead of aminodextran, but the polypeptide carrier must have at least 50 amino acid residues in the chain, preferably 100-5000 amino acid residues. At least some of the amino acids should be lysine residues or glutamate or aspartate residues. The pendant amines of lysine residues and pendant carboxylates of glutamine and aspartate are convenient for attaching a drug, toxin, immunomodulator, chelator, boron addend or other therapeutic agent. Examples of suitable polypeptide carriers include polylysine, polyglutamic acid, polyaspartic acid, copolymers thereof, and mixed polymers of these amino acids and others, e.g., serines, to confer desirable solubility properties on the resultant loaded carrier and immunoconjugate.

Conjugation of the intermediate conjugate with the antibody component is effected by oxidizing the carbohydrate portion of the antibody component and reacting the resulting aldehyde (and ketone) carbonyls with amine groups remaining on the carrier after loading with a drug, toxin, chelator, immunomodulator, boron addend, or other therapeutic agent. Alternatively, an intermediate conjugate can be attached to an oxidized antibody component via amine groups that have been introduced in the intermediate conjugate after loading with the therapeutic agent. Oxidation is conveniently effected either chemically, e.g., with NaIO₄ or other glycolytic reagent, or enzymatically, e.g., with neuraminidase and galactose oxidase. In the case of an aminodextran carrier, not all of the amines of the aminodextran are typically used for loading a therapeutic agent. The remaining amines of aminodextran condense with the oxidized antibody component to form Schiff base adducts, which are then reductively stabilized, normally with a borohydride reducing agent.

Analogous procedures are used to produce other immunoconjugates according to the invention. Loaded polypeptide carriers preferably have free lysine residues remaining for condensation with the oxidized carbohydrate portion of an antibody component. Carboxyls on the polypeptide carrier can, if necessary, be converted to amines by, e.g., activation with DCC and reaction with an excess of a diamine.

The final immunoconjugate is purified using conventional techniques, such as sizing chromatography on Sephacryl S-300.

Alternatively, immunoconjugates can be prepared by directly conjugating an antibody component with a therapeutic agent. The general procedure is analogous to the indirect method of conjugation except that a therapeutic agent is directly attached to an oxidized antibody component.

For application to linking MHC I/II peptide/B7 molecules to a latex which has previously conjugated to biotin, for avidin assisted linking to a multifunctional ligand, it will be appreciated that biotin can be conjugated to a part of a latex sphere which is then linked to MHC peptide and B7 molecules by placing the spheres in a confluent layer or in the spheres in a microwells such that only part of the sphere is exposed for conjugation and then coating the spheres onto avidin coated plates for the B7 and MHC linkage.

It will be appreciated that other therapeutic agents can be substituted for the chelators described herein. Those of skill in the art will be able to devise conjugation schemes without undue experimentation.

As a further illustration, a therapeutic agent can be attached at the hinge region of a reduced antibody component via disulfide bond formation. For example, the tetanus toxoid peptides can be constructed with a single cysteine residue that is used to attach the peptide to an antibody component. As an alternative, such peptides can be attached to the antibody component using a heterobifunctional cross-linker, such as N-succinyl 3-(2-pyridyldithio)proprionate (SPDP). Yu et al., Int. J. Cancer 56:244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995).

As described above, carbohydrate moieties in the Fc region of an antibody can be used to conjugate a therapeutic agent. However, the Fc region is absent if an antibody fragment is used as the antibody component of the immunoconjugate. Nevertheless, it is possible to introduce a carbohydrate moiety into the light chain variable region of an antibody or antibody fragment. See, for example, Leung et al., J. Immunol. 154:5919 (1995); Hansen et al., U.S. Pat. No. 5,443,953 (1995). The engineered carbohydrate moiety is then used to attach a therapeutic agent.

In addition, those of skill in the art will recognize numerous possible variations of the conjugation methods. For example, the carbohydrate moiety can be used to attach polyethyleneglycol in order to extend the half-life of an intact antibody, or antigen-binding fragment thereof, in blood, lymph, or other extracellular fluids. Moreover, it is possible to construct a “divalent immunoconjugate” by attaching therapeutic agents to a carbohydrate moiety and to a free sulfhydryl group. Such a free sulfhydryl group may be located in the hinge region of the antibody component.

Methods for determining the binding specificity of an antibody are well-known to those of skill in the art. General methods are provided, for example, by Mole, “Epitope Mapping,” in METHODS IN MOLECULAR BIOLOGY, VOLUME 10: IMMUNOCHEMICAL PROTOCOLS, Manson (ed.), pages 105-116 (The Humana Press, Inc. 1992). More specifically, competitive blocking assays for example to determine CD22 epitope specificity are described by Stein et al., Cancer Immunol. Immunother. 37:293 (1993), and by Tedder et al., U.S. Pat. No. 5,484,892 (1996).

In another aspect the invention is directed to a bispecific ligand, preferably a bispecifc antibody, comprising at least a first ligand, preferably an antibody component, which binds specifically to a first cell surface associated ligand and at least a second ligand, preferably a second antibody component which binds specifically to a second cell surface associated ligand on the same cell, and wherein the functional affinity of at least one and preferably both of said antibody components is selected so as to substantially limit functional binding unless both of said first and second antibody components are substantially contemporaneously bound to said cell. It is known to provide bifunctional ligands wherein functional binding, for example, to accomplish signal transduction, is predicated on both ligands being bound or cross-linking. However this effect is not contemplated to be predicated on differentially controlling the functional affinity of the respective ligands. According to a broad aspect of this invention (in which inclusion of a ligand which binds to a lymphatic vessel associated marker is optional), the invention excludes known such bispecific ligands which inherently have a suitable differential functional affinity. Such bispecific ligand are mentioned herein. By controlling the affinity of at least one of said ligands, for example where the functional affinity of one said ligands is substantially less than that of the other ligand the invention contemplates that a substantially greater percentage of the administered dose of the bispecific ligand will affect cells in which only both ligands are present, and/or that a reduced percentage of the dose administered will functionally bind to the cells in virtue only of the reduced functional affinity ligand. The invention also contemplates that functional affinity of one ligand is greatly increased to establish the functional affinity differential and that the functional affinity of both ligands is reduced relative to that of a standard, for example relative to that of a comparable ligands in hand or known in the art or identified by phage display, ribsome display or other comparable techniques using a single such ligand. The invention also contemplates that a microarray (or library) of bispecific ligands in which for example, the bispecific ligand is “tethered” (ie. immobilized) directly or indirectly in virtue of one or more amino acid residues which are positioned within the molecule to preferably minimally interfere with any binding, and in which the signal (eg its intensity) associated with a single ligand binding interaction can be differentiated from a two or more ligand interactions, for example cell surface binding (alternatively the ligands or cell may be immobilized) and that ribsome and phage display could be adapted to bispecific single domain antibodies constituting a single chain (see references herein) by elongating the end of the chain from which the molecule is tethered. The invention contemplates that the affinity of one such ligand may be fixed and that the variability in members of the library lies in the permutations of certain key residues to which binding is attributable which can readily be identified by persons skilled in the art. The invention also contemplates assessing single ligand binding capability of successful bi-ligand binders for example by blocking the other (non-assessed at that time) ligand (eg. with correlative ligand or a mimotope thereof) and for example determining limited or non-existent such binding to as well as using inclined ligand testing surfaces for washing over the correlative ligand, for example of defined surface area, including preferably defined lengths and widths and concentrations/distributions/amounts of the bound ligand, where the degree of incline is selected to roughly simulate the micro-environment of the comparable in vivo target, be it a stationary cell with a roughly defined average shear force of bathing fluids eg. within a tumor or in the lymphatic system, or a mobile cell within a vein, artery, or lymphatic vessel, including those of different sizes. The invention is also directed to a mthod of generating a target ligand or improving the target specificity of any ligand by using a population of variants of that ligand within a micro-environment simulated microarray system in which the at least one of the follwing factors is simulated: concentration or amount or distribution of correlative ligand, shear force and shape using length and width parameters to simulates intraluminal diameter and length. The invention also contemplates in the case of a multifunctional ligand or in the case of a bispecific or multispecfic ligand (as herein described) that the affinity of its component binding ligands may be selected for venous or arterial tageting or to accommodate lymphatic system targeting or targeting within or through tissues or combinations of the aforementioned eg. median, average or or weighted compromises to improve desired targeting. In a preferred embodiment the first ligand is selected on the basis of its ability to at least partially discriminate between a target population of cells (eg. a ligand that is “associated” with a target population of cells) and a non-target population of cells (in one embodiment it is selected so as to have no other effect other than binding for targeting purposes) and the second ligand is selected for its ability to modulate the activity of the targeted cell, optionally in virtue of binding alone eg. without delivering a payload (the term modulate referring broadly to any desired effect on the cell or its functionality) In this case the functional affinity for the ligand which is targeted for modulating the activity of the cell is selected so as to reduce the likelihood of binding unless binding has first or contemporaneously occurred to the first ligand targeted for selectivity (eg. the second ligand would have monovalent as opposed to divalent binding to the ligand required for selectivity and/or from 0.20 to 10-9 fold reduction in affinity (for example as measured by Biacore) relative to the binding affinity for the first ligand. This reduction in affinity is preferably greater than a 100% reduction in affinity (multiply by 0.1), preferably greater than a 200% reduction in affinity, preferably greater than a 300% reduction in affinity, preferably greater than a 400% reduction in affinity, preferably greater than a 500% reduction in affinity, preferably greater than a 600% reduction in affinity, preferably greater than a 700% reduction in affinity, preferably greater than a 800% reduction in affinity, preferably greater than a 900% reduction in affinity, preferably greater than a 1000% reduction in affinity, preferably greater than a 2000% reduction in affinity, preferably greater than a 3000% reduction in affinity, preferably greater than a 4000% reduction in affinity, preferably greater than a 5000% reduction in affinity, preferably greater than a 6000% reduction in affinity, preferably greater than a 7000% reduction in affinity, preferably greater than a 8000% reduction in affinity, preferably greater than a 9000% reduction in affinity, preferably greater than a 10000% reduction in affinity, preferably greater than a 20000% reduction in affinity, preferably greater than a 30000% reduction in affinity, preferably greater than a 40000% reduction in affinity, preferably greater than a 50000% reduction in affinity, preferably greater than a 60000% reduction in affinity, preferably greater than a 70000% reduction in affinity, preferably greater than a 80000% reduction in affinity, preferably greater than a 90000% reduction in affinity, preferably greater than a 100000% reduction in affinity, preferably greater than a 500000% reduction in affinity, preferably greater than a 1000000% reduction in affinity, preferably greater than a 10000000% reduction in affinity, preferably greater than a 20000000% reduction in affinity, preferably a greater than 3000000% reduction in affinity, preferably a greater than 40,000,000% reduction in affinity, preferably a greater than 50000000% reduction in affinity, preferably a greater than 60000000% reduction in affinity, preferably a greater than 70000000% reduction in affinity, preferably a greater than 80000000% reduction in affinity, preferably a greater than 90000000% reduction in affinity preferably a greater than 100,000,000% reduction in affinity, preferably a reduction in affinity of between one and two orders of magnitude, preferably a reduction in affinity of between two and three orders of magnitude, preferably a reduction in affinity of between three and four orders of magnitude, preferably a reduction in affinity of between four and five orders of magnitude, preferably a reduction in affinity of between five and six orders of magnitude, preferably a reduction in affinity of between six and seven orders of magnitude preferably a reduction in affinity of between seven and eight orders of magnitude, preferably a reduction in affinity of between eight and nine orders of magnitude, preferably a reduction in affinity of between nine and ten orders of magnitude.

It will be appreciated that a suitable reduction in affinity, if any, will depend on the valency of the respective first and second ligands and the selected affinity of the first ligand, which for example may have been augmented. The invention also contemplates a trispecific (and triavalent) ligand in which two ligands differently define its specificity to reduce the likelihood of an undesired effect attributable to the function exerting moiety binding alone. In terms of the physical constitution of a ligand having a trispecific binding capability, the invention also contemplates linking three monovalent dabs, MRUs or the like or mixed combinations thereof or two bivalent dabs, MRUs or the like or mixed combinations thereof (see WO 99/42077, U.S. Pat. No. 6,174,691, WO0029004, Camel single-domain antibodies as modular building units in J Biol Chem. Oct. 25, 2000 & Mulligan-Kehoe U.S. patents including U.S. Pat. No. 5,702,892, U.S. Pat. No. 5,824,520; se also U.S. Pat. No. 6,040,136 ) (in the latter case optionally one or both may be bispecific and linked by well known methods in the art (see WO 99/42077, Celltech's TFM, leucine zippers, U.S. Pat. No. 5,910,573, U.S. Pat. No. 5,892,020, EP 0654085B, see also EP 0318554B). The term functional binding is used to refer to binding which yields the desired effect, for example a therapeutic effect on a target cell population attributable to the binding to one or both ligands. Using the previous example, one ligand, eg. the first ligand, may be used to target activated immune cells, and the second ligand may be different and may upon being bound to, for example result in inactivation, anergy, apoptosis or reduced capacity for endothelial adhesion of the immune cell. In this case, the invention contemplates that the functional affinity of the antibody component which binds to the second ligand is selected such that binding is unlikely to occur without binding to the specificity dictating ligand, for example the ratio of targeted relative non-targeted cells affected by the dose administered is approximately 1.10 to 1, preferably approximately 1.15 to 1, more preferably approximately 1.20 to 1, more preferably approximately 1.25 to 1, more preferably approximately 1.30 to 1, more preferably approximately 1.35 to 1, more preferably approximately 1.40 to 1, more preferably approximately 1.45 to 1, more preferably approximately 1.50 to 1, more preferably approximately 1.55 to 1, more preferably approximately 1.60 to 1, more preferably approximately 1.60 to 1, more preferably approximately 1.65 to 1, more preferably approximately 1.70 to 1, more preferably approximately 1.75 to 1, more preferably approximately 1.80 to 1, more preferably approximately 1.85 to 1, more preferably approximately 1.90 to 1, more preferably approximately 1.95 to 1, more preferably approximately 2 to 1, more preferably greater than 2 to 1, more preferably approximately greater than 3 to 1, more preferably approximately greater than 4 to 1, more preferably greater than 5 to 1, more preferably greater than 6 to 1, more preferably greater than 7 to 1, more preferably greater than 8 to 1, more preferably greater than 9 to 1, more preferably greater than 10 to 1, more preferably greater than 20 to 1, more preferably greater than 30 to 1, more preferably greater than 40 to 1, more preferably greater than 50 to 1, more preferably greater than 60 to 1, more preferably greater than 70 to 1, more preferably greater than 80 to 1, more preferably greater than 90 to 1, more preferably greater than 100 to 1, more preferably greater than 500 to 1, more preferably greater than 1000 to 1, more preferably greater than 10,000 to 1, more preferably greater than 100,000 to 1, more preferably greater than 500,000 to 1 more preferably greater than 1,000,000 to 1.

It will be appreciated by persons skilled in the art that the foregoing aspects of the invention apply to a variety of different combinations of immune function or other therapeutic function exerting ligands and specificity dictating ligands including those involved in immune signaling, stimulatory, co-stimulatory, inhibitory, adhesion or other interactions, including without limitation, cytokine receptors, ligands associated with immune cell adhesion, ligands to which binding results in stimulation, activation, apoptosis, anergy or costimulation, or ligands which differentiate between different populations or subpopulations or immune cells (see eg. U.S. Pat. No. 6135941, WO 00/63251, WO 00/61132, U.S. Pat. No. 6,120,767), including sub-populations of B cells and T cells (see for example U.S. Pat. No. 6,197,524) activated versus non-activated lympocytes, diseased or disease-causing cells versus non-diseased/disease causing lymphocytes (see for example WO 01/13945A1, U.S. Pat. No. 6,132,980, ) and specific immune cell clones for example those having specific Ig type and MHC-peptide type ligands/and correlative ligands. Examples of such ligands include CCR5, CTLA-4, LFA-1, LFA-3. ICAMs eg. ICAM-1, CD2, CD3, CD4 (eg see U.S. Pat. No. 6,136,310), CD18, CD22, CD40, CD44; CD80, CD86, CD134 and CD154, to name only a few (see also U.S. Pat. No. 6,087,475: PF4A receptor) (see also Glennie M J et al. Clinical Trial of Antibody Therapy. Immunology Today August 2000, Vol. 21 (no. 8) p.406).

The invention also contemplates that the therapeutic function or immune function effecting ligand is also a specificity imparting ligand, which in the case of for example, an antigen presenting cell may be an antibody which recognizes and binds to a specific MHC peptide complex, as is established in the art (see pertinent Chames et al. references herein, see also WO 97/02342, Direct selection of a human antibody fragment directed against the tumor T-cell epitope HLA-A1-MAGE-A1 from a nonimmunized phage-Fab library. Proc Natl Acad Sci USA. Jul. 5, 2000; 97(14):7969-74). In this case it will be appreciated that the APC targeting ligand assist the particular MHC peptide binding antibody to bind to its target.

See also WO 97/07819 which is hereby disclaimed with respect to all relevant aspects of the invention herein insofar as inherently disclosed therein. See also U.S. Pat. No. 5,770,403 with respect to antibodies which bind to cytokines.

In one embodiment, the respective antibody components of the multispecific ligand recognize a substantially different subset of non-targetted tissues so that functional binding to a non-targetted tissue is substantially precluded. It will be appreciated that this strategy can be accomplished with two different antibodies have differing and preferably non-overlapping normal ie. non-targeted tissue distributions. In a preferred embodiment the target cell is a cancer cell and the respective first and second cell surface associated ligands are expressed on different subsets of normal cells, which are non-overlapping subsets, so as to minimize deleterious normal cell targeting and distibute the undesired effects or normal cell targeting (eg. with a toxin), to different cell populations. For example in the case of tumor cell targeting one or both ligands may be expressed exclusively on a single tumor type (eg. a human sarcoma or carcinoma, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (mycloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease) or a particular category of tumor types (eg. adenocarcinomas, tumors of neuroectodermal origin, or on multiple different tumor types or categories of tumor. One or both components (they may be the same or different) may be a dAb, a scFv, an Fab, a minibody moiety or a substantially intact antibody, for example both may be scFvs and the resulting product may be a diabody, triabody, or tetrabody. For example in a preferred embodiment the bispecific antibody comprises two dAb components comprising linked via a linker (see above) and having at least at least part of a constant region for fusion for example to a toxin (eg. at least a partial hinge region, and preferably also at least a partial CH2 domain (optionally also at least a partial CH3 domain). In another embodiment, a trispecific antibody or tetraspecific antibody with at least two different and preferably 3 or 4 subsets (preferably at least one or more of such subsets being non-overlapping subsets) of non-targeted cell reactivities may be employed in the form of a trispecific or tetraspecific antibody respectively whereby up to three or four different pairs of ligands are targeted, so as further minimize normal cell targeting and also preferably target a heterogenous population of cells within the same tumor. Ligands with distributions on normal tissues are well known, some being referenced herein, for example CEA, CD-20, P53, epidermal growth factor, including known multicarcinomic and pancarcinomic ligands (eg. see U.S. Pat. No. 5,171,665, U.S. Pat. No. 4,349,528.

The term functional binding is used to mean binding for the purpose of accomplishing the object of the binding, for example binding for a sufficient duration to inhibit or enhance a particular effect, such as cell killing, for example in the case where one both antibody components are selected for their ability to internalize, binding for a sufficient duration to permit internalization, for example to deliver a toxic payload. As discussed above, the term substantially in reference to therapeutic advantage is used to refer to a degree which provides a significant benefit from a therapeutic standpoint.

Examples of tumor associated antigens (eg. WO 01/21828) and targets and related antibodies are referenced throughout the disclosure and the foregoing aspect of the invention is for greater certainty directed to bispecific antibodies (including trispecific and tetraspecific antibodies, optionally including a component which also binds to a lymphatic vessel associated ligand), which target each of the combinations and permuations of the target cell (diseased, disease causing or immune) associated antigens, ligands, epitopes or receptors well known to those skilled in the art, herein directly or indirectly referenced or referenced in the materials herein incorporated by reference (ie. permutations and combinations of pairs or where a tri-or tetra-specific antibody is used possibly permutations of (3 or 4) groups of pairs including for example pairs in which one member is used for targeting and the second is used for modulation puposes such modulation including without limitation, simple binding eg. to deliver a payload, apoptosis inducing (eg. anti-fas), modified vascular adhesion properties (eg. anti-CD44), modified cytokine binding (anti-CCR5) etc.(re: relevant ligands/markers see also U.S. Pat. No. 6,010,902 and the references cited therein, Samter's Immunologic Diseases, Fifth and Sixth Edition, Lippincott, Frank Austen, MD Michael M. Frank, M D John P. Atkinson, MD Harvey I. Cantor, MD (6^{th}-ISBN: 0-7817-2120-2); Fundamental Virology, Third and Fourth Edition, Lippincott David M. Knipe, PhD Peter M. Howley, MD Diane E. Griffin, MD, PhD Robert A. Lamb, PhD, ScD Malcolm A. Martin, MD Bernard Roizman, ScD Stephen E. Straus, MD (4^{th}-ISBN: 0-7817-1833-3); Arthritis and Allied Conditions—A Textbook of Rheumatology, Thirteenth and Fourteenth Editions, William J. Koopman, MD 14^{th}:ISBN: 0-7817-2240-3, November 2000; Cancer—Principles and Practice of Oncology, Fifth and Sixth Editions, Lippincott, Vincent T. DeVita, Jr., MD Samuel Hellman, MD Steven A. Rosenberg, MD, PhD ISBN: 0-7817-2229-2; Dubois' Lupus Erythematosus, Fifth Edition, Daniel J. Wallace, MD ISBN: 0-683-08665-0, December 1996; Cytokine Therapeutics in Infectious Diseases, Steven M. Holland, MD, PhD, Lippincott, ISBN: 0-7817-1625-X, U.S. Pat. No. 6,054,561), in each of their permuatations of size/valency (ie. dabs, scFv, diabodies etc herein referenced) as applied to each of the applicable disease conditions herein referenced or otherwise known to those skilled in the art.

With respect to recombinant techniques for producing Fv fragments see also WO 88/01649, WO 88/06630, WO 88/07085, WO 88/07086, and WO 88/09344.

With respect to preparing ligands for specific MHC peptide complexes see also WO 01/22083; Direct selection of a human antibody fragment directed against the tumor T-cell epitope HLA-A1-MAGE-A1 from a nonimmunized phage-Fab library. Proc Natl Acad Sci USA. Jul. 5, 2000; 97(14):7969-74.

With respect to bispecific antigen binding constructs that are suitable for for binding to more than one antigen on the same cell see also Schmiedl A et al. Protein Eng October 2000 13(10):725-34.

A radiolabeled immunoconjugate may comprise an .alpha.-emitting radioisotope, a .B-emitting radioisotope, a gamma emitting radioisotope, an Auger electron emitter, a neutron capturing agent that emits alpha-particles or a radioisotope that decays by electron capture. Suitable radioisotopes include ¹⁹⁸Au, ³²p,. ¹²⁵I, ¹³¹I, ⁹⁰Y, ¹⁸⁶Re, ¹⁸⁸Re, ⁶⁷Cu, ²¹¹At, and the like.

As discussed above, a radioisotope can be attached to an antibody component directly or indirectly, via a chelating agent. For example, ⁶⁷Cu, considered one of the more promising radioisotopes for radioimmunotherapy due to its 61.5 hour half-life and abundant supply of beta particles and gamma rays, can be conjugated to an antibody component using the chelating agent, p-bromoacetamido-benzyl-tetraethylaminetetraacetic acid (TETA). Chase, “Medical Applications of Radioisotopes,” in Remington's Pharmaceutical Sciences, 18th Edition, Gennaro et al. (eds.), pages 624-652 (Mack Publishing Co. 1990) (see also 19^{th} edition of Reminton's). Alternatively, ⁹⁰Y, which emits an energetic beta particle, can be coupled to an antibody component using diethylenetriaminepentaacetic acid (DTPA). Moreover, a method for the direct radiolabeling of the antibody component with ¹³¹I is described by Stein et al., Antibody Immunoconj. Radiopharm. 4: 703 (1991) (see also U.S. Pat. No. 6,080,384).

Alternatively, boron addends such as carboranes can be attached to antibody components, as discussed above.

In addition, therapeutic immunoconjugates can comprise an immunomodulator moiety suitable for application for the purposes herein. Broadly speaking, the term “immunomodulator” includes cytokines, stem cell growth factors, lymphotoxins, such as tumor necrosis factor (TNF), and hematopoietic factors, such as interleukins (e.g., interleukin-1 (IL-1), IL-2, IL-3, IL6, IL-10 and IL-12), colony stimulating factors (e.g., granulocyte-colony stimulating factor (G-CSF) and granulocyte macrophage-colony stimulating factor (GM-CSF)), interferons (e.g., interferonsalpha, -beta and gamma.), the stem cell growth factor designated “S1 factor,” erythropoietin and thrombopoietin. Examples of suitable immunomodulator moieties include IL-2, IL-6, IL-10, IL12, interferon-gamma., TNF-alpha., and the like.

A related form of therapeutic protein is a fusion protein comprising an antibody moiety and an immunomodulator moiety.

Methods of making antibody-immunomodulator fusion proteins are known to those of skill in the art as discussed herein. For example, antibody fusion proteins comprising an interleukin-2 moiety are described by Boleti et al., Ann. Oncol. 6:945 (1995), Nicolet et al., Cancer Gene Ther. 2:161 (1995), Becker et al., Proc. Nat'l Acad. Sci. USA 93:7826 (1996), Hank et al., Clin. Cancer Res. 2:1951 (1996), and Hu et al., Cancer Res. 56:4998 (1996). In addition, Yang et al., Hum. Antibodies Hybridomas 6:129 (1995), describe a fusion protein that includes an F(ab′)₂ fragment and a tumor necrosis factor alpha moiety.

Such immunoconjugates and antibody-immunomodulator fusion proteins provide a means to deliver an immunomodulator to a target cell and are particularly useful against tumor cells. The cytotoxic effects of immunomodulators are well known to those of skill in the art. See, for example, Kle et al., “Lymphokines and Monokines,” in Biotechnology and Pharmacy, Pessuto et al. (eds.), pages 53-70 (Chapman & Hall 1993) as well as other references herein cited. As an illustration, interferons can inhibit cell proliferation by inducing increased expression of class I histocompatibility antigens on the surface of various cells and thus, enhance the rate of destruction of cells by cytotoxic T lymphocytes. Furthermore, tumor necrosis factors, such as TNF-alpha., are believed to produce cytotoxic effects by inducing DNA fragmentation.

Moreover, therapeutically useful immunoconjugates can be prepared in which an antibody component is conjugated to a toxin or a chemotherapeutic drug. Illustrative of toxins which are suitably employed in the preparation of such conjugates are ricin, abrin, ribonuclease, DNase I, Staphylococcal enterotoxin-A, pokeweed antiviral protein, gelonin, diphtherin toxin, Pseudomonas exotoxin, and Pseudomonas endotoxin. See, references herein as well as for example, Pastan et al., Cell 47:641 (1986), and Goldenberg, CA-A Cancer Journal for Clinicians 44:43 (1994). Other suitable toxins are known to those of skill in the art.

With to respect to bispecific antibody constructs which are capable of binding simultaneously to two ligands on the same cell see also WO96/32841. Various such constructs are known in the art.

In addition, therapeutically useful immunoconjugates can be obtained by conjugating photoactive agents or dyes to an antibody composite. Fluorescent and other chromogens, or dyes, such as porphyrins sensitive to visible light, have been used to detect and to treat lesions by directing the suitable light to the lesion. In therapy, this has been termed photoradiation, phototherapy, or photodynamic therapy (Jori et al. (eds.), Photodynamic Therapy of Tumors and Other Diseases (Libreria Progetto 1985); van den Bergh, Chem. Britain 22:430 (1986)). Moreover, monoclonal antibodies have been coupled with photoactivated dyes for achieving phototherapy. Mew et al., J. Immunol. 130:1473 (1983); idem., Cancer Res. 45:4380 (1985); Oseroff et al., Proc. Natl. Acad. Sci. USA 83:8744 (1986); idem., Photochem. Photobiol. 46:83 (1987); Hasan et al., Prog. Clin. Biol. Res. 288:471 (1989); Tatsuta et al., Lasers Surg. Med. 9:422 (1989); Pelegrin et al., Cancer 67:2529 (1991). However, these earlier studies did not include use of endoscopic therapy applications, especially with the use of antibody fragments or subfragments. Thus, the present invention contemplates the therapeutic use of immunoconjugates comprising photoactive agents or dyes.

Multimodal therapies are also contemplated within the present invention, including particularly for cancer, therapies which can be determined to be useful complementary therapies for the anti-metastatic embodiments of this invention such as anti-angiogenic Ab conjugates

In another form of multimodal therapy, subjects receive the multifunctional ligands of the present invention and standard cancer chemotherapy. For example, “CVB” (1.5 g/m.sup.2 cyclophosphamide, 200-400 mg/m² etoposide, and 150-200 mg/m² carmustine) is a regimen used to treat non-Hodgkin's lymphoma. Patti et al., Eur. J. Haematol. 51:18 (1993). Other suitable combination chemotherapeutic regimens are well-known to those of skill in the art. See, for example, Freedman et al., “Non-Hodgkin's Lymphomas,” in Cancer Medicine, Volume 2, 3rd Edition, Holland et al. (eds.), pages 2028-2068 (Lea & Febiger 1993). As an illustration, first generation chemotherapeutic regimens for treatment of intermediate-grade non-Hodgkin's lymphoma include C-MOPP (cyclophosphamide, vincristine, procarbazine and prednisone) and CHOP (cyclophosphamide, doxorubicin, vincristine, and prednisone). A useful second generation chemotherapeutic regimen is m-BACOD (methotrexate, bleomycin, doxorubicin, cyclophosphamide, vincristine, dexamethasone and leucovorin), while a suitable third generation regimen is MACOP-B (methotrexate, doxorubicin, cyclophosphamide, vincristine, prednisone, bleomycin and leucovorin). Additional useful drugs include phenyl butyrate and brostatin-1.

In general, the dosage of administered multifunctional ligands, immunoconjugates, and fusion proteins will vary depending upon such factors as the patient's age, weight, height, sex, general medical condition and previous medical history. Typically, it is desirable to provide the recipient with a dosage of antibody component, immunoconjugate or fusion protein which is generally at least in the range of from about 1 pg/kg to 10 mg/kg (amount of agent/body weight of patient), although a lower or higher dosage also may be administered as circumstances dictate, particularly to take advantage of the depot effect of the invention.

Administration of the invention including, immunoconjugates or fusion proteins to a patient can be intravenous, intraarterial, intraperitoneal, intramuscular, subcutaneous, intrapleural, intrathecal, by perfusion through a regional catheter, or by direct intralesional injection. When administering therapeutic proteins by injection, the administration may be by continuous infusion or by single or multiple boluses.

Those of skill in the art are aware that intravenous injection provides a useful mode of administration due to the thoroughness of the circulation in rapidly distributing antibodies. Intravenous administration, however, is subject to limitation by a vascular barrier comprising endothelial cells of the vasculature and the subendothelial matrix. Still, the vascular barrier is a more notable problem for the uptake of therapeutic antibodies by solid tumors. Lymphomas have relatively high blood flow rates, contributing to effective antibody delivery. Intralymphatic routes of administration, such as subcutaneous or intramuscular injection, or by catherization of lymphatic vessels, also provide a useful means of treating lymphomas. With regard to “low doses” of ¹³¹I-labeled immunoconjugates, the invention includes a dosage is in the range of 15 to 40 mCi, 20 to 30 mCi. In contrast, a preferred dosage of ⁹⁰Y-labeled immunoconjugates is in the range from 10 to 30 mCi, while the more preferable range is 10 to 20 mCi.

Immunoconjugates having a boron addend-loaded carrier for thermal neutron activation therapy will normally be effected in similar ways. However, it will be advantageous to wait until non-targeted immunoconjugate clears before neutron irradiation is performed. Clearance can be accelerated using an antibody that binds to the immunoconjugate. See U.S. Pat. No. 4,624,846 for a description of this general principle.

With respect enhancing the rate of clearance see also WO03/074569

The immunoconjugates, and fusion proteins of the present invention can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, REMINGTON'S PHARMACEUTICAL SCIENCES, 19th Ed. (1995).

For purposes of therapy, antibody components (or immunoconjugates/fusion proteins) and a pharmaceutically acceptable carrier are administered to a patient in a therapeutically effective amount. A combination of an antibody component, optionally with an immunoconjugate/fusion protein, and a pharmaceutically acceptable carrier is said to be administered in a “therapeutically effective amount” if the amount administered is physiologically significant. An agent is physiologically significant if its presence results in a detectable change in the physiology of a recipient patient. In one aspect, an agent is physiologically significant if its presence results in the inhibition of the growth of target tumor cells.

Yet another therapeutic method included in the invention is a method of treating cancer by administering to an animal suffering from cancer a pharmaceutically effective amount of one or more multifunctional ligands capable of binding to cancer cells, wherein the compound is associated with a substance capable of damaging cancer cells.

Pharmaceutical compositions herein described or alluded to include multifunctional ligands of the invention or therapeutics used in combination therapy which may be administered by a variety of routes of adminstration.

By administration of an “effective amount” is intended an amount of the compound that is sufficient to enhance or inhibit a response, is some embodiments particularly an immune response or cellular response to a multifunctional ligand. One of ordinary skill will appreciate that effective amounts of a multifunctional ligand can be determined empirically and may be employed in pure form or, where such forms exist, in pharmaceutically acceptable salt, ester or prodrug form. The multifunctional ligand may be administered in compositions in combination with one or more pharmaceutically acceptable excipients. It will be understood that, when administered to a human patient, the total daily usage of the compounds and compositions of the present invention will be decided by the attending physician within the scope of sound medical judgement. The specific therapeutically effective dose level for any particular patient will depend upon a variety of factors including the type and degree of the cellular response to be achieved; activity of the specific multifunctional ligand employed; the specific composition employed; the age, body weight, general health, sex and diet of the patient; the time of administration, route of administration, and rate of excretion of the agonist or antagonist; the duration of the treatment; drugs used in combination or coincidental with the specific agonist or antagonist; and like factors well known in the medical arts.

On administration parenterally, for example by i.v. drip or infusion, dosages optionally at leasr on the order of from 0.01 to 5 mg/kg/day, optionally 0.05 to 1.0 mg/kg/day and more preferably 0.1 to 1.0 mg/kg/day can be used. Suitable daily dosages for patients are thus on the order of from 2.5 to 500 mg p.o., optionally 5 to 250 mg p.o., optionally 5 to 100 mg p.o., or on the order of from 0.5 to 250 mg i.v., optionally 2.5 to 125 mg i.v. and optionally 2.5 to 50 mg i.v.

Dosaging may also be arranged in a patient specific manner to provide a predetermined concentration of an agonist or antagonist in the blood, as determined by the RIA technique. Thus patient dosaging may be adjusted to achieve regular on-going trough blood levels, as measured by RIA, optionally on the order of at least from 50 to 1000 ng/ml, preferably 150 to 500 ng/ml.

From above, pharmaceutical compositions are provided comprising an agonist or antagonist and a pharmaceutically acceptable carrier or excipient, which may be administered orally, rectally, parenterally, intracisternally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Optionally a composition for for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylceuulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

Some compositions herein descibed may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of one or therapeutic components herein described, it is desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

The multifunctional ligand can also be administered in the form of liposomes. As is known in the art, liposomes are generally derived from phospholipids or other lipid substances. Liposomes are formed by mono- or multi-lameflar hydrated liquid crystals that are dispersed in an aqueous medium. Any non-toxic, physiologically acceptable and metabolizable lipid capable of forming liposomes can be used. The present compositions in liposome form can contain, in addition to the agonist or antagonist, stabilizers, preservatives, excipients, and the like. The preferred lipids are the phospholipids and the phosphatidyl choaes (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

The present invention also contemplates a method of treatment in which immunomodulators are administered to prevent, mitigate or reverse radiation-induced or drug-induced toxicity of normal cells, and especially hematopoietic cells. Adjunct immunomodulator therapy allows the administration of higher doses of cytotoxic agents due to increased tolerance of the recipient mammal. Moreover, adjunct immunomodulator therapy can prevent, palliate, or reverse dose-limiting marrow toxicity. Examples of suitable immunomodulators for adjunct therapy include G-CSF, GM-CSF, thrombopoietin, IL-1, IL-3, IL-12, and the like. The method of adjunct immunomodulator therapy is disclosed by Goldenberg, U.S. Pat. No. 5,120,525.

For example, recombinant IL-2 may be administered intravenously as a bolus at 6 x 105 IU/kg or as a continuous infusion at a dose of 18×10⁶ IU/m²/d. Weiss et al., J. Clin. Oncol. 10:275 (1992). Alternatively, recombinant IL-2 may be administered subcutaneously at a dose of 12×10⁶ IU. Vogelzang et al., J. Clin. Oncol. 11:1809 (1993). Moreover, INF-.gamma. may be administered subcutaneously at a dose of 1.5×10⁶ U. Lienard et al., J. Clin. Oncol. 10:52 (1992). Furthermore, Nadeau et al., J. Pharmacol. Exp. Ther. 274:78 (1995), have shown that a single intravenous dose of recombinant IL-12 (42.5 .mu.g/kilogram) elevated IFN-.gamma. levels in rhesus monkeys.

Suitable IL-2 formulations include PROLEUKIN (Chiron Corp./Cetus Oncology Corp.; Emeryville, Calif.) and TECELEUKIN (Hoffmann-La Roche, Inc.; Nutley, N.J.). ACTIMMUNE (Genentech, Inc.; South San Francisco, Calif.) is a suitable INF-.gamma. preparation.

With respect to anti-CCR5 antibodies used to kill CCR5-expressing cells, with for example, bi-specific antibody chemokine fusions see Bruhl H. et al. J Immunol. Feb. 15, 2001 166(4): 2420-2426.

With respect to targeting IKAP proteins see for example U.S. Pat. No. 6,172,195.

With respect to pertinent diseased cells, disease causing cells and other suitable targets for immunotoxins, as well as optional toxins and methods of making and using immunotoxins and related technologies see for example US05980895 Immunotoxin containing a disulfide-stabilized antibody fragment joined to a Pseudomonas exotoxin that does not require proteolytic activation; US05686072 Epitope-specific monoclonal antibodies and immunotoxins and uses thereof; US04956453 Antihuman ovarian cancer immunotoxins and methods of thereof; US06146631 Immunotoxins comprising ribosome-inactivating proteins; US05756699 Immunotoxins comprising ribosome-inactivating Proteins; US05744580 Immunotoxins comprising ribosome-inactivating Proteins; US06146850 Proteins encoding gelonin sequences; US05837491 Polynucleotides encoding gelonin sequences; US05578706 Methods and compositions concerning homogenous immunotoxin preparations; US05185434 Prolonged-action immunotoxins containing a glycopeptide constituent which inactivates ribosomes, modified on its polysaccharide units; US04958009 Anti-human ovarian cancer immunotoxins and methods of use thereof; US05980896 Antibodies reactive with human carcinomas; US06074644 Nucleic acids encoding immunotoxins containing a disulfide-stabilized antibody fragment replacing half or more of domain IB of pseudomonas exotoxin, and methods of use of the encoded immunotoxins; US04981953 Immunotoxins, process for their preparation and pharmaceutical compositions in which they are present; US04980457 Cytotoxic conjugates which can be used in therapy and process or their preparation; US04545985 Pseudomonas exotoxin conjugate immunotoxins; US06020145 Methods for determining the presence of carcinoma using the antigen binding region of monoclonal antibody BR96; US05792458 Mutant diphtheria toxin conjugates; US05338542; US06051230 Compositions for targeting the vasculature of solid tumors; B3 antibody fusion proteins and their uses; US05990275 Linker and linked fusion polypeptides; US05981726 Chimeric and mutationally stabilized tumor-specific B1, B3 and B5 antibody fragments; immunotoxic fusion proteins; and uses thereof; US05965132 Methods and compositions for targeting the vasculature of solid tumors; US05889157 Humanized B3 antibody fragments, fusion proteins, and uses thereof US06027725 Multivalent antigen-binding proteins; EP00861091A1 IMMUNOTOXIN CONTAINING A DISULFIDE-STABILIZED ANTIBODY FRAGMENT US05776427 Methods for targeting the vasculature of solid tumors; US05772997 Monoclonal antibodies directed to the HER2 receptor US05665357 Antibodies recognizing tumor associated antigen CA 55.1; US05660827 Friedrich K, et al. A two-step selection approach for the identification of ligand-binding determinants in cytokine receptors. Anal Biochem. Mar. 15, 1999; 268(2):179-86. Krebs B, et al. Recombinant human single chain Fv antibodies recognizing human interleukin-6. Specific targeting of cytokine-secreting cells. J Biol Chem. Jan. 30, 1998; 273(5):2858-65; Wilbur D S, et al. Related Articles Biotin reagents for antibody pretargeting. 2. Synthesis and in vitro evaluation of biotin dimers and trimers for cross-linking of streptavidin. Bioconjug Chem. November-December 1997; 8(6):819-32.Ring D B, et al Antigen forks: bispecific reagents that inhibit cell growth by binding selected pairs of tumor antigens. Cancer Immunol Immunother. July 1994; 39(1):41-8. WO09942597A1 MONOVALENT, MULTIVALENT, AND MULTIMERIC MHC BINDING DOMAIN FUSION PROTEINS AND CONJUGATES, AND USES THEREFOR; EP00935607A2 SOLUBLE MONOVALENT AND MULTIVALENT MHC CLASS II FUSION PROTEINS, AND USES THEREFOR; WO09811914A1 TARGETING ANTIGENS TO THE MHC CLASS IPROCESSING PATHWAY WITH ANTHRAX TOXIN FUSION PROTEIN WO09728191A1 MHC COMPLEXES AND USES THEREOF; US05580756 B7IG fusion protein; US06143298 Soluble truncated forms of ICAM-1; US05852175 P-selectin glycoprotein ligand blocking antibodies US05800815Antibodies to P-selectin and their uses; US06037454 Humanized anti-CD11a antibodies; US06020152 Lymphocyte-associated cell surface protein US05807734 Monoclonal antibodies and FV specific for CD2antigen US05622701 Cross-reacting monoclonal antibodies specific for E- and P-selectin US05622700 Method for treating a LFA-1-mediated disorder; JP06209788A2 IMMUNOASSAY OF HUMAN SOLUBLE ICAM-1, ANTIBODY AND KIT FOR MEASUREMENT THEREOF; JP03072430A2 ANTIVIRAL AGENT BY USING FUNCTIONAL DERIVATIVE OF INTERCELLULAR ADHESIVE MOLECULE; JP01135724A2 TREATMENT FOR NONSPECIFIC INFLAMMATION; US06123915 Methods for using agents that bind to VCAM-1; WO09929706A2 DISALICYLATE ANALOG BASED SIALYL LEWIS×MIMETICS; WO09918442A1 DIAGNOSIS OF THROMBOTIC EVENTS BY DETECTING P-SELECTIN; US05877295 Antibodies which bind a subpopulation of Mac-1(CD11b/CD18) molecules which mediate neutrophil adhesion to ICAM-land fibrinogen; US05869460 Sulfated and phosphated saccharide derivatives, process for the preparation of the same and use thereof; US05858994 Carbohydrate conjugates as inhibitors of cell adhesion; US05811405 Multiply fucosylated dicarboxylic acids possessing anti adhesive properties; US05654282 Selectin binding glycopeptides; US05632991 Antibodies specific for E-selectin and the uses thereof; US05599676 Method for isolating a novel receptor for.alpha.4 integrins; US05580862 Sulfate ligands for L-selectins and methods of preventing sulfate addition; US05508387 Selectin binding glycopeptides; US06177547 Antibodies to P-selectin glycoprotein ligand US05827670 Methods of isolating and detecting bone marrow stromal cells with VCAM-1-specific antibodies; US05756095 Antibodies with specificity for a common epitope on E-selectin and L-selectin; US05565550 Antibodies to ICAM-2, and fragments thereof; US06099838 Pharmaceutical compositions comprising anti-CD45RB antibodies for the inhibition of T-cell mediated immune responses; US05595737 Methods for using monoclonal antibodies specific for cell-surface bound LAM-1; US05324510, US06183988 Leukocyte-specific protein and gene, and methods of use thereof; US05998598; US05997865 Agonist antibodies against the flk2/flt3 receptor and uses thereof; US05993816 Methods to inhibit humoral immune responses, immunoglobulin production and B cell activation with 5c8-specific antibodies; US05869453 Cytotoxic T-cell epitopes; US05861151 Soluble fusion molecules with binding specificity for cell adhesion molecules; US05843441 Use of endothelial-leukocyte adhesion molecule-1 specific antibodies in the treatment of asthma US05821332 Receptor on the surface of activated CD4+ T-cells: ACT-4; EP00868197A1 ANTI-SELECTIN ANTIBODIES FOR PREVENTION OF MULTIPLE ORGAN FAILURE AND ACUTE ORGAN DAMAGE; US05817515 Human B2 integrin alpha subunit antibodies; EP00528931B1 HUMANIZED CHIMERIC ANTI-ICAM-1 ANTIBODIES, METHODS OF PREPARATION AND USE; US05776775 Anti-LAM 1-3 antibody and hybridoma; US06063906 Antibodies to integrin alpha subunit; US05997865 Agonist antibodies against the flk2/flt3 receptor and uses thereof; US05993816 Methods to inhibit humoral immune responses, immunoglobulin production and B cell activation with 5c8-specific antibodies US05951982 Methods to suppress an immune response with variant CD44-specific antibodies US05843441 Use of endothelial-leukocyte adhesion molecule-1 specific antibodies in the treatment of asthma US05821332 Receptor on the surface of activated CD4+T-cells: ACT-4; US05821123 Modified antibody variable domains; EP00868197A1 ANTI-SELECTIN ANTIBODIES FOR PREVENTION OF MULTIPLE ORGAN FAILURE AND ACUTE ORGAN DAMAGE; US05817515 Human B2 integrin alpha subunit antibodies; EP00528931B1 HUMANIZED CHIMERIC ANTI-ICAM-1 ANTIBODIES, METHODS OF PREPARATION AND USE; US05776775 Anti-LAM 1-3 antibody and hybridoma; US05776755 FLT4, a receptor tyrosine kinase; US05730978 Inhibition of lymphocyte adherence with alpha.4&#223 1;-specific antibodies.

Examples of tumor specific antigens are numerous and are referred to in the hereinabove cited references and as well as the in the following references: US06132980 Oct. 17, 2000 Antibodies specific for TRP-2 a human tumor antigen recognized by cytotoxic T lymphocytes US06165464Monoclonal antibodies directed to the HER2 receptor, US05824311 Treatment of tumors with monoclonal antibodiesagainst oncogene antigens. US06140050 Oct. 31, 2000 Methods for determining breast cancer and melanoma by assaying for a plurality of antigens associated herewith; US06051226 MN-specific antibodies and their use in cancer treatment; US06020145Methods for determining the presence of carcinoma using the antigen binding region of monoclonal antibody BR96.; US05980896 Antibodies reactive with human carcinomas US05955075 Method of inhibiting tumor growth using antibodies to MN protein US05917124 Transgenic mouse model of prostate cancer; US05914389 Jun. 22, 1999 E6 associated protein US05912143 Jun. 15, 1999 Polynucleotides encoding a human mage protein homolog US05910626 Jun. 08, 1999 Acetyl-CoA carboxylase compositions and methods of use US05874560 Feb. 23, 1999 Melanoma antigens and their use in diagnostic and therapeutic methods US05872217 Feb. 16, 1999 Antibodies which specifically bind a cancer related antigen US05869636 Feb. 09, 1999 Immunoreactive peptide sequence from a 43 kD human cancer antigen US05869045 Feb. 09, 1999 Antibody conjugates reactive with human carcinomas US05866124; Feb. 02, 1999 Antiidiotypic antibodies for high molecular weight-melanoma associated by same US05847083 Dec. 08, 1998 Modified p53 US05844075 Melanoma antigens and their use in diagnostic and therapeutic methods US05843685 Dec. 01, 1998 Production of chimeric mouse-human antibodies with specificity to human tumor antigens US05843648 P15 and tyrosinase melanoma antigens and their use in diagnostic and therapeutic methods US05840854US05830470 Nov. 03, 1998 Humanized antibodies to ganglioside GM2US05830464 Nov. 03, 1998 US05808005Human carcinoma Bispecific molecules recognizing lymphocyte antigen CD2 and tumor antigens US05792456 Mutant BR96 antibodies reactive with human carcinomas US0578368US05773579 Lung cancer marker US05772997 Monoclonal antibodies directed to the HER2 receptor US05770374; US05705157 Methods of treating cancerous cells with anti-receptor antibodies US05695994 Dec. 09, 1997 Isolated cytolytic T cells specific for complexes of MAGE related peptides and HLA molecules US05693763 Dec. 02, 1997 Antibodies to human carcinoma antigen.

Tumor rejection antigens which correspond to amino acid sequences in tumor rejection antigen precursor bage, and uses thereof US05681701 Immortalized human fetal osteoblastic cells US05681562 Oct. 28, 1997 US05677171 Oct. 14, 1997 Monoclonal antibodies directed to the HER2receptor US05674486 Oct. 07, 1997US05665357 Sep. 09, 1997 Antibodies recognizing tumor associated antigen CA 55.1; Fonsatti E, et al Emerging role of protectin (CD59) in humoral immunotherapy of solid malignancies. Clin Ter. May-June 2000; 151(3): 187-93 Knuth A, et alCancer immunotherapy in clinical oncology. Cancer Chemother Pharmacol. 2000; 46 Suppl:S46-51: Sievers E L. Targeted therapy of acute myeloid leukemia with monoclonal antibodies and immunoconjugates. Cancer Chemother Pharmacol. 2000; 46 Suppl:S18-22. 172 van Spriel A B, et alImmunotherapeutic perspective for bispecific antibodies.Immunol Today. August 2000; 21(8):391-7 273: Green M C, et alMonoclonal antibody therapy for solid tumors. Cancer Treat Rev. August 2000; 26(4):269-86; Xiang J Targeting cytokines to tumors to induce active antitumor immune responses by recombinant fusion proteins. Hum Antibodies. 1999; 9(1):23-Engberg J, et alRecombinant antibodies with the antigen-specific, MHC restricted specificity of T cells: novel reagents for basic and clinical investigations and immunotherapy. Immunotechnology. March 1999; 4(3-4):273-8. O'Brien T J, Tet alMore than 15 years of CA 125: what is known about the antigen, its structure and its function.Int J Biol Markers. October-December 1998; 13(4):188-95. Sharifi J, et alImproving monoclonal antibody pharmacokinetics via chemical modification.Q J Nucl Med. December 1998; 42(4): 242-9.

Ligands on immune or other cells which may be targeted with bispecific ligands in which one ligand of the pair dictates specificity for a population of cells or particular sub-population of those cells and a second ligand with reduced functional affinity is used to effect a specific immune function include those referenced in the following patents and publications therein referenced: US06132992 Expression vectors encoding bispecific fusion proteins and methods of producing biologically active bispecific fusion proteins in a mammalian cell; Antibody heteroconjugates and bispecific antibodies for use in regulation of lymphocyte activity; WO09942077 COMPOSITIONS AND METHODS FOR REGULATING LYMPHOCYTE ACTIVATION; US059165600 Methods for inhibiting an immune response by blocking the GP39/CD40 and CTLA4/CD28/B7 pathways and compositions for use therewith; US05876718 Methods of inducing T cell non-responsiveness to transplanted tissues and of treating graft-versus-host-disease with anti-gp39 antibodies EP00445228B IMMUNOTHERAPY INVOLVING CD28 STIMULATION; US05709859 Mixed specificity fusion proteins; US05637481 Expression vectors encoding bispecific fusion proteins and methods of producing biologically active bispecific fusion proteins in a mammalian cell; WO09720048 MODIFIED SFV MOLECULES WHICH MEDIATE ADHESION BETWEEN CELLS AND USES THEREOF; EP00336379 Antibody heteroconjugates for use in regulation of lymphocyte activity; EP00537293 LIGAND FOR CD28 RECEPTOR ON B CELLS AND METHODS; US05182368 Ligands and methods for augmenting B-cell proliferation; WO09300431 CTL4A RECEPTOR, FUSION PROTEINS CONTAINING IT AND USES THEREOF; EP00445228 IMMUNOTHERAPY INVOLVING CD28 STIMULATION; Role of cellular adhesion molecules in HIV type 1 infection and their impact on virus neutralization. AIDS Res Hum Retroviruses. October 1998; 14 Suppl 3:S247-54 Cavenagh J D,et alAdhesion molecules in clinical medicine. Crit Rev Clin Lab Sci. September 1998; 35(5):415-59 Viney J L, Fong S. Beta 7 integrins and their ligands in lymphocyte migration to the gut. Chem Immunol. 1998; 71:64-76 Aplin A E, et alSignal transduction and signal modulation by cell adhesion receptors: the role of integrins, cadherins, immunoglobulin-cell adhesion molecules, and selectins. Pharmacol Rev. June 1998; 50(2):197-263

With respect to ascertaining important amino acid residues for receptor activation or binding see also Zang, Q., Springer, T. A. (2001). Amino Acid Residues in the PSI Domain and Cysteine-rich Repeats of the Integrin beta 2 Subunit That Restrain Activation of the Integrin alpha xbeta 2. J. Biol. Chem. 276: 6922-6929; Binding site on the murine IFN-gamma receptor for IFN-gamma has been identified using the synthetic peptide approach, The Journal of Immunology, Vol 151, Issue 11 6206-6213; The Journal of Immunology, Vol 143, Issue 11 3568-3579 The main immunogenic region of the nicotinic acetylcholine receptor. Identification of amino acid residues interacting with different antibodies, M Bellone et al.; Arend, W. P., Malyak, M., Guthridge, C. J., Gabay, C. (1998). INTERLEUKIN-1 RECEPTOR ANTAGONIST: Role in Biology. Annu. Rev. Immunol. 16: 27-55; The Journal of Immunology, Vol 155, Issue 10 4719-4725, Mapping of receptor binding sites on IL-1 beta by reconstruction of IL-1ra-like domains; The Journal of Immunology, 2000, 165: 6966-6974 Identification of Fetal Liver Tyrosine Kinase 3 (Flt3) Ligand Domain Required for Receptor Binding and Function Using Naturally Occurring Ligand Isoforms Waithaka Mwangi³, Wendy C. Brown and Guy H. Palmer.
³ The term small molecule typically connotes small synthetic or semi-synthetic organic molecules which are not macromolecules such as proteins, lipoproteins, glycoproteins, long chain fatty acids etc.

It will be appreciated that one or both ligand binding moieties may exert additional effector properties.

The invention also contemplates multifunctional ligands comprising various combinations and permutations of such ligands including pairs and three different such ligands including multifunctional ligands including such combinations and a ligand which binding to a lymphatic vessel associated ligand. Additional pertinent references pertaining to formation of antibody dimers, microarrays of (and tissue microarrays) proteins including heterofunctional proteins and recombinant, ligands having application to the invention, and phage or ribosome display strategies having relevance herein include Zhu H. et al. Protein arrays and microarrays.Curr Opin Chem Biol. February 2001; 5(1):40-5, references in IBC's conference on Protein Microarray Technology March 19-21 Santiago California; WO 99/06834, WO 99/19506; WO 97/02342, WO 00/63701; WO 99/40434; US 6,127,127; U.S. Pat. No. 6,146,830, WO 00/075298, U.S. Pat. No. 6,165,709 US06204023 Mar. 20, 2001 Modular assembly of antibody genes, antibodies prepared thereby and use; US05846818 Dec. 08, 1998 Pectate lyase signal sequence; US05698435 Dec. 16, 1997 Modular assembly of antibody genes, antibodies prepared thereby and use; US05698417 Dec. 16, 1997 Modular assembly of antibody genes, antibodies prepared thereby and use; US05693493 Dec. 02, 1997 Modular assembly of antibody genes, antibodies prepared thereby and use; US05514548 May. 07, 1996 Method for in vivo selection of ligand-binding proteins; US05648237 Jul. 15, 1997 Expression of functional antibody fragments; U.S. Pat. No. 0,618,034 In vitro scanning saturation mutagenesis of proteins; US06027933 Surface expression libraries of heteromeric receptors; US05910573 Jun. 08, 1999 Monomeric and dimeric antibody-fragment fusion proteins; US0615058311 Transgenic animals expressing artificial epitope-tagged proteins; US06132992 Expression vectors encoding bispecific fusion proteins and methods of producing biologically active bispecific fusion proteins in a mammalian cell; US06127524Binding molecules and computer-based methods of increasing the binding affinity thereof; US06071515 Dimer and multimer forms of single chain polypeptides; US06054297 Humanized antibodies and methods for making them; WO00004382 ARRAYS OF PROTEINS AND METHODS OF USE THEREOF; US06008023 Cytoplasmic expression of antibodies, antibody fragments and antibody fragment fusion proteins in E. coli; Phagemid for antibody screening; US05980895 Nov. 09, 1999 Immunotoxin containing a disulfide-stabilized antibody fragment joined to a Pseudomonas exotoxin that does not require proteolytic activation; US05962255 Methods for producing recombinant vectors; US05955341Heterodimeric receptor libraries using phagemids; WO09939210A1 HIGH DENSITY ARRAYS FOR PROTEOME ANALYSIS AND METHODS AND COMPOSITIONS THEREFOR; WO09931267 METHODS FOR THE SIMULTANEOUS IDENTIFICATION OF NOVEL BIOLOGICAL TARGETS AND LEAD STRUCTURES FOR DRUG DEVELOPMENT; US05869619 Modified antibody variable domains; US05855885Isolation and production of catalytic antibodies using phage technology; US05851801 Dec. 22, 1998 Method of preparing polypeptide binding compositions derived from immunoglobulin variable regions; US05849500 Phagemid for antibody screening;binding composition; US05837846 Nov. 17, 1998 Biosynthetic binding proteins for immuno-targeting; US05821337 Oct. 13, 1998 Immunoglobulin variants; US05821123Modified antibody variable domains; US05789655 Aug. 04, 1998 Transgenic animals expressing artificial epitope-tagged proteins; US05783384 Selection of binding-molecules; US05780225 Method for generating libaries of antibody genes comprising amplification of diverse antibody DNAs and methods for using these libraries for the production of diverse antigen combining molecules; US05770356 Phagemids coexpressing a surface receptor and a surface heterologous protein; WO09808603 ISOLATION OF IMMUNOGLOBULINS; US05716805 Methods of preparing soluble, oligomeric proteins; US05595898 Modular assembly of antibody genes, antibodies prepared thereby and use; US05582996 Bifunctional antibodies and method of preparing same; US05580717Recombinant library screening methods; Haab B B, Dunham M J, Brown P O Protein microarrays for highly parallel detection and quantitation of specific proteins and antibodies in complex solutions.Genome Biol. 2001; 2(2):: Moch H, Kononen T, Kallioniemi O P, Sauter G. Tissue microarrays

Borrebaeck C A.Antibodies in diagnostics—from immunoassays to protein chips.Immunol Today. August 2000; 21(8):379-82

Mendoza L G, et alHigh-throughput microarray-based enzyme-linked immunosorbent assay (ELISA). Biotechniques. October 1999; 27(4):778-80, 782-6, 788. Morozov V N, Morozova T Ya Electrospray deposition as a method for mass fabrication of mono- and multicomponent microarrays of biological and biologically active substances.Anal Chem. Aug. 1, 1999; 71(15):3110-7.619: Lueking A, et alProtein microarrays for gene expression and antibody screening. Anal Biochem. May 15, 1999; 270(1):103-11. Silzel J W, et alMass-sensing, multianalyte microarray immunoassay with imaging detection. Clin Chem. September 1998; 44(9):2036-43 Ekins R P. Ligand assays: from electrophoresis to miniaturized microarrays.Clin Chem. September 1998; 44(9):2015-30.

All publications referred to herein are indicative of the level of skill of those in the art to which the invention pertains.

With respect to lymphatic vessel associated ligands see also U.S. Pat. No. 5,776,755 (flt4), Mod Pathol February 2000; 13(2):180-5; EMBO J Mar. 15, 2001; 20(6):1223-1231, Nat Med February 2001: 7(2):199-205 Inhibition of lymphangiogenesis with resulting lymphedema in transgenic mice expressing soluble VEGF receptor-3 (VEGFR-3), J Pathol February 2001; 193(2):147-54 Localization of vascular endothelial growth factor-D in malignant melanoma suggests a role in tumour angiogenesis.

With respect to technologies having application for consideration herein see also Immunity April 2001; 14(4):437-46 The immunological barrier to xenotransplantation. Cascalho M, Platt J L.; WO 01/43779; WO 01/42285; WO 98/10795; WO 01/40803; WO 00/14212; Gastroenterology May 2001; 120(6):1330-8 An engineered human antibody to TNF (CDP571) for active Crohn's disease: a randomized double-blind placebo-controlled trial. Sandborn W J; WO 01/44282; WO 01/40309; WO 01/40274; WO 01/44300; Ann Rheum Dis May 2001; 60(5):433 Cancer and autoimmunity: autoimmune and rheumatic features in patients with malignancies, Abu-Shakra M, et al.; WO 01/40468; WO 01/40307; WO 01/42297; WO 01/42294; WO 01/42296; WO 01/40456; WO 01/40308; WO 01/42306; Curr Opin Immunol April 2001; 13(2):134-40 Immunity against cancer: lessons learned from melanoma. Houghton A N, Gold J S, Blachere N E.; WO 01/42288; WO 01/42288; WO 01/43771; WO 01/42308; WO 01/41804; WO 01/39722; WO 01/44808; WO 01/43770; WO 01/16166; WO 01/41803; WO 01/13110; WO 00/32752; WO 98/33528; WO 01/43695; J Am Pharm Assoc (Wash) May-June 2001; 41(3):383-91 Magic bullets finally find their mark.; Leukemia April 2001; 15(4):675-6; WO 01/44301; Anticancer Res January-February 2001; 21(1B):621-7 Immunotherapy for recurrent colorectal cancers with human monoclonal antibody SK-1. Koda K et al.; WO 01/10911; WO 01/42506; Int J Clin Pract April 2001; 55(3):211-6 Tumour necrosis factor as a therapeutic target in rheumatoid arthritis and other chronic inflammatory diseases: the clinical experience with infliximab; WO 01/44472; WO 01/40302; WO 01/40305)

With respect to surface plasmon resonance measurements of affinity see US6111652:High throughput surface plasmon resonance analysis system US06208422 Surface plasmon sensor EP01080365 SURFACE PLASMON RESONANCE SENSOR FOR THE SIMULTANEOUS MEASUREMENT OF A PLURALITY OF SAMPLES IN FLUID FORM WO00106236A HIGH THROUGHPUT ANALYSIS OF MOLECULAR INTERACTION USING SURFACE PLASMON RESONANCE High throughput surface plasmon resonance analysis system as well as Dimensions of antigen recognition and levels of immunological specificity. Adv Cancer Res. 2001;80:147-87. Use of optical biosensors for the study of mechanistically concerted surface adsorption processes. Anal Biochem. Jan. 15, 2001; 288(2):109-25 Experimental design for analysis of complex kinetics using surface plasmon resonance. Methods. March 2000; 20(3):310-8., Dmitriev D A, et al. Analysis of bispecific monoclonal antibody binding to immobilized antigens using an optical biosensor. Biochemistry(Mosc.(2002);67(12):1356-65.PMID:12600264; Bera T K, et al. A bivalent disulfide-stabilized Fv with improved antigen binding to erbB2.J Mol Biol.(1998);281(3):475-83.PMID:9698563; Piehler J, et al.Assessment of affinity constants by rapid solid phase detection of equilibrium binding in a flow system.J Immunol Methods.(1997);201(2):189-206.PMID:9050941; George A J, et al. Measurement of kinetic binding constants of a panel of anti-saporin antibodies using a resonant mirror biosensor.J Immunol Methods.(1995);183(1):51-63.PMID:7602139; Abraham R, et al. Screening and kinetic analysis of recombinant anti-CEA antibody fragments. J Immunol Methods. (1995);183(1):119-25.PMID:7602129; Altschuh D, et al. Determination of kinetic constants for the interaction between a monoclonal antibody and peptides using surface plasmon resonance. Biochemistry.(1992);31(27):6298-304.PMID: 1627568; Lipshultz C A et al. Experimental design for analysis of complex kinetics using surface plasmon resonance. Methods. March 2000; 20(3):310-8.PMID:10694453; Skeie GO, et al.,. Autoimmunity against the ryanodine receptor in myasthenia gravis. Acta Physiol Scand. March 2001; 171(3):379-84. Haufs M G, et al. Epidermolysis bullosa acquisita treated with basiliximab, an interleukin-2 receptor antibody. Acta Derm Venereol. January-February 2001; 81(1):72. Woo E Y, et al Regulatory CD4(+)CD25(+) T cells in tumors from patients with early-stage non-small cell lung cancer and late-stage ovarian cancer. Cancer Res. Jun. 15, 2001; 61(12):4766-72. Barrera P, et al. Effects of treatment with a fully human anti-tumour necrosis factor alpha monoclonal antibody on the local and systemic homeostasis of interleukin 1 and TNF alpha in patients with rheumatoid arthritis. Ann Rheum Dis. July 2001; 60(7):660-9. Nicholson J K, et al. CCR5 and CXCR4 expression on memory and naive T cells in HIV-1 infection and response to highly active antiretroviral therapy. J Acquir Immune Defic Syndr. Jun. 1, 2001; 27(2):105-15. Kung S K, et al. Characterization of four new monoclonal antibodies that recognize mouse natural killer activation receptors. Hybridoma. April 2001; 20(2):91-101. Bank I, et al. Differential expression and regulation of CD6 on T-cell subsets revealed by monoclonal antibody (MAb) CH11. Hybridoma. April 2001; 20(2):75-84.

With respect to making multifunctional ligands see also U.S. Pat. Nos. 5,731,168 and 5,821,333.

With respect to TNF and TNFR variants, and functional fragments thereof, for use as antibody targets and binding moieties with respect to various aspects of the invention herein see WO 00/67793, WO 01/30300, WO 01/49321, WO 00/62790, WO 01/03720, WO 00/60079, WO 97/46686, WO 01/41803, WO 01/38526, WO 01/37874, WO 01/12812, WO 01/12671, WO 01/05834, WO 01/03720, WO 00/77191 WO 00/73321, WO 00/71150, WO 00/67793, WO 00/67034, WO 00/66608, WO 00/66156., WO 01/24811, as well as references cited therein. Many other TNFR variants and TNF analogs are known in the art.

With respect to cytokines and cytokine receptors see also the latest editions of Cytokine Reference: A Compendium of Cytokines and Other Mediators of Host Defense by Joost J. Oppenheim (Editor), Jan Vilcek, Nicos A. Nicola (Editor); Cytokine Molecular Biology : A Practical Approach by Frances R. Balkwill (Editor), Fran Balkwill (Editor); Guidebook to Cytokines and Their Receptors by Nicos Nicola (Editor); The Cytokine Network and Immune Functions by Jacques Theze; Novel Cytokine Inhibitors by Gerry A. Higgs (Editor), Brian Henderson (Editor); Homology Folding of Proteins : Application to Cytokine Engineering by Subhashini Srinivasan; Cytokines and Cytokine Receptors (2001); International Review of Experimental Pathology : Cytokine-Induced Pathology, Part B: Inflammatory Cytokines, Receptors, and Disease by G. W. Richter, Kim Solez (Editor).

With respect to antibodies that bind to CCR5 see Mol Biol Cell February 2002; 13(2): 723-737.

With respect to variations in chemokine receptors, cytokine and other receptors that can be exploited according to one or more aspects of the invention herein see 1: Csaszar A, Abel T. Receptor polymorphisms and diseases.Eur J Pharmacol. Feb. 23, 2001; 414(1):9-22. 2: Gibejova A. Chemokine receptors.Acta Univ Palacki Olomuc Fac Med. 2000;143:9-18. 3: Nishimoto N, Kishimoto T, Yoshizaki K. Anti-interleukin 6 receptor antibody treatment in rheumatic disease.Ann Rheum Dis. November 2000; 59 Suppl 1:i21-7. 4: Aggarwal B B. Tumour necrosis factors receptor associated signalling molecules and their rolein activation of apoptosis, JNK and NF-kappaB.Ann Rheum Dis. November 2000; 59 Suppl 1:i6-16. 5: Grignani G, Maiolo A. Cytokines and hemostasis.Haematologica. September 2000; 85(9):967-72. 6: Idriss H T, Naismith J H. TNF alpha and the TNF receptor superfamily: structure-function relationship(s).Microsc Res Tech. Aug. 1, 2000; 50(3): 184-95. 7: van Deventer S J. Cytokine and cytokine receptor polymorphisms in infectious disease.Intensive Care Med. 2000;26 Suppl 1:S98-102. 8: Gessner A, Rollinghoff M. Biologic functions and signaling of the interleukin-4 receptor complexes.Immunobiology. January 2000; 201(3-4):285-307. 9: Platanias L C, Fish E N. Signaling pathways activated by interferons.Exp Hematol. November 1999; 27(11):1583-92. 10: Schwertschlag U S, Trepicchio W L, Dykstra K H, Keith J C, Turner K J, Dorner A J. Hematopoietic, immunomodulatory and epithelial effects of interleukin-11.Leukemia. September 1999; 13(9):1307-15. 11: Blasi F. The urokinase receptor. A cell surface, regulated chemokine.APMIS. January 1999; 107(1):96-101. 12: Izuhara K, Shirakawa T. Signal transduction via the interleukin-4 receptor and its correlation with atopy.Int J Mol Med. January 1999; 3(1):3-10. 13: Tsokos G C, Liossis S N. Lymphocytes, cytokines, inflammation, and immune trafficking.Curr Opin Rheumatol. September 1998; 10(5):417-25. 14: Morishita R, Nakamura S, Hayashi S, Aoki M, Matsushita H, Tomita N, Yamamoto K, Moriguchi A, Higaki J, Ogihara T. Contribution of a vascular modulator, hepatocyte growth factor (HGF), to the pathogenesis of cardiovascular disease.J Atheroscler Thromb. 1998;4(3):128-34. 15: Kashiwamura S, Okamura H.[IL-18 and IL-18 receptor].Nippon Rinsho. July 1998; 56(7):1798-806. Japanese.16: Paxton W A, Kang S. Chemokine receptor allelic polymorphisms: relationships to HIV resistance and disease progression.Semin Immunol. June 1998; 10(3):187-94. 17: Arend W P, Malyak M, Guthridge C J, Gabay C. Interleukin-1 receptor antagonist: role in biology.Annu Rev Immunol. 1998;16:27-55. 18: Camussi G, Lupia E. The future role of anti-tumour necrosis factor (TNF) products in the treatment of rheumatoid arthritis.Drugs. May 1998; 55(5):613-20. 19: Taga T, Kishimoto T. Gp130 and the interleukin-6 family of cytokines.Annu Rev Immunol. 1997; 15:797-819. 20: Paul W E. Interleukin 4: signalling mechanisms and control of T cell differentiation.Ciba Found Symp. 1997;204:208-16; discussion 216-9; 1.(WO 01/49321) TNF INHIBITORS FOR THE TREATMENT OF NEUROLOGICAL, RETINAL AND MUSCULAR DISORDERS2.(WO 01/46261) METHOD FOR TREATING INFLAMMATION3.(WO 01/40464) INTERLEUKIN-1-RECEPTOR ASSOCIATED KINASE-3 (IRAK3) AND ITS USE IN PROMOTION OR INHIBITION OF ANGIOGENESIS AND CARDIOVASCULARIZATION4.(WO 01/40464) INTERLEUKIN-1-RECEPTOR ASSOCIATED KINASE-3 (IRAK3) AND ITS USE IN PROMOTION OR INHIBITION OF ANGIOGENESIS AND CARDIOVASCULARIZATION5.(WO 01/30850) UMLR POLYPEPTIDES6.(WO 00/77195) NUCLEIC ACID ENCODING NOVEL EGF-LIKE GROWTH FACTORS7.(WO 00/74719) METHOD OF TREATING CARCINOMA USING ANTIBODY THERAPY AND AMELIORATING SIDE EFFECTS ASSOCIATED WITH SUCH THERAPY8.(WO 00/02582) TREATMENT OF CELIAC DISEASE WITH INTERLEUKIN-15 ANTAGONISTS9.(WO 99/47170) PREVENTIVES OR REMEDIES FOR INFLAMMATORY INTESTINAL DISEASES CONTAINING AS THE ACTIVE INGREDIENT IL-6 ANTAGONISTS10.(WO 99/46376) RECEPTOR FROM THE SUPERFAMILY OF TNT-RECEPTORS FROM THE HUMAN LUNG11.(WO 99/43809) PROTEASE-ACTIVATED RECEPTOR 4 AND USES THEREOF12.(WO 98/48017) FAMILY OF IMMUNOREGULATORS DESIGNATED LEUKOCYTE IMMUNOGLOBULIN-LIKE RECEPTORS (LIR)13.(WO 98/47923) IL-5R ANTAGONISTS FOR TREATMENT OF INFLAMMATION, ASTHMA AND OTHER ALLERGIC DISEASES 14.(WO 98/46620) A NOVEL HUMAN G-PROTEIN COUPLED RECEPTOR15.(WO 98/46265) METHODS FOR USING ANTAGONISTIC ANTI-AVB3 INTEGRIN ANTIBODIES16.(WO 98/36767) MODULATION OF THE HYPOTHALAMIC-PITUITARY-ADRENAL-ADIPOSE AXIS WITH LEPTIN RECEPTOR LIGANDS17.(WO 98/31809) HUMAN CC CHEMOKINE SLC18.(WO 98/30706) COMPOUNDS, COMPOSITIONS AND METHODS FOR THE ENDOCYTIC PRESENTATION OF IMMUNOSUPPRESSIVE FACTORS19.(WO 98/24817) NOVEL DNA, NOVEL PROTEIN, AND NOVEL ANTIBODY20.(WO 98/22499) NEURON AND NEURAL TUMOUR GROWTH REGULATORY SYSTEM, ANTIBODIES THERETO AND USES THEREOF21.(WO 98/19706) IDENTIFICATION OF UNIQUE BINDING INTERACTIONS BETWEEN CERTAIN ANTIBODIES AND THE HUMAN B7.1 AND B7.2 CO-STIMULATORY ANTIGENS22.(WO 98/18456) PROTEASE-ACTIVATED RECEPTOR 3 AND USES THEREOF23.(WO 98/14480) G PROTEIN-COUPLED RECEPTOR ANTAGONISTS 24.(WO 98/02541 ) GAMMA-HEREGULIN25.(WO 97/49818) G-BETA-GAMMA REGULATED PHOSPHATIDYLINOSITOL-3′ KINASE26.(WO 97/48804) TIE-2 RECEPTOR LIGANDS (TIE LIGAND-3; TIE LIGAND-4) AND THEIR USES27.(WO 97/41225) MAMMALIAN MIXED LYMPHOCYTE RECEPTORS, CHEMOKINE RECEPTORS [MMLR-CCR]28.(WO 97/24373) MONOCLONAL ANTIBODY ANTAGONISTS TO HAEMOPOIETIC GROWTH FACTORS29.(WO 97/21732) DESIGN OF HORMONE-LIKE ANTIBODIES WITH AGONISTIC AND ANTAGONISTIC FUNCTIONS, U.S. Pat. No. 6,235,880 Human sulfonylurea receptor U.S. Pat. No. 6,221,660 DNA encoding SNORF25 receptor U.S. Pat. No. 6,214,797 Urocortin peptides, nucleic acid encoding same methods for using same U.S. Pat. No. 6,214,344 Hepatocyte growth factor receptor antagonists and uses thereof U.S. Pat. No. 6,210,904 Anticoagulant test U.S. Pat. No. 6,207,152 Hepatocyte growth factor receptor antagonists and uses thereof U.S. Pat. No. 6,204,017 Polynucleotide encoding a histamine receptor U.S. Pat. No. 6,197,541 Recombinant thrombin receptors and assays using them U.S. Pat. No. 6,184,358 IP-10/Mig receptor designated CXCR3, antibodies, nucleic acids, and methods of use therefor U.S. Pat. No. 6,177,079 Antagonists of interleukin-156,177,078 Monoclonal antibody antagonists to IL-36,177,077 TNT inhibitors for the treatment of neurological disorders U.S. Pat. No. 6,171,815 Human sulfonylurea receptor U.S. Pat. No. 6,168,783 Antagonists of interleukin-156,166,185 Antibodies to human TIE-2 ligands U.S. Pat. No. 6,165,466 Antagonists of interleukin-156,162,431 Serine/threonine protein kinase U.S. Pat. No. 6,143,870 Thrombin receptor homolog U.S. Pat. No. 6,136,957 Antibodies which bind granulocyte-macrophane colony-stimulating factor receptor U.S. Pat. No. 6,124,101 Recombinant thrombin receptor and related pharmaceuticals U.S. Pat. No. 6,111,075 Protese-activated receptor PAR4 (ZCHEMR2) U.S. Pat. No. 6,103,874 Human KDEL receptor U.S. Pat. No. 6,096,873 Gamma-heregulin U.S. Pat. No. 6,086,874 Antitumor agent effect enhancer containing IL-6 antagonist 56,084,075 Agonist and antagonist antibodies to the chemokine receptor-2(CCR2) U.S. Pat. No. 6,063,596 G-protein coupled receptors associated with immune response U.S. Pat. No. 6,054,292 T-cell receptor protein U.S. Pat. No. 6,043,212 Recombinant C140 receptor, its agonists and antagonists, and nucleic acids encoding the receptor U.S. Pat. No. 6,033,869 Polynucleotide encoding a novel human cytokine/steroid receptor U.S. Pat. No. 6,024,936 Antibody-based method of localizing activated thrombin receptors U.S. Pat. No. 6,017,763 G-beta-gamma regulated phosphatidylinositol-3′ kinase U.S. Pat. No. 6,013,480 Antagonists of interleukin-156,013,479 Human Emr1-like G protein coupled receptor U.S. Pat. No. 5,994,097 Polynucleotide encoding human G-protein coupled receptor U.S. Pat. No. 5,985,828 Mammalian receptors for interleukin-10 (IL-10) U.S. Pat. No. 5,985,583 Cloning and expression of gonadotropin-releasing hormone receptor U.S. Pat. No. 5,977,072 High affinity immunoglobulin E receptor-like protein U.S. Pat. No. 5,976,852 K.kappa./.mu.-like protein tyrosine phosphatase, PTP .lambda. U.S. Pat. No. 5,976,815 Bioassay using ALK-7, a novel serine threonine kinase receptor U.S. Pat. No. 5,972,621 Methods of identifying compounds that modulate body weight using the OB receptor 5,965,709 IgE antagonists U.S. Pat. No. 5,965,365 Serine/threonine protein kinase U.S. Pat. No. 5,955,303 Human chemokine receptor-like protein U.S. Pat. No. 5,952,175 DNA encoding a human progesterone receptor complex p23-like protein U.S. Pat. No. 5,945,308 Human oxidized LDL receptor U.S. Pat. No. 5,942,606 Viral receptor protein U.S. Pat. No. 5,928,887 .kappa./.mu.-Like protein tyrosine phosphatase, PTP lambda. U.S. Pat. No. 5,912,144 Edg-1-receptor homolog U.S. Pat. No. 5,902,585 Methods of inducing T cell unresponsiveness to donor tissue or organ in a recipient with GP39 antagonists U.S. Pat. No. 5,892,014 DNA encoding a protease-activated receptor U.S. Pat. No. 35,891,720 Isolated DNA encoding a novel human G-protein coupled receptor U.S. Pat. No. 5,891,674 Insulin receptor tyrosine kinase substrate U.S. Pat. No. 5,891,638 Serine threonine kinase receptor, alk-75,888,811 Corticotropin-releasing hormone receptor U.S. Pat. No. 5,888,510 Chronic rheumatoid arthritis therapy containing IL-6 antagonist as effective component U.S. Pat. No. 5,886,148 Parathyroid hormone receptor U.S. Pat. No. 5,874,400 Recombinant C140 receptor, its agonists and antagonists, and nucleic acids encoding the receptor U.S. Pat. No. 5,874,273 G-beta-gamma regulated phosphatidylinositol-3′ kinase U.S. Pat. No. 5,874,224 Growth factor receptor binding protein U.S. Pat. No. 5,871,930 High affinity immunoglobulin E receptor-like protein U.S. Pat. No. 5,869,633 Thrombin receptor homolog polynucleotide U.S. Pat. No. 5,869,609 G protein coupled glutamate receptors U.S. Pat. No. 5,869,271 G-beta-gamma regulated phosphatidylinositol-3′ kinase U.S. Pat. No. 5,869,049 Methods of inducing T cell unresponsiveness to bone marrow with gp39 antagonists U.S. Pat. No. 5,863,796 Antibodies which specifically bind mammalian receptors for interleukin-10 (IL-10) U.S. Pat. No. 5,863,766 Human sigma receptor U.S. Pat. No. 5,859,201 G-beta-gamma regulated phosphatidylinositol-3′ kinase U.S. Pat. No. 5,856,448 Antibodies specifically reactive with thrombin receptor and its components U.S. Pat. No. 5,856,133 G-beta-gamma regulated phosphatidylinositol-3′ kinase U.S. Pat. No. 5,856,132 G-beta-gamma regulated phosphatidylinositol-3′ kinase U.S. Pat. No. 5,851,797Tie ligand-3, methods of making and uses thereof U.S. Pat. No. 5,840,853 Parathyroid hormone receptor and DNA encoding same U.S. Pat. No. 5,837,499 DNA encoding C5A receptor antagonists having substantially no agonist activity and methods of expressing same U.S. Pat. No. 5,834,240 DNA encoding a transforming growth factor-.beta. receptor associated protein U.S. Pat. No. 5,833,987 Treatment of T cell mediated autoimmune disorders U.S. Pat. No. 5,831,047 Oligonucleotide probes to L-AP4 sensitive glutamate receptor sequences U.S. Pat. No. 5,830,678 Method for identifying a target peptide that modulates the binding of epinectin ligand to integrin receptors U.S. Pat. No. 5,824,500 Nucleic acid encoding novel human KDEL receptor U.S. Pat. No. 5,817,480 DNA encoding a histamine H2 receptor U.S. Pat. No. 5,814,507 .kappa./.mu.-like protein tyrosine phosphatase, PTP .lambda. U.S. Pat. No. 5,814,464 Nucleic acids encoding TIE-2 ligand-25,811,245 Antibodies that specifically bind to ALK-7, a novel serine threonine kinase receptor U.S. Pat. No. 5,807,824C5 A receptor antagonists having substantially no agonist activity U.S. Pat. No. 5,795,966 Antagonists of interleukin-155,789,565 Serine threonine kinase receptor, ALK-7 U.S. Pat. No. 5,789,192 Mammalian receptors for interleukin-10 (IL-10) U.S. Pat. No. 5,763,575 Agonist and antagonist peptides of the C140 receptor U.S. Pat. No. 5,759,994 Recombinant thrombin receptor and related pharmaceuticals U.S. Pat. No. 5,750,366 Cloning and expression of gonadotropin-releasing hormone receptor U.S. Pat. No. 5,747,279 Nucleic acid molecules encoding kappa.sub.3 opioid receptors, receptors encoded thereby, and uses thereof U.S. Pat. No. 5,747,267 Method for identifying a G protein coupled glutamate receptor agonist and antagonist U.S. Pat. No. 5,738,999 L-AP4 sensitive glutamate receptors U.S. Pat. No. 5,730,976 Method for treating macrophage pathogen infections by TGF-Bantagonists U.S. Pat. No. 5,726,036 Granulocyte-macrophage colony-stimulating factor receptor and derivatives thereof U.S. Pat. No. 5,721,107 Antibodies to G protein coupled glutamate receptors U.S. Pat. No. 5,716,804 Mammalian interleukin-10 (IL-10) super-activating receptors;and variants U.S. Pat. No. 5,716,789 Method to determine ligands, agonist and antagonist of C140receptor U.S. Pat. No. 5,707,632 Receptors for fibroblast growth factors U.S. Pat. No. 5,688,768 Recombinant thrombin receptor and related pharmaceuticals U.S. Pat. No. 5,686,597 Thrombin receptor homolog U.S. Pat. No. 5,686,292 Hepatocyte growth factor receptor antagonist antibodies and uses thereof U.S. Pat. No. 5,683,884 Methods for identifying modulators of human calcitonin mediated metabolism U.S. Pat. No. 5,683,693 Method for inducing T cell unresponsiveness to a tissue or organ graft with anti-CD40 ligand antibody or soluble CD405,674,981 Human calcitonin receptor polypeptides U.S. Pat. No. 5,674,689 Human calcitonin receptor polypeptides and methods of use U.S. Pat. No. 5,646,036 Nucleic acids encoding hepatocyte growth factor receptor antagonist antibodies U.S. Pat. No. 5,629,283 Granulocyte-macrophage colony-stimulating factor receptor and derivatives thereof U.S. Pat. No. 5,622,839 Recombinant production of human calcitonin receptor polypeptides U.S. Pat. No. 5,614,609 Serine threonine kinase receptor U.S. Pat. No. 5,556,780 CDNAS encoding mouse and rat type-2 angiotensin II receptors and their expression in host cells U.S. Pat. No. 5,543,143 Method for activating macrophages/monocytes U.S. Pat. No. 5,516,894 A.sub.2b -adenosine receptors U.S. Pat. No. 5,514,555 Assays and therapeutic methods based on lymphocyte chemoattractants U.S. Pat. No. 5,506,107 Selecting ligand agonists and antagonists U.S. Pat. No. 5,494,806 DNA and vectors encoding the parathyroid hormone receptor, transformed cells, and recombinant production of PTHR proteins and peptides U.S. Pat. No. 5,451,658 Antagonists of human gamma interferon U.S. Pat. No. 5,441,935 Growth factor receptors U.S. Pat. No. 5,385,831 Method for producing a mammalian G protein coupled glutamate receptor U.S. Pat. No. 5,334,380 Anti-endotoxin, interleukin-1 receptor antagonist and anti-tumor necrosis factor antibody with arginine-free formulations for the treatment of hypotension U.S. Pat. No. 5,256,766 Recombinant thrombin receptor and related pharmaceuticals U.S. Pat. No. 5,177,190 Purified C5a receptor from human polymorphonuclear leukocytes U.S. Pat. No. 4,857,637 Methods and compositions for immunologically modulating growth hormone receptor activity; and references cited therein. (see also 1.(WO 01/49744) MOUSE G-PROTEIN COUPLED RECEPTOR MAS 2.(WO 01/49726) A NOVEL POLYPEPTIDE-HUMAN NATRIURETIC PEPTIDE RECEPTOR 18 AND THE POLYNUCLEOTIDE ENCODING SAID POLYPEPTIDE 3.(WO 01/49321) TNF INHIBITORS FOR THE TREATMENT OF NEUROLOGICAL, RETINAL AND MUSCULAR DISORDERS 4.(WO 01/00657) NOVEL INDOLE PEPTIDOMIMETICS AS THROMBIN RECEPTOR ANTAGONISTS 5.(WO 00/62790) SOLUBLE TUMOR NECROSIS FACTOR RECEPTOR TREATMENT OF MEDICAL DISORDERS 6.(WO 01/03720) PROMOTION OR INHIBITION OF ANGIOGENESIS AND CARDIOVASCULARIZATION BY TUMOR NECROSIS FACTOR LIGAND/RECEPTOR HOMOLOGS 7.(WO 01/46261) METHOD FOR TREATING INFLAMMATION 8.(WO 01/46191) 4-[ARYL(8-AZABICYCLO[3.2.1]OCTAN-3-YL)]AMINOBENZOIC ACID DERIVATIVES 9.(WO 01/46176) NON PEPTIDE TACHYKININ RECEPTOR ANTAGONISTS 10.(WO 01/45730) TWEAK RECEPTOR 11.(WO 01/45703) NITROSATED AND NITROSYLATED CYCLOOXYGENASE-2 INHIBITORS, COMPOSITIONS AND METHODS OF USE 12.(WO 01/40464) INTERLEUKIN-1-RECEPTOR ASSOCIATED KINASE-3 (IRAK3) AND ITS USE IN PROMOTION OR INHIBITION OF ANGIOGENESIS AND CARDIOVASCULARIZATION 13.(WO 01/44213) NEW P2X7 RECEPTOR ANTAGONISTS FOR USE IN THE TREATMENT OF INFLAMMATORY, IMMUNE OR CARDIOVASCULAR DISEASES 14.(WO 01/42268) DOG OREXIN 1 RECEPTOR 15.(WO 01/42208) CYCLOAMINE CCR5 RECEPTOR ANTAGONISTS 16.(WO 01/41752) ISOFORM SPECIFIC INHIBITION FOR TREATMENT OF PAIN AND REDUCTION OF ANESTHETIC THRESHOLD 17.(WO 01/03720) PROMOTION OR INHIBITION OF ANGIOGENESIS AND CARDIOVASCULARIZATION BY TUMOR NECROSIS FACTOR LIGAND/RECEPTOR HOMOLOGS 18.(WO 01/40464) INTERLEUKIN-1-RECEPTOR ASSOCIATED KINASE-3 (IRAK3) AND ITS USE IN PROMOTION OR INHIBITION OF ANGIOGENESIS AND CARDIOVASCULARIZATION 19.(WO 01/40259) MONKEY OREXIN 1 RECEPTOR 20.(WO 01/40252) MONKEY CALCIUM SENSING RECEPTOR 21.(WO 01/04139) HUMAN AXOR29 RECEPTOR 22.(WO 01/36480) MOUSE 7-TRANSMEMBRANE RECEPTOR, AXOR45 23.(WO 01/00656) NOVEL INDAZOLE PEPTIDOMIMETICS AS THROMBIN RECEPTOR ANTAGONISTS 24.(WO 00/67793) DEATH DOMAIN CONTAINING RECEPTOR 4 25.(WO 01/34645) MODULATING IL-13 ACTIVITY USING MUTATED IL-13 MOLECULES THAT ARE ANTAGONISTS OR AGONISTS OF IL-13 26.(WO 01/34138) COMPOSITIONS AND METHODS FOR TREATMENT OF NEUROLOGICAL DISORDERS AND NEURODEGENERATIVE DISEASES 27.(WO 01/32656) POLYMORPHIC FORM OF A TACHYKININ RECEPTOR ANTAGONIST 28.(WO 01/32166) NEW COMBINATION COMPRISING A &#946;2-ADRENORECEPTOR AGONIST AND A LEUKOTRIENE RECEPTOR ANTAGONIST 29.(WO 01/32163) NEW COMBINATION COMPRISING A BETA 2 (&#946;)2 ADRENO RECEPTOR AGONIST AND A LENKOTRIENE RECEPTOR ANTAGONIST 30.(WO 01/01922) USE OF SUBSTANCE P ANTAGONISTS FOR THE TREATMENT OF ADENOCARCINOMA 31.(WO 01/30850) UMLR POLYPEPTIDES 32.(WO 01/27153) A MURINE SEVEN-TRANSMEMBRANE RECEPTOR, MUS MUSCULUS MHNEAA81 33.(WO 01/25269) NOVEL HUMAN G-PROTEIN COUPLED RECEPTOR 34.(WO 01/24828) MODULATORS OF CYTOKINE MEDIATED SIGNALLING PATHWAYS AND INTEGRIN &#945;V&#946;3 RECEPTOR ANTAGONISTS FOR COMBINATION THERAPY 35.(WO 01/24798) USE OF CENTRAL CANNABINOID RECEPTOR ANTAGONIST FOR PREPARING MEDICINES 36.(WO 01/24797) INTEGRIN RECEPTOR ANTAGONISTS 37.(WO 00/68250) 7TM RECEPTOR RAT APJ 38.(WO 01/16121) HETEROCYCLIC COMPOUNDS AND METHODS OF USE THEREOF 39.(WO 01/14406) ANTIANDROGEN AGENTS 40.(WO 01/12671) HUMAN TUMOR NECROSIS FACTOR RECEPTOR TR16 41.(WO 01/10891) IL-16 ANTAGONISTS 42.(WO 01/10889) RAT-G-PROTEIN COUPLED RECEPTOR BRS3 43.(WO 01/10423) USE OF 5-HT3 RECEPTOR ANTAGONISTS FOR THE TREATMENT OF INFLAMMATIONS OF THE RESPIRATORY TRACT 44.(WO 01/07028) THE USE OF RETINOID RECEPTOR ANTAGONISTS IN THE TREATMENT OF PROSTATE CARCINOMA 45.(WO 01/05834) HUMAN TUMOR NECROSIS FACTOR RECEPTORS TR13 AND TR14 46.(WO 01/05783) BRADYKININ B1 RECEPTOR ANTAGONISTS 47.(WO 01/04139) POLYNUCLEOTIDE AND POLYPEPTIDE SEQUENCES OF HUMAN AXOR29 RECEPTOR AND METHODS OF SCREENING FOR AGONISTS AND ANTAGONISTS OF THE INTERACTION BETWEEN HUMAN AXOR29 RECEPTOR AND ITS LIGANDS 48.(WO 01/03720) PROMOTION OR INHIBITION OF ANGIOGENESIS AND CARDIOVASCULARIZATION BY TUMOR NECROSIS FACTOR LIGAND/RECEPTOR HOMOLOGS 49.(WO 01/01922) USE OF SUBSTANCE P ANTAGONISTS IN THE TREATMENT OF THE ADENOCARCINOMAS 50.(WO 01/00659) BENZIMIDAZOLONE PEPTIDOMIMETICS AS THROMBIN RECEPTOR ANTAGONISTS 51.(WO 01/00657) NOVEL INDOLE PEPTIDOMIMETICS AS THROMBIN RECEPTOR ANTAGONISTS 52.(WO 01/00656) NOVEL INDAZOLE PEPTIDOMIMETICS AS THROMBIN RECEPTOR ANTAGONISTS 53.(WO 01/00576) INDOLE AND INDAZOLE UREA-PEPTOIDS AS THROMBIN RECEPTOR ANTAGONISTS 54.(WO 01/00198) COMPOSITIONS AND METHODS OF TREATING CANCER USING COMPOSITIONS COMPRISING AN INHIBITOR OF ENDOTHELIN RECEPTOR ACTIVITY 55.(WO 00/78317) INTEGRIN RECEPTOR ANTAGONISTS 56.(WO 00/77195) NUCLEIC ACID ENCODING NOVEL EGF-LIKE GROWTH FACTORS 57.(WO 00/76502) METHODS AND COMPOSITIONS FOR TREATING RAYNAUD'S PHENOMENON AND SCLERODERMA 58.(WO 00/74719) METHOD OF TREATING CARCINOMA USING ANTIBODY THERAPY AND AMELIORATING SIDE EFFECTS ASSOCIATED WITH SUCH THERAPY 59.(WO 00/73321) HUMAN TUMOR NECROSIS FACTOR RECEPTOR TRIO 60.(WO 00/72801) ALPHA V INTEGRIN RECEPTOR ANTAGONISTS 61.(WO 00/71150) TUMOR NECROSIS FACTOR RECEPTOR 5 62.(WO 00/69831) SPIROIMIDAZOLIDINE DERIVATIVES, THEIR PREPARATION, THEIR USE AND PHARMACEUTICAL PREPARATIONS COMPRISING THEM 63.(WO 00/69820) CYCLIC AMINE DERIVATIVES AND THEIR USES 64.(WO 00/69463) COMPOSITIONS AND METHODS FOR TREATING CELL PROLIFERATION DISORDERS 65.(WO 00/69459) TREATMENT OF REFRACTORY HUMAN TUMORS WITH EPIDERMAL GROWTH FACTOR RECEPTOR ANTAGONISTS 66.(WO 00/68250) 7TM RECEPTOR RAT APJ 67.(WO 00/68244) 7TM RECEPTOR MOUSE APJ 68.(WO 00/67793) DEATH DOMAIN CONTAINING RECEPTOR 4 69.(WO 00/67034) METHODS OF USE OF THE TACI/TACI-L INTERACTION 70.(WO 00/67024) CANCER TREATMENT WITH ENDOTHELIN RECEPTOR ANTAGONISTS 71.(WO 00/66632) AGONISTS OR ANTAGONISTS FOR HAEMOPOIETIC GROWTH FACTORS 72.(WO 00/66522) GLUCOCORTICOID RECEPTOR MODULATORS 73.(WO 00/66156) DEATH DOMAIN CONTAINING RECEPTOR 5 74.(WO 00/64465) DEATH DOMAIN CONTAINING RECEPTORS 75.(WO 00/62790) SOLUBLE TUMOR NECROSIS FACTOR RECEPTOR TREATMENT OF MEDICAL DESORDERS 76.(WO 00/59532) THE USE OF DOMAINS OF TYPE IV COLLAGEN T INHIBIT ANGIOGENESIS AN TUMOUR GROWTH 77.(WO 00/56862) HUMAN TUMOR NECROSIS FACTOR RECEPTOR TR9 78.(WO 00/56405) HUMAN TUMOR NECROSIS FACTOR RECEPTOR-LIKE 2 79.(WO 00/54772) AMYOTROPIC LATERAL SCLEROSIS TREATMENT WITH A COMBINATION OF RILUZOLE AND AN AMPA RECEPTOR ANTAGONIST 80.(WO 00/53596) IMIDAZOLE COMPOUNDS SUBSTITUTED WITH A SIX OR SEVEN MEMBERED HETEROCYCLIC RING CONTAINING TWO NITROGEN ATOMS 81.(WO 00/53175) COMPOUNDS AND METHODS 82.(WO 00/52028) TUMOR NECROSIS FACTOR RECEPTORS 6&agr; and 6&bgr; 83.(WO 00/51974) ALPHA-AMINOACETIC ACID DERIVATIVES USEFUL AS ALPHA 4 BETA 7-RECEPTOR ANTAGONISTS 84.(WO 00/50459) HUMAN TUMOR NECROSIS FACTOR RECEPTOR-LIKE PROTEINS TR11, TR11SV1, AND TR11SV2 85.(WO 00/49170) MURINE 11c by RECEPTOR 86.(WO 00/48603) DIBENZO-AZEPINE DERIVATIVES AS &agr;V INTEGRIN RECEPTOR ANTAGONISTS 87.(WO 00/48597) SYSTEMIC USE OF 5-HT 3 RECEPTOR ANTAGONISTS AGAINST RHEUMATIC INFLAMMATORY PROCESSES 88.(WO 00/48581) USE OF 5-HT3 RECEPTOR ANTAGONISTS 89.(WO 00/46343) SCREENING ASSAY FOR ANTAGONISTS OF FGFR-MEDIATED MALIGNANT CELL TRANSFORMATION AND TUMOR FORMATION 90.(WO 00/46215) BENZAZEPINE DERIVATIVES AS ALPHA-V INTEGRIN RECEPTOR ANTAGONISTS 91.(WO 00/46197) INDOLE DERIVATIVES AND THEIR USE AS MCP-1 RECEPTOR ANTAGONISTS 92.(WO 00/44763) COMPOSITIONS FOR TREATING INFLAMMATORY RESPONSE 93.(WO 00/43031) TUMOR NECROSIS FACTOR ANTAGONISTS AND THEIR USE IN ENDOMETRIOSIS 94.(WO 00/42852) COMPOUNDS AND METHODS 95.(WO 00/40716) SOLUBLE RECEPTOR BR43×2 AND METHODS OF USING 96.(WO 00/40239) COMPOUNDS AND METHODS 97.(WO 00/39166) NOVEL HYALURONAN-BINDING PROTEINS AND ENCODING GENES 98.(WO 00/37462) NON-PEPTIDE NK 1 RECEPTORS ANTAGONISTS 99.(WO 00/35887) VITRONECTIN RECEPTOR ANTAGONIST PHARMACEUTICALS 100.(WO 00/35492) VITRONECTIN RECEPTOR ANTAGONIST PHARMACEUTICALS 51.(WO 01/00657) NOVEL INDOLE PEPTIDOMIMETICS AS THROMBIN RECEPTOR ANTAGONISTS 52.(WO 01/00656) NOVEL INDAZOLE PEPTIDOMIMETICS AS THROMBIN RECEPTOR ANTAGONISTS 53.(WO 01/00576) INDOLE AND INDAZOLE UREA-PEPTOIDS AS THROMBIN RECEPTOR ANTAGONISTS 54.(WO 01/00198) COMPOSITIONS AND METHODS OF TREATING CANCER USING COMPOSITIONS COMPRISING AN INHIBITOR OF ENDOTHELIN RECEPTOR ACTIVITY 55.(WO 00/78317) INTEGRIN RECEPTOR ANTAGONISTS 56.(WO 00/77195) NUCLEIC ACID ENCODING NOVEL EGF-LIKE GROWTH FACTORS 57.(WO 00/76502) METHODS AND COMPOSITIONS FOR TREATING RAYNAUD'S PHENOMENON AND SCLERODERMA 58.(WO 00/74719) METHOD OF TREATING CARCINOMA USING ANTIBODY THERAPY AND AMELIORATING SIDE EFFECTS ASSOCIATED WITH SUCH THERAPY 59.(WO 00/73321) HUMAN TUMOR NECROSIS FACTOR RECEPTOR TRIO 60.(WO 00/72801) ALPHA V INTEGRIN RECEPTOR ANTAGONISTS 61.(WO 00/71150) TUMOR NECROSIS FACTOR RECEPTOR 5 62.(WO 00/69831) SPIROIMIDAZOLIDINE DERIVATIVES, THEIR PREPARATION, THEIR USE AND PHARMACEUTICAL PREPARATIONS COMPRISING THEM 63.(WO 00/69820) CYCLIC AMINE DERIVATIVES AND THEIR USES 64.(WO 00/69463) COMPOSITIONS AND METHODS FOR TREATING CELL PROLIFERATION DISORDERS 65.(WO 00/69459) TREATMENT OF REFRACTORY HUMAN TUMORS WITH EPIDERMAL GROWTH FACTOR RECEPTOR ANTAGONISTS 66.(WO 00/68250) 7TM RECEPTOR RAT APJ 67.(WO 00/68244) 7TM RECEPTOR MOUSE APJ 68.(WO 00/67793) DEATH DOMAIN CONTAINING RECEPTOR 4 69.(WO 00/67034) METHODS OF USE OF THE TACI/TACI-L INTERACTION 70.(WO 00/67024) CANCER TREATMENT WITH ENDOTHELIN RECEPTOR ANTAGONISTS 71.(WO 00/66632) AGONISTS OR ANTAGONISTS FOR HAEMOPOIETIC GROWTH FACTORS 72.(WO 00/66522) GLUCOCORTICOID RECEPTOR MODULATORS 73.(WO 00/66156) DEATH DOMAIN CONTAINING RECEPTOR 5 74.(WO 00/64465) DEATH DOMAIN CONTAINING RECEPTORS 75.(WO 00/62790) SOLUBLE TUMOR NECROSIS FACTOR RECEPTOR TREATMENT OF MEDICAL DESORDERS 76.(WO 00/59532) THE USE OF DOMAINS OF TYPE IV COLLAGEN T INHIBIT ANGIOGENESIS AN TUMOUR GROWTH 77.(WO 00/56862) HUMAN TUMOR NECROSIS FACTOR RECEPTOR TR9 78.(WO 00/56405) HUMAN TUMOR NECROSIS FACTOR RECEPTOR-LIKE 2 79.(WO 00/54772) AMYOTROPIC LATERAL SCLEROSIS TREATMENT WITH A COMBINATION OF RILUZOLE AND AN AMPA RECEPTOR ANTAGONIST 80.(WO 00/53596) IMIDAZOLE COMPOUNDS SUBSTITUTED WITH A SIX OR SEVEN MEMBERED HETEROCYCLIC RING CONTAINING TWO NITROGEN ATOMS 81.(WO 00/53175) COMPOUNDS AND METHODS 82.(WO 00/52028) TUMOR NECROSIS FACTOR RECEPTORS 6&agr; and 6&bgr; 83.(WO 00/51974) ALPHA-AMINOACETIC ACID DERIVATIVES USEFUL AS ALPHA 4 BETA 7-RECEPTOR ANTAGONISTS 84.(WO 00/50459) HUMAN TUMOR NECROSIS FACTOR RECEPTOR-LIKE PROTEINS TR11, TR11SV1, AND TR11SV2 85.(WO 00/49170) MURINE 11c by RECEPTOR 86.(WO 00/48603) DIBENZO-AZEPINE DERIVATIVES AS &agr;V INTEGRIN RECEPTOR ANTAGONISTS 87.(WO 00/48597) SYSTEMIC USE OF 5-HT 3 RECEPTOR ANTAGONISTS AGAINST RHEUMATIC INFLAMMATORY PROCESSES 88.(WO 00/48581) USE OF 5-HT3 RECEPTOR ANTAGONISTS 89.(WO 00/46343) SCREENING ASSAY FOR ANTAGONISTS OF FGFR-MEDIATED MALIGNANT CELL TRANSFORMATION AND TUMOR FORMATION 90.(WO 00/46215) BENZAZEPINE DERIVATIVES AS ALPHA-V INTEGRIN RECEPTOR ANTAGONISTS 91.(WO 00/46197) INDOLE DERIVATIVES AND THEIR USE AS MCP-1 RECEPTOR ANTAGONISTS 92.(WO 00/44763) COMPOSITIONS FOR TREATING INFLAMMATORY RESPONSE 93.(WO 00/43031) TUMOR NECROSIS FACTOR ANTAGONISTS AND THEIR USE IN ENDOMETRIOSIS 94.(WO 00/42852) COMPOUNDS AND METHODS 95.(WO 00/40716) SOLUBLE RECEPTOR BR43×2 AND METHODS OF USING 96.(WO 00/40239) COMPOUNDS AND METHODS 97.(WO 00/39166) NOVEL HYALURONAN-BINDING PROTEINS AND ENCODING GENES 98.(WO 00/37462) NON-PEPTIDE NK 1 RECEPTORS ANTAGONISTS 99.(WO 00/35887) VITRONECTIN RECEPTOR ANTAGONIST PHARMACEUTICALS 100.(WO 00/35492) VITRONECTIN RECEPTOR ANTAGONIST PHARMACEUTICALS 101.(WO 00/35488) VITRONECTIN RECEPTOR ANTAGONIST PHARMACEUTICALS 102.(WO 00/35455) HETEROARYL-ARYL UREAS AS IGF-1 RECEPTOR ANTAGONISTS 103.(WO 00/32578) BENZIMIDAZOLE COMPOUNDS THAT ARE VITRONECTIN RECEPTOR ANTAGONISTS 104.(WO 00/28988) NITROSATED AND NITROSYLATED H2 RECEPTOR ANTAGONIST COMPOUNDS, COMPOSITIONS AND METHODS OF USE 105.(WO 00/27421) LOCAL USE OF SOLUBLE TUMOR NECROSIS RECEPTOR I (sTNFRI) FOR PROPHYLAXIS AND TREATMENT OF CORNEAL TRANSPLANT REJECTION AND OTHER DISORDERS OF THE EYE 106.(WO 00/25805) VASCULAR ENDOTHELIAL GROWTH FACTOR-LIKE PROTEIN FROM ORF VIRUS NZ2 BINDS AND ACTIVATES MAMMALIAN VEGF RECEPTOR-2 107.(WO 00/25745) IRRIGATION SOLUTION AND METHOD FOR INHIBITION OF PAIN AND INFLAMMATION 108.(WO 00/24395) NEW USE OF GLUTAMATE ANTAGONISTS FOR THE TREATMENT OF CANCER 109.(WO 00/23471) USE OF A CYTOKINE-PRODUCING LACTOCOCCUS STRAIN TO TREAT COLITIS 110.(WO 00/23469) FRAGMENTS OF INSULIN-LIKE GROWTH FACTOR BINDING PROTEIN AND INSULIN-LIKE GROWTH FACTOR, AND USES THEREOF 111.(WO 00/23438 ) N-(IMIDAZOLYLALKYL)SUBSTITUTED CYCLIC AMINES AS HISTAMINE-H 3 AGONISTS OR ANTAGONISTS 112.(WO 00/23113) PEPTIDE-BASED CARRIER DEVICES FOR STELLATE CELLS 113.(WO 00/23066) IRRIGATION SOLUTION AND METHOD FOR INHIBITION OF PAIN AND INFLAMMATION 114.(WO 00/23062) IRRIGATION SOLUTION AND METHOD FOR INHIBITION OF PAIN AND INFLAMMATION 115.(WO 00/20578) A METHOD OF MODULATING CELL SURVIVAL AND REAGENTS USEFUL FOR SAME 116.(WO 00/20389) NAPHTHALENECARBOXAMIDES AS TACHYKININ RECEPTOR ANTAGONISTS 117.(WO 00/20371) PROSTAGLANDIN RECEPTOR LIGANDS 118.(WO 00/20003) NAPHTHALENECARBOXAMIDES AS TACHYKININ RECEPTOR ANTAGONISTS 119.(WO 00/14109) BASIC PRODUCTS HAVING ANTAGONISTIC ACTIVITY ON THE NK-1 RECEPTOR AND THEIR USE IN PHARMACEUTICAL COMPOSITIONS 120.(WO 00/10391) THE USE OF ADENOSINE A3 RECEPTOR ANTAGONISTS TO INHIBIT TUMOR GROWTH 121.(WO 00/09503) INTEGRIN RECEPTOR ANTAGONISTS 122.(WO 00/09152) THERAPEUTIC CHEMOKINE RECEPTOR ANTAGONISTS 123.(WO 00/08001) SUBSTITUTED ISOXAZOLE AS ESTROGEN RECEPTOR MODULATORS 124.(WO 00/06169) INTEGRIN RECEPTOR ANTAGONISTS 125.(WO 00/03716) TOPICAL COMPOSITIONS COMPRISING AN OPIOID ANALGESIC AND AN NMDA ANTAGONIST 126.(WO 00/02859) N-SUBSTITUTED NAPHTHALENE CARBOXAMIDES AS NEUROKININ-RECEPTOR ANTAGONISTS 127.(WO 00/02582) TREATMENT OF CELIAC DISEASE WITH INTERLEUKIN-15 ANTAGONISTS 128.(WO 00/01802) PEPTIDE ANTAGONISTS OF THE HUMAN UROKINASE RECEPTOR AND METHOD FOR SELECTING THEM 129.(WO 00/00194) OPHTHALMIC USES OF PPARGAMMA AGONISTS AND PPARGAMMA ANTAGONISTS 130.(WO 99/65944) PEPTIDE INHIBITORS OF &agr;V&bgr;3 AND &agr;V&bgr;5 131 .(WO 99/62955) METHOD OF DESIGNING AGONISTS AND ANTAGONISTS TO EGF RECEPTOR FAMILY 132.(WO 99/60015) IMIDAZOLIDINE DERIVATIVES, THE PRODUCTION THEREOF, THEIR USE AND PHARMACEUTICAL PREPARATIONS CONTAINING THE SAME 133.(WO 99/59635) USE OF A COX-2 INHIBITOR AND A NK-1 RECEPTOR ANTAGONIST FOR TREATING INFLAMMATION 134.(WO 99/58142) USE OF ANTI-PROLACTIN AGENTS TO TREAT PROLIFERATIVE CONDITIONS 135.(WO 99/58097) USE OF ANTI-PROLACTIN AGENTS TO TREAT PROLIFERATIVE CONDITIONS 136.(WO 99/57245) METHODS OF SCREENING FOR AGONISTS AND ANTAGONISTS OF THE INTERACTION BETWEEN THE HUMAN KIAA0001 RECEPTOR AND LIGANDS THEREOF 137.(WO 99/51245) NON-PEPTIDE BRADYKININ RECEPTOR ANTAGONISTS FOR USE IN TREATING OPHTHALMIC DISEASES AND DISORDERS 138.(WO 99/50249) INTEGRIN ANTAGONISTS 139.(WO 99/49856) ANTAGONISTS FOR TREATMENT OF CD11/CD18 ADHESION RECEPTOR MEDIATED DISORDERS 140.(WO 99/47170) PREVENTIVES OR REMEDIES FOR INFLAMMATORY INTESTINAL DISEASES CONTAINING AS THE ACTIVE INGREDIENT IL-6 ANTAGONISTS 141.(WO 99/47158) THERAPEUTIC CHEMOKINE RECEPTOR ANTAGONISTS 142.(WO 99/46376) RECEPTOR FROM THE SUPERFAMILY OF TNT-RECEPTORS FROM THE HUMAN LUNG 143.(WO 99/45927) VITRONECTIN RECEPTOR ANTAGONISTS 144.(WO 99/45905) PROPHYLAXIS AND TREATMENT OF MIGRAINE HEADACHES WITH THROMBOXANE SYNTHETASE INHIBITORS AND/OR RECEPTOR ANTAGONISTS 145.(WO 99/44612) SUBSTITUTED QUINAZOLINES AND ANALOGS AND THE USE THEREOF 146.(WO 99/43809) PROTEASE-ACTIVATED RECEPTOR 4 AND USES THEREOF 147.(WO 99/42464) SUBSTITUTED IMIDAZO[1,2-a;3,4-a′]DIQUINOLINYLIUM INTERLEUKIN-8 RECEPTOR ANTAGONISTS 148.(WO 99/42463) SUBSTITUTED QUINOXALINE DERIVATIVES AS INTERLEUKIN-8 RECEPTOR ANTAGONISTS 149.(WO 99/42461) SUBSTITUTED QUINOXALINE DERIVATIVES AS INTERLEUKIN-8 RECEPTOR ANTAGONISTS 150.(WO 99/41257) GLUCOCORTICOID-SELECTIVE ANTIINFLAMMATORY AGENTS 151.(WO 99/41256) GLUCOCORTICOID-SELECTIVE ANTI-INFLAMMATORY AGENTS 152.(WO 99/40192) HUMAN RECEPTOR GPR14, AND A METHOD OF FINDING AGONIST AND ANTAGONIST TO HUMAN AND RAT GPR14 153.(WO 99/40091) BICYCLIC PYRIDINE AND PYRIMIDINE DERIVATIVES AS NEUROPEPTIDE Y RECEPTOR ANTAGONISTS 154.(WO 99/38532) METHODS FOR THE PREVENTION AND TREATMENT OF FIBROSIS AND SCLEROSIS 155.(WO 99/36541) INTERLEUKIN-1 RECEPTOR ANTAGONIST BETA (IL-1RA&bgr;) 156.(WO 99/33806) 4-[ARYL(PIPERIDIN-4-YL)] AMINOBENZAMIDES WHICH BIND TO THE DELTA-OPIOID RECEPTOR 157.(WO 99/31099) INTEGRIN RECEPTOR ANTAGONISTS 158.(WO 99/31061) INTEGRIN RECEPTOR ANTAGONISTS 159.(WO 99/30713) INTEGRIN RECEPTOR ANTAGONISTS 160.(WO 99/30709) INTEGRIN RECEPTOR ANTAGONISTS 161.(WO 99/29729) ANTAGONISTS OF NEUROPILIN RECEPTOR FUNCTIONAL AND USE THEREOF 162.(WO 99/27962) USE OF A FIBRINOGEN RECEPTOR-ANTAGONIST FOR PREVENTING DISSEMINATED INTRAVASCULAR COAGULATION 163.(WO 99/26945) 1,3,4-THIADIAZOLES AND 1,3,4-OXADIAZOLES AS &agr; v &bgr; 3 ANTAGONISTS 164.(WO 99/26943) THROMBIN RECEPTOR ANTAGONISTS 165.(WO 99/25857) TRANSGENIC MODELS OF INFLAMMATORY DISEASE 166.(WO 99/24471) OPIATE, CANNABINOID, AND ESTROGEN RECEPTORS 167.(WO 99/24423) PIPERIDINE DERIVATIVES AND THEIR USE AS TACHYKININ ANTAGONISTS 168.(WO 99/24421) IMIDAZOYLALKYL SUBSTITUTED WITH A FIVE, SIX OR SEVEN MEMBERED HETEROCYCLIC RING CONTAINING ONE NITROGEN ATOM 169.(WO 99/24406) PHENYL-ALKYL-IMIDAZOLES AS H3 RECEPTOR ANTAGONISTS 170.(WO 99/24405) H 3 RECEPTOR LIGANDS OF THE PHENYL-ALKYL-IMIDAZOLES TYPE 171.(WO 99/21555) ADENOSINE A3 RECEPTOR ANTAGONISTS 172.(WO 99/20758) HUMAN TUMOR NECROSIS FACTOR RECEPTOR-LIKE PROTEINS TR11, TR11SV1, AND TR11SV2 173.(WO 99/19462) ENHANCED IMMUNOGENIC CELL POPULATIONS PREPARED USING H2 RECEPTOR ANTAGONISTS 174.(WO 99/17773) COMPOUNDS AND METHODS 175.(WO 99/16465) METHOD FOR INHIBITING TUMOR ANGIOGENESIS IN A LIVING SUBJECT 176.(WO 99/11790) TUMOR NECROSIS FACTOR RECEPTOR ZTNFR-6 177.(WO 99/06049) INTEGRIN RECEPTOR ANTAGONISTS 178.(WO 99/04001) TUMOR NECROSIS FACTOR RECEPTOR ZTNFR-5 179.(WO 99/02499) QUINOLINE COMPOUNDS AND MEDICINAL USES THEREOF 180.(WO 99/01764) METHOD FOR RECOGNIZING AND DETERMINING GNRH RECEPTORS AND THE USE OF GNRH AGONISTS AND GNRH ANTAGONISTS AND OTHER GNRH RECEPTOR LIGANDS FOR THE TREATMENT WITH GNRH RECEPTORS OF TUMOURS ORIGINATING IN THE BRAIN AND/OR NERVOUS SYSTEM AND/OR MENINGES AND/OR OF KAPOSI SARCOMA 181.(WO 99/01444) POLYMORPHIC FORM OF THE TACHYKININ RECEPTOR ANTAGONIST 2-(R)-(1-(R) -(3,5-BIS(TRIFLUOROMETHYL) PHENYL)ETHOXY)-3-(S)-(4-FLUORO) PHENYL-4-(3-5 (-OXO-1 H,4H-1,2,4,-TRIAZOLO) METHYLMORPHOLINE 182.(WO 99/01127) COMPOUNDS AND METHODS 183.(WO 99/00406) CYCLIC AGONISTS AND ANTAGONISTS OF C5a RECEPTORS AND G PROTEIN-COUPLED RECEPTORS 184.(WO 98/58674) ANTI-TUMOUR PHARMACEUTICAL COMPOSITIONS CAPABLE OF REDUCING DRUG RESISTANCE IN TUMOUR CELLS 185.(WO 98/57647) COUP-TFII: AN ORPHAN NUCLEAR RECEPTOR REQUIRED FOR ANGIOGENESIS 186.(WO 98/56892) HUMAN TUMOR NECROSIS FACTOR RECEPTOR TR9 187.(WO 98/56779) 4-SULFINYL BENZAMIDES AS CALCITONIN GENE-RELATED PEPTIDE RECEPTOR ANTAGONISTS 188.(WO 98/55153) NON-STEROIDAL RADIOLABELED AGONIST/ANTAGONIST COMPOUNDS AND THEIR USE IN PROSTATE CANCER IMAGING 189.(WO 98/54325) HUMAN FRP AND FRAGMENTS THEREOF INCLUDING METHODS FOR USING THEM 190.(WO 98/54202) HUMAN TUMOR NECROSIS FACTOR RECEPTOR TRIO 191.(WO 98/54201) HUMAN TUMOR NECROSIS FACTOR RECEPTOR-LIKE PROTEIN 8 192.(WO 98/54187) SPIRO-AZACYCLIC DERIVATIVES AND THEIR USE AS THERAPEUTIC AGENTS 193.(WO 98/53069) GDNF RECEPTORS 194.(WO 98/49170) SPIRO-AZACYCLIC DERIVATIVES AND THEIR USE AS THERAPEUTIC AGENTS 195.(WO 98/48017) FAMILY OF IMMUNOREGULATORS DESIGNATED LEUKOCYTE IMMUNOGLOBULIN-LIKE RECEPTORS (LIR) 196.(WO 98/47923) IL-5R ANTAGONISTS FOR TREATMENT OF INFLAMMATION, ASTHMA AND OTHER ALLERGIC DISEASES 197.(WO 98/46751) OSTEOPROTEGERIN BINDING PROTEINS AND RECEPTORS 198.(WO 98/46620) A NOVEL HUMAN G-PROTEIN COUPLED RECEPTOR 199.(WO 98/46265) METHODS FOR USING ANTAGONISTIC ANTI-AVB3 INTEGRIN ANTIBODIES 200.(WO 98/43962 ) HETEROCYCLIC INTEGRIN INHIBITOR PRODRUGS 251.(WO 97/44333) 1,2,4-OXADIAZOLES AS ADHESION-RECEPTOR ANTAGONISTS 252.(WO 97/44329) DIARYLALKYL CYCLIC DIAMINE DERIVATIVES AS CHEMOKINE RECEPTOR ANTAGONISTS 253.(WO 97/41225) MAMMALIAN MIXED LYMPHOCYTE RECEPTORS, CHEMOKINE RECEPTORS [MMLR-CCR] 254.(WO 97/37655) &agr;v&bgr;3 ANTAGONISTS 255.(WO 97/35969) PEPTIDE LIGANDS OF THE UROKINASE RECEPTOR 256.(WO 97/34878) SUBSTITUTED 2,3-BENZODIAZEPIN-4-ONES AND THE USE THEREOF 257.(WO 97/33904) DEATH DOMAIN CONTAINING RECEPTORS 258.(WO 97/33887) SPIROCYCLE INTEGRIN INHIBITORS 259.(WO 97/33613) PARASITE-DERIVED ANTI-INFLAMMATORY IMMUNOMODULATORY PROTEIN 260.(WO 97/30991) NOVEL SUBSTITUTED N-METHYL-N-(4-(4-(1H-BENZIMIDAZOL-2-YL)[1,4]DIAZEPAN-1-YL)-2-(ARYL)BUTYL)BENZAMIDES USEFUL FOR THE TREATMENT OF ALLERGIC DISEASES 261.(WO 97/30990) NOVEL SUBSTITUTED N-METHYL-N-(4-(PIPERIDIN-1-YL)-2-(ARYL)BUTYL)BENZAMIDES USEFUL FOR THE TREATMENT OF ALLERGIC DISEASES 262.(WO 97/30989) NOVEL SUBSTITUTED N-METHYL-N-(4-(4-(1H-BENZIMIDAZOL-2-YL-AMINO)PIPERIDIN-1-YL)-2-(ARYL)BUTYL)BENZAMIDES USEFUL FOR THE TREATMENT OF ALLERGIC DISEASES 263.(WO 97/30079) PEPTIDE ANTAGONISTS OF CELLULAR MITOGENESIS AND MOTOGENESIS AND THEIR THERAPEUTIC USE 264.(WO 97/30069) 17-BETA-CYCLOPROPYL(AMINO/OXY) 4-AZA STEROIDS AS ACTIVE INHIBITORS OF TESTOSTERONE 5-ALPHA-REDUCTASE AND C17-20-LYASE 265.(WO 97/29775) COMPOSITIONS COMPRISING A CYCLOOXYGENASE-2 INHIBITOR AND A LEUKOTRIENE B 4 RECEPTOR ANTAGONIST 266.(WO 97/29079) NOVEL COMPOUNDS AND PHARMACEUTICAL USE THEREOF 267.(WO 97/28190) CYTOKINE ANTAGONISTS AND AGONISTS 268.(WO 97/24373) MONOCLONAL ANTIBODY ANTAGONISTS TO HAEMOPOIETIC GROWTH FACTORS 269.(WO 97/23480) NOVEL INTEGRIN RECEPTOR ANTAGONISTS 270.(WO 97/22604) NOVEL SUBSTITUTED 4-(1H-BENZIMIDAZOL-2-YL)[1,4]DIAZEPANES USEFUL FOR THE TREATMENT OF ALLERGIC DISEASES 271.(WO 97/21732) DESIGN OF HORMONE-LIKE ANTIBODIES WITH AGONISTIC AND ANTAGONISTIC FUNCTIONS 272.(WO 97/21702) 3-BENZYLAMINOPYRROLIDINES AND -PIPERIDINES AS TACHYKININ RECEPTOR ANTAGONISTS 273.(WO 97/21445 ) VASCULAR IRRIGATION SOLUTION AND METHOD FOR INHIBITION OF PAIN, INFLAMMATION, SPASM AND RESTENOSIS 274.(WO 97/20062) IL-12 P40 SUBUNIT FUSION POLYPEPTIDES AND USES THEREOF 275.(WO 97/19074) SUBSTITUTED 4-(1H-BENZIMIDAZOL-2-YL-AMINO)PIPERIDINES USEFUL FOR THE TREATMENT OF ALLERGIC DISEASES 276.(WO 97/19059) NOVEL SUBSTITUTED ARYL COMPOUNDS USEFUL AS MODULATORS OF ACETYLCHOLINE RECEPTORS 277.(WO 97/16442) SUBSTITUTED PYRIDYL PYRROLES, COMPOSITIONS CONTAINING SUCH COMPOUNDS AND METHODS OF USE 278.(WO 97/16202) CYTOKINES AND THEIR USE IN TREATMENT AND/OR PROPHYLAXIS OF BREAST CANCER 279.(WO 97/16159) ENHANCED ANTI-INFLAMMATORY ORAL COMPOSITION CONTAINING H 2 RECEPTOR ANTAGONIST AND ANTIMICROBIAL OILS 280.(WO 97/15298) COMBINATION OF LTD, RECEPTOR ANTAGONISTS WITH GLUCOCORTICOSTEROIDS 281.(WO 97/14671 ) CYCLOPENTYL TACHYKININ RECEPTOR ANTAGONISTS 282.(WO 97/13751) INDOLE CARBAMATES AS LEUKOTRIENE ANTAGONISTS 283.(WO 97/13514) NK-1 RECEPTOR ANTAGONISTS FOR PREVENTION OF NEUROGENIC INFLAMMATION IN GENE THERAPY 284.(WO 97/09046) COMPOUNDS AND METHODS 285.(WO 97/07135) BINDING OF OSTEOGENIC PROTEIN-I (OP-1) AND ANALOGS THEREOF TO THE CELL SURFACE RECEPTOR ALK-1 AND ANALOGS THEREOF)

With respect to clinical and pre-clinical trial development see Antibody Therapeutics Production, Clinical Trials, and Strategic Issues, By Rathin C. Das, Ph.D., M.B.A. & K. John Morrow, Jr., Ph.D., D&MD Publications October 2001, Chapter 6.

Entity Associated and Entity Specific Markers

The term marker is used broadly to refer to any ligand or binding site for a “targeting” or an “effector” moiety of a multispecific ligand or antibody of the invention and is primarily used herein to refer to ligands which are the target of a “targeting” moiety (most often though not exclusively referred to herein as a first ligand binding moiety).

The literature is replete with examples of such markers, as well as antibodies which recognize them. With respect to cancer markers and immune cell markers, etc. many of these are referred to in Cancer: Principles and Practice of Oncology 6^{th} Ed. De Vita et al. Eds Lippincott 2001 and some are summarized at pages 309-311, 3197.

With respect to markers for osteoclasts and antibodies that bind thereto see for example Endocrinology August 1989; 125(2):630-7; Endocrinology December 1990: 127(6): 3215-21; Lab Invest April; 60(4):532-8; Calcif Tissue Int August 1998; 63(2): 148-53. Such markers could be used for example to target RANK (associated with bone resorption etc.) on osteoclasts using a relatively low affinity second ligand binding moiety.

Other applications for consideration of multispecific ligands of the invention include particularly receptors associated with angiogenesis (eg. VEGFRs 1,2,3) such as KDR, FLK-1 and FLT-1, and various cancers cell types eg. HER-2 and EGF-R, including FGF-R, PDGF-R, Tek and Tie2. Numerous other examples are referred to specifically and through references to the literature herein cited. Suitable markers for many types of target entities eg. cells bearing such receptors are referred to or referenced herein or described in various subject reviews and texts herein cited, in connection with one or more aspects and embodiments of the invention described in this application, and many others are known to those skilled in the art and desribed in the literature including antibodies.

With respect to binding to biologic effector ligands the multispecific ligand may comprise a recombinatly produced receptor for such ligand.

As the invention contemplates that the multispecfic ligands herein may be used for cancer, it is contemplated that combination therapies with chemotherapeutic and biotherapeutic agents may be used to advantage. Such agents are well known to those skilled in the art and include for example, alkylating agents, cisplatin and its analogues, antimetabolites, topoisomerase interactive agents, antimicrotubule agents, interferons, interleukins, hormonal therapeutics, differentiation agents, antiangiogenesis agents (see Cancer: Principles and Practice of Oncology 6^{th} Ed. De Vita et al. Eds Lippincott 2001 pp.335-517).

With respect to ligands involved in mediating apoptosis see also WO 0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO016170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP 1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003 513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO018042 APOPTOSIS PROTEINS WO016180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP 1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6190661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP 1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO018042 APOPTOSIS PROTEINS WO016180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO018042 APOPTOSIS PROTEINS WO016180 CD40 LIGAND AND CD40 AGONIST COMPOSITIONS AND METHODS OF USE WO0116170 NOVEL CARD PROTEINS INVOLVED IN CELL DEATH REGULATION WO0144808 METHODS OF DIAGNOSIS AND TREATMENT BY BINDING P75/AIRM1 WO0144282 BCL-G POLYPEPTIDES, ENCODING NUCLEIC ACIDS AND METHODS OF USE U.S. Pat. No. 6,242,569 Regulators of apoptosis EP1106183 Antibodies to erbB2 and their therapeutic uses WO0136594 Mcl-1 GENE REGULATORY ELEMENTS AND A PRO-APOPTOTIC Mcl-1 VARIANT US2001001712 Monoclonal antibodies having property of causing apoptosis WO0134798 CLONING AND CHARACTERIZATION OF VIRAL IAP ASSOCIATED FACTOR (VIAF) IN SEVERAL ORGANISMS CZ20000907 Monoclonal antibody inducing apoptosis HU0003513 MONOCLONAL ANTIBODY INDUCING APOPTOSIS EP1094316 Method for the detection of DNA replicating cells U.S. Pat. No. 6,207,452 Antibody of the anti-proliferation domain of human Bcl-2 WO0123568 NOVEL MEMBERS OF THE IAP GENE FAMILY U.S. Pat. No. 6,190,661 Methods and compositions for the use of apurinic/apyrimidinic endonucleases EP1087993 FAS PEPTIDES AND ANTIBODIES FOR MODULATING APOPTOSIS WO0119861 APO-2 RECEPTOR ANTIBODIES U.S. Pat. No. 6,184,034 Deoxyribonuclease II proteins and cDNAS U.S. Pat. No. 6,172,211 Nucleic acid encoding tag7 polypeptide WO0118042 APOPTOSIS PROTEINS WO0116180 CD40 LIGAND AND CD40 AGONIST COMPOSITIONS AND METHODS OF USE WOO 16170 NOVEL CARD PROTEINS INVOLVED IN CELL DEATH REGULATION

As stated above, in a related but also independent aspect, the invention contemplates a method of screening for an antibody which preferentially binds to a ligand when bound to a first receptor relative to another second receptor by screening for antibodies (eg. by phage display, ribosome display, etc.) which bind to the ligand eg. a cytokine, when bound in situ to the first receptor, and selecting among them those that bind to the ligand eg. cytokine but do not bind (substractive screening) or bind with lesser affinity when bound to the cytokine to the second receptor, as well as to antibodies and multifunctional ligands created by this method (see also U.S. Pat. No. 6,046,048 and WO 99/12973 and references cited therein with respect to TNF family of receptors). Variations in the extracellular domains of such receptors are known and can be ascertained by methods known to those skilled in the art. Accordingly the invention is directed to an antibody characterized in that it binds to an epitope on the ligand which permits the ligand to bind, while the antibody is bound to it, to a first receptor but not a second receptor. In a preferred embodiment both are cell surface receptors. In a preferred embodiment the ligand is a natural ligand, preferably a growth factor, cytokine or chemokine.

In another embodiment one of the receptors is a soluble receptor. The invention is also directed to a method of evaluating the pleitropic effects of a natural ligand by administering the said antibody Including antigen binding fragments thereof and MRUs) and monitoring its effects. The invention contemplates that this antibody is a first or second moiety of a multifunctional ligand disclosed herein. Examples of receptors include the classes of VEGF receptors (see also 1:Sheppard D. Integrin-mediated activation of transforming growth factor-beta(1) in pulmonary fibrosis.Chest. July 2001; 120(1 Suppl):S49-53. Chow D, Ho J, Nguyen Pham T L, Rose-John S, Garcia K C. In vitro reconstitution of recognition and activation complexes between interleukin-6 and gp130.Biochemistry. Jun. 26, 2001; 40(25):7593-603. Kotenko S V, Izotova L S, Mirochnitchenko O V, Esterova E, Dickensheets H, Donnelly R P, Pestka S. Identification, cloning, and characterization of a novel soluble receptor that binds IL-22 and neutralizes its activity.J Immunol. Jun. 15, 2001; 166(12):7096-103. Gustin S E, Church A P, Ford S C, Mann D A, Carr P D, Ollis D L, Young I G. Expression, crystallization and derivatization of the complete extracellular domain of the beta(c) subunit of the human IL-5, IL-3 and GM-CSF receptors.Eur J Biochem. May 2001; 268(10):2905-1 1. McCall A M, Shahied L, Amoroso A R, Horak E M, Simmons H H, Nielson U, Adams G P, Schier R, Marks J D, Weiner L M. Increasing the affinity for tumor antigen enhances bispecific antibody cytotoxicity.J Immunol. May 15, 2001; 166(10):6112-7. Piehler J, Roisman L C, Schreiber G. New structural and functional aspects of the type I interferon-receptor interaction revealed by comprehensive mutational analysis of the binding interface.J Biol Chem. Dec. 22, 2000; 275(51):40425-33.DLINE]7: Wiesmann C, Muller Y A, de Vos A M. Ligand-binding sites in Ig-like domains of receptor tyrosine kinases.J Mol Med. 2000;78(5):247-60. Review.DLINE]8: Born T L, Smith D E, Garka K E, Renshaw B R, Bertles J S, Sims J E., Protein, Nucleotide Identification and characterization of two members of a novel class of the interleukin-1 receptor (IL-1R) family. Delineation of a new class of IL-1R-related proteins based on signaling.J Biol Chem. Sep. 29, 2000; 275(39):29946-54.DLINE]9: Xia X Z, Treanor J, Senaldi G, Khare S D, Boone T, Kelley M, Theill L E, Colombero A, Solovyev I, Lee F, McCabe S, Elliott R, Miner K, Hawkins N, Guo J, Stolina M, Yu G, Wang J, Delaney J, Meng S Y, Boyle W J, Hsu H., Protein, Nucleotide TACI is a TRAF-interacting receptor for TALL-1, a tumor necrosis factor family member involved in B cell regulation.J Exp Med. Jul. 3, 2000; 192(1):137-43.DLINE]10: Kumaran J, Colamonici O R, Fish E N. Structure-function study of the extracellular domain of the human type I interferon receptor (IFNAR)-1 subunit.J Interferon Cytokine Res. May 2000; 20(5):479-85.DLINE]11: Lu D, Kussie P, Pytowski B, Persaud K, Bohlen P, Witte L, Zhu Z. Identification of the residues in the extracellular region of KDR important for interaction with vascular endothelial growth factor and neutralizing anti-KDR antibodies.J Biol Chem. May 12, 2000; 275(19):14321-30.DLINE] 12: Bowie A, O'Neill L A. The interleukin-1 receptor/Toll-like receptor superfamily: signal generators for pro-inflammatory interleukins and microbial products.J Leukoc Biol. April 2000; 67(4):508-14. Review.DLINE]13: Kumar S, McDonnell P C, Lehr R, Tierney L, Tzimas M N, Griswold D E, Capper E A, Tal-Singer R, Wells G I, Doyle M L, Young P R., Protein, Nucleotide, OMIM Identification and initial characterization of four novel members of the interleukin-1 family.J Biol Chem. Apr. 7, 2000; 275(14):10308-14.DLINE]14: Fujio K, Nosaka T, Kojima T, Kawashima T, Yahata T, Copeland N G, Gilbert D J, Jenkins N A, Yamamoto K, Nishimura T, Kitamura T., Protein, Nucleotide Molecular cloning of a novel type 1 cytokine receptor similar to the common gamma chain.Blood. Apr. 1, 2000; 95(7):2204-10.DLINE]15: Touw I P, De Koning J P, Ward A C, Hermans M H. Signaling mechanisms of cytokine receptors and their perturbances in disease.Mol Cell Endocrinol. Feb. 25, 2000; 160(1-2):1-9. Review.DLINE]16: Chritton S L, Sheng M.CYRL, a novel cytokine receptor-like protein expressed in testis, lung, and spleen.Biochem Biophys Res Commun. Jan. 27, 2000; 267(3):697-702.DLINE]17: Li H, Chen J, Huang A, Stinson J, Heldens S, Foster J, Dowd P, Gurney A L, Wood W I. Free in PMC, Protein, Nucleotide, OMIM Cloning and characterization of IL-17B and IL-17C, two new members of the IL-17 cytokine family.Proc Natl Acad Sci USA. Jan. 18, 2000; 97(2):773-8.DLINE] 18: Gary-Gouy H, Bruhns P, Schmitt C, Dalloul A, Daeron M, Bismuth G. The pseudo-immunoreceptor tyrosine-based activation motif of CD5 mediates its inhibitory action on B-cell receptor signaling.J Biol Chem. Jan. 7, 2000; 275(1):548-56.DLINE]19: Wiesmann C, Ultsch M H, Bass S H, de Vos A M., Protein, Structure Crystal structure of nerve growth factor in complex with the ligand-binding domain of the TrkA receptor.Nature. Sep. 9, 1999; 401(6749):184-8.DLINE]20: Liu Y, Cruikshank W W, O'Loughlin T, O'Reilly P, Center D M, Kornfeld H. Identification of a CD4 domain required for interleukin-16 binding and lymphocyte activation.J Biol Chem. Aug. 13, 1999; 274(33):23387-95.DLINE]21: Donnelly R P, Dickensheets H, Finbloom D S. The interleukin-10 signal transduction pathway and regulation of gene expression in mononuclear phagocytes.J Interferon Cytokine Res. June 1999; 19(6):563-73. Review.DLINE]22: Kim H, Baumann H. Dual signaling role of the protein tyrosine phosphatase SHP-2 in regulating expression of acute-phase plasma proteins by interleukin-6 cytokine receptors in hepatic cells.Mol Cell Biol. August 1999; 19(8):5326-38.DLINE]23: Kernebeck T, Pflanz S, Muller-Newen G, Kurapkat G, Scheek R M, Dijkstra K, Heinrich P C, Wollmer A, Grzesiek S, Grotzinger J., Protein, Structure The signal transducer gp130: solution structure of the carboxy-terminal domain of the cytokine receptor homology region.Protein Sci. January 1999; 8(1):5-12.DLINE]24: Ernst M, Novak U, Nicholson S E, Layton J E, Dunn A R. The carboxyl-terminal domains of gp130-related cytokine receptors are necessary for suppressing embryonic stem cell differentiation. Involvement of STAT3.J Biol Chem. Apr. 2, 1999; 274(14):972 Hibi M, Hirano T. Signal transduction through cytokine receptors.Int Rev Immunol. 1998;17(1-4):75-102. Review.26: Staunton D, Hudson K R, Heath J K. The interactions of the cytokine-binding homology region and immunoglobulin-like domains of gp130 with oncostatin M: implications for receptor complex formation.Protein Eng. November 1998; 11(11):1093-102).

With respect to markers that are useful for differentiating between various populations and sub-populations of cells, see also Human IL-18 Receptor and ST2L Are Stable and Selective Markers for the Respective Type 1 and Type 2 Circulating Lymphocytes, Woon Ling Chan, Nada Pejnovic, Christine A. Lee, and Nadia A. Al-Ali, J Immunol 2001;167 1238-1244; CD4+CD25 high Regulatory Cells in Human Peripheral Blood, Clare Baecher-Allan, Julia A. Brown, Gordon J. Freeman, and David A. Hafler, J Immunol 2001;167 1245-1253.

In the preceding detailed description, reference was made to various methodologies known to those of skill in the art of molecular biology and immunology. Publications and other materials setting forth such known methodologies to which reference was made or is made below are incorporated herein by reference in their entireties along with references cited therein as though set forth in full.

Standard reference works setting forth the general principles of recombinant DNA technology include Watson, J. D. et al, Molecular Biology of the Gene, Volumes I and II, the Benjamin/Cummings Publishing Company, Inc., publisher, Menlo Park, Calif. (1987), Darnell, J. E. et al., Molecular Cell Biology, Scientific American Books, Inc., Publisher, New York, N.Y. (1986); Lewin, B. M. Genes II, John Wiley & Sons, publishers, New York, N.Y. (1985); Old, R. W., et al., Principles of Gene Manipulation: An Introduction to Genetic Engineering, 2d edition, University of California Press, publisher, Berkeley, Calif. (1981); Maniatis, T., et al., Molecular Cloning: A Laboratory Manual, 2nd Ed. Cold Spring Harbor Laboratory, publisher, Cold Spring Harbor, N.Y. (1989), and Current Protocols in Molecular Biology, Ausubel et al., Wiley Press, New York, N.Y. (1989). Standard reference works setting forth general principles and techniques of immunology include Handbook of Experimental Immunology Blackwell Science, Incorporated, ISBN:0632009756; Antibody Engineering Blackwell Science, Incorporated, ISBN:0632009756; Therapeutic Immunology ISBN: 086542375X Blackwell Science, Incorporated; Encyclopedia of Immunology (1998) Morgan Kaufmann Publishers, ISBN:0122267656; Immunology Mosby, Incorporated, ISBN:0723429189; Abbas A K. et al. Cellular & Molecular Immunology 4^{th} Ed. 2000 ISBN 0721650023; Breitling F. et al. Recombinant Antibodies 1999 ISBN 0-471-17847-0; Masseyeff R. et al. Methods of Immunological Analysis Wiley-VCH ISBN 3-527-27906-7, 1992; Mountain et al. Eds, Biotechnology 2^{nd} ed. Vol 5A 1998 ISBN 3-527-28315-3 Wiley-VCH; Campbell, A., “Monoclonal Antibody Technology,” in, Burdon, R., et al., eds, Laboratory Techniques in Biochemistry and Molecular Biology, Volume 13, Elsevier, Publisher, Amsterdam (1984);

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All publications referred to herein are indicative of the level of skill of those in the art to which the invention pertains. All publications are herein (as well as references cited therein) are incorporated by reference to the same extent as if each individual publications were specifically and individdually indicated to be incorporated by reference in its entirety.

The present invention, thus generally described, will be understood more readily by reference to the preceding and following examples, which are provided by way of illustration and are not intended to be limiting of the present invention.

In another aspect the invention is directed to multifunctional ligand comprising at least a first moiety which specifically binds to a ligand on the surface of a virus particle that is capable of infecting a mammalian and particularly a human cell including a cancer cell (excluding viruses which are known for use in gene therapy) and is preferably selected from the group consisting viruses which infect substantial populations of individuals including for example influenza virus and at least a second moiety which specifically recognizes a cancer cell, in one embodiment preferably a marker present on multiple different cancer types, especially cancer types that are individually or collectively most prevalent in the general population. In one embodiment such multifunctional ligand is a bispecific, trispecific or tetraspecific antibody. The invention contemplates that such a multifunctional ligand may be used to target such viruses to tumors in a manner which preferentially kills the cancer cells either through the action of the virus and/or by causing the immune response to the virus or virus infected cell to preferentially (relative to non-cancer cells) target the cancer cell for ablation. The invention is also directed to a method of treating cancer by retargeting virus with which an idividual is otherwise infected to the cancer cell eg. influenza. The invention also contemplates that the multifunctional ligand includes one or more effector moieties which assist in killing the virus and/or cancer cell or directing immune cells to the virus and/or cancer cell, if and when present in the individual, for example a moiety which specifically binds to such immune cell eg. a T cell, as discussed above. Accordingly, the invention also contemplates that such multifunctional ligand may be used to treat influenza virus infections and secondarily to act prophylactically as a sentinel against any cancer cells which might develop during the course of the viral infection or a period of immune suppression or increased succeptibility to infection or cancer, including for example, as experienced by individuals with a particular immune suppressive disorder or condition or under treatment with immune suppressive drugs, individuals at risk for cancer or recurrence of a cancer, individuals of a particular age group, individuals experiencing a period of unusual stress which increases their succeptibility to disease or infection. The invention also contemplates that such a moiety is used in concert with prior immunization against the virus, so that the augemented immune response to the virus benefits the treatment of the cancer cells (see for example U.S. Pat. No. 6,169,175 and the art cited therein). The invention also directed to such a virus (excluding viruses known for use in gene therapy applications eg. adenovirus) which is engineered to expresses on its surface a cancer targeting moiety such as a scFv (see for example EP 1038967, WO 94/10323 and the art cited therein). The invention is also directed to a method of identifying the expression or over-expression of cell surface markers associated with infection by such a virus, by substractive screening relative to markers also expressed on non-infected such cells, for example using phage display or the like. Such markers may be used for vaccine-type or other immunotherapeutic strategies. Anti-virus markers including influenza virus markers and methods of identifying new such markers are well known in the art (see for example U.S. Pat. No. 5,589,174) (see also The role of the antibody response in influenza virus infection., Gerhard W.,Curr Top Microbiol Immunol 2001;260:171-90, Fernandez-Sesma A, Schulman J L, Moran T M. A bispecific antibody recognizing influenza A virus M2 protein redirects effector cells to inhibit virus replication in vitro.J Virol. July 1996; 70(7):4800-4; Todorovska A, Roovers R C, Dolezal O, Kortt A A, Hoogenboom H R, Hudson P J. Design and application of diabodies, triabodies and tetrabodies for cancer targeting. J Immunol Methods. Feb. 1, 2001; 248(1-2):47-66., Staerz U D, Yewdell J W, Bevan M J. Hybrid antibody-mediated lysis of virus-infected cells. Eur J Immunol. April 1987; 17(4):571-4; Fernandez-Sesma A, Schulman J L, Moran T M. A bispecific antibody recognizing influenza A virus M2 protein redirects effector cells to inhibit virus replication in vitro. J Virol. July 1996; 70(7):4800-4.)

Methods and constructs for administrating DNA to a mammal for purposes of gene therapy are well described in the literature (see for example Bessis N et al. Gene therapy fro rheumatoid arthritis, J Gene Med. November-December 2002; 4(6):581-91 PMID:12439850; Martin K R et al. Gene delivery to the eye using adeno-associated viral vectors, Methods. October 2002; 28(2):267-75 PMID:12413426; Ruitenberg M J et al. Adeno-associated viral vectors as agents for gene delivery: application in disorders and trauma of the central nervous system, Methods. October 2002; 28(2):182-194 PMID:12413416; High K A, AAV-mediated gene transfer for hemophilia Ann NY Acad Sci. December 2001; 953:64-74 PMID:11795424; Rochlitz C F, Gene therapy of cancer, Swiss Med Wkly. Jan. 12, 2001; 131 (1-2):4-9 PMID:11205184; Wattanapitayakul S K, Bauer J A, Recent developments in gene therapy for cardiac disease, Biomed Pharmacother. October 2000; 54(10):487-504 PMID:1 1130847; Prud'homme G J, Gene therapy of autoimmune diseases with vectors encoding regulatory cytokines or inflammatory cytokine inhibitors. J Gene Med. July-August 2000; 2(4):222-32.

The invention is also directed to a multifunctional ligand having at least a tumor cell targeting moiety and a moiety which binds to a tumor antigen which is shed from a cancer cell. In a preferred embodiment, the tumor antigen binding moiety preferably does not recognize the portion of the antigen which is most immunogenic and leaves that portion exposed for recognition by the immune system. The invention contemplates generating such preferred antibody or fragment thereof by using an an immune complex between an antibody that binds to such immunogenic portion and the antigen as a target for phage display or generation or polyclonal sera. The invention also contemplates identifying antibodies which recognize immunogenic portions of the antigen by screening patient sera for antibodies which recognize the antigen. The invention also contemplates that such multifunctional ligand includes one or more effector moieties which assist in killing the cancer cell or directing immune cells to the cancer cell, for example a moiety which specifically binds to such immune cell eg. a T cell receptor, as discussed above.

Without limiting the generality of or applicability of the foregoing, and without being limited by or limiting the scope of the claims, various embodiments of the invention may be summarized for ease of reference as follows: 1. A multi specific ligand comprising at least a first ligand binding moiety which preferentially binds with a first affinity⁸ to a first ligand having a first biodistribution* and at least a second ligand binding moiety which preferentially binds with a second affinity to a second ligand having a second biodistribution which is different⁹ from that of the first ligand, and wherein the affinity of first and second ligand binding moieties are selected to bias the biodistribution of the multispecific ligand. 2. A multispecific ligand according to paragraph 1, wherein said multifunctional ligand comprises one or more ligand binding moieties which are antibodies. 3. A multispecfic ligand according to paragraph 1 or 2, wherein the affinity of said first ligand binding moiety for the first ligand is higher than the affinity of the second ligand binding moiety for the second ligand so as to bias the biodistribution of the multispecific ligand in favor of the the first ligand. 4. A multispecific ligand according to paragraph 3, wherein the first and second ligands have overlapping biodistributions.¹⁰ 5. A multispecfic ligand according to paragraph 4, wherein the first ligand is a target cell population associated ligand and wherein said second ligand is present on a broader population of cells and wherein the biodistribution of the multispecific ligand is skewed in favour of the target cell population. 6. A multispecfic ligand according to paragraph 1 or 5, wherein said first ligand is a marker associated with* one or more specific cell populations, infectious or parasitic agents, diseased cells, or disease associated* cells, optionally one of specific ligands herein mentioned or referenced or known to those skilled in the art. 7. A multispecific ligand according to paragraph 6, wherein said marker is a specific biological structure. 8. A multispecific ligand according to paragraph 6, wherein said marker is a specific receptor or receptor ligand. 9. A multispecific ligand according to paragraph 6, wherein said marker is a specific antigen. 10. A multi specific ligand according to paragraph 6, wherein said marker is a specific epitope. 11. A multispecific ligand according to paragraph 6, wherein said marker is a CD marker. 12. A multispecific ligand according to paragraph 6, wherein said marker is associated with a cancer cell or pre-cancerous cell. 13. A multispecific ligand according to paragraph 6, wherein said marker is associated with an autoimmune disorder or rheumatic disease. 14. A multispecific ligand according to paragraph 6, wherein said marker is associated with a specific tissue type. 15. A multispecific ligand according to paragraph 6, wherein said marker is associated with a specific organ. 16. A multispecific ligand according to paragraph 6, wherein said marker is associated with a cell or tissue of specific origin or class. 17. A multispecific ligand according to paragraph 6, wherein said marker is an MHC-peptide complex. 18. A multispecific ligand according to paragraph 6, wherein said marker is associated with a cell surface immunoglobulin. 19. A multispecific ligand according to paragraph 5 or 6, wherein said second ligand is a receptor, family of receptors or one or more particular receptor family members, optionally one of those specific receptors herein mentioned or referenced or known to those skilled in the art. The invention contemplates targeting any receptor present on any population of entities for which there is an entity associated marker. 20. A multispecific ligand according to paragraph 19, wherein said second ligand is a cell surface receptor chosen from a group comprising tyrosine kinase type receptors, serine kinase type receptors, heterotrimeric G-protein coupled receptors, receptors bound to tyrosine kinase, TNF family receptors, notch family receptors, guanylate cyclase types, tyrosine phosphatase types, decoy receptors, and adhesion receptors, optionally one of the specific receptors herein mentioned or referenced or known to those skilled in the art. 21. A multispecific ligand according to paragraph 19, wherein said receptor requires cross-linking for biological activity. 22. A multispecific ligand according to paragraph 5 or 6, wherein said second ligand is a cell surface receptor and wherein said second ligand binding moiety blocks said receptor. 23. A multi specific ligand according to paragraph 1, 5 or 6, wherein said second ligand is a receptor ligand and wherein said second ligand binding moiety blocks interaction with the corresponding receptor. 24. A multispecific ligand according to paragraph 5 or 6, wherein said second ligand is a cell surface receptor which initiates a signal transduction and wherein said second ligand binding moiety effects a signal transduction. 25. A multispecific ligand according to paragraph 5, 6 or 19, wherein said antibody comprises a first VH* which preferentially recognizes said first ligand and a second VH which preferentially recognizes said second ligand. 26. A multispecific ligand according to paragraph 25, wherein at least one of said first and second VHs require the cooperation of a VL for binding to their respective ligands. 27. A multispecific ligand according to paragraph 25, comprising a first VL associated with said first VH and a second VL associated with said second VH and wherein both said first and second VHs require the cooperation of a VL for binding to the first and second ligands, respectively, and wherein said first and second VLs are the same¹¹ or functionally interchangeable*. 28. A multispecific ligand according to paragraph 25 or 27, wherein said bispecific antibody is a four chain antibody. 29. A multispecific ligand according to paragraph 28, wherein said bispecfic antibody is a minibody, F(ab′)₂ or antibody devoid of a CH3 domain. 30. A multispecific ligand according to paragraph 25, wherein said bispecific antibody is a diabody. 31. A multispecific ligand according to paragraph 25 or 27, wherein said bispecific antibody is devoid of light chains. 32. A multispecific ligand according to paragraph 31, wherein said bispecific antibody comprises a pair of disulfide linked heavy chains or heavy chain portions each comprising at least a VH region, a hinge region and preferably, at least a portion of an Fc region at the carboxy terminus of the hinge region. 33. A multispecific ligand according to paragraph 31, wherein said bispecific antibody comprises a pair of VHs linked via a polypeptide linker. 34. A multispecific ligand according to paragraph 3, 5, 6 or 19 wherein the affinity of the first ligand binding moiety for the first ligand is at least approximately¹², one, two, three, four, five, six, seven or eight orders of magnitude greater than the affinity of said second ligand binding moiety for the second ligand. 35. A composition comprising a multispecific ligand in a pre-determined dosage, said multispecific ligand comprising a first ligand binding moiety which preferentially binds with a pre-selected first affinity to a first ligand having a first biodistribution and a second ligand binding moiety which preferentially binds with a pre-selected affinity to a second ligand having a second biodistribution, which is different from that of the first ligand, and wherein the affinity of the first and second ligand binding moieties are selected to bias the biodistribution of the multispecific ligand in favor of a selected location of one or both of the ligands¹³ such that a desired proportion of the dosage is delivered to the selected location. 36. A multispecific ligand according to paragraph 1 or 35, wherein the biodistributions of said first and second ligands overlap¹⁴ and wherein the affinities of the first and second ligand binding moieties are selected to bias the biodistribution of the multispecific ligand in favour of a target cell population on which both first and second ligands are bioavailable for recognition by the first and second ligand binding moieties, relative to one or more non-target cell populations. 37. A multispecific ligand according to paragraph 36, wherein the affinity of first ligand binding moiety for the first ligand is at least, approximately, one, two, three, four, five, six, seven or eight orders of magnitude greater than the affinity of the second ligand binding moiety for the second ligand. 38. A multispecific ligand according to paragraph 36 or 37, wherein first and second ligands are bioavailable for contemporanous* recognition by the first and second ligand binding moieties. 39. A method of controlling the biodistribution of a ligand which interacts with a target ligand present on a heterogenous population of ligand bearing entities, said method comprising using a multispecific ligand comprising at least a first ligand binding moiety which preferentially* binds with a pre-selected* first affinity¹⁵ to at least a first ligand associated with a target sub-population of said heterogeneous population on which said first ligand and target (second) ligand are bioavaible for contemporaneous recognition and a second ligand binding moiety which preferentially binds with a pre-selected lesser affinity to said target ligand, and wherein the affinity of first and second ligand binding moieties are selected to bias the biodistribution of the multispecific ligand in favor of said target sub-population of ligand bearing entities. 40. A method of testing the biological effects of limiting the biodistribution of a ligand which interacts with a target ligand present on a heterogenous population of ligand bearing entities, said method comprising the step of administering a multispecific ligand comprising at least a first ligand binding moiety which preferentially* binds with a pre-selected* first affinity¹⁶ to at least a first ligand associated with a target sub-population of said population of ligand bearing entities on which said first ligand and target (second) ligand are bioavaible for contemporanous recognition and a second ligand binding moiety which preferentially binds with a pre-selected lesser affinity to said target ligand, and wherein the affinity of first and second ligand binding moieties are selected to bias the biodistribution of the multispecific ligand in favor of said target sub-population of ligand bearing entities. 41. A multispecific ligand which preferentially binds to a target ligand on a selected sub-population of a heterogeneous population of cells bearing the target-ligand, the multispecific ligand comprising a first ligand binding moiety which preferentially binds to a cell sub-population associated ligand and a second ligand binding moiety which binds to the target-ligand, said first ligand binding moiety having an affinity for the sub-population associated ligand that is higher than the affinity of the second ligand binding moiety for the target ligand. 42. A multispecific ligand according to paragraph 41, wherein the affinity of said first ligand binding moiety for the cell sub-population associated ligand is approximately, one, two, three, four, five, six, seven or eight orders of magnitude greater than the affinity of said second ligand binding moiety for said target ligand. 43. A multispecific ligand according to paragraph 42, wherein said target ligand is a receptor or a receptor ligand¹⁷. 44. A multispecific ligand according to paragraph 42, wherein at least one of said first or second ligand binding moieties comprises an antibody heavy chain or functional portion(s) thereof including a VH or fragment thereof and an antibody light chain or functional portion(s) thereof including a VH or fragment thereof. 45. A method of selectively exerting a biological effect mediated through binding a target-ligand on a selected sub-population of a population of cells bearing the target-ligand, the method comprising the step of exposing the cells to a multispecific ligand comprising a first ligand binding moiety which preferentially binds to a cell sub-population associated ligand and a second ligand binding moiety which binds to the target ligand, said first ligand moiety having an affinity for the sub-population associated ligand that is higher than the affinity of the second ligand binding moiety for the target ligand. 46. A method according to paragraph 45, wherein the affinity of said first ligand binding moiety for the cell sub-population associated ligand is at least approximately, one, two, three, four, five, six, seven or eight orders of magnitude greater than the affinity of said second ligand binding moiety for said target ligand. 47. A method according to paragraph 45, wherein said target ligand is a receptor or a receptor ligand. 48. A method according to paragraph 45 wherein at least one of said first or second ligand binding moieties is an antigen binding fragment¹⁸ of an antibody. 49. A method of testing or controlling the biological effects of a ligand binding molecule by circumscribing its ability to bind to a diverse population of cells bearing a complementary target ligand, said method comprising using said ligand binding molecule together with a different ligand binding molecule which preferentially binds to another target ligand which is exclusively or preferentially associated with one or more sub-population(s) of said population of cells, and wherein the biological effects of said ligand binding molecule are controlled through prior association of said ligand binding molecule with said other ligand binding molecule to form a multispecific ligand and through the affinity of at least one of said ligand binding molecules being pre-selected to limit the propensity of said ligand binding molecule to bind to cells within said population of cells which do not preferentially express said other target ligand. 50. A method according to paragraph 49, wherein the affinity of said ligand binding molecule for said complementary target ligand is less than the affinity of the other ligand binding molecule for the other target ligand. 51. A method according to paragraph 49 or 50, wherein at least one of said first and second ligand binding molecules is an antibody*. 52. A method according to paragraph 49 wherein said first ligand binding molecule is an entity which exerts a biologic effect and said second ligand binding molecule is a multispecific ligand comprising a first ligand binding moiety that binds to a cell sub-population associated ligand and a second ligand binding moiety which binds to said entity. 53. A method according to paragraph 52 wherein said second ligand binging moiety is an antibody which binds to a pre-selected epitope on said entity and wherein the epitope of said second ligand binding moiety is selected on the basis of its proximity to a ligand binding portion of said entity such that the entity when bound to said multifunctional ligand has an affinity for said ligand which is less than the affinity of said first ligand binding moiety for said cell sub-population associated ligand. 54. A multispecific ligand according to paragraph 12, wherein said second ligand is a IL-8 receptor, a CCR7 receptor, a FAS receptor, or a CXCR4 receptor. 55. A multispecific ligand according to paragraph 6, wherein said marker is associated with an immune cell that is succeptible to viral infection. 56. A multispecific ligand according to paragraph 55, wherein said marker is CD4. 57. A multispecific ligand according to paragraph 55 or 56, wherein said second ligand is a CCR5 or CXCR4 receptor. 58. A multifunctional ligand according to paragraph 52, wherein said entity is a biologic effector ligand. 59. A multi specific ligand according to paragraph 36, wherein the affinities of said first and second ligand binding moieties are both selected to limit their individual ability to bind to the first and second ligands, respectively, and wherein their combined functional affinity biases the distribution of the multifunctional in favour of said target cell population. 60. An antibody which binds to an epitope on an entity which exerts a biologic effect via a binding interaction with a target ligand, said epitope being proximal to the binding site of said entity for the target ligand, such that the antibody bound to the entity reduces the affinity of the entity for its ligand without precluding its functional binding activity vis-á-vis said ligand. 61. A multispecific ligand comprising a first ligand binding moiety which binds with a pre-selected affinity to a target entity associated ligand and a second ligand binding moiety which binds with pre-selected affinity to an epitope on a biologic effector ligand which exerts a biologic effect via a binding interaction with a target ligand, said epitope being proximal to the binding site of said biologic effector ligand for the target ligand, such that the second ligand moiety bound to the biologic effector ligand reduces the affinity of the molecule for the target ligand without precluding its functional binding activity vis-a-vis said ligand and wherein said target ligand is present on a diverse population of entities consisting of the target entity and one or more non-target entities and wherein the affinity of the first ligand binding moiety for the target entity associated ligand is greater than that of the biologic effoctor ligand for the target ligand when bound to second ligand molecule, and wherein the affinity of the first ligand binding moiety is selected to bias the biodistribution of said biologic effector ligand in favour of the target entity relative to the non-target entit(ies). 62. A multispecific ligand according to paragraph 61, wherein first ligand binding moiety comprises a light chain linked to an antibody heavy chain portion comprising at least a VH, CH1 domain, hinge region and preferably at least a truncated Fc portion and said second ligand binding moiety comprises at least a VL linked to a heavy chain portion, optionally through a disulfide bond¹⁹ and wherein the heavy chain portion of said second ligand binding moiety is devoid of CH1 domain, and comprises a hinge region and preferably at least a truncated Fc portion, and wherein said heavy chain portions are linked via their respective hinge regions and optionally wherein the respective hinge regions are wholly or partially substituted or supplemented by a another linkage pair²⁰ eg. a leucine zipper. 63. A multispecific ligand according to paragraph 12, wherein said second ligand is a marker associated with a lymphatic endothelial cell. 64. A multispecific ligand comprising a first ligand binding moiety which preferentially binds to a lymphatic endothelial cell associated marker and a second moiety which exerts a biologic function²¹, optionally a therapeutic function, optionally an immune function*, optionally at least one of an immunizing²² function, a tolerizing function, a neutralizing²³ function, an immune mediating function, and immune modulating function^{24,} in relation* to an independent²⁵ entity, preferably within the lymphatic system. 65. A multispecific ligand according to paragraph 62, wherein the marker is selected to limit the ability of said endothelial cell to internalize said multispecific ligand. 66. A multifunctional ligand having, at least, a first portion which binds to a lymphatic vessel associated ligand and a second portion comprising an immune function exerting moiety. 67. A multifunctional ligand as defined in paragraph 66, wherein said first portion is an antibody. 68. A multifunctional ligand as defined in paragraph 66, wherein said immune function exerting moiety binds to a target ligand. 69. A multifunctional ligand as defined in paragraph 67, wherein said immune function exerting moiety comprises an antibody. 70. A multifunctional ligand as defined in paragraph 68, wherein said immune function exerting moiety comprises an antibody. 71. A multifunctional ligand as defined in paragraph 68, wherein said immune function exerting moiety binds to a ligand selected form the group consisting of CCR5, CTLA-4, LFA-1, ICAM-1, CD2, CD3, CD4, CD22, CD40, CD44; CD80, CD86, CD134 and CD154. 72. A multifunctional ligand as defined in paragraph 70, wherein said first portion binds to LYVE-1 or podoplantin. 73. A multifunctional ligand as defined in paragraph 70, wherein said immune function exerting moiety comprises an anti-idiotypic antibody. 74. A multifunctional ligand as defined in paragraph 73, wherein said anti-idiotypic antibody binds to an autoimmune antibody. 75. A multifunctional ligand as defined in paragraph 73, wherein said anti-idiotypic antibody mimics a cell surface expressed tumor antigen or a viral antigen. 76. A multifunctional ligand as defined in paragraph 70, wherein said immune function exerting moiety binds to a diseased cell. 77. A multifunctional ligand as defined in paragraph 70, wherein said immune function exerting moiety binds to an infectious agent or parasite. 78. A multifunctional ligand as defined in paragraph 76, wherein said diseased cell is a cancer cell. 79. A multifunctional ligand as defined in paragraph 76, wherein said diseased cell is a virally infected cell. 80. A multifunctional ligand as defined in paragraph 68, wherein said immune function exerting moiety binds to an immune cell. 81. A multifunctional ligand as defined in paragraph 69, 76 or 80 wherein said immune function exerting moiety binds with greater functional affinity to its target ligand than said first portion binds to its target ligand. 82. A multifunctional ligand as defined in paragraph 69, 76 or 80 wherein said immune function exerting moiety binds with greater affinity to its target ligand than said first portion binds to its target ligand. 83. A multifunctional ligand as defined in paragraph 69, 76 or 80, wherein said binds with greater avidity to its target ligand than said first portion binds to its target ligand. 84. A multifunctional ligand as defined in paragraph 80, wherein immune cell is associated with an autoimmune reaction. 85. A multifunctional ligand as defined in paragraph 80, wherein said immune cell is a CCR5-expressing cell. 86. A multifunctional ligand as defined in paragraph 78 or 80, wherein said second portion comprises an internalizing antibody and a cytotoxic component. 87. A multifunctional ligand as defined in paragraph 78 or 80, which is a bispecific antibody having a monovalent first portion and a monovalent second portion. 88. A multifunctional ligand as defined in paragraph 78 or 80, which is a bispecific antibody having a divalent first portion and a divalent second portion. 89. A multifunctional ligand as defined in paragraph 78 or 80, which is a trispecific antibody having a monovalent first portion and a second portion comprising a divalent immune function exerting moiety which binds to one or more target ligands on a target diseased cell or immune cell and a movovalent anti-CD3 or anti-CD28 antibody. 90. A multifunctional ligand as defined in paragraph 78 or 80, which is a trivalent trispecific antibody having a monovalent first portion and a second portion comprising a divalent immune function exerting moiety which binds to a target ligand on a target diseased or immune cell. 91. A multifunctional ligand as defined in paragraph 78, wherein said second portion comprises a cytokine component. 92. A multifunctional ligand as defined in paragraph 78, wherein said second portion comprises a cytotoxic component. 93. A multifunctional ligand as defined in paragraph 78, wherein said second portion comprises a ligand which is capable of binding to T cells. 94. A multifunctional ligand as defined in paragraph 87, wherein said ligand is an antibody which binds to T cells. 95. A multifunctional ligand as defined in paragraph 78, wherein said second portion comprises an anti-CD3 antibody or anti-CD28 antibody. 96. A multifunctional ligand as defined in paragraph 67, wherein second portion is a cytokine component. 97. A multifunctional ligand as defined in paragraph 67, wherein second portion is an anti-CD3 antibody or an anti-CD28 antibody. 98. A multifunctional ligand as defined in paragraph 13, wherein said second portion further comprises one or more components selected from the group consisting of a cytokine component, a cytotoxic component and an anti-CD3/CD28 component. 99. A multifunctional ligand as defined in paragraph 14, wherein said second portion further comprises one or more components selected from the group consisting of a cytokine component, a cytotoxic component and an anti-CD3/CD28 component. 100. A pharmaceutical composition comprising a multifunctional ligand as defined in paragraph 101. A pharmaceutical composition comprising a plurality of different multifunctional ligands. 102. A pharmaceutical composition as defined in paragraph 101, wherein said plurality of different multifunctional ligands exert a cooperative immune effect. 103. A pharmaceutical composition as defined in paragraph 101, wherein said plurality of different multifunctional ligands comprise a multifunctional ligand as described in paragraph 76 and at least one or both of the multifunctional ligands described in paragraph 95 or 96. 104. A method of inhibiting the formation of metastasis during the course of surgical removal of a tumor comprising administering to a patient prior to surgical treatment of the tumor site, a pharmacetical composition comprising a multifunctional ligand as described in paragraph 78. 105. An immunocytokine comprising an anti-idiotypic antibody which recognizes the paratope of an antibody which binds to a lymphatic vessel associated ligand and a cytokine fused therewith or conjugated thereto. 106. An immunocytokine as defined in paragraph 105, wherein said cytokine component comprises IL-2 or a functional fragment thereof and/or IL-12 or a functional fragment thereof. 107. An immunocytokine as defined in paragraph 42, wherein said cytokine component comprises TNF-α or a functional fragment thereof. 108. A bispecific antibody comprising an anti-idiotypic antibody which recognizes the paratope of an antibody which binds specifically to a lymphatic vessel associated ligand and an anti-CD3 antibody or an anti-CD28 antibody. 109. A multifunctional ligand according to paragraph 66 comprising one or more amino acids that are substituted for amino acids that contribute to an immunogenic epitope. 110. A multifunctional ligand having, at least, a first portion which binds to a lymphatic vessel associated ligand and a second portion comprising an independent therapeutic function exerting moiety. 111. A bispecific ligand comprising a first ligand which binds to a first target ligand and a second ligand which binds to a second target ligand, and wherein the affinity of said first ligand is selected to enable binding to the first target ligand independently of the ability of said second ligand to bind to the second target ligand and wherein the affinity of said second ligand is selected to substantially reduce the probability of its binding to the second target ligand without the first ligand binding first or substantially contemporaneously to the first target ligand. 112. A bispecific antibody comprising a first antibody component which binds to a first target ligand and a second antibody component which binds to a second target ligand, and wherein the affinity or avidity or both the affinity and avidity of said first antibody component are selected to enable binding to the first target ligand independently of the ability of said second antibody component to bind to the second target ligand and wherein the avidity or affinity or both the affinity and avidity of said second ligand are selected to substantially reduce the probability of its binding to the second target ligand without the first ligand binding first or substantially contemporaneously to the first target ligand. 113. A multispecific ligand comprising a first moiety which binds to a first target ligand and a second moiety which binds to a second target ligand, and wherein the affinity or avidity or both the affinity and avidity of said first moiety are selected to enable the first moiety to bind to the first target ligand independently of the ability of said second moiety to bind to the second target ligand and wherein the avidity or affinity or both the affinity and avidity of said second moiety are selected to substantially reduce the probability of its binding to the second target ligand without the first moiety, first or substantially contemporaneously, binding to the first target ligand. 114. A multispecific ligand according to paragraph 113, wherein both moieties bind to different target ligands on the same cell. 115. A multispecific ligand comprising a first moiety which binds to a first target ligand and a second moiety which binds to a second target ligand, and wherein the affinity or avidity or both the affinity and avidity of said first moiety and the avidity or affinity or both the affinity and avidity of said second moiety are selected to substantially reduce the probability of either moiety binding for a sufficient duration or series of durations to its respective target ligand to a accomplish a therapeutic function without the other moiety, first or substantially contemporaneously, binding to its respective target ligand 116. A multispecific ligand comprising a first moiety which specifically binds to a first target ligand on a first entity and a second moiety which specifically binds to a second target ligand on a second entity, and wherein the affinity or avidity or both the affinity and avidity of said first moiety are selected to enable the first moiety to bind to the first target ligand independently of the ability of said second moiety to bind to the second target ligand and wherein the avidity or affinity or both the affinity and avidity of said second moiety are selected to enable the second moiety to bind to the second entity in preference to the first moiety binding to the first entity when both first and second moieties are substantially contemporaneously bound to the respective first and second entities. 117. A multi specific ligand comprising a first moiety which specifically binds to a first target ligand on a first entity and a second moiety which specifically binds to a second target ligand on a second entity, and wherein the second entity binds to a third target ligand, and wherein the affinity or avidity or both the affinity and avidity of said first moiety are selected to enable the first moiety to bind to the first target ligand independently of the ability of said second moiety to bind to the second target ligand and wherein the avidity or affinity or both the affinity and avidity of said first moiety are selected to enable the first moiety to bind to the first entity in preference to the second moiety binding to the second entity when both first and second moieties are substantially contemporaneously bound to the respective first and second entities, and wherein the avidity or affinity or both the affinity and avidity of said second moiety are selected to enable the third target ligand to bind to the second entity in preference to the second moiety binding to the second entity when both said third target ligand and the second moiety are substantially contemporaneously bound to the second entity. 118. A multispecific ligand comprising at least a first ligand binding moiety which specifically binds to a first ligand having a first biodistribution and a second ligand binding moiety which specifically binds to a second ligand having a second biodistribution, and wherein the affinity of the first and second ligand binding moieties are different and selected to bias the biodistribution of the multispecific ligand, and wherein the affinity of the first ligand binding moiety for the first ligand is at least, approximately, one order of magnitude greater than that of the second ligand binding moiety for the second ligand. The affinity of the first ligand binding moiety for the first ligand is optionally at least, approximately, two orders of magnitude greater than that of the second ligand binding moiety for the second ligand. The affinity of the first ligand binding moiety for the first ligand is optionally at least, approximately, three orders of magnitude greater than that of the second ligand binding moiety for the second ligand. The affinity of the first ligand binding moiety for the first ligand is optionally at least, approximately, four orders of magnitude greater than that of the second ligand binding moiety for the second ligand. The affinity of the first ligand binding moiety for the first ligand is optionally at least, approximately, five orders of magnitude greater than that of the second ligand binding moiety for the second ligand. The affinity of the first ligand binding moiety for the first ligand is optionally at least, approximately, six orders of magnitude greater than that of the second ligand binding moiety for the second ligand. The affinity of the first ligand binding moiety for the first ligand is optionally at least, approximately, seven orders of magnitude greater than that of the second ligand binding moiety for the second ligand The affinity of the first ligand binding moiety for the first ligand is optionally at least, approximately, eight orders of magnitude greater than that of the second ligand binding moiety for the second ligand. 119. A multispecific ligand according to paragraph 118, wherein the first ligand is present on a first target cell population and wherein said second ligand is present on a second target cell population comprising the first target cell population and wherein the biodistribution of the multispecific ligand favours the first target cell population. 120. A multispecific ligand according to paragraph 119, wherein said multispecific ligand is capable of contemporaneously binding the first and second ligands on said target population. 121. A host cell or cell free expression medium comprising one or more polynucleotides, said one or more polynucleotides comprising one or more DNA sequences, said one or more DNA sequences comprising one or more polypeptides which are sufficient to constitute a multi specific ligand as defined in any of the preceding paragraphs. 122. A kit comprising one or more polynucleotides, said one or more polynucleotides comprising one or more DNA sequences, said one or more DNA sequences encoding one or more polypeptides which are sufficient to constitute a multispecific ligand as defined in any of the preceding paragraphs. 123. A liquid medium comprising comprising one or more polypeptides which are sufficeint to constitute a multispecific ligand as defined in any of the preceding paragraphs. 124. A liquid medium comprising one or more host cells, said one or more host cells comprising one or more polynucleotides, said one or more polynucleotides comprising one or more DNA sequences, said one or more DNA sequences encoding one or more polypeptides which are sufficeint to constitute a multispecific ligand as defined in any of the preceding paragraphs. 125. A substantially isolated polynuceotide comprising a DNA sequence encoding a polypeptide portion of a second ligand binding moiety as defined in any of the preceding claims, said polypeptide portion comprising a VH or VL, said second ligand binding moiety having a low affinity for said second ligand. 126. A substantially isolated polynucleotide according to paragraph 125, wherein said polynucleotide is a substantially isolated expression or cloning vector. 127. A method of making a multispecific ligand as defined in any of the preceding paragraphs comprising expressing at least one polynucleotide as defined in paragraph 122 or 125. 128. A pharmaceutical composition comprising a multispecific ligand as defined in any of the preceding paragraphs and a pharmaceutically acceptable excipient. 129. A therapeutic composition comprising a multispecific ligand as defined in any of the preceding paragraphs and a pharmaceutically acceptable excipient. 130. A method of treating a disease in a mammal comprising administering a therapeutically effective amount of a multispecific ligand according to any of the preceding claims. 131. A kit comprising a plurality of different multispecific ligands as defined herein.
⁸ Contrasted to functional affinity which may result from avidity

⁹see fn 8

¹⁰ The term epitope though technically understood to be specific for a given antibody, is used to refer to antigenic determinants that are situated proximally to one another so that two antibodies will be considered to bind to the same epitope if one competively inhibits the binding of the other through any probative competitive inhibition experiment known to those skilled in the art.

¹¹ have substantially the same amino acid composition ie. with possible exception of one or more additions, deletions or substitutions including conservative amino acid substitutions which do not substantially affect the specificity and amino acid composition of the paratope

¹² the term approximately in the context of orders of magnitude variations in affinity refers a variability that is up to a half an order or magnitude.

¹³ having regard to their respective bioavailabilities

¹⁴ The term “overlap” connotes that notwithstanding the difference in distributions of the first and second ligands the first and second ligands are bioavailable for recognition on the same entity. This term and related terms, exemplified below, are intended to exclude a situation where both ligands are preferentially expressed on substantially the same entity, for example two different tumor associated antigens associated differentially with a differentiated population of cells within a tumor, most particularly in the case where they are individually suitable targets for delivery of a toxic payload, and the terms “different” distributions and “heterogeneous” population are similarly understood to exclude such a common distribution, in the appreciation that the invention primarily represents an improved strategy for targeting two different ligands, in which one ligand has a broader distribution than the other or both have distributions that may overlap but are different from that of the target population. It will also be appreciated that the invention has particular application to a situation in which at least one of the non-target populations is one on which one of said first and second ligands is substantially represented (in contrast to one on which it simply enjoys limited expression).

¹⁵ Contrasted to functional affinity which may result from avidity

¹⁶ Contrasted to functional affinity which may result from avidity

¹⁷The term “receptor ligand” means a target ligand which is a ligand for a receptor, for example, a receptor on a cell or infectious agent or a receptor which circulates independently of another entity.

¹⁸ The term “antigen binding fragment” refers to a polypeptide or a plurality of associated polypeptides comprising one or more portions of an antibody including at least one VH or VL or a functional fragment thereof.

¹⁹ For example of the disulfide stabilized type developed by the NCI

²⁰ eg. fos-jun

²¹ The moiety that exerts a biologic function is understood to be a biologic effector in the sense that its intended interaction with an entity in the lymphatic system or elsewhere in the organism has a biological consequence.

²² For example using a toxin or immunogen fused or conjugated to (or having a corresponding ligand on the second binding moiety to which it binds) to an antibody which recognizes a lymphatic endothelial marker, for example an anthrax toxin fusion

²³ The tern neutralizing is used broadly to refer to any interposition, interference or impediment which affects the function of the target entity

²⁴ the terms modulating, mediating, neutralizing function etc. are not intended to be mutully exclusive and are each used broadly, for example the term modulating referring to effecting a change, and the term mediating preferably connoting an indirect effect achieved through the instrumentality of another entity, for example a cell, cytokine, chemokine etc.

²⁵ Ie. an entity other than the lymphatic endothelial cell and other than any cell to which the first moiety is anchored.

Notwithstanding any indication to the contrary, it will be appreciated that the references herein cited have application to multiple different subjects and any qualifying remarks as to the applicability of the references is to be understood as relating to each of the subjects for which references are herein provided, as limited only by the title and subject matter of the reference.

All publications and references therein cited are herein incorporated by reference to the same extent as if each of the individual publications were specifically and individually indicated to be incorporated by reference in its entirety.

### Appendix A

U.S. Pat. No. 6,103,234 Composition comprising an antibody to macrophage-derived inflammatory mediator (MIP-2) U.S. Pat. No. 6,090,383 Monoclonal antibodies to transforming growth factor-.beta. and methods of use U.S. Pat. No. 6,090,382 Human antibodies that bind human TNF.alpha. U.S. Pat. No. 6,086,874 Antitumor agent effect enhancer containing IL-6 antagonist U.S. Pat. No. 6,084,075 Agonist and antagonist antibodies to the chemokine receptor-2 (CCR2) U.S. Pat. No. 6,074,642 Use of antibodies specific to human complement component C5 for the treatment of glomerulonephritis U.S. Pat. No. 6,068,841 Antibodies to Fas-L for treatment of hepatitis U.S. Pat. No. 6,068,840 Recombinant CDR-grafted and chimeric anti-IL-8 antibodies containing CDRs or the variable region from the mouse monoclonal antibody, WS-4 U.S. Pat. No. 6,057,427 Antibody to cytokine response gene 2(CR2) polypeptide U.S. Pat. No. 6,056,957 Humanized monoclonal antibodies against human interleukin-5 U.S. Pat. No. 6,036,956 Method and dosage form using an antagonist to gamma interferon to control MHC-associated autoimmune disease U.S. Pat. No. 6,024,956 Method to treat inflammation with humanized anti-IL-8-antibodies U.S. Pat. No. 6,024,955 Peptides and monoclonal antibodies U.S. Pat. No. 6,019,969 Composition comprising antibody to macrophage-derived inflammatory mediator (MIP-.alpha. 1 and MIP-1.beta.) U.S. Pat. No. 6,001,357 Method of enhancing wound healing with anti-IL-5 antibody U.S. Pat. No. 5,998,586 Interleukin-5 specific recombinant antibodies U.S. Pat. No. 5,994,511 Anti-IgE antibodies and methods of improving polypeptides U.S. Pat. Nos. 5,989,584 5,980,894 Antibodies to leukemia inhibitory factor U.S. Pat. No. 5,976,534 Inhibition of intimal hyperplasia using antibodies to PDGF receptors and heparin U.S. Pat. No. 5,959,086 Antibodies specific for human pancreatitis associated protein U.S. Pat. No. 5,955,076 Immunoactive peptides and antibodies and their use in anti-allergy treatment U.S. Pat. No. 5,925,352 Method of treating inflammation with antibodies to neutrophil chemotactic factor U.S. Pat. No. 5,925,351 Soluble lymphotoxin-.beta. receptors and anti-lymphotoxin receptor and ligand antibodies as therapeutic agents for the treatment of immunological disease U.S. Pat. No. 5,919,452 Methods of treating TNF.alpha.-mediated disease using chimeric anti-TNF antibodies U.S. Pat. No. 5,916,805 Antithrombotic agent and anti-von Willebrand factor monoclonal antibody U.S. Pat. No. 5,911,988 Method for treating asthma using SCF antibody U.S. Pat. No. 5,888,510 Chronic rheumatoid arthritis therapy containing IL-6 antagonist as effective component U.S. Pat. No. 5,876,947 Monospecific antibody reactive with Fibrinogen and fibrinopeptide B U.S. Pat. No. 5,874,080 Anti-IL-8 monoclonal antibodies for treatment of asthma U.S. Pat. No. 5,863,537 Humanized monoclonal antibodies against human interleukin-4 U.S. Pat. No. 5,853,722 Use of C5-specific antibodies for reducing immune and hemostatic dysfunctions during extracorporeal circulation U.S. Pat. No. 5,837,239 Physiologically active substance designated as epimorphin genes encoding the same and antibodies thereto U.S. Pat. No. 5,837,232 Use of an interleukin-10 antagonist to treat a B cell mediated autoimmune disorder U.S. Pat. No. 5,833,986 Methods of inhibiting the growth of neoplasia using a monoclonal antibody against .alpha. platelet derived growth factor receptor U.S. Pat. No. 5,827,517 CCK antibodies used to improve feed efficiency U.S. Pat. No. 5,807,550 Antibodies specifically recognizing somatotropin binding proteins U.S. Pat. No. 5,783,187 Treatment of cell proliferative disorders using antibodies which bind connective tissue growth factor U.S. Pat. No. 5,783,185 Monoclonal antibodies to transforming growth factor-beta and methods of use U.S. Pat. No. 5,772,998 Monoclonal antibodies to transforming growth factor-beta and methods of use U.S. Pat. No. 5,741,488 Treatment of rheumatoid arthritis with anti-CD4 antibodies in conjunction with anti-TNF antibodies U.S. Pat. No. 5,730,975 Treatment of insulin resistance in obesity linked type II diabetes using antagonist to TNF-alpha function U.S. Pat. No. 5,723,126 Methods and compositions for therapeutic uses of a fibrin-specific monoclonal antibody U.S. Pat. No. 5,712,370 Erythropoietin (EPO) peptides and antibodies directed against these U.S. Pat. No. 5,705,154 Humanized monoclonal antibodies against human interleukin-4 U.S. Pat. No. 5,702,705 Antibody methods for the treatment of a hormone-mediated disease U.S. Pat. No. 5,700,466 Method of ameliorating or preventing septic shock using a monoclonal antibody specific to cachectin/tumor necrosis factor U.S. Pat. No. 5,698,197 Monoclonal antibody specific for advanced glycosylation end products in biological samples U.S. Pat. No. 5,698,196 Antibodies specific for neutrophic chemotactic factor U.S. Pat. No. 6,498,237 Tumor necrosis factor antibodies U.S. Pat. No. 6,486,304 Antibodies and methods of making antibodies to human thyroid protein zsig45 U.S. Pat. No. 6,482,408 Fibroblast growth factor 15 antibodies U.S. Pat. No. 6,468,532 Methods of treating inflammatory diseases with anti-IL-8 antibody fragment-polymer conjugates U.S. Pat. No. 6,458,355 Methods of treating inflammatory disease with anti-IL-8 antibody fragment-polymer conjugates U.S. Pat. No. 6,451,983 Tumor necrosis factor antibodies U.S. Pat. No. 6,451,545 Monoclonal antibody specific to anti-human fibrin monomer, process for producing the same, hybridomas, and immunoassay method U.S. Pat. No. 6,451,311 Anti-procalcitonin antibodies and the preparation and use thereof U.S. Pat. No. 6,448,380 Tumor necrosis factor antibodies U.S. Pat. No. 6,440,417 Antibodies to argatroban derivatives and their use in therapeutic and diagnostic treatments U.S. Pat. No. 6,436,401 Methods for alleviating symptoms associated with diabetes and diabetic neuropathy comprising administration of low levels of antibodies U.S. Pat. No. 6,416,758 Antibody conjugate kits for selectively inhibiting VEGF U.S. Pat. No. 6,416,757 Tumor necrosis factor antibodies U.S. Pat. No. 6,413,726 Antibodies that specifically bind Cytostatin III U.S. Pat. No. 6,395,274 Monoclonal antibodies specific for human interleukin-5 U.S. Pat. No. 6,383,484 Antibodies to truncated VEGF-D and thereof U.S. Pat. No. 6,375,949 Monoclonal antibody recognizing serum amyloid A U.S. Pat. No. 6,365,155 Ancrod-specific monoclonal antibodies, antibody fragments, mixtures or derivatives thereof and use of the same U.S. Pat. No. 6,355,245 C5-specific antibodies for the treatment of inflammatory diseases U.S. Pat. No. 6,346,246 Antibodies that bind human haemopoietic maturation factor U.S. Pat. No. 6,342,219 Antibody compositions for selectively inhibiting VEGF U.S. Pat. No. 6,331,301 Antibodies specific for vascular endothelial growth factor-B U.S. Pat. No. 6,328,963 Anti-human MP52 monoclonal antibody U.S. Pat. No. 6,300,477 Antibodies to human cystatin E U.S. Pat. No. 6,294,655 Anti-interleukin-1 receptor antagonist antibodies and uses thereof U.S. Pat. No. 6,280,731 Antithrombotic agent and anti-von willebrand factor monoclonal antibody U.S. Pat. No. 6,280,730 Antibodies that bind to .alpha.2-antiplasmin crosslinked to fibrin which do not inhibit plasma .alpha.2-antiplasmin U.S. Pat. No. 6,274,142 Methods of neutralizing coagulation with anti-tissue factor antibodies U.S. Pat. No. 6,270,766 Anti-TNF antibodies and methotrexate in the treatment of arthritis and crohn's disease U.S. Pat. No. 6,261,790 Monoclonal antibodies and antibody cocktail for detection of prion protein as an indication of transmissible spongiform encephalopathies U.S. Pat. No. 6,261,559 Methods of treating asthma with interleukin-9 antibodies U.S. Pat. No. 6,251,393 Conformation-specific anti-von Willebrand Factor antibodies U.S. Pat. No. 6,238,666 RANTES homolog antibody U.S. Pat. No. 6,235,281 Use of anti-TNF antibodies as drugs for the treatment of disorders with an elevated serum level of interleukin-6 U.S. Pat. No. 6,232,447 Antibody immunoreactive with a human cytokine designated LERK-6 U.S. Pat. No. 6,228,360 Antithrombotic agent and humanized anti-von Willebrand factor monoclonal antibody U.S. Pat. No. 6,218,516 Antibodies specific for the extracellular domain of NYK/flk-1 protein and uses thereof U.S. Pat. No. 6,200,559 Use of antibodies against MxA or MxB to determine levels of type I interferons in vivo U.S. Pat. No. 6,193,969 Antibody fragments in therapy U.S. Pat. No. 6,184,359 Antibodies to epithelium-derived T-cell factor U.S. Pat. No. 6,180,102 Monoclonal antibody to human Mx protein MxA U.S. Pat. No. 6,168,791 Antibodies that bind immunomodulators U.S. Pat. No. 6,165,465 Immunologic enhancement of the somatogenic effect of somatotropin with an antibody U.S. Pat. No. 6,143,874 Antibodies to the neurotrophic factor NNT-1 U.S. Pat. No. 6,133,426 Humanized anti-IL-8 monoclonal antibodies U.S. Pat. No. 6,124,439 OB polypeptide antibodies and method of making U.S. Pat. No. 6,117,985 Antibody compositions for preparing enriched cell preparations U.S. Pat. No. 6,117,980 Humanized anti-IL-8 monoclonal antibodies U.S. Pat. No. 6,114,507 Anti-Fas ligand antibody and assay method using the anti-Fas ligand antibody U.S. Pat. No. 6,113,897 Antibodies and their use 20020187150 Preventive and/or therapeutic agent for systemic lupus erythematosus comprising anti-IL-6 receptor antibody as an active ingredient 20020176858 Monoclonal antibodies to TGF-BETA 20020172677 Method to inhibit airway hyperresponsiveness using aerosolized T cell receptor antibodies 20020164327 Pharmaceutical composition of F(ab)2 antibody fragments and a process for the preparation thereof 20020160010 USE OF PREPARATIONS CONTAINING ANTI-CD44 ANTIBODIES IN THE TREATMENT OF CERTAIN TUMOURS AND THE SUPPRESSION OF IMMUNE REACTIONS 20020150882 Antibody specific to CD14 and uses thereof 20020146419 Methods of treating ankylosis with chimeric anti-TNF antibodies 20020141996 Methods of treating infections with chimeric anti-TNF antibodies 20020136723 Anti-TNF antibodies and methotrexate in the treatment of autoimmune disease 20020122797 ANTIBODIES TO VASCULAR ENDOTHELIAL CELL GROWTH FACTOR 20020119153 Antibody conjugate formulations for selectively inhibiting VEGF 20020119152 Anti-TNF alpha antibodies in therapy of asthma 20020114805 Methods of treating myelodysplastic syndrome with anti-TNF antibodies 20020106372 Anti-TNF antibodies and peptides of human tumor necrosis factor 20020098527 Gene recombinant antibody and antibody fragment thereof 20020091240 Humanized antibodies to gamma-interferon 20020082396 Reshaped human antibody to human interleukin-8 20020081300 METHODS FOR ALLEVIATING SYMPTOMS ASSOCIATED WITH DIABETES AND DIABETIC NEUROPATHY COMPRISING ADMINISTRATION OF LOW LEVELS OF ANTIBODIES 20020064528 Antibodies specific to KDR and uses thereof 20020051782 Anti-CCR2 antibodies and methods of use therefor 20020028204 Antithrombotic agent and anti-von willebrand factor monoclonal antibody 20020025317 Bispecific monoclonal antibodies to IL-12 and IL-18 20020022720 Anti-TNF antibodies and peptides of human tumor necrosis factor 20020015700 Anti-CCR2 antibodies and methods of use therefor 20020012909 Small functional units of antibody heavy chain variable regions 20020012665 Combined use of anti-cytokine antibodies or antagonists and anti-CD20 for treatment of B cell lymphoma 20020006405 SEPSIS REMEDY COMPRISING ANTI-IL-8 ANTIBODY AS ACTIVE INGREDIENT 20010019833 TNF receptor-specific antibodies 20010018052 Use of anti-coagulation factor antibodies as long-lasting protective agents 20010010819 Use of anti-TNF antibodies as drugs for the treatment of disorders with an elevated serum level of interleukin-6 20010006637 RHEUMATOID ARTHRITIS REMEDY CONTAINING ANTI-IL-8 ANTIBODY AS ACTIVE INGREDIENT U.S. Pat. No. 5,698,195 Methods of treating rheumatoid arthritis using chimeric anti-TNF antibodies U.S. Pat. No. 5,688,505 Method for treating cancer with monoclonal antibodies to oncofetal protein U.S. Pat. No. 5,670,150 Non-depleting CD4-specific monoclonal antibodies for the treatment of insulin-dependent diabetes mellitus (IDDM) U.S. Pat. No. 5,662,904 Anti-scarring compositions comprising growth factor neutralizing antibodies U.S. Pat. No. 5,656,272 Methods of treating TNF-.alpha.-mediated Crohn's disease using chimeric anti-TNF antibodies U.S. Pat. No. 5,654,407 Human anti-TNF antibodies U.S. Pat. No. 5,635,178 Inhibition of complement mediated inflammatory response using monoclonal antibodies specific for a component forming the C56-9 complex which inhibit the platelet or endothelial cell activating function of the C56-9 complex U.S. Pat. No. 5,627,073 Hybridomas producing antibodies to cardiac hypertrophy factor U.S. Pat. No. 5,616,321 Method of treating bacterial meningitis with anti-tumor necrosis factor antibody U.S. Pat. No. 5,610,281 Antibodies for modulating heterotypic E-cadherin interactions with human T lymphocytes U.S. Pat. No. 5,601,821 Immunoactive peptides and antibodies and their use in anti-allergy treatment U.S. Pat. No. 5,599,708 Osteoclast growth regulating factors and antibodies U.S. Pat. No. 5,582,862 Antibodies that bind to .alpha.2-antiplasmin crosslinked to fibrin which do not inhibit plasma .alpha.2-antiplasmin U.S. Pat. No. 5,580,561 Methods and pharmaceutical compositions for blocking suppression of immune defense mechanisms using an antibody, a factor, or an antisense peptide U.S. Pat. No. 5,573,762 Use of leukemia inhibitory factor specific antibodies and endothelin antagonists for treatment of cardiac hypertrophy U.S. Pat. No. 5,571,714 Monoclonal antibodies which bind both transforming growth factors beta. 1 and .beta.2 and methods of use U.S. Pat. No. 5,559,012 Therapeutic, IL-6 antibody kits, and process for their preparation U.S. Pat. No. 5,543,144 Treating hypersensitivities with anti-IGE monoclonal antibodies which bind to IGE-expressing B cells but not basophils U.S. Pat. No. 5,520,914 Antibodies to angiogenin: immunotherapeutic agents U.S. Pat. No. 5,496,549 Bispecific monoclonal antibodies, thrombolytic agent and method of cell lysis U.S. Pat. No. 5,487,892 Method for treating thrombotic disease using a fibrin specific monoclonal antibody U.S. Pat. No. 5,478,926 Monoclonal antibody against human IgE U.S. Pat. No. 5,334,380 Anti-endotoxin, interleukin-1 receptor antagonist and anti-tumor necrosis factor antibody with arginine-free formulations for the treatment of hypotension U.S. Pat. No. 5,279,956 Activated protein C polypeptides and anti-peptide antibodies, diagnostic methods and systems for inhibiting activated protein C U.S. Pat. No. 5,237,054 Stabilized aqueous composition containing antibodies U.S. Pat. No. 5,225,540 Monoclonal antibodies to tissue plasminogen activator (T-PA) which prolong its functional half-life U.S. Pat. No. 5,196,324 Monoclonal antibodies reactive with a human atheroma associated antigen U.S. Pat. No. 5,183,657 Antibodies for use in antilymphocyte antibody therapy U.S. Pat. No. 5,149,786 Dopamine releasing protein and antibody U.S. Pat. No. 5,124,439 Antibodies against fibrin; immunogenic peptides suitable for preparing the antibodies, method for determining fibrin and pharmaceutical preparation based on the antibodies U.S. Pat. No. 5,120,834 Fibrin-specific monoclonal antibody U.S. Pat. No. 5,116,613 Antibody-thrombolytic agent product and method of use U.S. Pat. No. 5,109,115 Monoclonal antibody specific for bombesin U.S. Pat. No. 5,055,289 Interferon antibody therapeutic compositions having an extended serum half-life U.S. Pat. No. 4,963,657 Monoclonal antibodies to the light chain region of human factor XII and methods of preparing and using the same U.S. Pat. No. 4,940,782 Monoclonal antibodies against IgE-associated determinants, hybrid cell lines producing these antibodies, and use therefore U.S. Pat. No. 4,927,916 Method of producing fibrin-specific monoclonal antibodies lacking fibrinogen-cross-reactivity using fibrin-specific peptides U.S. Pat. No. 4,916,070 Fibrin-specific antibodies and method of screening for the antibodies U.S. Pat. No. 4,912,201 Monoclonal antibodies and antisera to intragonadal regulatory protein U.S. Pat. No. 4,879,112 Monoclonal antibodies agaisnt GnRH U.S. Pat. No. 4,851,334 Monoclonal antibodies specific to in vivo fragments derived from human fibrinogen, human fibrin I or human fibrin II U.S. Pat. No. 4,780,401 Novel monoclonal antibodies to human renin and hybridoma cells, processes for their preparation and their applications U.S. Pat. No. 4,764,475 Pancreas dependant immunoassay for determining subpopulations of monoclonal antibodies to somatostatin. U.S. Pat. No. 4,676,981 Monoclonal antibodies against GnRH U.S. Pat. No. 4,599,229 Method of promoting animal growth using antibodies against somatostatin U.S. Pat. No. 4,565,687 Monoclonal antibodies specific for the unbound beta. subunit of human chorionic gonadotropin

### Appendix B

20020028436 Anti-CCR2 antibodies and methods of use therefor 20020028178 Treatment of B cell malignancies using combination of B cell depleting antibody and immune modulating antibody related applications 20020018775 MONOCLONAL ANTIBODIES TO STEM CELL FACTOR RECEPTORS 20020015703 Antagonists of tweak and of tweak receptor and their use to treat immunological disorders 20020004587 Multivalent antibodies and uses therefor 20010041178 Preventing airway mucus production by administration of EGF-R antagonists 20010036919 Preventing airway mucus production by administration of EGF-R antagonists 20010036459 Enhancement of antibody-mediated immune responses 20020028436 Anti-CCR2 antibodies and methods of use therefor 20020028178 Treatment of B cell malignancies using combination of B cell depleting antibody and immune modulating antibody related applications 20020018775 MONOCLONAL ANTIBODIES TO STEM CELL FACTOR RECEPTORS 20020015703 Antagonists of tweak and of tweak receptor and their use to treat immunological disorders 20020004587 Multivalent antibodies and uses therefor 20010041178 Preventing airway mucus production by administration of EGF-R antagonists 20010036919 Preventing airway mucus production by administration of EGF-R antagonists 20010036459 Enhancement of antibody-mediated immune responses U.S. Pat. No. 5,736,138 Monoclonal antibodies with specific binding against membrane proteins on human cells, and pharmaceutical compositions containing them U.S. Pat. No. 5,736,137 Therapeutic application of chimeric and radiolabeled antibodies to human B lymphocyte restricted differentiation antigen for treatment of B cell lymphoma U.S. Pat. No. 5,730,979 LO-CD2a antibody and uses thereof for inhibiting T cell activation and proliferation U.S. Pat. No. 5,730,978 Inhibition of lymphocyte adherence with .alpha.4.beta.1-specific antibodies U.S. Pat. No. 5,730,975 Treatment of insulin resistance in obesity linked type II diabetes using antagonist to TNF-alpha function U.S. Pat. No. 5,725,856 Monoclonal antibodies directed to the HER2 receptor U.S. Pat. No. 5,720,954 Monoclonal antibodies directed to the HER2 receptor U.S. Pat. No. 5,717,073 Anti-gp130 monoclonal antibodies U.S. Pat. No. 5,717,072 Antibodies that are immunoreactive with interleukin-4 receptors U.S. Pat. No. 5,714,146 IL-4 bone therapy U.S. Pat. No. 5,709,858 Antibodies specific for Rse receptor protein tyrosine kinase U.S. Pat. No. 5,705,157 Methods of treating cancerous cells with anti-receptor antibodies U.S. Pat. No. 5,693,322 Enhanced intercellular interaction by associational antibody molecules U.S. Pat. No. 5,690,933 Monoclonal antibodies for inducing tolerance U.S. Pat. No. 5,688,504 Anti-receptor and growth blocking antibodies to the vitamin B.sub.12/transcobalamin II receptor and binding sites U.S. Pat. No. 5,686,292 Hepatocyte growth factor receptor antagonist antibodies and uses thereof U.S. Pat. No. 5,683,693 Method for inducing T cell unresponsiveness to a tissue or organ graft with anti-CD40 ligand antibody or soluble CD40 U.S. Pat. No. 5,679,346 Methods of blocking adhesion with anti-lami-3 antibody U.S. Pat. No. 5,670,150 Non-depleting CD4-specific monoclonal antibodies for the treatment of insulin-dependent diabetes mellitus (IDDM) U.S. Pat. No. 5,667,978 Antibody to the neural cell adhesion molecule and methods of use U.S. Pat. No. 5,667,781 Enhanced inhibition of tumor cell proliferation using a combination of two monoclonal antibodies to the human transferrin receptor U.S. Pat. No. 5,652,110 Antibodies to .alpha.v.beta.3 integrin U.S. Pat. No. 5,652,109 Antibodies to .alpha.v.beta.3 integrin U.S. Pat. No. 5,651,969 Antibody to the neural cell adhesion molecule and methods of use U.S. Pat. No. 5,635,601 Beta-8 integrin subunit antibodies U.S. Pat. No. 5,635,177 Protein tyrosine kinase agonist antibodies U.S. Pat. No. 5,632,991 Antibodies specific for E-selectin and the uses thereof U.S. Pat. No. 5,622,701 Cross-reacting monoclonal antibodies specific for E- and P-selectin U.S. Pat. No. 5,620,889 Human anti-Fas IgG1 monoclonal antibodies U.S. Pat. No. 5,620,687 Inhibition of intimal hyperplasia using antibodies to PDGF beta receptors U.S. Pat. No. 5,582,826 Monoclonal antibodies which bind the gamma chain of human interleukin-2 receptor U.S. Pat. No. 5,578,704 Antibody to osteoclast alphavbeta3 ntegrin U.S. Pat. No. 5,571,513 Anti-gp130 monoclonal antibodies U.S. Pat. No. 5,558,864 Humanized and chimeric anti-epidermal growth factor receptor monoclonal antibodies U.S. Pat. No. 5,545,405 Method for treating a mammal suffering from cancer with a cho-glycosylated antibody U.S. Pat. No. 5,545,404 Method for treating a mammal suffering from a T-cell medicated disorder with a CHO-Glycosylated antibody U.S. Pat. No. 5,545,403 Method for treating a mammal by administering a CHO-glycosylated antibody U.S. Pat. No. 5,498,499 Peptides and antibodies that inhibit platelet adhesion U.S. Pat. No. 5,486,361 Hybridomas and monoclonal antibodies that speifically bind to GPIB on platelets and inhibit the binding of thrombin to platelets U.S. Pat. No. 5,474,771 Murine monoclonal antibody (5c8) recognizes a human glycoprotein on the surface of T-lymphocytes, compositions containing same U.S. Pat. No. 5,424,066 Method for increasing CD4+ cell numbers through the use of monoclonal antibodies directed against self-reactive, CD4 specific cytotoxic T-cells U.S. Pat. No. 5,418,135 Method of inhibiting binding of PDGF to a PDGF receptor by biosynthetic PDGF antagonists U.S. Pat. No. 5,378,464 Modulation of inflammatory responses by administration of GMP-140 or antibody to GMP-140 U.S. Pat. No. 5,359,037 Antibodies to TNF binding protein I U.S. Pat. No. 5,273,743 Trifunctional antibody-like compounds as a combined diagnostic and therapeutic agent U.S. Pat. No. 5,223,426 Monoclonal antibodies reactive with defined regions of the T-cell antigen receptor U.S. Pat. No. 5,182,107 Transferrin receptor specific antibody-neuropharmaceutical or diagnostic agent conjugates U.S. Pat. No. 5,101,017 Antibodies for providing protection against P. vivax malaria infection U.S. Pat. No. 5,084,391 Monoclonal antibody to the interleukin-2-receptor and its use U.S. Pat. No. 5,028,424 Antibodies to receptor and antigen for natural killer and non-specific cytotoxic cells U.S. Pat. No. 6,056,956 Non-depleting anti-CD4 monoclonal antibodies and tolerance induction U.S. Pat. No. 6,051,228 Antibodies against human CD40 U.S. Pat. No. 6,048,526 Monoclonal antibodies reactive with defined regions of the T cell antigen receptor U.S. Pat. No. 6,033,876 Anti-CD30 antibodies preventing proteolytic cleavage and release of membrane-bound CD30 antigen U.S. Pat. No. 6,030,796 Monoclonal antibody to a human MDR1 multidrug resistance gene product, and uses U.S. Pat. No. 6,015,560 Antibody recognizing endothelial cell ligand for leukocyte CR3 6,015,559 Fas antagonists U.S. Pat. No. 6,015,555 Transferrin receptor specific antibody-neuropharmaceutical or diagnostic agent conjugates U.S. Pat. No. 6,011,138 Gamma-1 anti-human CD23 monoclonal antibodies U.S. Pat. No. 6,007,816 Methods of using CD44-specific antibodies U.S. Pat. No. 6,004,552 Methods of blocking B cell proliferation using anti-CD40 monoclonal antibodies U.S. Pat. No. 6,001,358 Humanized antibodies to human gp39, compositions containing thereof U.S. Pat. No. 6,001,356 Method of inhibiting tissue destruction in autoimmune disease using anti-CD44 antibodies U.S. Pat. No. 5,997,867 Method of using humanized antibody against CD18 U.S. Pat. No. 5,997,866 Panel of antibodies for detecting cadherins, catenins and plaque proteins in tissues and method of using the panel U.S. Pat. No. 5,997,865 Agonist antibodies against the flk2/flt3 receptor and uses thereof U.S. Pat. No. 5,994,515 Antibodies directed against cellular coreceptors for human immunodeficiency virus and methods of using the same U.S. Pat. No. 5,993,816 Methods to inhibit humoral immune responses, immunoglobulin production and B cell activation with 5c8-specific antibodies U.S. Pat. No. 5,985,280 Diagnosis and/or therapy of tumours using monoclonal antibodies specific for the human IL-4 receptor U.S. Pat. No. 5,985,279 Humanized antibody against CD18 U.S. Pat. No. 5,985,278 Anti-.alpha.V-integrin monoclonal antibody U.S. Pat. No. 5,985,276 Destruction of contaminating tumor cells in stem cell transplants using bispecific antibodies U.S. Pat. No. 5,980,893 Agonist murine monoclonal antibody as a stimulant for megakaryocytopoiesis U.S. Pat. No. 5,980,892 Monoclonal antibodies reactive with defined regions of the T cell antigen receptor U.S. Pat. No. 5,976,533 Monoclonal antibodies reactive with defined regions of the T cell antigen receptor U.S. Pat. No. 5,976,532 Method of antithrombotic therapy using anti-GPIIb/IIIa antibodies or fragments thereof, including c7E3 U.S. Pat. No. 5,968,512 Antibody recognizing endothelial cell ligand for leukocyte CR3 U.S. Pat. No. 5,968,511 ErbB3 antibodies U.S. Pat. No. 5,961,974 Monoclonal antibodies to CD40 ligand, pharmaceutical composition comprising the same and hybridomas producing the same U.S. Pat. No. 5,959,084 Bispecific antibodies, methods of production and uses thereof U.S. Pat. No. 5,958,410 Therapy of sarcoidosis U.S. Pat. No. 5,951,983 Methods of inhibiting T cell mediated immune responses with humanized LO-CD2A-specific antibodies U.S. Pat. No. 5,951,982 Methods to suppress an immune response with variant CD44-specific antibodies U.S. Pat. No. 5,942,229 Method for prolonged suppression of humoral immune response to a thymus-dependent antigen therapeutic agent U.S. Pat. No. 5,925,351 Soluble lymphotoxin-.beta. receptors and anti-lymphotoxin receptor and ligand antibodies as therapeutic agents for the treatment of immunological disease U.S. Pat. No. 5,922,847 Methods of purifying hematopoietic cells using an antibody to a stem cell factor receptor U.S. Pat. No. 5,919,911 Monoclonal antibodies to stem cell factor receptors U.S. Pat. No. 5,919,453 Monoclonal antibodies against the interferon receptor, with neutralizing activity against type I interferon U.S. Pat. No. 5,916,561 Monoclonal antibody against CD44v6 U.S. Pat. No. 5,914,112 Anti-CD 18 antibodies in stroke U.S. Pat. No. 5,912,172 Endowing lymphocytes with antibody specificity U.S. Pat. No. 5,906,936 Endowing lymphocytes with antibody specificity U.S. Pat. No. 5,902,585 Methods of inducing T cell unresponsiveness to donor tissue or organ in a recipient with GP39 antagonists U.S. Pat. No. 5,902,584 Antibodies which bind the G-CSF receptor extracelluar domain and methods of treatment U.S. Pat. No. 5,897,861 Bispecific reagents for AIDS therapy U.S. Pat. No. 5,891,996 Humanized and chimeric monoclonal antibodies that recognize epidermal growth factor receptor (EGF-R); diagnostic and therapeutic use U.S. Pat. No. 5,891,434 Monoclonal antibodies to the APO-1 antigen U.S. Pat. No. 5,889,160 Human IL-2 receptor .gamma.-chain molecule antibody U.S. Pat. No. 5,888,508 Monoclonal antibodies against leukocyte adhesion receptor .beta.-chain methods of producing these antibodies and use therefore U.S. Pat. No. 5,885,575 Antibodies that react with variant CD44 surface proteins U.S. Pat. No. 5,885,574 Antibodies which activate an erythropoietin receptor U.S. Pat. No. 5,885,573 Methods and materials for modulation of the immunosuppressive activity and toxicity of monoclonal antibodies U.S. Pat. No. 5,882,644 Monoclonal antibodies specific for the platelet derived growth factor beta. receptor and methods of use thereof U.S. Pat. No. 5,880,267 Trophinin and trophinin-assisting protein agonists, antibodies and antagonists U.S. Pat. No. 5,876,950 Monoclonal antibodies specific for different epitopes of human GP39 and methods for their use in diagnosis and therapy U.S. Pat. No. 5,876,718 Methods of inducing T cell non-responsiveness to transplanted tissues and of treating graft-versus-host-disease with anti-gp39 antibodies U.S. Pat. No. 5,874,082 Humanized anti-CD40 monoclonal antibodies and fragments capable of blocking B cell proliferation U.S. Pat. No. 5,871,732 Anti-CD4 antibody homologs useful in prophylaxis and treatment of AIDS, ARC and HIV infection U.S. Pat. No. 5,869,049 Methods of inducing T cell unresponsiveness to bone marrow with gp39 antagonists U.S. Pat. No. 5,863,796 Antibodies which specifically bind mammalian receptors for interleukin-10 (IL-10) U.S. Pat. No. 5,856,448 Antibodies specifically reactive with thrombin receptor and its components U.S. Pat. No. 5,853,721 Antibody to interleukin-12 receptor U.S. Pat. No. 5,852,176 Antibodies to receptors for human interleukin-12 U.S. Pat. No. 5,846,535 Methods for reducing tumor cell growth by using antibodies with broad tumor reactivity and limited normal tissue reactivity U.S. Pat. No. 5,844,093 Anti-EGFR single-chain Fvs and anti-EGFR antibodies U.S. Pat. No. 5,843,441 Use of endothelial-leukocyte adhesion molecule-1 specific antibodies in the treatment of asthma U.S. Pat. No. 5,843,439 Therapeutic application of chimeric and radiolabeled antibodies to human B lymphocyte restricted differentiation antigen for treatment of B cell lymphoma U.S. Pat. No. 5,843,398 Radioimmunotherapy of lymphoma using anti-CD20 antibodies U.S. Pat. No. 5,840,301 Methods of use of chimerized, humanized, and single chain antibodies specific to VEGF receptors U.S. Pat. No. 5,840,299 Humanized antibodies against leukocyte adhesion molecule VLA-4 U.S. Pat. No. 5,833,988 Transferrin receptor specific antibody-neuropharmaceutical or diagnostic agent conjugates U.S. Pat. No. 5,833,986 Methods of inhibiting the growth of neoplasia using a monoclonal antibody against alpha. platelet derived growth factor receptor U.S. Pat. No. 5,830,471 Methods and compositions comprising anti-lam-1 antibodies U.S. Pat. No. 5,830,469 Fas antagonists and uses thereof U.S. Pat. No. 5,824,311 Treatment of tumors with monoclonal antibodies against oncogene antigens U.S. Pat. No. 5,821,123 Modified antibody variable domains U.S. Pat. No. 5,820,859 Method of targeting a therapeutic agent to cells expressing the erb B-3 receptor U.S. Pat. No. 5,817,311 Methods of inhibiting T-cell medicated immune responses with LO-CD2a-specific antibodies U.S. Pat. No. 5,808,002 Stem cell factor receptor-(c-kit)-specific monoclonal antibody A3C6E2 U.S. Pat. No. 5,800,815 Antibodies to P-selectin and their uses U.S. Pat. No. 5,795,572 Monoclonal antibodies and FV specific for CD2 antigen U.S. Pat. No. 5,788,966 Antibody which is directed against and inhibits collagen binding to a VLA-I epitope and uses thereof U.S. Pat. No. 5,785,967 Antibodies immunoreactive with leukemia inhibitory factor receptors U.S. Pat. No. 5,783,186 Antibody-induced apoptosis U.S. Pat. No. 5,780,027 Methods of treatment of down syndrome by interferon antagonists U.S. Pat. No. 5,777,084 Antibody BV10A4H2 specific for human FLT3/FLK2 receptor and mybridoma U.S. Pat. No. 5,776,457 Antibodies to human PF4A receptor and compositions thereof U.S. Pat. No. 5,776,456 Therapeutic application of chimeric and radiolabeled antibodies to human B lymphocyte restricted differentiation antigen for treatment of B cell lymphoma U.S. Pat. No. 5,772,997 Monoclonal antibodies directed to the HER2 receptor U.S. Pat. No. 5,770,196 Modified antibody variable domains and therapeutic uses thereof U.S. Pat. No. 5,770,195 Monoclonal antibodies directed to the her2 receptor U.S. Pat. No. 5,766,947 Monoclonal antibodies reactive with an epitope of a V.beta.3.1 variable region of a T cell receptor U.S. Pat. No. 5,762,933 Method for preventing and treating graft failure in a human patient using a monoclonal antibody specific for leucocyte functional antigen LFA-1 U.S. Pat. No. 5,762,932 Combined treatment of iron depletion and IgG antibody U.S. Pat. No. 5,756,095 Antibodies with specificity for a common epitope on E-selectin and L-selectin U.S. Pat. No. 5,753,225 Antibodies that mimic actions of neurotrophins U.S. Pat. No. 5,747,037 Anti-GP39 antibodies U.S. Pat. No. 5,747,032 Monoclonal antibody to human granulocyte macrophage colony stimulating factor U.S. Pat. No. 5,741,488 Treatment of rheumatoid arthritis with anti-CD4 antibodies in conjunction with anti-TNF antibodies U.S. Pat. No. 5,739,289 Monoclonal antibody to human cell adhesion molecule U.S. Pat. No. 4,954,617 Monoclonal antibodies to FC receptors for immunoglobulin G on human mononuclear phagocytes U.S. Pat. No. 4,943,533 Hybrid cell lines that produce monoclonal antibodies to epidermal growth factor receptor U.S. Pat. No. 4,935,234 Method of reducing tissue damage at an inflammatory site using a monoclonal antibody U.S. Pat. No. 4,908,203 Method for inducing HIV neutralizing antibodies using an internal image idiotope U.S. Pat. No. 4,863,726 Combination therapy using immunotoxins with interleukin-2 U.S. Pat. No. 4,859,449 Modified antibodies for enhanced hepatocyte clearance U.S. Pat. No. 4,840,793 Method of reducing tissue damage at an inflammatory site using a monoclonal antibody U.S. Pat. No. 4,797,277 Method for reperfusion therapy U.S. Pat. No. 4,786,590 Diagnostic and therapeutic aspects of receptor-mediated leukemogenesis U.S. Pat. No. 4,550,086 Monoclonal antibodies that recognize human T cells U.S. Pat. No. 4,434,156 Monoclonal antibodies specific for the human transferrin receptor glycoprotein U.S. Pat. No. 6,291,239 Monoclonal antibody U.S. Pat. No. 6,287,537 Radioimmunotherapy of lymphoma using anti-CD20 antibodies U.S. Pat. No. 6,274,143 Methods of delaying development of HMFG-associated tumors using anti-idiotype antibody 11D10 U.S. Pat. No. 6,261,560 Method for inhibiting muscle protein proteolysis with antibodies to interleukin-6 receptor U.S. Pat. No. 6,252,050 Method for making monoclonal antibodies and cross-reactive antibodies obtainable by the method U.S. Pat. No. 6,251,395 Methods of inhibiting inflammation at the site of a central nervous system injury with alphaD-specific antibodies U.S. Pat. No. 6,232,123 Monoclonal antibodies against leucocyte-specific G protein-coupled receptors U.S. Pat. No. 6,217,866 Monoclonal antibodies specific to human epidermal growth factor receptor and therapeutic methods employing same U.S. Pat. No. 6,214,344 Hepatocyte growth factor receptor antagonists and uses thereof U.S. Pat. No. 6,214,342 Method for increasing mean survival times of transplants with LFA-1-specific antibodies U.S. Pat. No. 6,210,671 Humanized antibodies reactive with L-selectin U.S. Pat. No. 6,210,670 Cross-reacting monoclonal antibodies specific for E-selectin and P-selectin U.S. Pat. No. 6,210,668 Destruction of contaminating tumor cells in stem cell transplants using bispecific antibodies U.S. Pat. No. 6,207,156 Specific antibodies and antibody fragments U.S. Pat. No. 6,207,155 Method of eosinophil depletion with antibody to CCR 3 receptor U.S. Pat. No. 6,207,152 Hepatocyte growth factor receptor antagonists and uses thereof U.S. Pat. No. 6,200,567 Therapeutic agents as cytokine antagonists and agonists U.S. Pat. No. 6,200,559 Use of antibodies against MxA or MxB to determine levels of type I interferons in vivo U.S. Pat. No. 6,197,297 Monoclonal antibody specific to polypeptide which induces interferon-gamma production U.S. Pat. No. 6,187,308 Monoclonal antibodies against leukocyte adhesion receptor .beta.-chain, methods of producing these antibodies and use therefor U.S. Pat. No. 6,183,744 Immunotherapy of B-cell-malignancies using anti-CD22 antibodies U.S. Pat. No. 6,177,078 Monoclonal antibody antagonists to IL-3 U.S. Pat. No. 6,171,588 Anti-.alpha.v.beta.3 integrin antibody antagonists U.S. Pat. No. 6,165,464 Monoclonal antibodies directed to the HER2 receptor U.S. Pat. No. 6,160,099 Anti-human .alpha..sub.v .beta..sub.3 and .alpha..sub.v .beta..sub.5 antibodies U.S. Pat. No. 6,143,873 Monoclonal anti-multiple drug resistance antibody, methods of production and uses thereof U.S. Pat. No. 6,143,559 Methods for the production of chicken monoclonal antibodies U.S. Pat. No. 6,143,297 Methods of promoting immunopotentiation and preparing antibodies with anti-CD3 antibodies U.S. Pat. No. 6,143,292 Allogeneic cell therapy for cancer following allogeneic stem cell transplantation U.S. Pat. No. 6,143,273 Therapeutic composition containing antibodies to soluble polypeptide fractions of LAG-3 protein U.S. Pat. No. 6,136,309 Antibodies against the interferon (IFN) alpha./.beta. receptor (IFNAR2) that preferentially block the activity of IFN-.alpha. U.S. Pat. No. 6,129,915 Epidermal growth factor receptor antibodies U.S. Pat. No. 6,123,939 Anti-neoplastic drugs in cancer therapy U.S. Pat. No. 6,117,985 Antibody compositions for preparing enriched cell preparations U.S. Pat. No. 6,113,901 Methods of stimulating or enhancing the immune system with anti-CD3 antibodies U.S. Pat. No. 6,113,900 Use of IL-2 receptor-targeted therapeutics to inhibit allograft rejection and treat autoimmune disorders U.S. Pat. No. 6,113,898 Human B7.1-specific primatized antibodies and transfectomas expressing said antibodies U.S. Pat. No. 6,106,834 Use of anti-CD45 leukocyte antigen antibodies for immunomodulation U.S. Pat. No. 6,106,833 Bispecific antibodies, methods of production and use thereof U.S. Pat. No. 6,099,841 Hepatocyte growth factor receptor agonists and uses thereof U.S. Pat. No. 6,099,838 Pharmaceutical compositions comprising anti-CD45RB antibodies for the inhibition of T-cell mediated immune responses U.S. Pat. No. 6,096,311 Methods for use of monoclonal antibodies specific for the high affinity Fc receptor for immunoglobulin G U.S. Pat. No. 6,090,365 Radioimmunotherapy of lymphoma using anti-CD20 antibodies U.S. Pat. No. 6,086,877 Therapeutic agent for rheumatic disease U.S. Pat. No. 6,086,874 Antitumor agent effect enhancer containing IL-6 antagonist U.S. Pat. No. 6,084,075 Agonist and antagonist antibodies to the chemokine receptor-2 (CCR2) U.S. Pat. No. 6,083,904 Therapeutic and diagnostic methods and compositions based on notch proteins and nucleic acids U.S. Pat. No. 6,072,037 Antibodies to the IL-17 receptor U.S. Pat. No. 6,066,719 Antibody fragments U.S. Pat. No. 6,056,959 CD40 antigen antibody complex U.S. Pat. No. 6,506,383 Methods of suppressing immune responses to transplanted tissues and organs with gp39-specific antibodies U.S. Pat. No. 6,506,382 Method for inhibiting reperfusion injury using antibodies to P-selectin glycoprotein ligand U.S. Pat. No. 6,500,428 Antibodies to a lymphocyte surface receptor that binds CAML and methods of use thereof U.S. Pat. No. 6,491,916 Methods and materials for modulation of the immunosuppresive activity and toxicity of monoclonal antibodies U.S. Pat. No. 6,491,915 Anti-CCR2 antibodies and methods of use therefor U.S. Pat. No. 6,488,930 Anti-CCR4 antibodies and methods of use therefor U.S. Pat. No. 6,461,612 Anti-idiotypic antibody and its use in diagnosis and therapy in HIV-related disease U.S. Pat. No. 6,458,933 Therapeutic using a bispecific antibody U.S. Pat. No. 6,458,356 Antibody-induced apoptosis U.S. Pat. No. 6,458,353 Anti-CCR2 antibodies and methods of use therefor U.S. Pat. No. 6,455,043 Combination therapies for B-cell lymphomas comprising administration of anti-CD20 antibody U.S. Pat. No. 6,455,042 Method of treating ulcerative colitis or crohn's disease by administering an antibody to.alpha.E.beta.7 integrin U.S. Pat. No. 6,451,981 Antibodies against lymphocyte-associated cell surface protein LAM-1 U.S. Pat. No. 6,451,310 Method for inhibiting an allergic response with a 5c8-specific antibody U.S. Pat. No. 6,440,418 Methods of treating autoimmune diseases with gp39-specific antibodies U.S. Pat. No. 6,440,417 Antibodies to argatroban derivatives and their use in therapeutic and diagnostic treatments U.S. Pat. No. 6,432,405 Method of inhibiting HIV infection with CD44 and anti-CD44 antibodies U.S. Pat. No. 6,432,404 Methods of inhibiting locomotor damage following spinal cord injury with .alpha. D-specific antibodies U.S. Pat. No. 6,420,126 Tumor specific internalizing antigens and methods for targeting therapeutic agents U.S. Pat. No. 6,413,514 Methods of using antibodies against human CD40 U.S. Pat. No. 6,406,695 Treatment of hepatitis C by administration of anti-IL-2 receptor monoclonal antibody U.S. Pat. No. 6,406,694 Anti-CCR2 antibodies U.S. Pat. No. 6,403,091 Methods for inhibiting the rejection of a transplant organ in a subject with 5C8-specific antibodies U.S. Pat. No. 6,399,063 Monoclonal antibodies directed to the HER2 receptor U.S. Pat. No. 6,399,061 Chimeric and radiolabelled antibodies specific to human CD20 antigen and use thereof for treatment of B-cell lymphoma U.S. Pat. No. 6,395,272 Therapeutic compounds comprised of anti-Fc receptor antibodies U.S. Pat. No. 6,392,020 Monoclonal antibody to the clonotypic structure of a T-cell receptor, a pharmaceutical compostion and a diagnostic reagent comprising the same U.S. Pat. No. 6,387,371 Monoclonal antibodies directed to the HER2 receptor U.S. Pat. No. 6,380,363 Antibodies to the Ob receptor U.S. Pat. No. 6,379,668 Use of anti-CD45 leukocyte antigen antibodies for immunomodulation U.S. Pat. No. 6,375,950 Methods for inducing T cell unresponsiveness to donor tissue or organ in a recipient with gp39 antagonists U.S. Pat. No. 6,365,157 Monoclonal antibodies specific to VEGF receptors and uses thereof U.S. Pat. No. 6,355,780 Antibodies to the death domain motifs of regulatory proteins U.S. Pat. No. 6,348,194 Tumor specific internalizing antigens and methods for targeting therapeutic agents U.S. Pat. No. 6,342,219 Antibody compositions for selectively inhibiting VEGF U.S. Pat. No. 6,340,459 Therapeutic applications for the anti-T-BAM (CD40-L) monoclonal antibody 5C8 in the treatment of reperfusion injury in non-transplant recipients U.S. Pat. No. 6,331,302 Protein tyrosine kinase agonist antibodies U.S. Pat. No. 6,329,510 Anti-CCR1 antibodies and methods of use therefor U.S. Pat. No. 6,329,159 Anti-GPR-9-6 antibodies and methods of identifying agents which modulate GPR-9-6 function U.S. Pat. No. 6,328,964 Method to treat multiple sclerosis with GP39-specific antibodies U.S. Pat. No. 6,319,499 Methods for activating an erythropoietin receptor using antibodies U.S. Pat. No. 6,315,998 Methods of blocking B-cell activation using anti-CD40 monoclonal antibodies U.S. Pat. No. 6,312,694 Cancer treatment methods using therapeutic conjugates that bind to aminophospholipids U.S. Pat. No. 6,312,693 Antibodies against human CD40 U.S. Pat. No. 6,312,692 Method of treating graft-versus-host disease with anti-GP39 antibodies and bone marrow cells U.S. Pat. No. 6,312,691 Lymphotoxin-.alpha./.beta. complexes and anti-lympotoxin-.beta. receptor antibodies as anti-tumor agents U.S. Pat. No. 6,312,690 Monoclonal recombinant anti-rhesus D (D7C2) antibody U.S. Pat. No. 6,312,689 Anti-CCR2 antibodies and methods of use therefore U.S. Pat. No. 6,309,639 Method for inhibiting an inflammatory response using antibodies to P-selectin glycoprotein ligand U.S. Pat. No. 6,306,393 Immunotherapy of B-cell malignancies using anti-CD22 antibodies

### Appendix C

Bruhl H, et alDepletion of CCR5-Expressing Cells with Bispecific Antibodies and Chemokine Toxins: A New Strategy in the Treatment of Chronic Inflammatory Diseases and HIV. J Immunol. Feb. 15, 2001; 166(4):2420-2426; Tesch H, et alTreatment of patients with malignant lymphomas with monoclonal antibodies. Bone Marrow Transplant. May 2000; 26 Suppl 2:S50-3; Jung G, Brandl M, Eisner W, Fraunberger P, Reifenberger G, Schlegel U, Wiestler O D, Reulen H J, Wilmanns W. Local immunotherapy of glioma patients with a combination of 2 bispecific antibody fragments and resting autologous lymphocytes: Evidence for in situ t-cell activation and therapeutic efficacy. Int J Cancer. Jan. 15, 2001; 91(2):225-230; Kan K S, Anderson V A, Leong W S, Smith A M, Worth A T, Stevenson G T. Thioether-Bonded Constructs of Fab′gamma and Fcgamma Modules Utilizing Differential Reduction of Interchain Disulfide Bonds J Immunol. Jan. 15, 2001; 166(2):1320-1326; Cao Y, Suresh M R. Bispecific MAb aided liposomal drug delivery. J Drug Target. 2000; 8(4):257-66; Schoonjans R, et al Mertens N. Fab chains As an efficient heterodimerization scaffold for the production of recombinant bispecific and trispecific antibody derivatives J Immunol. Dec. 15, 2000; 165(12):7050-7.; Schmiedl A, et al Expression of a bispecific dsFv-dsFv′ antibody fragment in escherichia coli Protein Eng. October 2000; 13(10):725-34. Rohrbach F, et al Construction and characterization of bispecific costimulatory molecules containing a minimized CD86 (B7-2) domain and single-chain antibody fragments for tumor targeting Clin Cancer Res. November 2000; 6(11):4314-22. Schoonjans R, Efficient heterodimerization of recombinant bi- and trispecific antibodies Bioseparation. 2000; 9(3):179-83. Wallace P K, et al Production of macrophage-activated killer cells for targeting of glioblastoma cells with bispecific antibody to FcgammaRI and the epidermal growth factor receptor Cancer Immunol Immunother. November 2000; 49(9):493-503.Bioconjug Chem. November 2000; 11(6):842-54. Conrath K E, et al Camel single-domain antibodies as modular building units in. J Biol Chem. Oct. 25, 2000 [Cancer Immunol Immunother. October 2000; 49(8):441-8. Talac R, Nelson H. Current perspectives of bispecific antibody-based immunotherapy J Biol Regul Homeost Agents. July 2000; 14(3):175-81. Tomlinson I, Holliger P. Methods for generating multivalent and bispecific antibody fragments Methods Enzymol. 2000; 326:461-79 Morimoto K, Inouye K. Application of bispecific F(ab′) fragments prepared from IgMs against carcinoembryonic antigen and alkaline phosphatase Clin Chem. September 2000; 46(9):1492-3. Cochlovius B, et al Cure of Burkitt's lymphoma in severe combined immunodeficiency mice by T cells, tetravalent CD3×CD19 tandem diabody, and CD28 costimulation. Cancer Res. Aug. 15, 2000; 60(16):4336-41. Takemura Si, et al. Construction of a diabody (small recombinant bispecific antibody) using a refolding system Protein Eng. August 2000; 13(8):583-8. Katzenwadel A, et alConstruction and in vivo evaluation of an anti-PSA×anti-CD3 bispecific antibody for the immunotherapy of prostate cancer. Anticancer Res. May-June 2000; 20(3A): 1551-5. Yoshida H, Katayose Y, Suzuki M, Matsuno S, Kudo T, Kumagai I. Functional Fv fragment of an antibody specific for CD28: Fv-mediated co-stimulation of T cells. FEBS Lett. Jul. 7, 2000; 476(3):266-71. 6 38: Zeidler R, et The Fc-region of a new class of intact bispecific antibody mediates activation of accessory cells and NK cells and induces direct phagocytosis of tumour cells. Br J Cancer. July 2000; 83(2):261-6. 0 39: Hillairet et al Enhanced targeting specificity to tumor cells by simultaneous recognition of two antigens. Bioconjug Chem. July-August 2000; 11(4):452-60. Weiner L M. Bispecific antibodies in cancer therapy. Cancer J Sci Am. May 2000; 6 Suppl 3:S265-71 Zangemeister-Wittke U, Leech S H, Olie R A, Zuo Z, et al An efficient route to the production of an IgG-like bispecific antibody. Protein Eng. May 2000; 13(5):361-7 Helfrich W, et al A rapid and versatile method for harnessing scFv antibody fragments with various biological effector functions. J Immunol Methods. Apr. 3, 2000; 237(1-2):131-45Lu D, et al Acquired antagonistic activity of a bispecific diabody directed against two different epitopes on vascular endothelial growth factor receptor 2. J Immunol Methods. Nov. 19, 1999; 230(1-2):159-71. Ohmi Y, et al Tumor-specific targeting of T helper type 1 (Th1) cells by anti-CD3× anti-c-ErbB-2 bispecific antibody. Cancer Immunol Immunother. November 1999; 48(8):456-62. Kipriyanov S M, et al G, Schuhmacher J, Cochlovius B, Von der Lieth C W, Matys E R, Little M. Bispecific tandem diabody for tumor therapy with improved antigen binding and pharmacokinetics. J Mol Biol. Oct. 15, 1999; 293(1):41-56.Segal D M, Weiner G J, Weiner L M. Bispecific antibodies in cancer therapy. Curr Opin Immunol. October 1999; 11(5):558-62 4 88: Hudson P J. Recombinant antibody constructs in cancer therapy. Curr Opin Immunol. October 1999; 11(5):548-57 Alt M, et al Novel tetravalent and bispecific IgG-like antibody molecules combining single-chain diabodies with the immunoglobulin gamma1 Fc or CH3 region. FEBS Lett. Jul. 2, 1999; 454(1-2):90-4. Kontermann R E, Muller R. Intracellular and cell surface displayed single-chain diabodies. J Immunol Methods. Jun. 24, 1999; 226(1-2):179-88. Morimoto K, Inouye K. Method for the preparation of bispecific F(ab′)2mu fragments from mouse monoclonal antibodies of the immunoglobulin M class and characterization of the fragments. J Immunol Methods. Apr. 22, 1999; 224(1-2):43-50. Chaudri Z N, et al Dual specificity antibodies using a double-stranded oligonucleotide bridge. FEBS Lett. Apr. 30, 1999; 450(1-2):23-6.: Somasundaram C, et al Development of a trispecific antibody conjugate that directs two distinct tumor-associated antigens to CD64 on myeloid effector cells. Hum Antibodies. 1999; 9(1):47-54.Robert B, et al Tumor targeting with newly designed biparatopic antibodies directed against two different epitopes of the carcinoembryonic antigen (CEA). Int J Cancer. Apr. 12, 1999; 81(2):285-91. Kreutz F T, et al Efficient bispecific monoclonal antibody purification using gradient thiophilic affinity chromatography. J Chromatogr B Biomed Sci Appl. Sep. 4, 1998; 714(2):161-70. Muller K M, et al A dimeric bispecific miniantibody combines two specificities with avidity. FEBS Lett. Jul. 31, 1998; 432(1-2):45-9. Kreutz F T, et al A new method to generate quadromas by electrofusion and FACS sorting. Hybridoma. June 1998; 17(3):267-73Production of bispecific and trispecific F(ab)2 and F(ab)3 antibody derivatives. Methods Mol Biol. 1998; 80:121-34. Merchant A M, et Protein, Nucleotide An efficient route to human bispecific IgG. Nat Biotechnol. July 1998; 16(7):677-8 1. Zhu Z, et al High level secretion of a humanized bispecific diabody from Escherichia coli. Biotechnology (NY). February 1996; 14(2):192-6. Rapid assay of phage-derived recombinant human fabs as bispecific antibodies. Biotechnology (NY). November 1995; 13(11):1221-4. McGuinness B T, et Phage diabody repertoires for selection of large numbers of bispecific antibody fragments. Nat Biotechnol. September 1996; 14(9):1149-54Helfrich W, et al Construction and characterization of a bispecific diabody for retargeting T cells to human carcinomas. Int J Cancer. Apr. 13, 1998; 76(2):232-9. Muller K M, et al The first constant domain CH1 and CL of an antibody used as heterodimerization domain for bispecific miniantibodies. FEBS Lett. Jan. 30, 1998; 422(2):259-64. 223: FitzGerald K, et al Improved tumour targeting by disulphide stabilized diabodies expressed in Pichia pastoris. Protein Eng. October 1997; 10(10): 1221-5. Coloma M J, Morrison S L. Design and production of novel tetravalent bispecific antibodies. Nat Biotechnol. February 1997; 15(2): 159-63. Hoogenboom H R. Mix and match: building manifold binding sites. Nat Biotechnol. February 1997; 15(2):125-6. Ridgway J B, et al ‘Knobs-into-holes’ engineering of antibody CH3 domains for heavy chain heterodimerization. Protein Eng. July 1996; 9(7):617-21. Reinartz H W, et al Bispecific multivalent antibody studied by real-time interaction analysis for the development of an antigen-inhibition enzyme-linked immunosorbent assay. Analyst. June 1996; 121(6):767-71. Tibben J G, et al Pharmacokinetics, biodistribution and biological effects of intravenously administered bispecific monoclonal antibody OC/TR F(ab′)2 in ovarian carcinoma patients. Int J Cancer. May 16, 1996; 66(4):477-83. de Kruif J, Logtenberg T. Leucine zipper dimerized bivalent and bispecific scFv antibodies from a semi-synthetic antibody phage display library. J Biol Chem. Mar. 29, 1996; 271(13):7630-4. Ruf J, et al The molecular recognition theory applied to bispecific antibodies . . . . Nat Med. December 1995; 1(12):1222. 447: Grant S D, et al Expression of monovalent and bivalent antibody fragments in Escherichia coli. J Hematother. October 1995; 4(5):383-8. Dubel S, et al Protein, Nucleotide Bifunctional and multimeric complexes of streptavidin fused to single chain antibodies (scFv). J Immunol Methods. Jan. 27, 1995; 178(2):201-9. Kostelny S A, et al Protein, Nucleotide Formation of a bispecific antibody by the use of leucine zippers. J Immunol. Mar. 1, 1992; 148(5):1547-53 Pack P, Pluckthun A. Miniantibodies: use of amphipathic helices to produce functional, flexibly linked dimeric FV fragments with high avidity in Escherichia coli. Biochemistry. Feb. 18, 1992; 31(6):1579-84 Smith W, et alImmunoglobulins secreted by a hybrid-hybridoma: analysis of chain assemblies. Hybridoma. February 1992; 11(1):87-98. Suresh M R,et al Bispecific monoclonal antibodies from hybrid hybridomas. Methods Enzymol. 1986; 121:210-28. Paulus H. Preparation and biomedical applications of bispecific antibodies. Behring Inst Mitt. December 1985; (78): 118-32. Brennan M, et al Preparation of bispecific antibodies by chemical recombination of monoclonal immunoglobulin GI fragments. Science. Jul. 5, 1985; 229(4708):81-3.

### Appendix D

Target cell-associated markers and their tissue distribution and antibodies thereto are well known to those skilled in the art and described in standard reference works in immunology, rheumatology, pathology, internal medicine, experimental medicine, cell biology and clinical and experimental laboratory manuals, and source works on CD antigens. For example with respect to cells of hemapoietic origin see Cell and Molecular Immunology Fourth Ed. (supra) Appendix II, Manual Of Clinical Chemical Immunology Sixth Ed. Rose N R et al Eds. Antibodies to CD antigens are described at the following exemplary websites (most of whom also publish product catalogs and lists):
http://www.biosource.com/
http://www1.amershambiosciences.com/aptrix/upp01077.nsf/Content/Products
http://www.sigmaa1drich.com/
http://www.upstatebiotech.com/
http://www.promega.com/Default.htm
http://www.accuratechemical.com/accuratechemical/
www.researchd.com/rdicdabs/cdindex.htm
http://home.labvelocity.com/scientists/index.jhtml
http://www.mh-hannover.de/aktuelles/projekte/hlda7/main/
http://southernbiotech.com/catalog.html
http://www.prosci-inc.com/
http://www.abcam.com/
http://www.usbio.net/
http://www.mabtech.com/
http://www.antibodies-probes.com/
http://www.labvision.com/
http://www.crpinc.com/products/index.html

With respect to first and second target ligands see also US Application 20020197655; US 20020168362; Methods Mol Biol 2001; 166:177-92; U.S. Pat. No. 6,071,517; U.S. Pat. No. 5,897,861; U.S. Pat. No. 6,096,311; U.S. Pat. No. 5,922,845, Journal of Immunological Methods February 2001 Vol. 248(1-2) page 1-200; Mol Immunol May 1999; 36(7):433-45; US6051227 US06143297 US05977318 US05968510 US05885796 US05885579 US05869050 Methods of blocking T-cell activation using anti-B7 monoclonal antibodies; US05851795 US05747034 US06113901 US05844095 US06090914 US05718883 Transgenic animal model for autoimmune diseases; US05855887 US05811097 US06084067 EP01073741A2 US06130316 US06068984 Antibodies to lymphocyte activation antigens uses therefor; US05766570 Lymphocyte activation antigens and thereto; US05434131 US05316920 Lymphocyte activation antigen HB 15, a member of the immunoglobulin superfamily; US06111090 Mammalian cell surface antigens; US06083751 independent cytotoxic T cells; US05977303 Mammalian cell surface antigens; US05738852 Methods of enhancing antigen-specific T cell responses US05714667, U.S. Pat. No. 6,500,674 Method for the diagnosis of brain/neurological disease using monoclonal antibodies specific for PHF-tau, hybridomas secreting them, and antigen recognition by these antibodies and their applications U.S. Pat. No. 6,294,167 Pharmaceutical compositions for immunotherapy containing antibodies which specifically recognize the MHCII antigen of a patient to be treated U.S. Pat. No. 6,258,564 Antibodies, production method of the antibodies, hybridomas which produce the antibodies, production method the hybirdomas and antigen proteins recognized by the antibodies U.S. Pat. No. 6,255,107 Antibodies, production method of the antibodies, hybridomas which produce the antibodies, production method of the hybridomas and antigen proteins recognized by the antibodies U.S. Pat. No. 6,245,897 Monoclonal antibody recognizing cell surface antigen CD14 U.S. Pat. No. 6,193,948 Antigen recognized by patients with antibody associated paraneoplastic sensory neuronopathy, DNA encoding same and uses thereof U.S. Pat. No. 6,132,980 Antibodies specific for TRP-2 a human tumor antigen recognized by cytotoxic T lymphocytes U.S. Pat. No. 6,074,833 Osteoblast and fibroblast antigen and antibodies recognizing it U.S. Pat. No. 6,008,024 Monoclonal antibodies specific for PHF-tau, hybridomas secreting them, antigen recognition by these antibodies and their applications U.S. Pat. No. 5,985,541 Peptide capable of being recognized by antibodies recognizing the C33 antigen of hepatitis C virus U.S. Pat. No. 5,925,526 Antigen recognized by patients with antibody associated cerebellar degeneration, DNA encoding same and uses thereof U.S. Pat. No. 5,861,257 Monoclonal antibodies directed against the microtubule-associated protein tau, hybridomas secreting these antibodies, antigen recognition by these monoclonal antibodies and their applications U.S. Pat. No. 5,807,705 Antigen recognized by patients with antibody associated paraneoplastic sensory neuronopathy, DNA encoding same and uses thereof U.S. Pat. No. 5,773,292 Antibodies binding portions, and probes recognizing an antigen of prostate epithelial cells but not antigens circulating in the blood U.S. Pat. No. 5,703,213 Monoclonal antibodies which recognize an adenocarcinoma cell antigen U.S. Pat. No. 5,668,013 Antigen recognized by patients with antibody associated paraneoplastic cerebellar degeneration, DNA encoding same and uses thereof U.S. Pat. No. 5,665,357 Antibodies recognizing tumor associated antigen CA 55.1 U.S. Pat. No. 5,614,371 Ri fusion antigen recognized by antibodies associated with paraneoplastic opsoclonus and methods of use thereof U.S. Pat. No. 5,603,934 Antigen recognized by patients with antibody associated paraneoplastic sensory neuronopathy U.S. Pat. No. 5,597,707 Tumor associated antigen recognized by the murine monoclonal antibody L6, its oligonucleotide sequence and methods for their use U.S. Pat. No. 5,595,738 CTAA 81AV78, the antigen recognized by human monoclonal antibody 81AV78 U.S. Pat. No. 5,556,947 Monoclonal antibody recognizing a surface molecule on a subset of antigen-stimulated T cells and on certain malignancies of T and B cell origin U.S. Pat. No. 5,411,884 Monoclonal antibody L53 which recognizes a human tumor-associated antigen U.S. Pat. No. 5,212,085 SF-25 Colon adenocarcinoma antigen, and antibodies with recognize this antigen U.S. Pat. No. 5,075,219 Monoclonal antibody which recognizes a specific glycoprotein of a human milk-fat globule membrane mucin antigen and said mucin antigen U.S. Pat. No. 4,772,552 Monoclonal antibody which recognizes a 200-220 KD antigen on natural killer cells.

### EXAMPLE

This example is presented for illustrative purposes and is not limiting. Dual affinity ligands (T84-9F11) may be produced using other antibodies, screening techniques and genetic engineering procedures that are well known to those skilled in the art.

Selection of Ligands

In a preferred embodiment of this invention a high affinity first ligand is derived from an antibody specific for a tumor antigen. In this example the antibody is T84.66A3.1A.1F2 (T84) (ATCC No. HB-8747) having a specificity for CEA antigen with a binding affinity of 2.6×10¹⁰ M/I. The lower affinity second ligand is the monoclonal antibody 9F11ESB4 (9F11) (R. Niv, Y. G. Assaraf, D. Segal, E. Pirak and Y. Reiter (2001) Int. J. Cancer 94:864-872) having a specificity for an external domain of human P-glycoprotein (Pgp). Both antibodies are murine IgG1 heavy chains and kappa light chains.

Using standard antibody molecular cloning and engineering techniques (T. Maniatis, J. Sambrook, E. F. Fritsch (eds.) (1989) Molecular Cloning: A Laboratory Manual. Cold Spring Harbor Laboratory Press, Cold Spring Harbor; C. A. K. Borrebaeck (ed) (1995) Antibody Engineering, Oxford University Press, Oxford; R. Kontermann and S Dubel (eds) (2001) Antibody Engineering, Springer-Verlag, Berlin) the cDNA sequences for both antibodies are obtained and the four variable domains amplified and cloned by PCR reactions for assembly into a bispecific single-chain antibody. Since the expression and activity of the final product may be dependent on the VL-VH or VH-VL configuration both are constructed. The constructs have the configurations of VH(T84)-15 amino acid linker-VL (T84)-15 amino acid linker-VH (9F11)-15 amino acid linker-VL (9F11)-myc tag-His tag and VL (T84)-15 amino acid linker-VH (T84)-15 amino acid linker-VL (9F11)-15 amino acid linker-VH(9F11)-myc tag-His tag. (for discussions on linker designs and single chain configurations see for example J. Kriangkum, B. Xu, L. P. Nagata, R. E. Fulton, M. R. Suresh (2001) Biomol Eng 18: 31-40; R. Kontermann and S Dubel (eds) (2001) Antibody Engineering, Springer-Verlag, Berlin) There is a ribosome-binding site and pelB leader sequence upstream of the first antibody-coding domain. Specific sets of PCR primers, each 20-30 nucleotides long, are designed for each variable domain and the two orientations.

Generation of Bispecific Single-Chain Antibodies in the VH-VL Configuration

The VH and VL domains of the T84 antibody are amplified first. For the VH domain the T84-VH-back primer anneals to the vector backbone and the T84-VH-for primer anneals in the 3′ region and introduces a unique restriction site, at the 3′ end of the VH. The VL fragment requires the primers T84-VL-back which anneals to the 5′ region of the VL domain and introduces a terminal and matching restriction site to the VH segment, the 15 amino acid linker encoding sequence and a SacI site in the VL domain and the forward primer, T84-VL-for, which anneals to the vector backbone. Amplify the VH and VL fragments with the appropriate primers using approximately 25 cycles with annealing temperatures of 50-55 C. The products are about 350 bp in size and should be gel purified. Digest the amplified products with the appropriate enzymes in accordance with the manufacturers instructions. SfiI and NotI are used to digest the vector pUC119mycHis (N. M. Low, P. Holliger and G. Winter (1996) J. Mol. Biol 260:359-368, which contains a PelB leader and appropriate restriction sites in the multiple cloning site. Treat the digested vector with calf intestine alkaline phosphatase to dephosphorylate. Ligate the VH, VL and vector fragments at 15 C overnight using a ratio of 2:2:1 in a total volume of 20 ul. Transform the competent cells with 10 ul of the ligation mixture and plate cells according to standard protocols (T. Maniatis, J. Sambrook, E. F. Fritsch (eds.) (1989) Molecular Cloning: A Laboratory Manual. Cold Spring Harbor Laboratory Press, Cold Spring Harbor). Positive clones are screened by PCR using the T84-VH-back and T84-VL-for primers running 30 cycles with an annealing temperature of 50 C and a 1 minute extension time. The products are analyzed on a 1% agarose gel and the correct product has a size of approximately 900 bp.

Positive clones are analyzed for expression by growing induced bacterial cultures overnight and collecting cell pellets and culture supernatants by standard procedures. The pellets are processed for immunoblots and probed with anti-Myc tag antibody to determine expression of the full-length construct. The supernatants are added to ELISA plates coated with purified CEA and developed with anti-Myc tag antibody to determine antigen-binding capability.

Having obtained a functional VH-VL single chain T84 antibody, the 9F11 antibody is similarly cloned by PCR amplification. During amplification an additional 15 amino acid linker is introduced upstream of 9F11 VH region for the purpose of linking the two scFvs. These amplification products are then ligated downstream of the T84 construct.

Positive clones are identified by PCR using the T84-VH-back and 9F11-VL-for primers. The ELISA screen of culture supernatant is done for both CEA and Pgp.

Generation of Bispecific Single-Chain Diabodies in the VL-VH Configuration

The same procedures, starting materials, vectors, linkers and restriction sites are used, but different primers are designed to generate the VL-VH configuration instead.

Optimization of Relative Binding Affinities

Having obtained a full-length functional bispecific single chain diabody of T84 and 9F11 it was desirable to optimize the relative affinities between the T84 (higher affinity) and 9F11 (lower affinity) arms. Optimization is accomplished by random site-directed mutagenesis in a phage expression system.

The construct is digested from the pUC119mycHis vector with SfiI and Not I digestion and ligated into the vector pCANTAB6 digested with the same enzymes. The CDR 3 loops usually contribute most of the binding affinity of an antibody, thus they are mutated in the T84 arm to generate higher affinity variants. Whereas the CDR2 loops are small and rarely contribute to binding affinities, thus they are mutated in the 9F11 arm to generate mutants that may lower the antibody affinity, possibly by decreasing the on rate. Random mutations are introduced essentially as described by Nielsen and Marks (U. B. Nielsen and J. D. Marks, in R. Kontermann and S Dubel (eds) (2001) Antibody Engineering, Springer-Verlag, Berlin, pp: 515-539).

The phage library is first panned for high affinity antibodies binding to CEA, i.e. the T84 ligand. Purified CEA is attached to sepharose beads (Pharmacia, Uppsala) in accordance with the manufacturer's instructions. The antigen coated beads and phage are mixed in suspension with gentle stirring for 1 h at 4 C then place in a disposable chromatography column. Bound phage particles are differentially eluted using an increasing concentration of soluble CEA or salt.

The highest affinity antibodies (last to elude) are then tested for their ability to still bind Pgp. Sepharose beads are coated with purified Pgp and mixed in suspension with the phage with gentle stirring for 1 h at 4 C. The beads are pelleted and washed by repetitive centrifugation and after the final wash bound phage eluted with soluble Pgp or salt. The eluted phages are plated and clones randomly selected for ranking by binding kinetics. Each clone is grown overnight and a final culture volume of approximately 50 ml and periplasmic material is collected by osmotic shock using standard procedures (T. Maniatis, J. Sambrook, E. F. Fritsch (eds.) (1989) Molecular Cloning: A Laboratory Manual. Cold Spring Harbor Laboratory Press, Cold Spring Harbor) and then prepared for analysis using a BIAcore apparatus as suggested by the manufacturer. Purified Pgp or CEA antigens are immobilized on the BIAcore sensor chip starting with a concentration of 10 ug/ml in 10 mM NaAc, pH 4.5. The binding kinetics for each clone is determined for both Pgp and CEA and clones selected where the CEA ligand has the higher affinity and affinity differences between the two ligands are 2, 4, 6 and 8 orders of magnitude. Clones in which the on rate of the 9F11 (anti-Pgp) arm is reduced are preferentially selected.

Characterization of Binding to Human Cells

To determine that the dual ligand is fully capable of binding to both targets microtitre plates are coated with one of Colo205 (CEA+, Pgp−; ATCC #: CCL-222), 2780^{ADR} (CEA−, Pgp+; R. Niv, Y. G. Assaraf, D. Segal, E. Pirak and Y. Reiter (2001) Int. J. Cancer 94:864-872) or LS1034 (CEA+, Pgp+; ATCC #CRL-2158) cells and probed with the T84-9F11. The bound T84-9F11 is detected by standard ELISA procedures using anti-Myc tag antibodies for detection. Having confirmed binding to the cells it is necessary to demonstrate that the binding to Pgp is antagonistic to its function. Each cell line is grown in quadruplets in cell culture dishes and treated with combinations of T84-9F11 and drug as shown in table 1.

TABLE 1
Drug-T84-9F11 interaction test grid.

PLATE

| TEST ITEM | A | B | C | D |
|---|---|---|---|---|
| T84-9F11 | + | + | − | − |
| Drug | + | − | + | − |

The cells are challenged with various drugs, including MTX, vinblastine, taxol, actinomycin D, AZT, Reserpine, and others as described in the literature (Graaf et al. (1996) Proc Natl Acad Sci USA 93:1238-1242; Park et al. (2003) Cytometry 53A:67-78). The T84-9F11 increases sensitivity to the drug treatment for the Pgp positive cell lines but not the Pgp negative Colo205 cells.

Improved Specificity of Dual Affinity Ligand

The T84-9F11 is designed to improve the targeting specificity of the lower affinity ligand, in this example the Pgp, within patients. To test this feature a similar experiment to that described above is done but Nude mice are used instead of cell cultures. Each of the three cell types are sub-cutaenously implanted into the scruff of Nude and allowed to grow (14-21 days) before treatment is initiated with drug and/or T84-9F11 as defined in Table 1. In this case mice receive T84-9F11 24-48 hours prior to drug. Tumor growth is measured on a weekly basis for a period up to 10 weeks. Pgp positive tumors are be more sensitive to drug in the presence of the T84-9F11. At the end of each experiment representative tumors are excised and processed for immunohistological staining with T84, 9F11 and non-specific controls to verify the status of Pgp expression and to evaluate the tumor morphology.

A second set of animal experiments is done in which the Nude mice are implanted, on opposite sites of thorax with two different cell types, LS1034 on one and either Colo205 or 2780^{ADR} on the other. Colo205 are Pgp negative and unaffected by the antibody treatment whereas the 2780^{ADR} and LS1034 are Pgp positive and drug sensitivity is increased in the presence of the antagonistic T84-9F11. However since LS1034 is also positive for CEA, to which the high affinity arm of the T84-9F11 binds, it has an increased sensitivity to drug in the presence of T84-9F11. Using standard practices, dose-ranging studies are also completed to determine the best treatment models. Tumor samples are excised at the end of all experiments and analyzed as previously described.

1. A composition comprising a multispecific ligand comprising at least a first ligand binding moiety which specifically binds to a first ligand having a first biodistribution and a second ligand binding moiety which specifically binds to a second ligand having a second biodistribution different from that of the first ligand, and wherein the affinity of the first and second ligand binding moieties are different and selected to bias the biodistribution of the multispecific ligand.

2. The composition according to claim 1, wherein the multispecific ligand comprises a bispecific antibody.

3. The composition according to claim 2, wherein the affinity of said first ligand binding moiety for the first ligand is higher than the affinity of the second ligand binding moiety for the second ligand and wherein the biodistribution of the multispecific ligand favours the first ligand.

4. The composition according to claim 3, wherein the first ligand is present on a first target cell population and wherein said second ligand is present on a second target cell population comprising the first target cell population and wherein the biodistribution of the multispecific ligand favours the first target cell population.

5. The composition according to claim 1, wherein said first ligand is a cell surface marker associated with one or more specific cell populations, infectious or parasitic agents, diseased cells, or disease-associated cells.

6. The composition according to claim 5, wherein said marker is a CD marker.

7. The composition according to claim 5, wherein said marker is specifically associated with a cancer cell or pre-cancerous cell.

8. The composition according to claim 5, wherein said marker is associated with an immune cell that is susceptible to viral infection.

9. The composition according to claim 5, wherein said marker is specifically associated with an autoimmune disorder or rheumatic disease.

10. The composition according to claim 5, wherein said marker is associated with a specific tissue type.

11. The composition according to claim 5, wherein said second ligand is a cell surface receptor, a family of cell surface receptors or one or more particular cell surface receptor family members.

12. The composition according to claim 11, wherein said second ligand is a cell surface receptor.

13. The composition according to claim 12, wherein said second ligand is a cell surface receptor is selected from the group consisting of tyrosine kinase type receptors, serine kinase type receptors, heterotrimeric G-protein coupled receptors, receptors bound to tyrosine kinase, TNF family receptors, notch family receptors, guanylate cyclase types, tyrosine phosphatase types, decoy receptors, and adhesion receptors.

14. The composition according to claim 12, wherein said second ligand is an IL-8 receptor, a CCR7 receptor, a FAS receptor, or a CXCR4 receptor.

15. The composition according to claim 12, wherein said receptor requires cross-linking for biological activity.

16. The composition according to claim 12, wherein binding of said second ligand binding moiety to said cell surface receptor blocks said receptor.

17. The composition d according to claim 12, wherein binding of said second ligand binding moiety to said cell surface receptor activates said receptor.

18. The composition according to claim 12, wherein said cell surface receptor initiates a signal transduction and wherein binding of said second ligand binding moiety to said cell surface receptor effects a signal transduction.

19. The composition according to claim 5, wherein said antibody comprises a first VH which specifically recognizes said first ligand and a second VH which specifically recognizes said second ligand.

20. The composition according to claim 19, wherein at least one of said first and second VHs require a VL for binding to its ligand.
